(19) United States
(12) Patent Application Publication
(10) Pub. No.: US 2025/0215082 A1
(43) Pub. Date: Jul. 3, 2025

(54) VARIANT CH3 DOMAINS ENGINEERED FOR PREFERENTIAL CH3 HETERODIMERIZATION, MULTI-SPECIFIC ANTIBODIES COMPRISING THE SAME, AND METHODS OF MAKING THEREOF

(71) Applicant: ADIMAB, LLC, Lebanon, NH (US)

(72) Inventors: Caitlin STEIN, Lebanon, NH (US); Robert PEJCHAL, Lebanon, NH (US); Julia MCCREARY, Lebanon, NH (US); Kyle BARLOW, Lebanon, NH (US); Arvind SIVASUBRAMANIAN, Lebanon, NH (US); Michael Benjamin BATTLES, Lebanon, NH (US); Beth H. SHARKEY, Lebanon, NH (US)

(21) Appl. No.: 18/727,720

(22) PCT Filed: Jan. 11, 2023
(86) PCT No.: PCT/US2023/060471

§ 371 Date: Jul. 10, 2024

Related U.S. Application Data
(60) Provisional application No. 63/298,321, filed on Jan. 11, 2022.

(51) Int. Cl. C07K 16/28; C07K 16/32

(52) U.S. Cl. CPC C07K 16/2809; C07K 16/2878; C07K 16/2887; C07K 16/32; C07K 2317/31; C07K 2317/526; C07K 2317/569; C07K 2317/94

(57) ABSTRACT

Variant CH3 domain polypeptides are provided that preferentially form CH3-CH3 heterodimers over CH3-CH3 homodimers. Such variant CH3 domains can be used to promote desired Fc pairing, thus providing for efficient development of bispecific and multispecific antibodies as well as Fc fusions of different formats. Methods of producing bispecific antibodies using such variant CH3 domains and for producing libraries containing such variant CH3 domains are also provided.

### RELATED APPLICATIONS

This application claims priority to U.S. Provisional Application No. 63/298,321 filed on Jan. 11, 2022, entitled “CH3 DOMAIN VARIANTS ENGINEERED FOR PREFERENTIAL CH3 HETERODIMERIZATION AND MULTI-SPECIFIC ANTIBODIES COMPRISING THE SAME”, the contents of which are incorporated by reference in their entirety herein.

### REFERENCE TO AN ELECTRONIC SEQUENCE LISTING

The contents of the electronic sequence listing (1160430.003613.xml; Size: 494,063 bytes; and Date of Creation: Jan. 10, 2023) is herein incorporated by reference in its entirety.

### FIELD OF THE INVENTION

The present invention relates to variant CH3 domains which in association with other variant CH3 domains promote Fc heterodimerization via preferential pairing, and polypeptides, molecules, and multi-specific antibodies or antigen-binding antibody fragments, and compositions comprising any of the foregoing. The present invention further relates to polynucleotides encoding such one or more variant CH3 domains, polypeptides, molecules, multi-specific antibodies or antigen-binding antibody fragments, and compositions and libraries comprising any of the foregoing. The present invention further relates to methods of generating libraries comprising variant CH3 domains and methods of using these libraries to identify variant CH3 domains which in association with other variant CH3 domains promote Fc heterodimerization. The present invention further relates to methods of screening for variant CH3 domain combinations (sets) that promote Fc heterodimerization, methods of producing heteromeric molecules such as multi-specific antibodies or antigen-binding antibody fragments comprising said variant CH3 domain sets, and heteromeric molecules such as multi-specific antibodies and antigen-binding antibody fragments wherein Fc heterodimerization is promoted using said variant CH3 domain sets.

### BACKGROUND OF THE INVENTION

There are ongoing efforts to develop antibody therapeutics that have more than one antigen binding specificity, e.g., bispecific antibodies. Bispecific antibodies can be used to interfere with multiple surface receptors associated with cancer, autoimmune diseases, inflammation, or other diseases and conditions. Bispecific antibodies can also be used to place targets into close proximity and modulate protein complex formation or drive contact between cells. Production of bispecific antibodies was first reported in the early 1960s (Nisonoff et al., Arch Biochem Biophys 1961 93(2): 460-462) and the first monoclonal bispecific antibodies were generated using hybridoma technology in the 1980s (Milstein et al., Nature 1983 305(5934): 537-540). Interest in bispecific antibodies has increased significantly in the last decade due to their therapeutic potential and bispecific antibodies are now used in the clinic, e.g., blinatumomab and emicizumab have been approved for treatment of particular cancers (see Sedykh et al., Drug Des Devel Ther 12:195-208 (2018) and Labrijn et al. Nature Reviews Drug Discovery 18:585-608 (2019), for recent reviews of bispecific antibody production methods and features of bispecific antibodies approved for medical use).

While bispecific antibodies have shown considerable benefits over monospecific antibodies, broad commercial application of bispecific antibodies has been hampered by the lack of efficient/low-cost production methods, the lack of stability of bispecific antibodies, and the lack of long half-lives in humans. A bispecific antibody can be formed by co-expressing two different heavy chains and two different light chains. However, because heavy chains bind light chains in a relatively promiscuous manner, co-expression of two heavy chains and two light chains can lead to a mixture of sixteen possible combinations, representing ten different antibodies only one of which corresponds with the desired bispecific antibody (maximal yield 12.5% in the mixture if there is perfect promiscuity). Even if a first heavy-light chain pair having a first specificity and a second heavy-light chain pair having a second specificity different from the first specificity are produced separately and then mixed for heavy chain-heavy chain pairing, three possible combinations are possible, only one of which corresponds to the desired bispecific antibody (maximal yield 50% in the mixture if there is perfect promiscuity). This mispairing (also referred to as the chain-association issue) pauses a major challenge in manufacturing bispecific antibodies, and a variety of technologies have been developed to address the issue.

One strategy used to alleviate heavy chain-heavy chain mispairing is to design a bispecific antibody having common heavy chains, i.e., two identical heavy chains and two different light chains (see e.g., Fischer et al., Nature Commun. 6:6113 (2015)). This obviates the need for eliminating mispaired antibody products. However, this strategy requires identifying two antibodies having different specificity but the same heavy chain, i.e., only differing in the light chain, which is difficult and tends to compromise the specificity of each binding arm and substantially reduces diversity (see, e.g., Wang et al., MABS 10(8):1226-1235 (2018)). leucine zippers (see, e.g., Kostelny et al., J. Immunol, 148(5):1547-1553 (1992)).

Another strategy is to modify the antibody constant region to reduce the occurrence of chain-heavy chain mispairing. Many engineering efforts have been made in the CH3 domain to facilitate CH3 heterodimerization. Such technologies include: the “knobs-into-holes” (“KiH”) engineering such as the “W-SAV” substitution (see, e.g., Atwell S. et al., J Mol Biol. 1997 Jul. 4; 270(1):26-35. and U.S. Pat. No. 5,731,168 (Genentech)); the “HA-TF” substitution (see, e.g., Moore G. et al., mAbs 2011 November-December; 3(6): 546-557. and U.S. Pat. No. 10,472,427 (Xencor)); the “VYAV-VLLW” substitution (see, e.g., Von Kreudenstein T. S. et al., MAbs 2013; 5:646-54 and U.S. Pat. No. 9,499,634 (Zymeworks)); the “7.8.60” and “20.8.34” designs (see, e.g., Lraver-Fay A. et al., Structure. 2016 Apr. 5; 24(4): 641-651 and U.S. Pat. No. 10,774,156 (University of North Carolina at Chapel Hill and Eli Lilly)); electrostatic complementarity by charge swap substitutions such as “DD-KK” (see, e.g., Gunasekaran K. et al., J Biol Chem 2010; 285:19637-46 and U.S. Pat. No. 8,592,562 (Amgen)); and the “EW-RVT” substitution (see, e.g., Choi H-J. et al., Mol Cancer Ther. 2013 December; 12(12):2748-59. and U.S. Pat. No. 9,951,145B2 (Ajou University)). Additional examples of CH3 modification include those described in: U.S. Pat. No. 10,597,464 (Genmab); U.S. Ser. No. 16/482,137 (Centrymed); U.S. Pat. No. 9,562,109 (Zymeworks); U.S. Ser. No. 15/409,456 (Zymeworks); U.S. Pat. No. 9,624,291 (Ramot at Tel Aviv University); PCT/EP2019/083638 (Morphosys); U.S. Pat. No. 9,605,084 (Xencor); U.S. Ser. No. 16/062,405 (Alphamab); U.S. Ser. No. 15/997,222 (Janssen); U.S. Ser. No. 14/989,648 (Zymeworks); U.S. Ser. No. 13/892,198 (Zymeworks); U.S. Ser. No. 15/586,686 (Hoffmann La Roche); U.S. Pat. No. 9,308,258 (Amgen); U.S. Pat. No. 9,200,060 (Amgen); U.S. Ser. No. 15/554,022 (Laboratoire Francais); U.S. Pat. No. 9,574,010 (Zymeworks); PCT/US2019/023382 (Dana-Farber Cancer Institute); U.S. Ser. No. 13/814,657 (MedImmune); U.S. Ser. No. 11/228,026 (Xencor); PCT/US2017/045139 (Merrimack); U.S. Ser. No. 16/244,378 (Hoffmann La Roche); and Brinkmann U. et al, MAbs. 2017 February-March; 9(2): 182-212 (review).

Although these CH3 modifications increase the propensity to form CH3 heterodimers, there is still the need for improvement, especially given the prevalent interest in developing improved multispecific antibodies for use in human therapy.

### SUMMARY OF THE INVENTION

Provided herein are methods of producing a heteromeric molecule such as a multi-specific antibody or antigen-binding antibody fragment. Such a method may be driven by cFAE. Optionally, the heteromeric molecule may comprise IgG, further optionally an IgG1, IgG2, IgG3 or IgG4 constant regions.

In some embodiments, the heteromeric molecule which is to be produced or intended to be produced may comprise (A) a first polypeptide comprising a first variant CH3 domain polypeptide (or immunoglobulin heavy chain polypeptide comprising said first variant CH3 domain polypeptide), the first variant CH3 domain polypeptide comprising a T366V substitution, according to EU numbering; and
(B) a second polypeptide comprising a second variant CH3 domain polypeptide (immunoglobulin heavy chain polypeptide comprising said first variant CH3 domain polypeptide), the second variant CH3 domain polypeptide comprising a Y407V substitution according to EU numbering.

In such a heteromeric molecule, the first polypeptide and the second polypeptide may be bound to or paired with each other optionally via at least one disulfide bond.

In some embodiments, the method may comprise (i) incubating in a reducing environment or condition (such as in a solution comprising a reducing agent) (i-1) a first parent molecule comprising at least two of the first polypeptides bound to or paired with each other optionally via at least one disulfide bond and (i-2) a second parent molecule comprising at least two of the second polypeptides bound to or paired with each other optionally via at least one disulfide bond.

In this step (i), when the heteromeric molecule is a multi-specific antibody or antigen-binding antibody fragment, the parent molecules may be corresponding monospecific parent antibodies (such as IgG) and the parent antibodies may be incubated in a reducing condition, and the pairing (e.g., the disulfide bond) between the heavy chains in each of the parent antibodies may be dissociated but not between the heavy and light chains.

In some embodiments, the method may then comprise (ii) placing the incubation product of step (i) in a less reducing or non-reducing environment, thereby forming the heteromeric molecule. In some embodiments, when a reducing agent is present in the reducing environment, this step (ii) may remove the reducing agent.

In certain embodiments, the first variant CH3 domain polypeptide may be derived from a CH3 domain of a human IgG and/or the second variant CH3 domain polypeptide may be derived from a CH3 domain of a human IgG. In particular embodiments, the T366V substitution may be relative to a CH3 domain of a human IgG and/or the Y407V substitution may be relative to a CH3 domain of a human IgG.

In certain embodiments, the first variant CH3 domain polypeptide may be derived from a CH3 domain of a human IgG1 and/or the second variant CH3 domain polypeptide may be derived from a CH3 domain of a human IgG1. In particular embodiments, the T366V substitution may be relative to SEQ ID NO: 1, 2, 3, or 4 and/or the Y407V substitution may be relative to SEQ ID NO: 1, 2, 3, or 4.

In certain embodiments, the first variant CH3 domain polypeptide may be derived from a CH3 domain of a human IgG2 and/or the second variant CH3 domain polypeptide may be derived from a CH3 domain of a human IgG2. In particular embodiments, the T366V substitution may be relative to SEQ ID NO: 722 and/or the Y407V substitution may be relative to SEQ ID NO: 722.

In certain embodiments, the first variant CH3 domain polypeptide may be derived from a CH3 domain of a human IgG3 and/or the second variant CH3 domain polypeptide may be derived from a CH3 domain of a human IgG3. In particular embodiments, the T366V substitution may be relative to SEQ ID NO: 723 and/or the Y407V substitution may be relative to SEQ ID NO: 723.

In certain embodiments, the first variant CH3 domain polypeptide may be derived from a CH3 domain of a human IgG4 and/or the second variant CH3 domain polypeptide may be derived from a CH3 domain of a human IgG4. In particular embodiments, the T366V substitution may be relative to SEQ ID NO: 724 and/or the Y407V substitution may be relative to SEQ ID NO: 724.

In certain embodiments, the heteromeric molecule may comprise one or more of the following features: (A) the first polypeptide further comprises a first antigen-binding domain; (B) the second polypeptide further comprises a second antigen-binding domain; (C) the heteromeric molecule further comprises a third polypeptide optionally comprising a third antigen-binding domain, optionally wherein the third polypeptide is bound to or paired with the first polypeptide; and/or (D) the heteromeric molecule further comprises a fourth polypeptide optionally comprising a fourth antigen-binding domain, optionally wherein the fourth polypeptide is bound to or paired with the second polypeptide.

In certain embodiments of the method of producing, the heteromeric molecule may further comprise (C) a third polypeptide optionally comprising a third antigen-binding domain; and/or (D) a fourth polypeptide optionally comprising a fourth antigen-binding domain.

In certain embodiments of the method of producing, the heteromeric molecule may be a multi-specific antibody or antigen-binding antibody fragment, which may optionally comprise any of the structures shown in FIGS. 2-8, optionally wherein the heteromeric molecule comprises (a) an IgG or (b) an IgG and one or more scFvs directly or indirectly conjugated to the IgG, further optionally comprising IgG1, IgG2, IgG3 or IgG4 constant regions.

In certain embodiments of the method of producing, the heteromeric molecule may be a multi-specific antibody or antigen-binding antibody fragment comprising one or more of the following features (I) and (II):

(I) (I-1-i) the first polypeptide comprises a first antigen-binding domain which forms a first antigen-binding site specific for a first epitope and/or (I-1-ii) the heteromeric molecule comprises a third polypeptide comprising a third antigen-binding domain which forms a third antigen-binding site specific for a third epitope, optionally wherein the first epitope is the same as or different from the third epitope; or (I-2) the first polypeptide comprises a first antigen-binding domain and the heteromeric molecule comprises a third polypeptide comprising a third antigen-binding domain, wherein the first antigen-binding domain and the third antigen-binding domain form a first antigen-binding site specific for a first epitope; and/or

(II) (II-1-i) the second polypeptide comprises a second antigen-binding domain which forms a second antigen-binding site specific for a second epitope and/or (I-1-ii) the heteromeric molecule comprises a fourth polypeptide comprising a fourth antigen-binding domain which forms a fourth antigen-binding site specific for a fourth epitope, optionally wherein the second epitope is the same as or different from the fourth epitope; or (II-2) the second polypeptide comprises a second antigen-binding domain and the heteromeric molecule comprises a fourth polypeptide comprising a fourth antigen-binding domain, wherein the second antigen-binding domain and the fourth antigen-binding domain form a second antigen-binding site specific for a second epitope.

In some embodiments, the incubating in step (i) may be performed at a temperature between about 15° C. and about 40° C., between about 20° C. and about 40° C., between about 25° C. and about 35° C., between about 28° C. and about 32° C., or between about 29° C. and about 31° C., or at about 30° C.

In certain embodiments, the incubating in step (i) may be performed for about 30 minutes to about 20 hours, for about 1 hour to about 15 hours, for about 2 hours to about 10 hours, for about 3 hours to about 7 hours, or for about 4 hours to about 6 hours, or for about 5 hours.

In particular embodiments, the incubating in step (i) may be performed at about 30° C. for about 5 hours.

In some embodiments, the reducing environment may comprise at least one reducing agent, optionally at least one mildly reducing agent.

In certain embodiments, the reducing environment may comprise at least one reducing agent selected from 2-mercaptoethylamine (2-MEA), β-mercapto-ethanol (BME), L-cysteine, dithiothreitol (DTT), or dithionite.

In certain embodiments, the reducing agent may not be glutathione.

In certain embodiments, the reducing environment may comprise at least one reducing agent selected from about 25 to about 125 mM, about 50 mM to about 100 mM, about 70 to about 80 mM, or about 75 mM of 2-MEA, about 20 to about 500 μM, about 40 to about 250 μM, about 80 to about 150 μM, about 90 to about 120 μM, or about 100 μM of BME, about 20 to about 500 μM, about 40 to about 250 μM, about 80 to about 150 μM, about 90 to about 120 μM, or about 100 μM of L-cysteine, about 15 to about 400 μM, about 20 to about 200 μM, about 25 to about 100 μM, about 30 to about 70 μM, or about 50 μM of DTT, or about 20 to about 500 μM, about 40 to about 250 μM, about 80 to about 150 μM, about 90 to about 120 μM, or about 100 μM of dithionite.

In particular embodiments, the reducing environment may comprise at least 2-MEA, optionally at about 75 mM.

In some embodiments, in the first and/or second antibodies to be incubated in step (i), the at least two of the first polypeptides may be bound to or paired with each other via at least one disulfide bond and/or the at least two of the second polypeptides may be bound to or paired with each other via at least one disulfide bond.

In some embodiments, the first antibody and/or the second antibody may be produced in a mammalian cell, a yeast cell, an insect cell, a plant cell, or a bacterial cell.

In some embodiments, the first antibody and/or the second antibody may be produced in a Chinese hamster ovary (CHO) cell or a Human embryonic kidney (HEK) cell.

In some embodiments of the method of producing, the placing in step (ii) may be performed by buffer exchange optionally wherein the buffer may be exchanged into phosphate buffered saline (PBS).

In some embodiments the placing in step (ii) may be performed by buffer exchange via desalting optionally into PBS.

In some embodiments the placing in step (ii) may be performed by buffer exchange via diafiltration optionally into PBS.

In some embodiments the placing in step (ii) may be performed by addition of an oxidizing agent.

In some embodiments, the method of producing may further comprise (iii) incubating the product of step (ii) in the less reducing or non-reducing environment.

In certain embodiments, the incubating may be performed at a temperature between about 1° C. and about 20° C., between about 2° C. and about 10° C., between about 3° C. and about 5° C., or at about 4° C. In certain embodiments, the incubating may be performed for about 12 hours to about 154 hours, for about 24 hours to about 96 hours, for about 36 hours to about 72 hours, or for about 48 hours. In particular embodiments, the incubating may be performed the incubating may be performed at about 4° C. for about 48 hours.

In some embodiments, the method of producing may further comprise (iv) analyzing the amount of the multi-specific antibody or antigen-binding antibody fragment in the product of step (ii) and/or step (iii) and/or purifying the multi-specific antibody or antigen-binding antibody fragment from the product of step (ii) and/or step (iii).

In particular embodiments, the analyzing and/or purifying is performed via chromatography, optionally LC-MS, IEX, and/or SEC.

In some embodiments, the heteromeric molecule produced is a multi-specific antibody. The first polypeptide may include a first antibody heavy chain and the second polypeptide may include a second antibody heavy chain, wherein the first antibody heavy chain is associated with a first antibody light chain and the second antibody heavy chain is associated with a second antibody light chain. The multi-specific antibody may include a third polypeptide that includes a third antigen-binding domain. The third antigen binding domain may be associated with the first antibody heavy chain, the second antibody heavy chain, the first antibody light chain, or the second antibody light chain. The multi-specific antibody may further include a fourth polypeptide that includes a fourth antigen-binding domain. The fourth antigen binding domain may be associated with the first antibody heavy chain, the second antibody heavy chain, the first antibody light chain, or the second antibody light chain.

The association of the third and/or fourth antigen binding domain(s) may be via a flexible linker. In certain embodiments, the flexible linker may comprise or consist of: (i) the amino acid sequence selected from the group consisting of GGGGS (SEQ ID NO: 715), GGGS (SEQ ID NO: 716), GGGGGS (SEQ ID NO: 717), G, GG, GGG, GS, SG, GGS, GSG, SGG, GSS, SGS, and SSG; (ii) multiple repeats, optionally two, three, four, or five repeats, of the amino acid sequence selected from the group consisting of SEQ ID NO: 715, SEQ ID NO: 716, SEQ ID NO: 717, G, GG, GGG, GS, SG, GGS, GSG, SGG, GSS, SGS, and SSG; (iii) a (G5S)n linker, a (G4S)n linker, a (G3S)n linker, a (G2S)n linker, a (GS)n linker, or a (G)n linker, wherein n is a natural number, optionally selected from 1-20, further optionally 2, 3, 4, or 5; and/or (iv) the amino acid sequence of GGGGSGGGGS (SEQ ID NO: 718) or GGGGSGGGGSGGGGS (SEQ ID NO: 719).

The third and/or fourth antigen-binding domain(s) may include a Fab or single chain Fv (scFv). The third and/or fourth antigen-binding domain(s) may include a scFv, wherein the scFv includes a heavy chain variable domain and a light chain variable domain, wherein the heavy chain variable domain and light chain variable domain are linked by a disulfide bond and/or a linker. In certain embodiments, such a linker may comprise or consist of: (i) the amino acid sequence selected from the group consisting of GGGGS (SEQ ID NO: 715), GGGS (SEQ ID NO: 716), GGGGGS (SEQ ID NO: 717), G, GG, GGG, GS, SG, GGS, GSG, SGG, GSS, SGS, and SSG; (ii) multiple repeats, optionally two, three, four, or five repeats, of the amino acid sequence selected from the group consisting of SEQ ID NO: 715, SEQ ID NO: 716, SEQ ID NO: 717, G, GG, GGG, GS, SG, GGS, GSG, SGG, GSS, SGS, and SSG; (iii) a (G5S)n linker, a (G4S)n linker, a (G3S)n linker, a (G2S)n linker, a (GS)n linker, or a (G)n linker, wherein n is a natural number, optionally selected from 1-20, further optionally 2, 3, 4, or 5; and/or (iv) the amino acid sequence of GGGGSGGGGS (SEQ ID NO: 718) or GGGGSGGGGSGGGGS (SEQ ID NO: 719). The multi-specific antibody may be a biparatopic antibody.

In some embodiments, the first parent molecule includes a first IgG and the second parent molecule includes a second IgG. Each of the at least two of the first polypeptides of the first IgG may include a first antibody heavy chain including a first antigen-binding domain which forms a first antigen-binding site for a first epitope. Each of the at least two of the second polypeptides of the second IgG may include a second antibody heavy chain including a second antigen binding domain which forms a second antigen-binding site for a second epitope. The first epitope and the second epitope may be part of different antigens. The first epitope and the second epitope may be part of the same antigen. The heteromeric molecule may be an IgG that includes the first antibody heavy chain and the second antibody heavy chain.

In some embodiments, the method may comprise producing a plurality of multi-specific antibodies and/or antigen-binding antibody fragments, using a method of producing a heteromeric molecule as described above.

In some embodiments, said T366V substitution is the only substitution in the first variant CH3 domain polypeptide. In certain embodiments, said T366V substitution is the only substitution relative to a CH3 domain of a human IgG. In particular embodiments, said T366V substitution is the only substitution relative to a CH3 domain of a human IgG1, optionally relative to the amino acid sequence of SEQ ID NO: 1, 2, 3, or 4. In particular embodiments, said T366V substitution is the only substitution relative to a CH3 domain of a human IgG2, optionally relative to the amino acid sequence of SEQ ID NO: 722. In particular embodiments, said T366V substitution is the only substitution relative to a CH3 domain of a human IgG3, optionally relative to the amino acid sequence of SEQ ID NO: 723. In particular embodiments, said T366V substitution is the only substitution relative to a CH3 domain of a human IgG4, optionally the amino acid sequence of SEQ ID NO: 724. In some embodiments, said Y407V substitution is the only substitution in the second variant CH3 domain polypeptide. In certain embodiments, said Y407V substitution is the only substitution relative to a CH3 domain of a human IgG. In particular embodiments, said Y407V substitution is the only substitution relative to a CH3 domain of a human IgG1, optionally relative to the amino acid sequence of SEQ ID NO: 1, 2, 3, or 4. In particular embodiments, said Y407V substitution is the only substitution relative to a CH3 domain of a human IgG2, optionally relative to the amino acid sequence of SEQ ID NO: 722. In particular embodiments, said Y407V substitution is the only substitution relative to a CH3 domain of a human IgG3, optionally relative to the amino acid sequence of SEQ ID NO: 723. In particular embodiments, said Y407V substitution is the only substitution relative to a CH3 domain of a human IgG4, optionally the amino acid sequence of SEQ ID NO: 724.

In some embodiments, the first and second variant CH3 domain polypeptides may be further modified to comprise one or more variant CH3 domain sets, optionally any of the variant CH3 domain sets described herein, such as but not limited to any of the variant CH3 domain sets in any of the Tables disclosed herein.

In some embodiments, the heteromeric molecule may comprise one or more CH2 domains. In certain embodiments, one or more of said CH2 domains may comprise one or more amino acid modifications. In certain embodiments, one or more of said CH2 domains may comprise one or more Fc-silencing modifications. In certain embodiments, one or more of said CH2 domains may comprise one or more FcRn affinity-enhancing or reducing and/or half-life-extending or reducing modifications. In particular embodiments, one or more of said CH2 domains may comprise any of the following modifications, according to EU numbering: L234A, L235A, and P329A substitutions; L234A, L235A, and P329G substitutions; L234A and L235A substitutions; D265A and P329A substitutions; N297A substitution; M252Y, S254T, and T256E substitutions; and/or M428L and N434S substitutions.

In yet another aspect, provided herein are heteromeric molecules such as multi-specific (e.g., bispecific) antibodies and antigen-binding antibody fragments produced by a method of producing described herein. Optionally, such a multi-specific antibody or antigen-binding antibody fragment may comprise an IgG, further optionally an IgG1, IgG2, IgG3 or IgG4.

In some embodiments, the multi-specific antibody or antigen-binding antibody fragment may comprise a structure according to any of the structures described herein or shown in FIGS. 2-8.

### BRIEF DESCRIPTION OF THE DRAWINGS

FIG. 1A-1C provide schematics which overall show the benefit of heterodimerizing CH3 domains. The bispecific antibody of interest comprises: (a) a half antibody specific to epitope A, comprising a heavy chain A (comprising a VH (solid black)) and a light chain A (comprising a VL (horizontal stripe); and (b) a half antibody specific to epitope B, comprising a heavy chain B (comprising a VH (checker)) and a light chain B (comprising a VL (vertical stripe)).

FIG. 1A shows an exemplary production of such a bispecific antibody, when the heavy chain A, light chain A, heavy chain B, and light chain A all comprise wild-type constant domains. When such four chains are co-expressed, co-provided, or mixed at approximately a 1:1:1:1 ratio, ten different antibody products can be generated with the respective percentages as shown, if there is perfect promiscuity in inter-heavy-light chain pairing and inter-heavy-heavy chain pairing. Approximately 12.5% of the products will correspond to the bispecific antibody of interest (boxed).

FIG. 1B shows an exemplary production of a bispecific antibody similar to FIG. 1A, except that CH3 domain of the heavy chain A (CH3 domain A (diagonal stripe)) and the CH3 domain of the heavy chain B (CH3 domain B (dotted)) are variant CH3 domains that differ from each other and preferentially form a heterodimer (i.e., heterodimer between CH3 domain A and CH3 domain B). Pre-existing heavy chain CH3 heterodimerizing technologies include those listed in Table 1, such as the “knobs-into-holes” technology (see, e.g., U.S. Pat. No. 5,731,168). When such heavy chain A, light chain A, heavy chain B, and light chain B are co-expressed, co-provided, or mixed at approximately a 1:1:1:1 ratio, and if CH3 domain A and CH3 domain B exclusively allows heavy-heavy hetero pairing, four different antibody products can be generated with the respective percentages as shown. Approximately 25% of the products will correspond to the bispecific antibody of interest (boxed).

FIG. 1C provides two schematics (left and right) which overall show the benefit of heterodimerizing CH3 domains when producing a full-size antibody (of IgG, IgE, or IgD) from two of already-formed half antibodies. Such production methods include but are not limited to methods relying on Fab arm exchange (FAE) or controlled FAE (cFAE). The bispecific antibodies may be produced by combining the half antibody specific to epitope A and the half antibody specific to epitope B. When both heavy chains A and B comprise a wild-type CH3 domain (left schematic), only 50% of the products (if there is perfect promiscuity in inter-half-antibody pairing) are the bispecific antibody of interest. However, as shown in the right scheme, when CH3 domain A (diagonal stripe) and CH3 domain B (dotted)variant CH3 domains differ from each other and preferentially form a CH3-CH3 heterodimer (i.e., heterodimer between CH3 domain A and CH3 domain B), the products are more skewed to the bispecific antibody of interest. In the most ideal CH3 domain sets, which only form heterodimers and no homodimers, 100% of the products will be the bispecific antibody of interest. Even if it is not 100%, variant CH3 domains that provide heterodimers at more than 50% facilitate efficient manufacturing of bispecific antibodies.

Black solid is VH (specific to epitope A) of heavy chain A (“VH domain A”), horizontal stripe is VL (specific to epitope A) of light chain A (“VL domain A”), checker is VH (specific to epitope B) of heavy chain B (“VH domain B”), vertical stripe is VL (specific to epitope B) of light chain B (“VL domain B”), diagonal stripe is variant CH3 domain in heavy chain A (CH3 domain A), and dotted is variant CH3 domain in heavy chain B (CH3 domain B), in an exemplary multi-specific antibody, wherein CH3 domain A and CH3 domain B preferentially form a CH3 hetero dimer (i.e., results in >50% CH3 heterodimers). These definitions apply to all FIGs, unless otherwise noted.

FIGS. 2-8 provide exemplary and non-limiting embodiments of various multi-specific antibody structures with which the variant CH3 domains disclosed herein may be used. In FIGS. 2-8, the following applies unless otherwise indicated: (1) Each domain is presented as a rectangle with the text therein showing the domain name (e.g., CH3, VH1, etc); (2) a set of multiple domains connected with each other represents a polypeptide (e.g., a heavy chain polypeptide, a light chain polypeptide, etc); (3) the direction of domains within a polypeptide is according to the direction of the text showing domain names, from the N-terminus to the C-terminus; (4) a linker or a hinge may be used between domains as necessary and a disulfide bond(s) may exist between polypeptides (and/or within a domain), perhaps to allow correct formation of the antigen-binding site(s), even when the FIGS do not explicitly show a linker, a hinge, or a disulfide bond; (5) a CH2 and/or CH3 domain(s) shown in figures may be omitted whenever possible and, when appropriate, may be replaced with a hinge or a linker; (6) diagonal stipe and dotted are variant CH3 domains which preferentially form a CH3-CH3 heterodimer, which may be a variant CH3 domain disclosed herein; (7) rectangles with no pattern (i.e., open) are domains which individually may comprise a corresponding wild-type sequence or may comprise one or more amino acid substitutions relative to a wild-type sequence; (8) CH1, CH2, and CH3 domains may individually be of any (heavy chain) isotype; (9) when more than one CH1 domains are present in a structure, the CH1 domains may or may not be of the same isotype, when more than one CH2 domains are present in a structure, the CH2 domains may or may not be of the same isotype, and when more than one CH3 domains are present in a structure, the CH3 domains may or may not be of the same isotype; (10) light chain constant (CL) domain may be is a kappa CL domain or a lambda CL domain; (11) when more than one CL domains are present in a structure, all CL domains may be kappa CLs or all CL domains may be lambda CLs, or alternatively one CL may be a kappa CL and another CL may be a lambda CL domain; (12) when both kappa and lambda CL domains are present, the CH1 domains paired to the CL domains may, in some instances, be variant CH1 domains, one of which may be a variant CH1 that preferentially binds to a kappa CL and another CH1 domain may be a variant CH1 that preferentially binds to a lambda CL (having kappa and lambda CLs and kappa-preferring CH1 and lambda-preferring CH1 in a molecule potentially allows for efficient manufacturing); (13) VH-1 and VL-1 form an antigen-binding site for a first epitope, VH-2 and VL-2 form an antigen-binding site for a second epitope, VH-3 and VL-3 form an antigen-binding site for a third epitope, VH-4 and VL-4 form an antigen-binding site for a fourth epitope, VH-5 and VL-5 form an antigen-binding site for a fifth epitope, and VH-6 and VL-6 form an antigen-binding site for a sixth epitope; (14) all of the first through sixth epitopes may be different from each other, or not all of the first through sixth epitopes may be different from each other; and (15) in a given VH-VL pair, the VL may be omitted even if VL is shown in FIGS, if the VH alone gives sufficient specificity to a cognate antigen (i.e., nanobody).

FIG. 2 provides some exemplary and non-limiting embodiments of various multi-specific antibody structures with which the variant CH3 domains disclosed herein may be used. The antibody on the top left (boxed) is an exemplary basic full-size bispecific antibody, in which hinges or disulfide bods are not explicitly shown. The boxed antibody may, for example, comprise a hinge between CH1-1 and CH2-1 and between CH1-2 and CH2-2 and a disulfide bond(s) (dashed line(s)) may be present between the hinges (top center). Alternatively, the boxed antibody may, for example, comprise a hinge between CH1-1 and CH2-1 and between CH1-2 and CH2-2 and a disulfide bond (dashed line(s)) may be present between hinges, between CL-1 and hinge, and between CL-2 and hinge (top right). Hinges and disulfide bonds, such as those shown in top middle and top right antibody structures may be present, even if not explicitly shown, in any structures shown in FIGS and described herein. In some variants of the boxed antibody, the CH2 domains may be absent (middle left) or the CH1 and CH2 domains may be absent (bottom left), and the hinges and disulfide bonds may be present as shown in middle center, middle right, bottom middle, or bottom right. Although not explicitly shown, any CH1 and/or CH2 domains may be omitted as appropriate, in any of the structures in FIGS. 3-8 or variations thereof.

FIG. 3 provides variations of antibody structures shown in FIG. 2. In FIG. 3A, VH and VL positions are varied relative to the FIG. 2 structures. In FIG. 3B, CH1 and CL positions are varied relative to the FIG. 2 structures. Equivalent variations (switching VH-VL positions or CH1-CL positions) depicted in FIG. 3 may be further applied to any structures shown in FIGS. 3-8 or variations thereof as appropriate, even if not explicitly shown.

FIG. 4 provides variations of the boxed antibody structure of FIG. 2. Specifically, a VH-VL pair specific to a third epitope and a VH-VL pair specific to a fourth epitope are added to the N-terminus of the heavy and light chains in different orientations. Although both a VH-VL pair specific to a third epitope and a VH-VL pair specific to a fourth epitope are depicted, if desired one pair may be added. Equivalent variations (addition of one or more VH-VL pairs) depicted in FIG. 4 may be further applied to any structures shown in FIGS. 3-8 or variations thereof as appropriate, even if not explicitly shown.

FIG. 5 provide additional variations of the boxed antibody structure of FIG. 2. Similar to structures in FIG. 4, a VH-VL pair specific to a third epitope and a VH-VL pair specific to a fourth epitope are added in different orientations, the order of VH and VL on light chains differ from that in FIG. 4. Equivalent variations (addition of one or more VH-VL pairs) depicted in FIG. 4 may be further applied to any structures shown in FIGS. 2-8 or variations thereof as appropriate, even if not explicitly shown.

FIG. 6 provide further variations of the boxed antibody structure of FIG. 2. Specifically, in FIGS. 6A-6D, a scFv specific to a third epitope and a scFv specific to a fourth epitope are added. Although two scFvs are depicted, if desired one scFv may be added. In FIG. 6A, the scFvs are added to the C-terminus of the heavy chains. The four structures in FIG. 6A differ by the VH-VL order within each scFv. In FIG. 6B, the scFvs are added to the C-terminus of the light chains. The four structures in FIG. 6B differ by the VH-VL order within each scFv. In FIG. 6C, the scFvs are added to the N-terminus of the heavy chains. The four structures in FIG. 6C differ by the VH-VL order within each scFv. In FIG. 6D, the scFvs are added to the N-terminus of the light chains. The four structures in FIG. 6D differ by the VH-VL order within each scFv. Although not shown in FIGS. 6A-6D, the two scFvs may be added to different positions (e.g., one at the C-end of a heavy chain and one at the N-end of a light chain). In FIG. 6E, four scFvs are added to the N-terminus of the heavy and light chains. The four structures in FIG. 6C differ by the VH-VL order within each scFv. Equivalent variations (addition of one or more scFvs) depicted in FIG. 6 may be further applied to any structures shown in FIGS. 2-8 or variations thereof as appropriate, even if not explicitly shown.

FIGS. 7A-7B provide further variations of the boxed antibody structure of FIG. 2. Specifically, a VH-VL pair specific to a third epitope and a VH-VL pair specific to a fourth epitope are added to the C-terminus of the heavy and light chains in different orientations. Although both a VH-VL pair specific to a third epitope and a VH-VL pair specific to a fourth epitope are depicted, only one pair may be added if desired. Equivalent variations (addition of one or more VH-VL pairs) depicted in FIG. 7A-7B may be further applied to all other structures shown in FIGS. 3-8 or variations thereof as appropriate, even if not explicitly shown.

FIGS. 8A-8E provide additional exemplary and non-limiting embodiments of various multi-specific antibody fragment structures with which the variant CH3 domains disclosed herein may be used and which does not comprise a conventional antibody's VH-VL antigen binding site but rather comprise one or more of scFvs. In FIG. 8A, the antibody on the left (boxed) is an exemplary basic bispecific antibody fragment comprising a first heavy chain comprising a scFv (comprising VH-1 and VL-1) specific to a first epitope and a second heavy chain comprising a second scFv (comprising VH-2 and VL-2) specific to a second epitope. Light chains may be absent. Variants thereof which lack CH2 domains (center) or CH1 and CH2 domains (right) are also provided. FIGS. 8B-8E provide further variations of the antibody structures of FIG. 8A, which comprise further scFvs. In FIG. 8B, a third scFv (comprising VH-3 and VL-3) specific to a third epitope and a fourth scFv (comprising VH-4 and VL-4) specific to a fourth epitope are added to the N-terminus of the heavy chains. In FIG. 8C, a third scFv (comprising VH-3 and VL-3) specific to a third epitope and a fourth scFv (comprising VH-4 and VL-4) specific to a fourth epitope are added to the C-terminus of the heavy chains. In FIG. 8D, a first light chain comprising CL-1 and a second light chain comprising CL-2 are added, and a third scFv (comprising VH-3 and VL-3) specific to a third epitope and a fourth scFv (comprising VH-4 and VL-4) specific to a fourth epitope are added to the N-terminus of the light chains. In FIG. 8E, a fifth scFv (comprising VH-5 and VL-5) specific to a fifth epitope and a sixth scFv (comprising VH-6 and VL-6) specific to a sixth epitope are added to the C-terminus of the heavy chains. Although not explicitly shown, the VH-VL order within a scFv may be switched if desired.

FIGS. 9A-9B show the variant CH3 domain selection proof-of-concept (POC) study in Example 1, in which two heterodimer technologies (KiH and EW-RVT) were assessed as controls. FIG. 9A provides a schematic of selection of heterodimer-preferring variant CH3 domains by flow cytometry. High FLAG expressors display more modified Fc that contain CH3 heterodimers. The population from the library (Knob_{HIS}Hole_{FLAG}:EW_{HIS}RVT_{FLAG}:WT_{HIS}−WT_{FLAG}=1:1:10,000) that stained exceptionally high with an anti-FLAG antibody (indicating expression of heterodimer-preferring variant CH3 domains) is selected and sorted. FIG. 9B provides exemplary flow plots from rounds of selection, which demonstrated enrichment for the control heterodimers (KiH and EW-RVT). The variant CH3 domain library of Knob_{HIS}Hole_{FLAG}:EW_{HIS}RVT_{FLAG}:WT_{HIS}−WT_{FLAG}=1:1:10,000 were sorted for high Flag expressors for two rounds. Sequencing after round 1 (R1) gave 1 of 91 with KiH mutations. Sequencing after round 2 (R2) gave 2 of 91 with KiH mutations and 2 with EW-RVT mutations.

FIGS. 10A-10D show representative data from Cycle 1 of the variant CH3 domain selection in Example 2. FIG. 10A provides three library designs in which the KiH amino acid positions (position 366 in a first heavy chain and positions 366, 368, and 407 in a second heavy chain) are variegated. In the first library, both the Knob and Hole positions are variegated; the strand having the Hole variegation encodes a FLAG tag and the strand having the Knob variegation encodes a HIS tag. In the second library, both the Knob and Hole positions are variegated; the strand having the Hole variegation encodes a HIS tag and the strand having the Knob variegation encodes a FLAG tag. In the third library, Hole positions but not the Knob positions are variegated; the strand having the Hole variegation encodes a FLAG tag and the strand without variegation encodes a HIS tag. DNA sequence variations for each library are provided using site-saturation mutagenesis (SSM). “X” in white text with black background represents variegation. FIG. 10B provides exemplary flow plots from the six rounds of selection performed using the first library. FIG. 10C provides exemplary flow plots from the six rounds of selection using the second library. FIG. 10D provides exemplary flow plots from the six rounds of selection using the third library.

FIGS. 11A-11C show exemplary AlphaLISA® analyses of identified variant CH3 domains. FIG. 11A (left) provides a schematic of CH3 heterodimer detection by AlphaLISA®. AlphaLISA® was used to determine relative degree of heterodimerization of Fc fragments, by specifically detecting modified Fc comprising a heterodimer CH-CH3 set due to the proximity between a HIS×6-tagged polypeptide and a FLA-tagged polypeptide. FIG. 11A (right) provides the results from several samples in a POC set, showing clear differences in the photon counts between the pre-existing heterodimerizing variant CH3 domain sets (KiH and EW-RVT) and the WT CH3 domain sets, regardless of which strand contained the FLAG tag. FIG. 11B provides a graph showing AlphaLISA® values (photon counts, fold over background (FOB) (“buffer only”, i.e., no Fc, was used as background)) for variant CH3 domains positive controls (KiH and EW-RVT, indicated with arrow), negative (WT/WT, indicated with arrow) controls, and the variant CH3 domains identified in Example 2 (bars without arrow). Some of the identified variant CH3 domains identified herein demonstrated equivalent or superior heterodimerization (see bars to the left of EW-RVT). FIG. 11C provides a graph in which AlphaLISA® values are plotted against anti-FLAG antibody staining during the last round of flow cytometry-based selection, showing good correlation. The anti-FLAG FOB and AlphaLISA® FOB values for “T366V-HIS; T366 L368 Y407V-FLAG” were 622 and 86, respectively. “T366V-HIS; T366 L368 Y407V-FLAG” was also found in the reverse orientation (i.e., “T366 L368 Y407V-HIS T366V-FLAG”). The anti-FLAG FOB and AlphaLISA® FOB values for “T366 L368 Y407V-HIS T366V-FLAG” were 588 and 39, respectively.

FIGS. 12A-12B show exemplary size exclusion chromatography (SEC) analyses of variant CH3 domains. FIG. 12A provides the results from control samples. FIG. 12B provides the results from identified variant CH3 domains. All tested CH3 sets resulted in uniform distributions, meaning that there is low aggregation.

FIGS. 13A-13C show exemplary ion exchange (IEX) analyses of variant CH3 domains. FIG. 13A provides the results from control samples, showing peaks corresponding to different antibody species. FIG. 13B provides the results for the outputs from identified variant CH3 domains. CH3 sets V-V, L-V, L-M, I-F, and W-SG showed similar chromatographs to that of EW-RVT, having a sharp single peak. FIG. 13C provides SEC and IEX data in parallel for samples, W-SY and SEL-L, which had low AlphaLISA® values. Low AlphaLISA® values correlated with poor SEC and IEX chromatograms.

FIGS. 14A-14C show production of bispecific antibodies (BsAbs) comprising variant CH3 domains, with an addition of the 354/349 disulfide bond substitutions (S354C and Y349C), in HEK293 cells in Example 6. FIG. 14A provides schematics of different anti-CD3/anti-HER2 BsAbs produced. Nivolumab (Nivo) was used as a control. FIG. 14B provides exemplary SEC chromatograms for each BsAb. FIG. 14C provides exemplary IEX chromatograms for each BsAb.

FIGS. 15A-15D show subsequent library generation and screening. FIG. 15A provides exemplary flow plots from rounds of selection, enriching heterodimerizing variant CH3 domains from the library. FIG. 15B shows selection criteria applied to the set of 430 variant CH3 domains obtained from Cycle 2 step 1 to enrich for those variant CH3 domains with improved contact percentage across interface, AlphaLISA® values, and Rosetta scores. Sequences were also checked to ensure a diversity of mutational positions. FIGS. 15C and 15D provide t-SNE plots. These plots were used to ensure a diversity of substitutions in the variant CH3 domains selected for further production and characterization. Each point represents a set of substituted positions, and points closer together on the plot contain similar substituted positions.

FIG. 16 shows heterodimerization and stability characterization of 48 variant CH3 domains. In the graph, % monomer full size modified Fc (measured by SEC, showing % unaggregated modified Fcs) is plotted against % heterodimer modified Fc for 48 variant CH3 domains and controls. Based on these results, five variant CH3 domain sets (“nominated”), which are shown in Table 8, were selected as Cycle 2 outputs. Nominated clones showed heterodimerization and stability similar to controls.

FIGS. 17A-17J show characterization of exemplary BsAbs comprising variant CH3 domains produced in HEK293 cells. FIG. 17A shows schematics of exemplary antibodies produced. For each variant CH3 domain, several different structures were produced: three anti-CD3/anti-HER2 BsAbs (one in Orientation 1, one in Orientation 2, and one in Orientation 1 in which the 354/349 substitutions were further added to the CH3 set), two anti-CD20/anti-CD3 BsAbs (one in Orientation 1 and one in Orientation 2), and one anti-HEL/anti-BCMA BsAbs (anti-BCMA binding moiety is a nanobody). Sequences are provided in Appendix Tables A-D. Structures in the dotted box were compared to evaluate heterodimerization efficiency, 17B shows heterodimerization was consistent between CH3 orientations and variable regions. Percent (%) heterodimer values for anti-CD3/anti-HER2 BsAbs (lacking 354/349 substitutions) are provided. FIG. 17C shows IEX chromatographs for different BsAbs and compares the % heterodimer values measured by IEX and LC-MS. When the BsAb contained a nanobody as one of the two antigen-binding domains, IEX resulted in poor resolution (the chromatogram for BCMA VHH×HEL). FIG. 17D compares the % heterodimer values measured by IEX and LC-MS for different BsAbs that do not contain the 354/349 substitutions. IEX % Heterodimer and LCMS % Heterodimer values show good correlation (data points not including BsAbs containing a nanobody in one Fab arm). FIG. 17E compares the % heterodimer values measured by IEX and LC-MS for different BsAbs that contain the 354/349 substitutions. FIG. 17F compares the % heterodimer values measured by LC-MS between BsAbs that contain and do not contain the 354/349 substitutions. The 354/349 substitutions seem to improve heterodimerization (measured by LC-MS) for most CH3 sets including the wild-type set. FIG. 17G compares AlphaLISA® values to the % heterodimer values measured by LC-MS or by IEX for BsAbs (CD3×HER2 BsAbs and HEL×BCMA Fab-VHH BsAbs) that contain and do not contain the 354/349 substitutions. The heterodimerization rank orders determined by LC-MS and by IEX were same. FIG. 17H compares % heterodimer values measured by IEX, LC-MS, and AlphaLISA® among LWG and/or SIG sets in Orientation 1, Orientation 2, and Orientation 1 with additional 354/349 substitutions. FIG. 17I compares stability of different bsAbs (not including bsAbs containing a nanobody in one arm) as defined by % monomer full Ab measured by SEC on Day 0 (the day of HEK production) and changes in % monomer full Ab (A % monomer full Ab) by Day 14. % Monomer full Ab values were very low on Day 0 for all BsAbs tested, and little increase in % monomer full Ab values was observed after 14 days, indicating minimum aggregation. FIG. 17J compares the production yield of different BsAbs (with or without the 354/349 substitutions) in HEK293 cells.

FIGS. 18A-18G show comparison of anti-CD3/anti-HER2 BsAbs comprising different CH3 sets (WT, pre-existing CH3 heterodimerizing set, Cycle 1 output, Cycle 2 output, or combination thereof, with or without the CH3 disulfide bond substitutions (i.e., the 354/349 substitutions). FIG. 18A compares % heterodimer values measured by LC-MS and IEX for different BsAbs without the 354/349 substitutions, which show good correlation. FIG. 18B compares the rank order of heterodimerization potential determined by % heterodimer values measured by LC-MS and IEX for different BsAbs with the 354/349 substitutions. The rank orders determined by the two different methods (LC-MS and IEX) were same. FIG. 18C shows % heterodimer values measured by LC-MS and IEX for different BsAbs, with and without the 354/349 substitutions, and reveals that irrespective of the presence or absence of the 354/349 substitutions, the LWG-SIG set consistently provided high % heterodimer values. FIG. 18D shows % monomer full Ab values measured by SEC for different BsAbs with and without the 354/349 substitutions. As shown some BsAbs, such as DVG-VSY and RG-FG, showed higher % monomer full Ab values, indicating less aggregation compared to existing variant CH3 domains (KiH, EW-RVT, or ZW1). FIG. 18E provides a graph of % monomer full Ab values measured by SEC plotted against % heterodimer values as measured by LC-MS for different BsAbs with and without the 354/349 substitutions and reveals that the 354/349 substitutions overall increased % monomer full Ab values and % heterodimer values. FIG. 18F compares production yields in HEK293 cells for different BsAbs with and without the 354/349 substitutions and shows that no substitution set appeared to negatively affect production yields. FIG. 18G compares the LWG-SIG set and its variant LWG-IG based on % heterodimer values measured by IEX, and LC-MS, % monomer full Ab values measured by SEC, and production yield in HEK cells. The profiles were similar between LWG-SIG and LWG-IG, consistent with the Rosetta heterodimer scores.

FIG. 19 provides a summary of exemplary CH3 domain sets (“CH3 Set Names”) identified herein that preferentially form CH3-CH3 heterodimers over homodimers and, thus, promote desired Fc pairing.

The amino acid substitutions (positions and amino acid residues) for respective CH3 sets listed in FIG. 19 may be found, for example, in Appendix Tables E-G. These amino acid substitutions may be incorporated into any CH3 domain sequence. Exemplary variant CH3 domain sequences, in which the CH3 substitution sets listed in FIG. 19 are incorporated into the reference CH3 domain sequence of SEQ ID NO: 1, are also shown in in Appendix Tables E-G. The exemplary variant CH3 domain sequences are the sequences used in Examples herein. SEQ ID NOS assigned to those exemplary variant CH3 domain sequences are also shown in FIG. 19.

FIG. 20 provides exemplary Tm2 values for Fc only constructs comprising different CH3 sets (WT, pre-existing, Cycle 1 output, or Cycle 2 output CH3 heterodimerizing set, with or without the CH3 disulfide bond substitutions (the 354/349 substitutions), as shown in Table 15), measured by DSC. Open circles represent constructs without the 354/349 substitutions, and filled circles represent constructs with the 354/349 substitutions.

FIG. 21 provides ADI-64950 CH3-CH3 interface in its electron density. (a) representative electron density in the region of interest for the crystal structure of the IgG1 Fc-only construct, ADI-64950, which comprises (i) Chain A comprising T366S, L368I, and Y407G and (ii) Chain B comprising S364L, T366W, and K409G, where Chain B also contains the Fc-III knockout substitutions: M252E, I253A, and Y436A. Chain A carbon atoms are colored white, Chain B carbon atoms are colored light grey, nitrogen atoms are colored dark grey, oxygen atoms are colored black, and sulfur atoms are colored very dark grey. Protein is shown in stick representation. The 2Fo-Fc electron density map is shown as a grey mesh contoured at 1.06 with a 2.0 Å carve. Data for this crystal structure extend to 2.70 Å near-atomic resolution. (b) Table comparing the interface statistics as generated from PISA for ADI-64950 (SIG-LWG) and wild-type IgG1 (WT; PDB ID: 5JII).

FIG. 22 provides polar contacts at the ADI-64950 CH3-CH3 interface. (a) Polar contacts at the CH3-CH3 interface between Chain A and Chain B. Chain A carbon atoms are colored white, Chain B carbon atoms are colored light grey, nitrogen atoms are colored dark grey, and oxygen atoms are colored black. Protein backbone is shown in cartoon representation and residues of interest are shown in stick representation. Polar contacts are shown as black dotted lines. (b) Table comparing the polar interactions as generated from PyMol for ADI-64950 (SIG-LWG) and wild-type IgG1 (WT; PDB ID: 5JII).

FIG. 23 shows several residues in the potential ADI-64950 homodimeric off-products are predicted to sterically clash with each other, reducing propensity for mispairing. (a-d) views of the pairing interface surrounding the region of interest. Alignment of Chains A to Chain B (a) and Chains B to Chain A (b-d) reveal steric clash at the CH3-CH3 interface of several residues at substituted and unsubstituted positions for these potential off-products, including (a) Lys409 and Phe405, (b) Asp356 and Tyr349, (c) T366W and Tyr407, and (d) the orthologous set of T366W and Tyr407. Chain A carbon atoms are colored white, Chain B carbon atoms are colored light grey, nitrogen atoms are colored dark grey, and oxygen atoms are colored black. Protein backbone is shown in cartoon representation, residues of interest are shown in stick representation, and side chains involved in clashes are shown with a transparent molecular surface.

FIG. 24A-24B show comparison of exemplary results obtained in Example 14 with an antibody comprising (i) two same CH3 domains belonging to the WT, R-L, V-V, QR-F, or RG-FG set and (ii) the variable domains of ADI-29235 (open) or ADI-26908 (filled). FIG. 24A compares the production yield of the antibodies produced in CHO cells. FIG. 24B provides a graph of % monomer full-size Ab values measured by SEC.

FIG. 25A-25B show exemplary results comparing FAE outputs with respective FAE inputs in Example 15. FIG. 25A compares the protein recovery rates after FAE reaction steps for producing the indicated bsAbs comprising the WT, R-L, V-V, or QR-F set. FIG. 25B provides IEX results for the R-L, V-V, and QR-F sets, with each panel showing an overlay of (i) chromatograms of FAE inputs (blue and red) each containing the monospecific parent antibody having two of the same, indicated CH3 domains and (ii) a chromatogram of the corresponding FAE reaction output (green).

FIG. 26A-26D show exemplary results comparing FAE outputs with respective FAE inputs in Example 16. FIG. 26A provides exemplary SDS-PAGE results comparing the protein quality between the FAE inputs and outputs. The FAE reaction was carried out for producing the indicated bsAbs comprising the WT, R-L, or V-V set. FIG. 26B provides exemplary LC-MS results, with each panel showing an overlay of (i) chromatograms of FAE inputs (blue and red) each containing the monospecific parent antibody having two of the same, indicated CH3 domains and (ii) a chromatogram of the corresponding FAE reaction output (black). FIG. 26C provides exemplary binding kinetic curves comparing binding to either HER2 or CD3 by the indicated monospecific antibodies in the FAE inputs and the indicated bsAbs in the corresponding FAE outputs. FIG. 26D provides exemplary binding kinetic curves comparing simultaneous binding to HER2 and CD3 by the indicated monospecific antibodies in the FAE inputs and the indicated bsAbs in the corresponding FAE outputs. “HER2→CD3” indicates that the test antibodies were exposed to HER2 first and then CD3. “CD3→HER2” indicates that the test antibodies were exposed to CD3 first and then HER2.

FIG. 27A-27E show exemplary results from the GSH challenge experiments comparing the V-V set to the R-L set, as described in Example 17. FIG. 27A provides a schematic of the GSH challenge experiments. In Step 1, an anti-HER2, CH3 hetero IgG1 comprising a test CH3 set and an anti-CD3, CH3 hetero IgG1 comprising the test CH3 set are generated via FAE using 2-MEA. In Step 2, the anti-HER2, CH3 hetero IgG1 from Step 1 is placed in a mildly reducing environment containing GSH with an anti-CD3 IgG1 comprising (i) two same CH3 domains which are same as one of the test CH3 set, (ii) two same CH3 domains which are same as the other of the test CH3 set, or (iii) the test CH3 set (i.e., the anti-HCD3, CH3 hetero IgG1 from Step 1), and whether chain recombination occurs are evaluated by IEX. FIG. 27B provides exemplary IEX results of FAE in Step 1 with R-L and V-V sets. Each graph panel shows an overlay of a chromatogram of a FAE output and chromatograms of the two FAE input antibodies. FIG. 27C-27E provide exemplary IEX results of GSH challenge in Step 2 with R-L and V-V sets. Each graph panel shows an overlay of a chromatogram of a GSH challenge product and chromatograms of the two GSH challenge input antibodies. I.e., the input antibodies are: the anti-HER2, CH3 hetero antibody; and an anti-CD3 IgG1 comprising (i) two same CH3 domains which are same as one of the test CH3 set (FIG. 27C) (ii) two same CH3 domains which are same as the other of the test CH3 set (FIG. 27D), or (iii) the test CH3 set (i.e., the anti-HCD3, CH3 hetero IgG1 from Step 1 (FIG. 27E).

### DETAILED DESCRIPTION OF THE INVENTION

### Definitions

Unless defined otherwise, all technical and scientific terms used herein have the same meaning as commonly understood by one of ordinary skill in the art to which this disclosure belongs.

As used herein, the term “about,” when used in reference to a particular recited numerical value, means that the value may vary from the recited value by no more than 1%. For example, as used herein, the expression “about 100” includes 99 and 101 and all values in between (e.g., 99.1, 99.2, 99.3, 99.4, etc.).

It is understood that aspects and embodiments of the disclosure described herein include “comprising,” “consisting,” and “consisting essentially of” aspects and embodiments.

The term “antibody” is used herein in the broadest sense and encompasses various antibody structures, including but not limited to monoclonal antibodies, polyclonal antibodies, multispecific antibodies (e.g., bispecific antibodies), and/or antibody fragments (preferably those fragments that exhibit the desired antigen-binding activity, which is also referred to as “antigen-binding antibody fragments”). A “full antibody”, “full Ab”, “full size antibody”, “full size Ab”, “full-length antibody”, “intact antibodies”, or “whole antibody”, or the like, encompasses molecules having a structure substantially similar to a native antibody and, in case of IgG, IgD, or IgE, comprises two immunoglobulin heavy chains and two immunoglobulin light chains. An “antigen-binding fragment” or “antigen-binding antibody fragment” refers to a portion of an intact antibody or to a combination of portions derived from an intact antibody or from intact antibodies and binds the antigen(s) to which the intact antibody or antibodies bind.

An “antigen-binding fragment of an antibody” or “antigen-binding antibody fragment” includes any naturally occurring, enzymatically obtainable, synthetic, or genetically engineered polypeptide or glycoprotein that comprises an antibody domain (e.g., a VH domain or a CH3 domain) specifically binds an antigen to form a complex. Exemplary antibody fragments include, but are not limited to: Fv; fragment antigen-binding (“Fab”) fragment; Fab′ fragment; Fab′ containing a free sulfhydryl group (‘Fab′-SH’); F(ab′)₂ fragment; diabodies; linear antibodies; single-chain antibody molecules (e.g. single-chain variable fragment (“scFv”), nanobody or VHH, or VH or VL domains only); and monospecific or multispecific compounds formed from one or more of antibody fragments such as the foregoing. In some embodiments, the antigen-binding fragments of the bispecific antibodies described herein are scFvs or nanobodies. In preferred embodiments, an antigen-binding fragment comprises a CH3 domain set which preferentially form a CH3-CH3 heterodimer.

As with full antibody molecules, antigen-binding fragments may be mono-specific or multispecific (e.g., bispecific, trispecific, tetraspecific, etc). A multispecific antigen-binding fragment of an antibody may comprise at least two different variable domains, wherein each variable domain is capable of specifically binding to a separate antigen or to a different epitope of the same antigen.

A “monoclonal antibody” or “mAb” refers to an antibody obtained from a population of substantially homogeneous antibodies, i.e., the individual antibodies comprising the population are identical and/or bind the same epitope, except for possible variant antibodies (e.g., containing a naturally occurring mutation(s) and/or substitution(s) or arising during production of a monoclonal antibody preparation), such variants generally being present in minor amounts. In contrast to polyclonal antibody preparations, which typically include different antibodies directed against different determinants (epitopes), each monoclonal antibody of a monoclonal antibody preparation is directed against a single determinant on an antigen.

A “multispecific antibody”, which may also be referred to as “multispecific compound” herein, refers to an antibody comprising at least two different antigen binding domains that recognize and specifically bind to at least two different antigens and/or at least two different epitopes. In some embodiments, a multispecific antibody contains (1) a first heavy chain and a first light chain, which form a cognate pair and bind to a first antigen, and (2) a second heavy chain and a second light chain, which form a cognate pair and bind to a second antigen.

A “bispecific antibody”, which may also be referred to as “bispecific compound” herein, is a type of multispecific antibody and refers to an antibody comprising two different antigen binding domains which recognize and specifically bind to at least two different antigens or at least two epitopes. The at least two epitopes may or may not be within the same antigen. A bispecific antibody may target, for example, two different surface receptors on the same or different (e.g., an immune cell and a cancer cell) cells, two different cytokines/chemokines, a receptor and a ligand.

In some embodiments, the at least two different antigens may be selected from the following antigens (or the at least two different epitopes may be the epitopes with in any of the following antigens): CD3; 0772P (CA125, MUC16; GenBank accession no. AF36148); adipophilin (perilipin-2, Adipose differentiation-related protein, ADRP, ADFP, MGC10598; NCBI Reference Sequence: NP-001113.2); AIM-2 (Absent In Melanoma 2, PYHIN4, Interferon-Inducible Protein AIM2; NCBI Reference Sequence: NP-004824.1); ALDH1 A1 (Aldehyde Dehydrogenase 1 Family, Member A1, ALDH1, PUMB1, Retinaldehyde Dehydrogenase 1, ALDC, ALDH-E1, ALHDII, RALDH 1, EC 1.2.1.36, ALDH11, HEL-9, HEL-S-53e, HEL12, RALDH1, Acetaldehyde Dehydrogenase 1, Aldehyde Dehydrogenase 1, Soluble, Aldehyde Dehydrogenase, Liver Cytosolic, ALDH Class 1, Epididymis Luminal Protein 12, Epididymis Luminal Protein 9, Epididymis Secretory Sperm Binding Protein Li 53e, Retinal Dehydrogenase 1, RaIDH1, Aldehyde Dehydrogenase Family 1 Member A1, Aldehyde Dehydrogenase, Cytosolic, EC 1.2.1; NCBI Reference Sequence: NP-000680.2); alpha-actinin-4 (ACTN4, Actinin, Alpha 4, FSGS1, Focal Segmental Glomerulosclerosis 1, Non-Muscle Alpha-Actinin 4, F-Actin Cross-Linking Protein, FSGS, ACTININ-4, Actinin Alpha4 Isoform, alpha-actinin-4; NCBI Reference Sequence: NP-004915.2); alpha-fetoprotein (AFP, HPAFP, FETA, alpha-1-fetoprotein, alpha-fetoglobulin, Alpha-1-fetoprotein, Alpha-fetoglobulin, HP; GenBank: AAB58754.1); Amphiregulin (AREG, SDGF, Schwannoma-Derived Growth Factor, Colorectum Cell-Derived Growth Factor, AR, CRDGF; GenBank: AAA51781.1); ARTC1 (ART1, ADP-Ribosyltransferase 1, Mono(ADP-Ribosyl)Transferase 1, ADP-Ribosyltransferase C2 And C3 Toxin-Like 1, ART2, CD296, RT6, ADP-Ribosyltransferase 2, GPI-Linked NAD(P)(+)-Arginine ADP-Ribosyltransferase 1, EC 2.4.2.31, CD296 Antigen; NP); ASLG659; ASPHD1 (Aspartate Beta-Hydroxylase Domain Containing 1, Aspartate Beta-Hydroxylase Domain-Containing Protein 1, EC 1.14.11., GenBank: AA144153.1); B7-H4 (VTCN1, V-Set Domain Containing T Cell Activation Inhibitor 1, B7H4, B7 Superfamily Member 1, Immune Costimulatory Protein B7-H4, B7h.5, T-Cell Costimulatory Molecule B7x, B7S1, B7X, VCTN1, H4, B7 Family Member, PRO1291, B7 Family Member, H4, T Cell Costimulatory Molecule B7x, V-Set Domain-Containing T-Cell Activation Inhibitor 1, Protein B7S1; GenBank: AAZ17406.1); BAFF-R (TNFRSF13C, Tumor Necrosis Factor Receptor Superfamily, Member 13C, BAFFR, B-Cell-Activating Factor Receptor, BAFF Receptor, BLyS Receptor 3, CVID4, BROMIX, CD268, B Cell-Activating Factor Receptor, prolixin, Tumor Necrosis Factor Receptor Superfamily Member 13C, BR3, CD268 Antigen; NCBI Reference Sequence: NP-443177.1); BAGE-1; BCLX (L); BCR-ABL fusion protein (b3a2); beta-catenin (CTNNB1, Catenin (Cadherin-Associated Protein), Beta 1, 88 kDa, CTNNB, MRD19, Catenin (Cadherin-Associated Protein), Beta 1 (88 kD), armadillo, Catenin Beta-1; GenBank: CAA61107.1); BING-4 (WDR46, WD Repeat Domain 46, C6orf11, BING4, WD Repeat-Containing Protein BING4, Chromosome 6 Open Reading Frame 11, FP221, UTP7, WD Repeat-Containing Protein 46; NP); BMPR1 B (bone morphogenetic protein receptor-type IB, GenBank accession no. NM-00120; NP); B-RAF (Brevican (BCAN, BEHAB, GenBank accession no. AF22905); Brevican (BCAN, Chondroitin Sulfate Proteoglycan 7, Brain-Enriched Hyaluronan-Binding Protein, BEHAB, CSPG7, Brevican Proteoglycan, Brevican Core Protein, Chondroitin Sulfate Proteoglycan BEHAB; GenBank: AAH27971.1); CALCA (Calcitonin-Related Polypeptide Alpha, CALC1, Calcitonin 1, calcitonin, Alpha-Type CGRP, Calcitonin Gene-Related Peptide I, CGRP-I, CGRP, CGRP1, CT, KC, Calcitonin/Calcitonin-Related Polypeptide, Alpha, katacalcin; NP); CASP-5 (CASP5, Caspase 5, Apoptosis-Related Cysteine Peptidase, Caspase 5, Apoptosis-Related Cysteine Protease, Protease ICH-3, Protease TY, ICE(rel)-111, ICE(rel)III, ICEREL-III, ICH-3, caspase-5, TY Protease, EC 3.4.22.58, ICH3, EC 3.4.22; NP); CASP-8; CD19 (CD19-B-lymphocyte antigen CD19 isoform 2 precursor, B4, CVID3 [Homo sapiens], NCBI Reference Sequence: NP-001761.3); CD20 (CD20-B-lymphocyte antigen CD20, membrane-spanning 4-domains, subfamily A, member 1, B1, Bp35, CD20, CVID5, LEU-16, MS4A2, S7; NCBI Reference Sequence: NP-690605.1); CD21 (CD21 (CR2 (Complement receptor or C3DR (C3d/Epstein Barr virus receptor) or Hs.73792 GenBank accession no. M2600); (CD22 (B-cell receptor CD22-B isoform, BL-CAM, Lyb-8, LybB, SIGLEC-2, FLJ22814, GenBank accession No. AK02646); CD22; CD33 (CD33 Molecule, CD33 Antigen (Gp67), Sialic Acid Binding Ig-Like Lectin 3, Sialic Acid-Binding Ig-Like Lectin 3, SIGLEC3, gp67, SIGLEC-3, Myeloid Cell Surface Antigen CD33, p67, Siglec-3, CD33 Antigen; GenBank: AAH28152.1); CD45; CD70 (CD70-tumor necrosis factor (ligand) superfamily, member 7; surface antigen CD70; Ki-24 antigen; CD27 ligand; CD27-L; tumor necrosis factor ligand superfamily member 7; NCBI Reference Sequence for species Homo sapiens: NP-001243.1); CD72 (CD72 (B-cell differentiation antigen CD72, Lyb-; 359 aa, μl: 8.66, MW: 40225, TM: 1 [P] Gene Chromosome: 9p13.3, GenBank accession No. NP-001773.); CD79a (CD79a (CD79A, CD79a, immunoglobulin-associated alpha, a B cell-specific protein that covalently interacts with Ig beta (CD79B) and forms a complex on the surface with Ig M molecules, transduces a signal involved in B-cell differentiation), μl: 4.84, MW: 25028 TM: 2 [P] Gene Chromosome: 19q13.2, GenBank accession No. NP-001774.1); CD79b (CD79b (CD79B, CD79b, IGb (immunoglobulin-associated beta), B29, GenBank accession no. NM-000626 or 1103867); Cdc27 (Cell Division Cycle 27, DOS1430E, D17S978E, Anaphase Promoting Complex Subunit 3, Anaphase-Promoting Complex Subunit 3, ANAPC3, APC3, CDC27Hs, H-NUC, CDC27 Homolog, Cell Division Cycle 27 Homolog (S. cerevisiae), HNUC, NUC2, Anaphase-Promoting Complex, Protein 3, Cell Division Cycle 27 Homolog, Cell Division Cycle Protein 27 Homolog, Nuc2 Homolog; GenBank: AAH11656.1); CDK4 (Cyclin-Dependent Kinase 4, Cell Division Protein Kinase 4, PSK-J3, EC 2.7.11.22, CMM3, EC 2.7.11; NCBI Reference Sequence: NP-000066.1); CDKN2A (Cyclin-Dependent Kinase Inhibitor 2A, MLM, CDKN2, MTS1, Cyclin-Dependent Kinase Inhibitor 2A (Melanoma, P16, Inhibits CDK4), Cyclin-Dependent Kinase 4 Inhibitor A, Multiple Tumor Suppressor 1, CDK4I, MTS-1, CMM2, P16, ARF, INK4, INK4A, P14, P14ARF, P16-INK4A, P16INK4, P16INK4A, P19, P19ARF, TP16, CDK4 Inhibitor P16-INK4, Cell Cycle Negative Regulator Beta, p14ARF, p16-INK4, p16-INK4a, p161NK4A, p19ARF; NP); CEA; CLL1 (CLL-1 (CLEC12A, MICL, and DCAL, encodes a member of the C-type lectin/C-type lectin-like domain (CTL/CTLD) superfamily. Members of this family share a common protein fold and have diverse functions, such as cell adhesion, cell-cell signaling, glycoprotein turnover, and roles in inflammation and immune response. The protein encoded by this gene is a negative regulator of granulocyte and monocyte function. Several alternatively spliced transcript variants of this gene have been described, but the full-length nature of some of these variants has not been determined. This gene is closely linked to other CTL/CTLD superfamily members in the natural killer gene complex region on chromosome 12p13 (Drickamer, K Curr. Opin. Struct. Biol. 9:585-90 [1999]; van Rhenen, A, et al., Blood 110:2659-66 [2007]; Chen C H, et al. Blood 107:1459-67 [2006]; Marshall A S, et al. Eur. J. Immunol. 36:2159-69 [2006]; Bakker A B, et al Cancer Res. 64:8443-50 [2004]; Marshall A S, et al J. Biol. Chem. 279:14792-80, 2004. CLL-1 has been shown to be a type II transmembrane receptor comprising a single C-type lectin-like domain (which is not predicted to bind either calcium or sugar), a stalk region, a transmembrane domain and a short cytoplasmic tail containing an ITIM motif); CLPP (Caseinolytic Mitochondrial Matrix Peptidase Proteolytic Subunit, Endopeptidase Clp, EC 3.4.21.92, PRLTS3, ATP-Dependent Protease ClpAP (E. coli), ClpP (Caseinolytic Protease, ATP-Dependent, Proteolytic Subunit, E. coli) Homolog, ClpP Caseinolytic Peptidase, ATP-Dependent, Proteolytic Subunit Homolog (E. coli), ClpP Caseinolytic Protease, ATP-Dependent, Proteolytic Subunit Homolog (E. coli), human, Proteolytic Subunit, ATP-Dependent Protease ClpAP, Proteolytic Subunit, Human, ClpP Caseinolytic Peptidase ATP-Dependent, Proteolytic Subunit, ClpP Caseinolytic Peptidase, ATP-Dependent, Proteolytic Subunit Homolog, ClpP Caseinolytic Protease, ATP-Dependent, Proteolytic Subunit Homolog, Putative ATP-Dependent Clp Protease Proteolytic Subunit, Mitochondrial; NP); COA-1; CPSF; CRIPTO (CRIPTO (CR, CR1, CRGF, CRIPTO, TDGF1, teratocarcinoma-derived growth factor, GenBank accession no. NP-003203 or NM-00321); Cw6; CXCR5 (Burkitt's lymphoma receptor 1, a G protein-coupled receptor that is activated by the CXCL13 chemokine, functions in lymphocyte migration and humoral defense, plays a role in HIV-2 infection and perhaps development of AIDS, lymphoma, myeloma, and leukemia); 372 aa, μl: 8.54 MW: 41959 TM: 7 [P] Gene Chromosome: 11q23.3, GenBank accession No. NP-001707.); CXORF61 CXORF61-chromosome X open reading frame 61[Homo sapiens], NCBI Reference Sequence: NP-001017978.1); cyclin D1 (CCND1, BCL1, PRAD1, D11S287E, B-Cell CLL/Lymphoma 1, B-Cell Lymphoma 1 Protein, BCL-1 Oncogene, PRAD1 Oncogene, Cyclin D1 (PRAD1: Parathyroid Adenomatosis 1), G1/S-Specific Cyclin D1, Parathyroid Adenomatosis 1, U21B31, G1/S-Specific Cyclin-D1, BCL-1; NCBI Reference Sequence: NP-444284.1); Cyclin-A1 (CCNA1, CT146, Cyclin A1; GenBank: AAH36346.1); dek-can fusion protein; DKK1 (Dickkopf WNT Signaling Pathway Inhibitor 1, SK, hDkk-1, Dickkopf (Xenopus laevis) Homolog 1, Dickkopf 1 Homolog (Xenopus laevis), DKK-1, Dickkopf 1 Homolog, Dickkopf Related Protein-1, Dickkopf-1 Like, Dickkopf-Like Protein 1, Dickkopf-Related Protein 1, Dickkopf-1, Dkk-1; GenBank: AAQ89364.1); DR1 (Down-Regulator Of Transcription 1, TBP-Binding (Negative Cofactor 2), Negative Cofactor 2-Beta, TATA-Binding Protein-Associated Phosphoprotein, NC2, NC2-BETA, Protein Dr1, NC2-beta, Down-Regulator Of Transcription 1; NCBI Reference Sequence: NP-001929.1); DR13 (Major Histocompatibility Complex, Class II, DR Beta 1, HLA-DR1B, DRw10, DW2.2/DR2.2, SS1, DRB1, HLA-DRB, HLA Class II Histocompatibility Antigen, DR-1 Beta Chain, Human Leucocyte Antigen DRB1, Lymphocyte Antigen DRB1, MHC Class II Antigen, MHC Class II HLA-DR Beta 1 Chain, MHC Class II HLA-DR-Beta Cell Surface Glycoprotein, MHC Class II HLA-DRw10-Beta, DR-1, DR-12, DR-13, DR-14, DR-16, DR-4, DR-5, DR-7, DR-8, DR-9, DR1, DR12, DR13, DR14, DR16, DR4, DR5, DR7, DRB, DR9, DRw11, DRw8, HLA-DRB2, Clone P2-Beta-3, MHC Class II Antigen DRB1*1, MHC Class II Antigen DRB1*10, MHC Class II Antigen DRB1*11, MHC Class II Antigen DRB1*12, MHC Class II Antigen DRB1*13, MHC Class II Antigen DRB1*14, MHC Class II Antigen DRB1*15, MHC Class II Antigen DRB1*16, MHC Class II Antigen DRB1*3, MHC Class II Antigen DRB1*4, MHC Class II Antigen DRB1*7, MHC Class II Antigen DRB1*8, MHC Class II Antigen DRB1*9; NP); E16 (E16 (LAT1, SLC7A5, GenBank accession no. NM-00348); EDAR (EDAR-tumor necrosis factor receptor superfamily member EDAR precursor, EDA-A1 receptor; downless homolog; ectodysplasin-A receptor; ectodermal dysplasia receptor; anhidrotic ectodysplasin receptor 1, DL; ECTD10A; ECTD10B; ED1R; ED3; ED5; EDA-A1R; EDA1R; EDA3; HRM1 [Homo sapiens]; NCBI Reference Sequence: NP-071731.1); EFTUD2 (Elongation Factor Tu GTP Binding Domain Containing 2, Elongation Factor Tu GTP-Binding Domain-Containing Protein 2, hSNU114, SNU114 Homolog, U5 SnRNP-Specific Protein, 116 KDa, MFDGA, KIAA0031, 116 KD, U5 SnRNP Specific Protein, 116 KDa U5 Small Nuclear Ribonucleoprotein Component, MFDM, SNRNP116, Snrp116, Snu114, U5-116KD, SNRP116, U5-116 KDa; GenBank: AAH02360.1); EGFR (Epidermal Growth Factor Receptor, ERBB, Proto-Oncogene C-ErbB-1, Receptor Tyrosine-Protein Kinase ErbB-1, ERBB1, HER1, EC 2.7.10.1, Epidermal Growth Factor Receptor (Avian Erythroblastic Leukemia Viral (V-Erb-B) Oncogene Homolog), Erythroblastic Leukemia Viral (V-Erb-B) Oncogene Homolog (Avian), PlG61, Avian Erythroblastic Leukemia Viral (V-Erb-B) Oncogene Homolog, Cell Growth Inhibiting Protein 40, Cell Proliferation-Inducing Protein 61, mENA, EC 2.7.10; GenBank: AAH94761.1); EGFR-G719A; EGFR-G719C; EGFR-G719S; EGFR-L858R; EGFR-L861 Q; EGFR-57681; EGFR-T790M; Elongation factor 2 (EEF2, Eukaryotic Translation Elongation Factor 2, EF2, Polypeptidyl-TRNA Translocase, EF-2, SCA26, EEF-2; NCBI Reference Sequence: NP-001952.1); ENAH (hMena) (Enabled Homolog (Drosophila), MENA, Mammalian Enabled, ENA, NDPP1, Protein Enabled Homolog; GenBank: AAH95481.1)—results for just “ENAH” not “ENAH (hMena)”; EpCAM (Epithelial Cell Adhesion Molecule, M4S1, MIC18, Tumor-Associated Calcium Signal Transducer 1, TACSTD1, TROP1, Adenocarcinoma-Associated Antigen, Cell Surface Glycoprotein Trop-1, Epithelial Glycoprotein 314, Major Gastrointestinal Tumor-Associated Protein GA733-2, EGP314, KSA, DIAR5, HNPCC8, Antigen Identified By Monoclonal Antibody AUA1, EGP-2, EGP40, ESA, KS1/4, MK-1, Human Epithelial Glycoprotein-2, Membrane Component, Chromosome 4, Surface Marker (35 kD Glycoprotein), EGP, Ep-CAM, GA733-2, M1S2, CD326 Antigen, Epithelial Cell Surface Antigen, hEGP314, KS 1/4 Antigen, ACSTD1; GenBank: AAH14785.1); EphA3 (EPH Receptor A3, ETK1, ETK, TYRO4, HEK, Eph-Like Tyrosine Kinase 1, Tyrosine-Protein Kinase Receptor ETK1, EK4, EPH-Like Kinase 4, EC 2.7.10.1, EPHA3, HEK4, Ephrin Type-A Receptor 3, Human Embryo Kinase 1, TYRO4 Protein Tyrosine Kinase, hEK4, Human Embryo Kinase, Tyrosine-Protein Kinase TYRO4, EC 2.7.10; GenBank: AAH63282.1); EphB2R; Epiregulin (EREG, ER, proepiregulin; GenBank: AAI36405.1); ETBR (EDNRB, Endothelin Receptor Type B, HSCR2, HSCR, Endothelin Receptor Non-Selective Type, ET-B, ET-BR, ETRB, ABCDS, WS4A, ETB, Endothelin B Receptor; NP); ETV6-AML1 fusion protein; EZH2 (Enhancer Of Zeste Homolog 2 (Drosophila), Lysine N-Methyltransferase 6, ENX-1, KMT6 EC 2.1.1.43, EZH1, WVS, Enhancer Of Zeste (Drosophila) Homolog 2, ENX1, EZH2b, KMT6A, WVS2, Histone-Lysine N-Methyltransferase EZH2, Enhancer Of Zeste Homolog 2, EC 2.1.1; GenBank: AAH10858.1); FcRH1 (FCRL1, Fc Receptor-Like 1, FCRH1, Fc Receptor Homolog 1, FcR-Like Protein 1, Immune Receptor Translocation-Associated Protein 5, IFGP1, IRTA5, hIFGP1, IFGP Family Protein 1, CD307a, Fc Receptor-Like Protein 1, Immunoglobulin Superfamily Fc Receptor, Gp42, FcRL1, CD307a Antigen; GenBank: AAH33690.1); FcRH2 (FCRL2, Fc Receptor-Like 2, SPAP1, SH2 Domain-Containing Phosphatase Anchor Protein 1, Fc Receptor Homolog 2, FcR-Like Protein 2, Immunoglobulin Receptor Translocation-Associated Protein 4, FCRH2, IFGP4, IRTA4, IFGP Family Protein 4, SPAP1A, SPAP1 B, SPAP1C, CD307b, Fc Receptor-Like Protein 2, Immune Receptor Translocation-Associated Protein 4, Immunoglobulin Superfamily Fc Receptor, Gp42, SH2 Domain Containing Phosphatase Anchor Protein 1, FcRL2, CD307b Antigen; GenBank: AAQ88497.1); FcRH5 (FCRL5, Fc Receptor-Like 5, IRTA2, Fc Receptor Homolog 5, FcR-Like Protein 5, Immune Receptor Translocation-Associated Protein 2, BXMAS1, FCRH5, CD307, CD307e, PRO820, Fc Receptor-Like Protein 5, Immunoglobulin Superfamily Receptor Translocation Associated 2 (IRTA2), FCRL5, CD307e Antigen; GenBank: AAI01070.1); FLT3-ITD; FN1 (Fibronectin 1, Cold-Insoluble Globulin, FN, Migration-Stimulating Factor, CIG, FNZ, GFND2, LETS, ED-B, FINC, GFND, MSF, fibronectin; GenBank: AAI43764.1); G250 (MN, CAIX, Carbonic Anhydrase IX, Carbonic Dehydratase, RCC-Associated Protein G250, Carbonate Dehydratase IX, Membrane Antigen MN, Renal Cell Carcinoma-Associated Antigen G250, CA-IX, P54/58N, pMW1, RCC-Associated Antigen G250, Carbonic Anhydrase 9; NP); —alias results for “G250” not “G250/MN/CAIX”; GAGE-1,2,8; GAGE-3,4,5,6,7; GDNF-Ra1 (GDNF family receptor alpha 1; GFRA1; GDNFR; GDNFRA; RETL1; TRNR1; RET1 L; GDNFR-alpha1; GFR-ALPHA-; U95847; BC014962; NM-145793 NM-005264); GEDA (GenBank accession No. AY26076); GFRA1-GDNF family receptor alpha-1; GDNF receptor alpha-1; GDNFR-alpha-1; GFR-alpha-1; RET ligand 1; TGF-beta-related neurotrophic factor receptor 1 [Homo sapiens]; ProtKB/Swiss-Prot: P56159.2; glypican-3 (GPC3, Glypican 3, SDYS, Glypican Proteoglycan 3, Intestinal Protein OCI-5, GTR2-2, MXR7, SGBS1, DGSX, OCI-5. SGB, SGBS, Heparan Sulphate Proteoglycan, Secreted Glypican-3, OCI5; GenBank: AAH35972.1); GnTVf; gp100 (PMEL, Premelanosome Protein, SILV, D12S53E, PMEL17, SIL, Melanocyte Protein Pmel 17, Melanocytes Lineage-Specific Antigen GP100, Melanoma-Associated ME20 Antigen, Silver Locus Protein Homolog, ME20-M, ME20M, P1, P100, Silver (Mouse Homolog) Like, Silver Homolog (Mouse), ME20, SI, Melanocyte Protein Mel 17, Melanocyte Protein PMEL, Melanosomal Matrix Protein17, Silver, Mouse, Homolog Of; GenBank: AAC60634.1); GPC; GPNMB (Glycoprotein (Transmembrane) Nmb, Glycoprotein NMB, Glycoprotein Nmb-Like Protein, osteoactivin, Transmembrane Glycoprotein HGFIN, HGFIN, NMB, Transmembrane Glycoprotein, Transmembrane Glycoprotein NMB; GenBank: AAH32783.1); GPR172A (G protein-coupled receptor 172A; GPCR41; FLJ11856; D15Ertd747e); NP-078807.1; NM-024531.3); GPR19 (G protein-coupled receptor 19; Mm.478; NP-006134.1; NM-006143.2); GPR54 (KISS1 receptor; KISS1R; GPR54; HOT7T175; AXOR1; NP-115940.2; NM-032551.4); HAVCR1 (Hepatitis A Virus Cellular Receptor 1, T-Cell Immunoglobulin Mucin Family Member 1, Kidney Injury Molecule 1, KIM-1, KIM1, TIM, TIM-1, TIM1, TIMD-1, TIMD1, T-Cell Immunoglobulin Mucin Receptor 1, T-Cell Membrane Protein 1, HAVCR, HAVCR-1, T Cell Immunoglobin Domain And Mucin Domain Protein 1, HAVcr-1, T-Cell Immunoglobulin And Mucin Domain-Containing Protein 1; GenBank: AAH13325.1); HER2 (ERBB2, V-Erb-B2 Avian Erythroblastic Leukemia Viral Oncogene Homolog 2, NGL, NEU, Neuro/Glioblastoma Derived Oncogene Homolog, Metastatic Lymph Node Gene 19 Protein, Proto-Oncogene C-ErbB-2, Proto-Oncogene Neu, Tyrosine Kinase-Type Cell Surface Receptor HER2, MLN 19, pI85erbB2, EC 2.7.10.1, V-Erb-B2 Avian Erythroblastic Leukemia Viral Oncogene Homolog 2 (Neuro/Glioblastoma Derived Oncogene Homolog), CD340, HER-2, HER-2/neu, TKRI, C-Erb B2/Neu Protein, herstatin, Neuroblastoma/Glioblastoma Derived Oncogene Homolog, Receptor Tyrosine-Protein Kinase ErbB-2, V-Erb-B2 Erythroblastic Leukemia Viral Oncogene Homolog 2, Neuro/Glioblastoma Derived Oncogene Homolog, MLN19, CD340 Antigen, EC 2.7.10; NP); HER-2/neu-alias of above; HERV-K-MEL; HLA-DOB (Beta subunit of MHC class II molecule (la antigen) that binds peptides and presents them to CD4+T lymphocytes); 273 aa, μl: 6.56, MW: 30820.TM: 1 [P] Gene Chromosome: 6p21.3, GenBank accession No. NP-002111); hsp70-2 (HSPA2, Heat Shock 70 kDa Protein 2, Heat Shock 70 kD Protein 2, HSP70-3, Heat Shock-Related 70 KDa Protein 2, Heat Shock 70 KDa Protein 2; GenBank: AAD21815.1); IDO1 (Indoleamine 2,3-Dioxygenase 1, IDO, INDO, Indoleamine-Pyrrole 2,3-Dioxygenase, IDO-1, Indoleamine-Pyrrole 2,3 Dioxygenase, Indolamine 2,3 Dioxygenase, Indole 2,3 Dioxygenase, EC 1.13.11.52; NCBI Reference Sequence: NP-002155.1); IGF2B3; IL13Ralpha2 (IL13RA2, Interleukin 13 Receptor, Alpha 2, Cancer/Testis Antigen 19, Interleukin-13-Binding Protein, IL-13R-alpha-2, IL-13RA2, IL-13 Receptor Subunit Alpha-2, IL-13R Subunit Alpha-2, CD213A2, CT19, IL-13R, IL13BP, Interleukin 13 Binding Protein, Interleukin 13 Receptor Alpha 2 Chain, Interleukin-13 Receptor Subunit Alpha-2, IL13R, CD213a2 Antigen; NP); IL20Ru; Intestinal carboxyl esterase; IRTA2 (alias of FcRH5); Kallikrein 4 (KLK4, Kallikrein-Related Peptidase 4, PRSS17, EMSP1, Enamel Matrix Serine Proteinase 1, Kallikrein-Like Protein 1, Serine Protease 17, KLK-L1, PSTS, AI2A1, Kallikrein 4 (Prostase, Enamel Matrix, Prostate), ARM1, EMSP, Androgen-Regulated Message 1, Enamel Matrix Serine Protease 1, kallikrein, kallikrein-4, prostase, EC 3.4.21.-, Prostase, EC 3.4.21; GenBank: AAX30051.1); KIF20A (Kinesin Family Member 20A, RAB6KIFL, RAB6 Interacting, Kinesin-Like (Rabkinesin6), Mitotic a; LAGE-1; LDLR-fucosyltransferase AS fusion protein; Lengsin (LGSN, Lengsin, Lens Protein With Glutamine Synthetase Domain, GLULD1, Glutamate-Ammonia Ligase Domain-Containing Protein 1, LGS, Glutamate-Ammonia Ligase (Glutamine Synthetase) Domain Containing 1, Glutamate-Ammonia Ligase (Glutamine Synthase) Domain Containing 1, Lens Glutamine Synthase-Like; GenBank: AAF61255.1); LGRS (leucine-rich repeat-containing G protein-coupled receptor 5; GPR49, GPR6; NP-003658.1; NM-003667.2; LY64 (Lymphocyte antigen 64 (RP10, type I membrane protein of the leucine rich repeat (LRR) family, regulates B-cell activation and apoptosis, loss of function is associated with increased disease activity in patients with systemic lupus erythematosis); 661 aa, μl: 6.20, MW: 74147 TM: 1 [P] Gene Chromosome: 5q12, GenBank accession No. NP-005573.; Ly6E (lymphocyte antigen 6 complex, locus E; Ly67, RIG-E, SCA-2, TSA-; NP-002337.1; NM-002346.2); Ly6G6D (lymphocyte antigen 6 complex, locus G6D; Ly6-D, MEGT; NP-067079.2; NM-021246.2); LY6K (lymphocyte antigen 6 complex, locus K; LY6K; HSJ001348; FLJ3522; NP-059997.3; NM-017527.3); LyPD1-LY6/PLAUR domain containing 1, PHTS [Homo sapiens], GenBank: AAH17318.1); MAGE-A1 (Melanoma Antigen Family A, 1 (Directs Expression Of Antigen MZ2-E, MAGE1, Melanoma Antigen Family A 1, MAGEA1, Melanoma Antigen MAGE-1, Melanoma-Associated Antigen 1, Melanoma-Associated Antigen MZ2-E, Antigen MZ2-E, Cancer/Testis Antigen 1.1, CT1.1, MAGE-1 Antigen, Cancer/Testis Antigen Family 1, Member 1, Cancer/Testis Antigen Family 1, Member 1, MAGE1A; NCBI Reference Sequence: NP-004979.3); MAGE-A10 (MAGEA10, Melanoma Antigen Family A, 10, MAGE10, MAGE-10 Antigen, Melanoma-Associated Antigen 10, Cancer/Testis Antigen 1.10, CT1.10, Cancer/Testis Antigen Family 1, Member 10, Cancer/Testis Antigen Family 1, Member 10; NCBI Reference Sequence: NP-001238757.1); MAGE-A12 (MAGEA12, Melanoma Antigen Family A, 12, MAGE12, Cancer/Testis Antigen 1.12, CT1.12, MAGE12F Antigen, Cancer/Testis Antigen Family 1, Member 12, Cancer/Testis Antigen Family 1, Member 12, Melanoma-Associated Antigen 12, MAGE-12 Antigen; NCBI Reference Sequence: NP-001159859.1); MAGE-A2 (MAGEA2, Melanoma Antigen Family A, 2, MAGE2, Cancer/Testis Antigen 1.2, CT1.2, MAGEA2A, MAGE-2 Antigen, Cancer/Testis Antigen Family 1, Member 2, Cancer/Testis Antigen Family 1, Member 2, Melanoma Antigen 2, Melanoma-Associated Antigen 2; NCBI Reference Sequence: NP-001269434.1); MAGE-A3 (MAGEA3, Melanoma Antigen Family A, 3, MAGE3, MAGE-3 Antigen, Antigen MZ2-D, Melanoma-Associated Antigen 3, Cancer/Testis Antigen 1.3, CT1.3, Cancer/Testis Antigen Family 1, Member 3, HIPS, HYPD, MAGEA6, Cancer/Testis Antigen Family 1, Member 3; NCBI Reference Sequence: NP-005353.1); MAGE-A4 (MAGEA4, Melanoma Antigen Family A, 4, MAGE4, Melanoma-Associated Antigen 4, Cancer/Testis Antigen 1.4, CT1.4, MAGE-4 Antigen, MAGE-41 Antigen, MAGE-X2 Antigen, MAGE4A, MAGE4B, Cancer/Testis Antigen Family 1, Member 4, MAGE-41, MAGE-X2, Cancer/Testis Antigen Family 1, Member 4; NCBI Reference Sequence: NP-001011550.1); MAGE-A6 (MAGEA6, Melanoma Antigen Family A, 6, MAGE6, MAGE-6 Antigen, Melanoma-Associated Antigen 6, Cancer/Testis Antigen 1.6, CT1.6, MAGE3B Antigen, Cancer/Testis Antigen Family 1, Melanoma Antigen Family A 6, Member 6, MAGE-3b, MAGE3B, Cancer/Testis Antigen Family 1, Member 6; NCBI Reference Sequence: NP-787064.1); MAGE-A9 (MAGEA9, Melanoma Antigen Family A, 9, MAGE9, MAGE-9 Antigen, Melanoma-Associated Antigen 9, Cancer/Testis Antigen 1.9, CT1.9, Cancer/Testis Antigen Family 1, Member 9, Cancer/Testis Antigen Family 1, Member 9, MAGEA9A; NCBI Reference Sequence: NP-005356.1); MAGE-C1 (MAGEC1, Melanoma Antigen Family C, 1, Cancer/Testis Antigen 7.1, CT7.1, MAGE-C1 Antigen, Cancer/Testis Antigen Family 7, Member 1, CT7, Cancer/Testis Antigen Family 7, Member 1, Melanoma-Associated Antigen C1; NCBI Reference Sequence: NP-005453.2); MAGE-C2 (MAGEC2, Melanoma Antigen Family C, 2, MAGEE1, Cancer/Testis Antigen 10, CT10, HCA587, Melanoma Antigen, Family E, 1, Cancer/Testis Specific, Hepatocellular Carcinoma-Associated Antigen 587, MAGE-C2 Antigen, MAGE-E1 Antigen, Hepatocellular Cancer Antigen 587, Melanoma-Associated Antigen C2; NCBI Reference Sequence: NP-057333.1); mammaglobin-A (SCGB2A2, Secretoglobin, Family 2A, Member 2, MGB1, Mammaglobin 1, UGB2, Mammaglobin A, mammaglobin-A, Mammaglobin-1, Secretoglobin Family 2A Member 2; NP); MART2 (H HAT, Hedgehog Acyltransferase, SKI1, Melanoma Antigen Recognized By T-Cells 2, Skinny Hedgehog Protein 1, Skn, Melanoma Antigen Recognized By T Cells 2, Protein-Cysteine N-Palmitoyltransferase HHAT, EC 2.3.1.-; GenBank: AAH39071.1); M-CSF (CSF1, Colony Stimulating Factor 1 (Macrophage), MCSF, CSF-1, lanimostim, Macrophage Colony-Stimulating Factor 1, Lanimostim; GenBank: AAH21117.1); MCSP (SMCP, Sperm Mitochondria-Associated Cysteine-Rich Protein, MCS, Mitochondrial Capsule Selenoprotein, HSMCSGEN1, Sperm Mitochondrial-Associated Cysteine-Rich Protein; NCBI Reference Sequence: NP-109588.2); XAGE-1b/GAGED2a; WT1 (Wilms Tumor 1, WAGR, GUD, WIT-2, WT33, Amino-Terminal Domain Of EWS, NPHS4, Last Three Zinc Fingers Of The DNA-Binding Domain Of WT1, AWT1, Wilms Tumor Protein, EWS-WT1; GenBank: AAB33443.1); VEGF; Tyrosinase (TYR; OCAIA; OCA1A; tyrosinase; SHEP; NP-000363.1; NM-000372.4; GenBank: AAB60319.1); TrpM4 (BR22450, FLJ20041, TRPM4, TRPM4B, transient receptor potential cation channel, subfamily M, member 4, GenBank accession no. NM-01763); TRP2-INT2; TRP-2; TRP-1/gp75 (Tyrosinase-Related Protein 1, 5,6-Dihydroxyindole-2-Carboxylic Acid Oxidase, CAS2, CATB, TYRP, OCAS, Catalase B, b-PROTEIN, Glycoprotein 75, EC 1.14.18., Melanoma Antigen Gp75, TYRP1, TRP, TYRRP, TRP1, SHEP11, DHICA Oxidase, EC 1.14.18, GP75, EC 1.14.18.1; Triosephosphate isomerase (Triosephosphate isomerase 1, TPID, Triose-Phosphate Isomerase, HEL-S-49, TIM, Epididymis Secretory Protein Li 49, TPI, Triosephosphate Isomerase, EC 5.3.1.1; TRAG-3 (CSAG Family Member 2, Cancer/Testis Antigen Family 24, CSAG3B, Member 2, CSAG Family Member 3B, Cancer/Testis Antigen Family 24 Member 2, Cancer/Testis Antigen 24.2, Chondrosarcoma-Associated Gene 2/3 Protein, Taxol-Resistant-Associated Gene 3 Protein, Chondrosarcoma-Associated Gene 2/3 Protein-Like, CT24.2, Taxol Resistance Associated Gene 3, TRAG-3, CSAG3A, TRAG3;); TMEM46 (shisa homolog 2 (Xenopus laevis); SHISA; NP-001007539.1; NM-001007538.1; TMEM118 (ring finger protein, transmembrane2; RNFT2; FLJ1462; NP-001103373.1; NM-001109903.1; TMEFF1 (transmembrane protein with EGF-like and two follistatin-like domains 1; Tomoregulin-; H7365; C9orf2; C90RF2; U19878; X83961; NM-080655; NM-003692; TGF-betaRII (TGFBR2, Transforming Growth Factor, Beta Receptor II (70/80 kDa), TGFbeta-RII, MFS2, tbetaR-II, TGFR-2, TGF-Beta Receptor Type IIB, TGF-Beta Type II Receptor, TGF-Beta Receptor Type-2, EC 2.7.11.30, Transforming Growth Factor Beta Receptor Type IIC, AAT3, TbetaR-II, Transforming Growth Factor, Beta Receptor II (70-80 kD), TGF-Beta Receptor Type II, FAA3, Transforming Growth Factor-Beta Receptor Type II, LDS1 B, HNPCC6, LDS2B, LDS2, RITC, EC 2.7.11, TAAD2; TENB2 (TMEFF2, tomoregulin, TPEF, HPP1, TR, putative transmembrane proteoglycan, related to the EGF/heregulin family of growth factors and follistatin); 374 aa, NCBI Accession: AAD55776, AAF91397, AAG49451, NCBI RefSeq: NP-057276; NCBI Gene: 23671; OMIM: 605734; SwissProt Q9UIK5; GenBank accession No. AF179274; AY358907, CAF85723, CQ782436; TAG-2; TAG-1 (Contactin 2 (Axonal), TAG-1, AXT, Axonin-1 Cell Adhesion Molecule, TAX, Contactin 2 (transiently Expressed), TAXI, Contactin-2, Axonal Glycoprotein TAG-1, Transiently-Expressed Axonal Glycoprotein, Transient Axonal Glycoprotein, Axonin-1, TAX-1, TAG1, FAMES; PRF: 444868); SYT-SSX1 or -SSX2 fusion protein; survivin; STEAP2 (HGNC 8639, IPCA-1, PCANAP1, STAMP1, STEAP2, STMP, prostate cancer associated gene 1, prostate cancer associated protein 1, six transmembrane epithelial antigen of prostate 2, six transmembrane prostate protein, GenBank accession no. AF45513; STEAP1 (six transmembrane epithelial antigen of prostate, GenBank accession no. NM-01244; SSX-4; SSX-2 (SSX2, Synovial Sarcoma, X Breakpoint2, X Breakpoint 2, SSX, X Breakpoint 2B, Cancer/Testis Antigen 5.2, X-Chromosome-Related 2, Tumor Antigen HOM-MEL-40, CT5.2, HD21, Cancer/Testis Antigen Family 5, HOM-MEL-40, Isoform B, Cancer/Testis Antigen Family 5 member 2a, member 2a, Protein SSX2, Sarcoma, Sarcoma, Synovial, X-Chromosome-Related 2, synovial, Synovial Sarcoma, X Breakpoint 2B, Synovial Sarcomam, SSX2A; Sp17; SOX10 (SRY (Sex Determining Region Y)-Box 10, mouse, PCWH, DOM, WS4, WS2E, WS4C, Dominant Megacolon, mouse, Human Homolog Of, Dominant Megacolon, SRY-Related HMG-Box Gene 10, Human Homolog Of, transcription Factor SOX-10; GenBank: CAG30470.1); SNRPD1 (Small Nuclear Ribonucleoprotein D1, Small Nuclear Ribonucleoprotein D1, Polypeptide 16 kDa, Polypeptide (16 kD), SNRPD, HsT2456, Sm-D1, SMD1, Sm-D Autoantigen, Small Nuclear Ribonucleoprotein D1 Polypeptide 16 kDa Pseudogene, SnRNP Core Protein D1, Small Nuclear Ribonucleoprotein Sm D1; SLC35D3 (Solute Carrier Family 35, Member D3, FRCL1, Fringe Connection-Like Protein 1, bA55K22.3, Frc, Fringe-Like 1, Solute Carrier Family 35 Member D3; NCBI GenBank: NC-000006.11 NC-018917.2 NT-025741.16); SIRT2 (Sirtuin 2, NAD-Dependent Deacetylase Sirtuin-2, SIRL2, Silent Information Regulator 2, Regulatory Protein SIR2 Homolog 2, Sir2-Related Protein Type 2, SIR2-Like Protein 2, Sirtuin Type 2, Sirtuin (Silent Mating Type Information Regulation 2 Homolog) 2 (S. cerevisiae), Sirtuin-2, Sirtuin (Silent Mating Type Information Regulation 2, S. cerevisiae, Homolog) 2, EC 3.5.1., SIR2; GenBank: AAK51133.1); Sema 5b (FLJ10372, KIAA1445, Mm.42015, SEMA5B, SEMAG, Semaphorin 5b Hlog, sema domain, seven thrombospondin repeats (type 1 and type 1-like), Transmembrane Domain™ and short cytoplasmic domain, (semaphorin) 5B, GenBank accession no. AB04087; secernin 1 (SCRN1, SES1, KIAA0193, secerin-1; GenBank: EAL24458.1); SAGE (SAGE1, Sarcoma Antigen 1, Cancer/Testis Antigen 14, CT14, Putative Tumor Antigen; NCBI Reference Sequence: NP-061136.2); RU2AS (KAAG1, Kidney Associated Antigen 1, RU2AS, RU2 Antisense Gene Protein, Kidney-Associated Antigen 1; GenBank: AAF23613.1); RNF43-E3 ubiquitin-protein ligase RNF43 precursor [Homo sapiens], RNF124; URCC; NCBI Reference Sequence: NP-060233.3; RhoC (RGS5 (Regulator Of G-Protein Signaling 5, MSTP032, Regulator Of G-Protein Signalling 5, MSTP092, MST092, MSTP106, MST106, MSTP129, MST129; GenBank: AAB84001.1); RET (ret proto-oncogene; MEN2A; HSCR1; MEN2B; MTC1; PTC; CDHF12; Hs.168114; RET51; RET-ELE; NP-066124.1; NM-020975.4); RBAF600 (UBR4, Ubiquitin Protein Ligase E3 Component N-Recognin 4, Zinc Finger, UBR1 Type 1, ZUBRI, E3 Ubiquitin-Protein Ligase UBR4, RBAF600, 600 KDa Retinoblastoma Protein-Associated Factor, Zinc Finger UBR1-Type Protein 1, EC 6.3.2., N-recognin-4, KIAA0462, p600, EC 6.3.2, KIAA1307; GenBank: AAL83880.1); RAGE-1 (MOK, MOK Protein Kinase, Renal Tumor Antigen, RAGE, MAPK/MAK/MRK Overlapping Kinase, Renal Tumor Antigen 1, Renal Cell Carcinoma Antigen, RAGE-1, EC 2.7.11.22, RAGE1; UniProtKB/Swiss-Prot: Q9UQ07.1); RAB38/NY-MEL-1 (RAB38, NY-MEL-1, RAB38, Member RAS Oncogene Family, Melanoma Antigen NY-MEL-1, Rab-Related GTP-Binding Protein, Ras-Related Protein Rab-38, rrGTPbp; GenBank: AAH15808.1); PTPRK (DJ480J14.2.1 (Protein Tyrosine Phosphatase, Receptor Type, K R-PTP-KAPPA, Protein Tyrosine Phosphatase Kappa, Protein Tyrosine Phosphatase Kappa), Protein Tyrosine Phosphatase, Receptor Type, K, Protein-Tyrosine Phosphatase Kappa, Protein-Tyrosine Phosphatase, Receptor Type, Kappa, R-PTP-kappa, Receptor-Type Tyrosine-Protein Phosphatase Kappa, EC 3.1.3.48, PTPK; GenBank: AA144514.1); PSMA; PSCA hIg (2700050C12Rik, C530008016Rik, RIKEN cDNA 2700050C12, RIKEN cDNA 2700050C12 gene, GenBank accession no. AY358628); PSCA (Prostate stem cell antigen precursor, GenBank accession no. AJ29743; PRDX5 (Peroxiredoxin 5, EC 1.11.1.15, TPx Type VI, B166, Antioxidant Enzyme B166, HEL-S-55, Liver Tissue 2D-Page Spot 71 B, PMP20, Peroxisomal Antioxidant Enzyme, PRDX6, Thioredoxin Peroxidase PMP20, PRXV, AOEB166, Epididymis Secretory Protein Li 55, Alu Co-Repressor 1, Peroxiredoxin-5, Mitochondrial, Peroxiredoxin V, prx-V, Thioredoxin Reductase, Prx-V, ACR1, Alu Corepressor, PLP; GenBank: CAG33484.1); PRAME (Preferentially Expressed Antigen In Melanoma, Preferentially Expressed Antigen Of Melanoma, MAPE, 01P-4, OIPA, CT130, Cancer/Testis Antigen 130, Melanoma Antigen Preferentially Expressed In Tumors, Opa-Interacting Protein 4, Opa-Interacting Protein 01P4; GenBank: CAG30435.1); pml-RARalpha fusion protein; PMEL17 (silver homolog; SILV; D12S53E; PMEL17; SI; SIL); ME20; gp10 BC001414; BT007202; M32295; M77348; NM-006928; PBF (ZNF395, Zinc Finger Protein 395, PRF-1, Huntington disease regulatory, HD Gene Regulatory Region-Binding Protein, Region-Binding Protein 2, Protein 2, Papillomavirus Regulatory Factor 1, HD-Regulating Factor 2, Papillomavirus-Regulatory Factor, PRF1, HDBP-2, Si-1-8-14, HDBP2, Huntington'S Disease Gene Regulatory Region-Binding Protein 2, HDRF-2, Papillomavirus Regulatory Factor PRF-1, PBF; GenBank: AAH01237.1); PAX5 (Paired Box 5, Paired Box Homeotic Gene 5, BSAP, Paired Box Protein Pax-5, B-Cell Lineage Specific Activator, Paired Domain Gene 5, Paired Box Gene 5 (B-Cell Lineage Specific Activator Protein), B-Cell-Specific Transcription Factor, Paired Box Gene 5 (B-Cell Lineage Specific Activator); PAP (REG3A, Regenerating Islet-Derived 3 Alpha, INGAP, PAP-H, Hepatointestinal Pancreatic Protein, PBBCGF, Human Proislet Peptide, REG-Ill, Pancreatitis-Associated Protein 1, Regi, Reg III-Alpha, hepatocarcinoma-intestine-pancreas, Regenerating Islet-Derived Protein III-Alpha, Pancreatic Beta Cell Growth Factor, HIP, PAP Homologous Protein, HIP/PAP, Proliferation-Inducing Protein 34, PAP1, Proliferation-Inducing Protein 42, REG-3-alpha, Regenerating Islet-Derived Protein 3-Alpha, Pancreatitis-Associated Protein; GenBank: AAH36776.1); p53 (TP53, Tumor Protein P53, TPR53, P53, Cellular Tumor Antigen P53, Antigen NY-CO-13, Mutant Tumor Protein 53, Phosphoprotein P53, P53 Tumor Suppressor, BCC7, Transformation-Related Protein 53, LFS1, tumor Protein 53, Li-Fraumeni Syndrome, Tumor Suppressor P53; P2X5 (Purinergic receptor P2X ligand-gated ion channel 5, an ion channel gated by extracellular ATP, may be involved in synaptic transmission and neurogenesis, deficiency may contribute to the pathophysiology of idiopathic detrusor instability); 422 aa), μl: 7.63, MW: 47206 TM: 1 [P] Gene Chromosome: 17p13.3, GenBank accession No. NP-002552.; OGT (0-Linked N-Acetylglucosamine (GlcNAc) Transferase, O-GlcNAc Transferase P110 Subunit, 0-Linked N-Acetylglucosamine (GlcNAc) Transferase (UDP-N-Acetylglucosamine:Polypeptide-N-Acetylglucosaminyl Transferase, UDP-N-Acetylglucosamine-Peptide N-Acetylglucosaminyltransferase 110 KDa Subunit, UDP-N-Acetylglucosamine:Polypeptide-N-Acetylglucosaminyl Transferase, Uridinediphospho-N-Acetylglucosamine:Polypeptide Beta-N-Acetylglucosaminyl Transferase, 0-GlcNAc Transferase Subunit P110, EC 2.4.1.255, 0-Linked N-Acetylglucosamine Transferase 110 KDa Subunit, EC 2.4.1, HRNT1, EC 2.4.1.186, 0-GLCNAC; GenBank: AAH38180.1); 0A1 (Osteoarthritis QTL 1, OASD; GenBank: CAA88742.1); NY-ESO-1/LAGE-2 (Cancer/Testis Antigen 1 B, CTAG1 B, NY-ESO-1, LAGE-2, ESO1, CTAG1, CTAG, LAGE2B, Cancer/Testis Antigen 1, Autoimmunogenic Cancer/Testis Antigen NY-ESO-1, Ancer Antigen 3, Cancer/Testis Antigen 6.1, New York Esophageal Squamous Cell Carcinoma 1, L Antigen Family Member 2, LAGE2, CT6.1, LAGE2A; GenBank: AAI30365.1); NY-BR-1 (ANKRD30A, Ankyrin Repeat Domain 30A, Breast Cancer Antigen NY-BR-1, Serologically Defined Breast Cancer Antigen NY-BR-1, Ankyrin Repeat Domain-Containing Protein 30A; NCBI Reference Sequence: NP-443723.2); N-ras (NRAS, Neuroblastoma RAS Viral (V-Ras) Oncogene Homolog, NRAS1, Transforming Protein N-Ras, GTPase NRas, ALPS4, N-Ras Protein Part 4, NS6, Oncogene Homolog, HRAS1; GenBank: AAH05219.1); NFYC (Nuclear Transcription Factor Y, Gamma, HAP5, HSM, Nuclear Transcription Factor Y Subunit C, Transactivator HSM-1/2, CCAAT Binding Factor Subunit C, NF-YC, CCAAT Transcription Binding Factor Subunit Gamma, CAAT Box DNA-Binding Protein Subunit C, Histone H1 Transcription Factor Large Subunit 2A, CBFC, Nuclear Transcription Factor Y Subunit Gamma, CBF-C, Transactivator HSM-1, H1TF2A, Transcription Factor NF-Y, C Subunit; neo-PAP (PAPOLG, Poly(A) Polymerase Gamma, Neo-Poly(A) Polymerase, Nuclear Poly(A) Polymerase Gamma, Polynucleotide Adenylyltransferase Gamma, SRP RNA 3′ Adenylating Enzyme/Pap2, PAP-gamma, Neo-PAP, SRP RNA 3′-Adenylating Enzyme, PAP2, EC 2.7.7.19, PAPG; NCBI Reference Sequence: NP-075045.2); NCA (CEACAM6, GenBank accession no. M1872); Napi3b (NAPI-3B, NPTIIb, SLC34A2, solute carrier family 34 (sodium phosphate), member 2, type II sodium-dependent phosphate transporter 3b, GenBank accession no. NM-00642); Myosin class I; MUM-3; MUM-2 (TRAPPC1, Trafficking Protein Particle Complex 1, BETS, BETS Homolog, MUM2, Melanoma Ubiquitous Mutated 2, Multiple Myeloma Protein 2, Trafficking Protein Particle Complex Subunit 1; MUM-If; Mucin (MUC1, Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC1/ZD, Tumor-Associated Epithelial Membrane Antigen, DF3 Antigen, Tumor-Associated Mucin, episialin, EMA, H23 Antigen, H23AG, Mucin-1, KL-6, Tumor Associated Epithelial Mucin, MUC-1, Episialin, PEM, CD227 Antigen; UniProtKB/Swiss-Prot: P15941.3); MUCSAC (Mucin SAC, Oligomeric Mucus/Gel-Forming, Tracheobronchial Mucin, MUC5, TBM, Mucin 5, Subtypes A And C, Tracheobronchial/Gastric, leB, Gastric Mucin, Mucin SAC, Oligomeric Mucus/Gel-Forming Pseudogene, Lewis B Blood Group Antigen, LeB, Major Airway Glycoprotein, MUC-SAC, Mucin-5 Subtype AC, Tracheobronchial; MUC1 (Mucin 1, Cell Surface Associated, PEMT, PUM, CA 15-3, MCKD1, ADMCKD, Medullary Cystic Kidney Disease 1 (Autosomal Dominant), ADMCKD1, Mucin 1, Transmembrane, CD227, Breast Carcinoma-Associated Antigen DF3, MAM6, Cancer Antigen 15-3, MCD, Carcinoma-Associated Mucin, MCKD, Krebs Von Den Lungen-6, MUC-1/SEC, Peanut-Reactive Urinary Mucin, MUC-1/X, Polymorphic Epithelial Mucin, MUC1/ZD, Tumor-Associated Epithelial Membrane Antigen, DF3 Antigen, Tumor-Associated Mucin, episialin, EMA, h23 Antigen, H23AG, mucin-1, KL-6, Tumor Associated Epithelial Mucin, MUC-1, Episialin, PEM, CD227 Antigen; MSG783 (RNF124, hypothetical protein FLJ20315, GenBank accession no. NM-01776; MRP4-multidrug resistance-associated protein 4 isoform 3, MOAT-B; MOATB [Homo sapiens]; NCBI Reference Sequence: NP-001288758.1; MPF (MPF, MSLN, SMR, megakaryocyte potentiating factor, mesothelin, GenBank accession no. NM-00582; MMP-7 (MMP7, matrilysin, MPSL1, matrin, Matrix Metalloproteinase 7 (Matrilysin, Uterine), Uterine Matrilysin, Matrix Metalloproteinase-7, EC 3.4.24.23, Pump-1 Protease, Matrin, Uterine Metalloproteinase, PUMP1, MMP-7, EC 3.4.24, PUMP-1; GenBank: AAC37543.1); MMP-2 (MMP2, Matrix Metallopeptidase 2 (Gelatinase A, 72 kDa Gelatinase, 72 kDa Type IV Collagenase), MONA, CLG4A, Matrix Metalloproteinase 2 (Gelatinase A, 72 kD Gelatinase, 72 kD Type IV Collagenase), CLG4, 72 kDa Gelatinase, 72 kDa Type IV Collagenase), Matrix Metalloproteinase-2, MMP-II, 72 KDa Gelatinase, Collagenase Type IV-A, MMP-2, Matrix Metalloproteinase-II, TBE-1, Neutrophil Gelatinase, EC 3.4.24.24, EC 3.4.24; GenBank: AAH02576.1); and Meloe;

In some embodiments, the at least two different antigens may be selected from the following antigens (or the at least two different epitopes may be the epitopes with in any of the following antigens): 17-IA, 4-1BB, 4Dc, 6-keto-PGFla, 8-iso-PGF2a, 8-oxo-dG, Al Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcRl, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gpl30, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gpl20, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gpl20, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGGI, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-R1, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCRI, XCRI, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

In certain embodiments, the multispecific (e.g., bispecific) antibody according to the present disclosure may have a first antigen binding domain having specificity for CD3 and a second binding domain having specificity for a second antigen selected from the group consisting of: 17-IA, 4-1BB, 4Dc, 6-keto-PGFla, 8-iso-PGF2a, 8-oxo-dG, A1 Adenosine Receptor, A33, ACE, ACE-2, Activin, Activin A, Activin AB, Activin B, Activin C, Activin RIA, Activin RIA ALK-2, Activin RIB ALK-4, Activin RIIA, Activin RUB, ADAM, ADAM10, ADAM12, ADAM15, ADAM17/TACE, ADAM8, ADAM9, ADAMTS, ADAMTS4, ADAMTS5, Addressins, aFGF, ALCAM, ALK, ALK-1, ALK-7, alpha-1-antitrypsin, alpha-V/beta-1 antagonist, ANG, Ang, APAF-1, APE, APJ, APP, APRIL, AR, ARC, ART, Artemin, anti-Id, ASPARTIC, Atrial natriuretic factor, av/b3 integrin, Axl, b2M, B7-1, B7-2, B7-H, B-lymphocyte Stimulator (BlyS), BACE, BACE-1, Bad, BAFF, BAFF-R, Bag-1, BAK, Bax, BCA-1, BCAM, Bel, BCMA, BDNF, b-ECGF, bFGF, BID, Bik, BIM, BLC, BL-CAM, BLK, BMP, BMP-2 BMP-2a, BMP-3 Osteogenin, BMP-4 BMP-2b, BMP-5, BMP-6 Vgr-1, BMP-7 (OP-1), BMP-8 (BMP-8a, OP-2), BMPR, BMPR-IA (ALK-3), BMPR-IB (ALK-6), BRK-2, RPK-1, BMPR-II (BRK-3), BMPs, b-NGF, BOK, Bombesin, Bone-derived neurotrophic factor, BPDE, BPDE-DNA, BTC, complement factor 3 (C3), C3a, C4, C5, C5a, CIO, CA125, CAD-8, Calcitonin, cAMP, carcinoembryonic antigen (CEA), carcinoma-associated antigen, Cathepsin A, Cathepsin B, Cathepsin C/DPPI, Cathepsin D, Cathepsin E, Cathepsin H, Cathepsin L, Cathepsin O, Cathepsin S, Cathepsin V, Cathepsin X/Z/P, CBL, CCI, CCK2, CCL, CCL1, CCL11, CCL12, CCL13, CCL 14, CCL15, CCL16, CCL17, CCL18, CCL19, CCL2, CCL20, CCL21, CCL22, CCL23, CCL24, CCL25, CCL26, CCL27, CCL28, CCL3, CCL4, CCL5, CCL6, CCL7, CCL8, CCL9/10, CCR, CCR1, CCR10, CCR10, CCR2, CCR3, CCR4, CCR5, CCR6, CCR7, CCR8, CCR9, CD1, CD2, CD4, CD5, CD6, CD7, CD8, CD10, CD11a, CD11b, CD11c, CD13, CD14, CD15, CD16, CD18, CD19, CD20, CD21, CD22, CD23, CD25, CD27L, CD28, CD29, CD30, CD30L, CD32, CD33 (p67 proteins), CD34, CD38, CD40, CD40L, CD44, CD45, CD46, CD49a, CD52, CD54, CD55, CD56, CD61, CD64, CD66e, CD74, CD80 (B7-1), CD89, CD95, CD123, CD137, CD138, CD140a, CD146, CD147, CD148, CD152, CD164, CEACAM5, CFTR, cGMP, CINC, Clostridium botulinum toxin, Clostridium perfringens toxin, CKb8-1, CLC, CMV, CMV UL, CNTF, CNTN-1, COX, C-Ret, CRG-2, CT-1, CTACK, CTGF, CTLA-4, CX3CL1, CX3CR1, CXCL, CXCL1, CXCL2, CXCL3, CXCL4, CXCL5, CXCL6, CXCL7, CXCL8, CXCL9, CXCL10, CXCL11, CXCL12, CXCL13, CXCL14, CXCL15, CXCL16, CXCR, CXCR1, CXCR2, CXCR3, CXCR4, CXCR5, CXCR6, cytokeratin tumor-associated antigen, DAN, DCC, DcR3, DC-SIGN, Decay accelerating factor, des(1-3)-IGF-I (brain IGF-1), Dhh, digoxin, DNAM-1, Dnase, Dpp, DPPIV/CD26, Dtk, ECAD, EDA, EDA-A1, EDA-A2, EDAR, EGF, EGFR (ErbB-1), EMA, EMMPRIN, EN A, endothelin receptor, Enkephalinase, eNOS, Eot, eotaxinl, EpCAM, Ephrin B2/EphB4, EPO, ERCC, E-selectin, ET-1, Factor IIa, Factor VII, Factor VIIIc, Factor IX, fibroblast activation protein (FAP), Fas, FcRl, FEN-1, Ferritin, FGF, FGF-19, FGF-2, FGF3, FGF-8, FGFR, FGFR-3, Fibrin, FL, FLIP, Flt-3, Flt-4, Follicle stimulating hormone, Fractalkine, FZD1, FZD2, FZD3, FZD4, FZD5, FZD6, FZD7, FZD8, FZD9, FZD10, G250, Gas 6, GCP-2, GCSF, GD2, GD3, GDF, GDF-1, GDF-3 (Vgr-2), GDF-5 (BMP-14, CDMP-1), GDF-6 (BMP-13, CDMP-2), GDF-7 (BMP-12, CDMP-3), GDF-8 (Myostatin), GDF-9, GDF-15 (MIC-1), GDNF, GDNF, GFAP, GFRa-1, GFR-alpha1, GFR-alpha2, GFR-alpha3, GITR, Glucagon, Glut 4, glycoprotein IIb/IIIa (GP IIb/IIIa), GM-CSF, gpl30, gp72, GRO, Growth hormone releasing factor, Hapten (NP-cap or NIP-cap), HB-EGF, HCC, HCMV gB envelope glycoprotein, HCMV) gH envelope glycoprotein, HCMV UL, Hemopoietic growth factor (HGF), Hep B gpl20, heparanase, Her2, Her2/neu (ErbB-2), Her3 (ErbB-3), Her4 (ErbB-4), herpes simplex virus (HSV) gB glycoprotein, HSV gD glycoprotein, HGFA, High molecular weight melanoma-associated antigen (HMW-MAA), HIV gpl20, HIV IIIB gp 120 V3 loop, HLA, HLA-DR, HM1.24, HMFG PEM, HRG, Hrk, human cardiac myosin, human cytomegalovirus (HCMV), human growth hormone (HGH), HVEM, 1-309, IAP, ICAM, ICAM-1, ICAM-3, ICE, ICOS, IFNg, Ig, IgA receptor, IgE, IGF, IGF binding proteins, IGF-1R, IGFBP, IGF-I, IGF-II, IL, IL-1, IL-1R, IL-2, IL-2R, IL-4, IL-4R, IL-5, IL-5R, IL-6, IL-6R, IL-8, IL-9, IL-10, IL-12, IL-13, IL-15, IL-18, IL-18R, IL-23, interferon (INF)-alpha, INF-beta, INF-gamma, Inhibin, iNOS, Insulin A-chain, Insulin B-chain, Insulin-like growth factor 1, integrin alpha2, integrin alpha3, integrin alpha4, integrin alpha4/beta1, integrin, alpha4/beta7, integrin alpha5 (alphaV), integrin alpha5/beta1, integrin alpha5/beta3, integrin alpha6, integrin beta1, integrin beta2, interferon gamma, IP-10, 1-TAC, JE, Kallikrein 2, Kallikrein 5, Kallikrein 6, Kallikrein 11, Kallikrein 12, Kallikrein 14, Kallikrein 15, Kallikrein LI, Kallikrein L2, Kallikrein L3, Kallikrein L4, KC, KDR, Keratinocyte Growth Factor (KGF), laminin 5, LAMP, LAP, LAP (TGF-1), Latent TGF-1, Latent TGF-1 bpl, LBP, LDGF, LECT2, Lefty, Lewis-Y antigen, Lewis-Y related antigen, LFA-1, LFA-3, Lfo, LIF, LIGHT, lipoproteins, LIX, LKN, Lptn, L-Selectin, LT-a, LT-b, LTB4, LTBP-1, Lung surfactant, Luteinizing hormone, Lymphotoxin Beta Receptor, Mac-1, MAdCAM, MAG, MAP2, MARC, MCAM, MCAM, MCK-2, MCP, M-CSF, MDC, Mer, METALLOPROTEASES, MGDF receptor, MGMT, MHC (HLA-DR), MIF, MIG, MIP, MIP-1-alpha, MK, MMAC1, MMP, MMP-1, MMP-10, MMP-11, MMP-12, MMP-13, MMP-14, MMP-15, MMP-2, MMP-24, MMP-3, MMP-7, MMP-8, MMP-9, MPIF, Mpo, MSK, MSP, mucin (Mucl), MUC18, Muellerian-inhibitin substance, Mug, MuSK, NAIP, NAP, NCAD, N-Cadherin, NCA 90, NCAM, NCAM, Neprilysin, Neurotrophin-3, -4, or -6, Neurturin, Neuronal growth factor (NGF), NGFR, NGF-beta, nNOS, NO, NOS, Npn, NRG-3, NT, NTN, OB, OGGI, OPG, OPN, OSM, OX40L, OX40R, p150, p95, PADPr, Parathyroid hormone, PARC, PARP, PBR, PBSF, PCAD, P-Cadherin, PCNA, PDGF, PDGF, PDK-1, PECAM, PEM, PF4, PGE, PGF, PGI2, PGJ2, PIN, PLA2, placental alkaline phosphatase (PLAP), P1GF, PLP, PP14, Proinsulin, Prorelaxin, Protein C, PS, PSA, PSCA, prostate specific membrane antigen (PSMA), PTEN, PTHrp, Ptk, PTN, R51, RANK, RANKL, RANTES, RANTES, Relaxin A-chain, Relaxin B-chain, renin, respiratory syncytial virus (RSV) F, RSV Fgp, Ret, Rheumatoid factors, RLIP76, RPA2, RSK, S100, SCF/KL, SDF-1, SERINE, Serum albumin, sFRP-3, Shh, SIGIRR, SK-1, SLAM, SLPI, SMAC, SMDF, SMOH, SOD, SPARC, Stat, STEAP, STEAP-II, TACE, TACI, TAG-72 (tumor-associated glycoprotein-72), TARC, TCA-3, T-cell receptors (e.g., T-cell receptor alpha/beta), TdT, TECK, TEM1, TEM5, TEM7, TEM8, TERT, testicular PLAP-like alkaline phosphatase, TfR, TGF, TGF-alpha, TGF-beta, TGF-beta Pan Specific, TGF-beta RI (ALK-5), TGF-beta RII, TGF-beta RIIb, TGF-beta RIII, TGF-beta1, TGF-beta2, TGF-beta3, TGF-beta4, TGF-beta5, Thrombin, Thymus Ck-1, Thyroid stimulating hormone, Tie, TIMP, TIQ, Tissue Factor, TMEFF2, Tmpo, TMPRSS2, TNF, TNF-alpha, TNF-alpha beta, TNF-beta2, TNFc, TNF-RI, TNF-RII, TNFRSF10A (TRAIL R1 Apo-2, DR4), TNFRSFIOB (TRAIL R2 DR5, KILLER, TRICK-2A, TRICK-B), TNFRSF10C (TRAIL R3 DcR1, LIT, TRID), TNFRSF10D (TRAIL R4 DcR2, TRUNDD), TNFRSF11A (RANK ODF R, TRANCE R), TNFRSF11B (OPG OCIF, TR1), TNFRSF12 (TWEAK R FN14), TNFRSF13B (TACI), TNFRSF13C (BAFF R), TNFRSF14 (HVEM ATAR, HveA, LIGHT R, TR2), TNFRSF16 (NGFR p75NTR), TNFRSF17 (BCMA), TNFRSF18 (GITR AITR), TNFRSF19 (TROY TAJ, TRADE), TNFRSF19L (RELT), TNFRSFIA (TNF RI CD120a, p55-60), TNFRSFIB (TNF RII CD120b, p75-80), TNFRSF26 (TNFRH3), TNFRSF3 (LTbR TNF RIII, TNFC R), TNFRSF4 (OX40 ACT35, TXGP1 R), TNFRSF5 (CD40 p50), TNFRSF6 (Fas Apo-1, APT1, CD95), TNFRSF6B (DcR3 M68, TR6), TNFRSF7 (CD27), TNFRSF8 (CD30), TNFRSF9 (4-1BB CD137, ILA), TNFRSF21 (DR6), TNFRSF22 (DcTRAIL R2 TNFRH2), TNFRST23 (DcTRAIL RI TNFRH1), TNFRSF25 (DR3 Apo-3, LARD, TR-3, TRAMP, WSL-1), TNFSF10 (TRAIL Apo-2 Ligand, TL2), TNFSF11 (TRANCE/RANK Ligand ODF, OPG Ligand), TNFSF12 (TWEAK Apo-3 Ligand, DR3 Ligand), TNFSF13 (APRIL TALL2), TNFSF13B (BAFF BLYS, TALL1, THANK, TNFSF20), TNFSF14 (LIGHT HVEM Ligand, LTg), TNFSF15 (TL1A/VEGI), TNFSF18 (GITR Ligand AITR Ligand, TL6), TNFSFIA (TNF-a Conectin, DIF, TNFSF2), TNFSF1B (TNF-b LTa, TNFSF1), TNFSF3 (LTb TNFC, p33), TNFSF4 (OX40 Ligand gp34, TXGP1), TNFSF5 (CD40 Ligand CD154, gp39, HIGM1, IMD3, TRAP), TNFSF6 (Fas Ligand Apo-1 Ligand, APT1 Ligand), TNFSF7 (CD27 Ligand CD70), TNFSF8 (CD30 Ligand CD153), TNFSF9 (4-1BB Ligand CD137 Ligand), TP-1, t-PA, Tpo, TRAIL, TRAIL R, TRAIL-RI, TRAIL-R2, TRANCE, transferring receptor, TRF, Trk, TROP-2, TSG, TSLP, tumor-associated antigen CA 125, tumor-associated antigen expressing Lewis Y related carbohydrate, TWEAK, TXB2, Ung, uPAR, uPAR-1, Urokinase, VCAM, VCAM-1, VECAD, VE-Cadherin, VE-cadherin-2, VEFGR-1 (flt-1), VEGF, VEGFR, VEGFR-3 (fit-4), VEGI, VIM, Viral antigens, VLA, VLA-1, VLA-4, VNR integrin, von Willebrands factor, WIF-1, WNT1, WNT2, WNT2B/13, WNT3, WNT3A, WNT4, WNT5A, WNT5B, WNT6, WNT7A, WNT7B, WNT8A, WNT8B, WNT9A, WNT9A, WNT9B, WNT10A, WNT10B, WNT11, WNT16, XCL1, XCL2, XCRI, XCRI, XEDAR, XIAP, XPD, CTLA4 (cytotoxic T lymphocyte antigen-4), PD1 (programmed cell death protein 1), PD-L1 (programmed cell death ligand 1), LAG-3 (lymphocyte activation gene-3), TIM-3 (T cell immunoglobulin and mucin protein-3), receptors for hormones, and growth factors.

In particular embodiments, combinations of antigens that may be targeted by a bispecific (or multispecific) antibody may include but are not limited to: CD3 and Her2; CD3 and Her3; CD3 and EGFR; CD3 and CD19; CD3 and CD20; CD3 and EpCAM; CD3 and CD33; CD3 and PSMA; CD3 and CEA; CD3 and gp100; CD3 and gpA33; CD3 and B7-H3; CD64 and EGFR; CEA and HSG; TRAIL-R2 and LTbetaR; EGFR and IGFR; VEGFR2 and VEGFR3; VEGFR2 and PDGFR alpha; PDGFRalpha and PDGFR beta; EGFR and MET; EGFR and EDV-miR16; EGFR and CD64; EGFR and Her2; EGFR and Her3; Her2 domain ECD2 and Her2 domain ECD4; Her2 and Her3; IGF-1R and HER3; CD19 and CD22; CD20 and CD22; CD30 and CD16A; FceRI and CD32B; CD32B and CD79B; BCMA and HEL; MP65 and SAP-2; IL-17A and IL-23; IL-1alpha and IL-1beta; IL-12 and IL-18; VEGF and osteopontin; VEGF and Ang-2; VEGF and PDGFRbeta; VEGF and Her2; VEGF and DLL4; FAP and DR5; FcgRII and IgE; CEA and DTPA; CEA and IMP288; and LukS-PV and LukF-PV.

A “different antigen” may refer to different and/or distinct proteins, polypeptides, or molecules; as well as different and/or distinct epitopes, which epitopes may be contained within one protein, polypeptide, or molecule. Consequently, a bispecific antibody may bind to two epitopes on the same polypeptide.

The term “epitope” is used herein in the broadest sense and encompasses a region or regions of an antigen interacting with a corresponding paratope. Protein or peptide epitopes may include amino acid residues interacting directly with a paratope (e.g., through hydrogen bonding or hydrophobic interactions) and amino acid residues that do not (e.g., those residues contributing generally to epitope conformation). Epitopes may be defined as structural and/or functional. Functional epitopes are generally epitopes with residues directly contributing to some function of the antigen (e.g., affinity for another protein or enzymatic activity). Structural epitopes are epitopes with residues contributing to antigen structure that may not significantly contribute to antigen function. Epitopes may also be conformational, that is, composed of non-linear amino acids. In certain embodiments, epitopes may include determinants that are chemically active surface groupings of molecules such as amino acids, sugar side chains, phosphoryl groups, or sulfonyl groups, and, in certain embodiments, may have specific three-dimensional structural characteristics, and/or specific charge characteristics. A single antigen may have more than one epitope. Thus, different antibodies may bind to different areas on an antigen and may have different biological effects. Multi-specific antibodies may include multiple antigen-binding sites that bind to different epitopes of the same antigen. Bispecific antibodies binding to different epitopes of the same antigen are referred to herein as “biparatopic” antibodies. The term “epitope” also refers to a site on an antigen to which B and/or T cells respond. It also refers to a region of an antigen that is bound by an antibody.

In some instances, a full-size antibody comprises four polypeptide chains: two heavy (H) chains and two light (L) chains interconnected by disulfide bonds. Each heavy chain comprises a variable region, such as a heavy chain variable region (“VH”) (also referred to as heavy chain variable domain), and a heavy chain constant region (“CH”). In case of an intact antibody, a CH comprises domains CH1, CH2 and CH3. In case of an antibody fragment, a CH may comprise CH1, CH2, and/or CH3 domains, and in some preferred embodiments, the CH comprises at least a CH1 domain. The variant CH3 domains disclosed herein may be used in combination with one or more wild-type CH2 and/or CH3 domains or CH2 and/or CH3 domains comprising one or more amino acid substitutions, e.g., those that alter or improve antibodies' stability and/or effector functions. Each light chain comprises a variable region, such as a light chain variable region (“VL”) (also referred to as light chain variable domain), and a light chain constant region (“CL”). The VH and VL regions, can be further subdivided into regions of hypervariability, termed complementarity determining regions (CDRs), interspersed with regions that are more conserved, termed framework regions (FRs). Each VH and VL comprises three CDRs and four FRs, arranged from amino-terminus to carboxy-terminus in the following order: FR1, CDR1, FR2, CDR2, FR3, CDR3, FR4. In certain embodiments of the disclosure, the FRs of the antibody (or antigen-binding fragment thereof) may be identical to the human germline sequences or may be naturally or artificially modified. An amino acid consensus sequence may be defined based on a side-by-side analysis of two or more CDRs. Accordingly, the CDRs in a heavy chain are designated “CDRH1”, “CDRH2”, and “CDRH3”, respectively, and the CDRs in a light chain are designated “CDRL1”, “CDRL2”, and “CDRL3”. In other instances, an antibody may comprise multimers thereof (e.g., IgM) or antigen-binding fragments thereof.

The numbering of amino acid residues in antibody variable and constant domains may be performed by the EU-index or EU numbering system, as described in Kabat et al., Sequences of Proteins of Immunological Interest, 5th Ed. Public Health Service, National Institutes of Health, Bethesda, Md. (1991). The EU numbering system is used in the present specification unless otherwise specified.

According to IMGT (the international ImMunoGeneTics information system for immunoglobulins or antibodies, T cell receptors, MH, immunoglobulin superfamily IgSF and MhSF), the CH1 domain is the amino acid positions (or simply referred to as “positions” herein) 118-215 (EU numbering) and the hinge region is the amino acid positions 216-230 (EU numbering). The term “CH1 domain” is used in a broad sense herein to refer to a heavy chain region comprising at least seven consecutive amino acid positions of the heavy chain positions 118-215 (EU numbering)) and in some instances also comprising a portion of the hinge region (a portion of heavy chain positions 216-230 (EU numbering)) is included (e.g., up to position 218). A CH1 domain reference sequence, corresponding to the amino acid positions 118-220 according to EU numbering, is provided herein as SEQ ID NO: 6, which corresponds to the CH1 domain sequence of human IgG1 Allotype “IGHG1*01 (J00228)”, “IGHG1*04 (JN582178)”, or “IGHG1*07” and is an exemplary amino acid sequence of a wild-type (WT) CH1 domain.

### CH1 Domain Reference Sequence:

(SEQ ID NO: 6)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKKV
EPKSC (positions 118-220 according to EU
numbering)

Alternative CH1 domain reference sequences of human IgG1 include but are not limited to SEQ ID NO: 5, which corresponds to the CH1 domain sequence of human IgG1 Allotype “IGHG1*03 (Y14737)” or “IGHG1*08”.

Alternative CH1 domain reference sequence (214R relative to SEQ ID NO: 6):

(SEQ ID NO: 5)
ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSC (positions 118-220 according to EU
numbering)

These CH1 domain reference sequences are intended to be exemplary as Applicant intends for “CH1 domain” sequences to include any naturally occurring CH1 domain allotype or allelic variant.

According to IMGT, the CH2 domain is the amino acid positions (or simply referred to as “positions” herein) 231-340 (EU numbering). The term “CH2 domain” is used in a broad sense herein to refer to a heavy chain region comprising at least seven consecutive amino acid positions of the heavy chain positions 231-340 (EU numbering)). A CH2 domain reference sequence, corresponding to the amino acid positions 231-340 according to EU numbering, is provided herein as SEQ ID NO: 7, which is an exemplary amino acid sequence of a wild-type (WT) CH2 domain.

### CH2 Domain Reference Sequence:

(SEQ ID NO: 7)
APELLGGPSVFLFPPKPKDTLMISRTPEVTCVVVDVSHEDPEVKFNWYV
DGVEVHNAKTKPREEQYNSTYRVVSVLTVLHQDWLNGKEYKCKVSNKAL
PAPIEKTISKAK.

Again the recited CH2 domain reference sequence is intended to be exemplary as Applicant intends for “CH2 domain” sequences to include any naturally occurring CH2 domain allotype or allelic variant.

According to IMGT, the CH3 domain is the amino acid positions (or simply referred to as “positions” herein) 341-446 (EU numbering). The term “CH3 domain” is used in a broad sense herein to refer to a heavy chain region comprising at least seven consecutive amino acid positions of the heavy chain positions 341-446 (EU numbering)). A CH3 domain reference sequence, corresponding to the amino acid positions 341-446 according to EU numbering, is provided herein as SEQ ID NO: 1, which corresponds to the CH3 domain sequence of human IgG1 Allotype “IGHG1*01 (J00228)” or “IGHG1*08” and is an exemplary amino acid sequence of a wild-type (WT) CH3 domain.

### CH3 Domain Reference Sequence:

(SEQ ID NO: 1)
GQPREPQVYTLPPSRDELTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

Alternative CH3 domain reference sequences of human IgG1 may include but are not limited to SEQ ID NO: 2, which corresponds to the CH3 domain sequence of human IgG1 Allotype “IGHG1*03 (Y14737)”, SEQ ID NO: 3, which corresponds to the CH3 domain sequence of human IgG1 Allotype “IGHG1*04 (JN582178)”, and SEQ ID NO: 4, which corresponds to the CH3 domain sequence of human IgG1 Allotype “IGHG1*07”.

Alternative CH3 domain reference sequence (356E and 358M relative to SEQ ID NO: 1):

(SEQ ID NO: 2)
GQPREPQVYTLPPSREEMTKNQVSLTCLVKGFYPSDIAVEWESNGQPEN
NYKTTPPVLDSDGSFFLYSKLTVDKSRWQQGNVFSCSVMHEALHNHYTQ
KSLSLSPG.

Alternative CH3 domain reference sequence (422I relative to SEQ ID NO: 1):

(SEQ ID NO: 3)
GQPREPQVYTLPPSRDELTKNQVSLTCLVKGFYPSDIAVEWESNGQPEN
NYKTTPPVLDSDGSFFLYSKLTVDKSRWQQGNIFSCSVMHEALHNHYTQ
KSLSLSPG.

Alternative CH3 domain reference sequence (431G relative to SEQ ID NO: 1):

(SEQ ID NO: 4)
GQPREPQVYTLPPSRDELTKNQVSLTCLVKGFYPSDIAVEWESNGQPEN
NYKTTPPVLDSDGSFFLYSKLTVDKSRWQQGNVFSCSVMHEGLHNHYTQ
KSLSLSPG.

Furthermore, CH3 domain reference sequences of human IgG2, IgG3, and IgG4 include but are not limited to SEQ ID NOS: 722, 723, and 724, respectively.

### CH3 Domain Reference Sequence of Human IgG2:

(SEQ ID NO: 722)
GQPREPQVYTLPPSREEMTKNQVSLTCLVKGFYPSDISVEWESNGQPEN
NYKTTPPMLDSDGSFFLYSKLTVDKSRWQQGNVFSCSVMHEALHNHYTQ
KSLSLSPG

### CH3 Domain Reference Sequence of Human IgG3:

(SEQ ID NO: 723)
GQPREPQVYTLPPSREEMTKNQVSLTCLVKGFYPSDIAVEWESSGQPEN
NYNTTPPMLDSDGSFFLYSKLTVDKSRWQQGNIFSCSVMHEALHNRFTQ
KSLSLSPG

### CH3 Domain Reference Sequence of Human IgG4:

(SEQ ID NO: 724)
GQPREPQVYTLPPSQEEMTKNQVSLTCLVKGFYPSDIAVEWESNGQPEN
NYKTTPPVLDSDGSFFLYSRLTVDKSRWQEGNVFSCSVMHEALHNHYTQ
KSLSLSLG

These CH3 domain reference sequences are intended to be exemplary as Applicant intends for “CH3 domain” sequences to include any naturally occurring CH3 domain allotype or allelic variant.

Accordingly, an amino acid modification(s) in variant CH3 domain polypeptides according to the present disclosure may be relative to and/or incorporated to any parent CH3 domain polypeptides, for example but not limited to a wild-type sequence, such as SEQ ID NO: 1 or any allelic variants thereof, such as SEQ ID NO: 2, 3, or 4, or 722, 723, or 724.

There are two major CL isotypes, kappa (“κ”) and lambda (“λ”), and such CL domains are referred to herein as kappa CL domain (“CLκ” domain) and lambda CL domain (“CLλ” domain).

According to IMGT, the CLκ domain is the amino acid positions 108-214 (EU numbering). The term “CLκ domain” is used in a broad sense herein to refer to a light chain region comprising at least seven consecutive amino acid positions of the kappa light chain positions 108-214 (EU numbering). A CLκ domain reference sequence, corresponding to the amino acid positions 108-214 (EU numbering), is provided herein as SEQ ID NO: 8, which is an exemplary amino acid sequence of a wild-type (WT) CLκ domain.

### CLκ Domain Reference Sequence:

(SEQ ID NO: 8)
RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC (positions 108 to 214 according to EU
numbering).

According to IMGT, the CLλ domain is the amino acid positions 107-215 (EU numbering). The term “CLλ domain” is used in a broad sense herein to refer to a light chain region comprising at least seven consecutive amino acid positions of the lambda light chain positions 107-215 (EU numbering). A CLλ domain reference sequence, corresponding to the amino acid positions 107-215 (EU numbering), is provided herein as SEQ ID NO: 9, which is an exemplary amino acid sequence of a wild-type (WT) CLλ domain.

### CLλ Domain Reference Sequence:

(SEQ ID NO: 9)
GQPKAAPSVTLFPPSSEELQANKATLVCLISDFYPGAVTVAWKADSSPV
KAGVETTTPSKQSNNKYAASSYLSLTPEQWKSHRSYSCQVTHEGSTVEK
TVAPTECS (positions 107 to 215 according to EU
numbering).

Various standard sequences (corresponding to different allotypes) of the constant domains of human IgG1, IgG2, IgG3, and IgG4 are known in the field and may be found for example in Vidarsson et al., Front Immunol. 2014 Oct. 20; 5:520 and U.S. Pat. No. 9,150,663, the disclosures of which are hereby incorporated by reference herein in their entirety herein. Again these reference sequences are intended to be exemplary as Applicant intends for human IgG1, IgG2, IgG3, and IgG4 sequences to include any naturally occurring human IgG1, IgG2, IgG3, and IgG4 allotype.

The term “cognate pair” or “cognate pairing” used herein refers to a pair or pairing of two antibody chains (e.g., a heavy chain and a light chain), each containing a variable region (e.g., a VH and a VL, respectively), in which the combination of the variable regions provides intended binding specificity to an epitope or to an antigen. The term “non-cognate pair” or “non-cognate pairing” used herein refers to a pair or pairing of two antibody chains (e.g., a heavy chain and a light chain) each containing a variable region (e.g., a VH and a VL, respectively), in which the combination of the variable regions does not provide intended binding specificity to an epitope or to an antigen.

Provided herein are engineered variant CH3 domains containing at least one amino acid substitution that prevents or reduces formation of CH3-CH3 homodimers and preferentially forms CH-CH3 homodimers.

The term “CH3 domain set” or “CH3 set” are used interchangeably to refer to a combination of two CH3 domains. When a CH3 set comprises two non-wildtype CH3 domain (i.e., two variant CH3 domains), such a CH3 set may also be referred to as a “variant CH3 domain set” (or “CH3 domain variant set”) or a “variant CH3 set” (or “CH3 variant set”). “CH3 Set Name” is given to each “CH3 set” based on the amino acid substitutions contained in the CH3 domains of the set. The set of the substitutions included in the CH3 domains of a set may be referred to as “CH3 substitution set”.

The “CH3 Set Names” used herein are named by the amino acid positions (according to EU numbering) substituted in the CH3 domain of each chain with a dash to separate heavy chains. For example, the “W-SG” set has W in the CH3 domain (at position 366) of a first heavy chain (referred to as Chain A in FIG. 19 and Appendix Tables), along with S and G in the CH3 domain (at positions 366 and 407) of a second heavy chain (referred to as Chain B in FIG. 19 and Appendix Tables). The two chains are referred to as Chain A and Chain B in FIG. 19 and Appendix Tables, but the chain names are interchangeable. For example, the “W-SG” set may have W in the CH3 domain (at position 366) of a second heavy chain (or “Chain B”), along with S and G in the CH3 domain (at positions 366 and 407) of the first heavy chain (or “Chain A”). “(349/354)” and “(354/349)” refer to the inter-CH3 domain disulfide bond-allowing substitutions. For example, “(349/354)” indicates the presence of the Y349C substitution in the CH3 of Chain A and S354C substitution in the CH3 of Chain B. Similarly, “(354/349)” indicates the presence of the S354C substitution in the CH3 of Chain A and Y349C substitution in the CH3 of Chain B. Therefore, “W-SG (354/349)” means that the S354C substitution is present in the CH3 which has the “W” substation (at position 366) and the Y349C substitution is present in the CH3 which has the “SG” substitutions (at positions 366 and 407).

As used herein, the term “variant CH3 domain” (also referred to as “variant CH3 domain polypeptide”, “CH3 domain variant”, or “CH3 domain variant polypeptide”) are used interchangeably to refer to a CH3 domain which has an amino acid sequence in which one or more amino acid substitutions are made to a CH3 domain sequence. The CH3 sequence to which an amino acid substitution(s) are made include but is not limited to the reference CH3 domain sequence SEQ ID NO: 1. In the libraries screened to identify the described variant CH3 domains, the nucleic acid sequence encoding SEQ ID NO: 1 was variegated. The term “Fab-arm exchange” or “FAE” as used herein refer to the process in which a half molecule (i.e., a pair of one heavy chain and one light chain, also referred to as a “half antibody” or “half IgG” when the antibody is an IgG) of an Ig molecule (e.g., IgG, IgE, or IgD) recombine with another half molecule of another Ig molecule. FAE was originally found to naturally occur in human IgG4 molecules and that FAE may be mimicked in vitro by the addition of mild reducing agents (van der Neut Kolfschoten et al. Science. 2007 Sep. 14; 317(5844):1554-1557). Site-directed mutagenesis studies replacing IgG4 amino acid residues with their IgG1 counterpart residues identified that FAE in humans may be driven by residues S228 located in the IgG4 core hinge (van der Neut Kolfschoten et al. Science. 2007 Sep. 14; 317(5844):1554-1557; Labrijn et al. Nat Biotechnol. 2009 August; 27(8):767-771.) and R409 in the IgG4 CH3 domain (Labrijn et al. J Immunol. 2011 Sep. 15; 187(6):3238-46.). Later it was discovered that an IgG1 CH3 domain in which position 409 is substituted to R and another IgG1 CH3 domain in which position 405 is substituted to L preferentially pair with each other and that use of such CH3 may be useful for manufacturing bispecific IgG1 molecules (Labrijn et al. Proc Natl Acad Sci USA. 2013 Mar. 26; 110(13):5145-50).

The term “controlled FAE” or “cFAE” as used herein refers to FAE that is artificially promoted by a set of engineered CH3 domains that preferentially form heterodimers. cFAE may be particularly useful for efficiently manufacturing bispecific antibodies. For example, when an antibody of interest comprises: (a) a half antibody specific to epitope A, comprising a heavy chain A (comprising a VH) and a light chain A (comprising a VL); and (b) a half antibody specific to epitope B, comprising a heavy chain B (comprising a VH) and a light chain B (comprising a VL), (a) antibody A comprising two of the half antibody specific to epitope A and (b) antibody B comprising two of the half antibody specific to epitope B may be first produced. Antibodies A and B may be then placed together under a mildly reducing condition, which allows for reduction of disulfide bonds between heavy chains, resulting in half antibody molecules. If heavy chain A comprises an engineered CH3 domain A and heavy chain B comprises an engineered CH3 domain B and CH3 domains A and B preferentially form CH3-CH3 heterodimers, upon removal of the mildly reducing condition, heterodimers between heavy chains A and B are formed preferentially over heavy chain A homodimers and heavy chain B homodimers due to cFAE, resulting in more of the bispecific antibody of interest than antibodies A and B.

The term “half molecule” or “half antibody” when referring to IgG, IgE, or IgD, which may also be referred to as “half IgG”, “half IgE”, or “half IgD”, respectively, refers to a set of one heavy chain and one light chain of the referenced antibody.

By “preferentially” form heterodimers, or “preferential” formation of heterodimers when referring to a CH3 domain, it is meant that formation of a heterodimer with another, non-identical CH3 domain occurs more (i.e., more frequently or at a higher chance) than formation of a homodimer with another, identical CH3 domain. When referring to a set of two CH3 domains different from each other (a first CH3 domain and a second CH3 domain), it is meant that more heterodimers (of the first and second CH3 domains) are formed than homodimers (dimers of first CH3 domains and dimers of second CH3 domains). For example, when a first CH3 domain and a second CH3 domain different from the first CH3 domain are mixed or co-expressed or co-provided at approximately 1:1 ratio, the % dimers formed between the first and second CH3 domains among CH3 dimers is higher than 50%. The % CH-CH3 heterodimers (also referred to as, e.g., % heterodimer” or “% heterodimers”) or the degree of heterodimerization may be quantified by any available assays, such as but not limited to, AlphaLISA®, liquid chromatography-mass spectrometry (LC-MS), ion exchange chromatography (IEX), or flow cytometry. The % heterodimers when the CH3 domains comprising the CH3 substitution sets disclosed here may be about 55%, about 60%, about 65%, about 70%, about 75%, about 80%, about 85%, about 90%, about 95%, about 96%, about 97%, about 98%, about 99%, or about 100%. In some preferred embodiments, the % heterodimers may be about 70% or higher. In some more preferred embodiments, the % heterodimers may be about 75% or higher. In some more preferred embodiments, the % heterodimers may be about 80% or higher. In some more preferred embodiments, the % heterodimers may be about 85% or higher. In some more preferred embodiments, the % heterodimers may be about 90% or higher. In some more preferred embodiments, the % heterodimers may be about 95% or higher. In some more preferred embodiments, the % heterodimers may be 90%, 91%, 92%, 93%, 94%, 95%, 96%, 97%, 98% or 99%. In some more preferred embodiments, the % heterodimers may be about 100%.

The CH3 substitution sets or CH3 sets and/or antibodies comprising such a CH3 set may be further evaluated based on an additional property or properties, such as but not limited to: the degree of aggregation (e.g., presence of multimers of a full antibody) and/or the amount of half antibody (i.e., one CH3 in a molecule or one heavy chain in a molecule), both of which may be quantified by, e.g., chromatography such as size exclusion chromatography (SEC) or electrophoresis such as SDS-PAGE; melting temperature (Tm), which may be measured by, e.g., Differential scanning fluorimetry (DSF); production yields in an appropriate cell type (e.g., HEK293 cells or yeast cells); “pI”, isoelectric point (“pI”); the level of interaction with polyspecificity reagent (“PSR”), which may be measured as in WO2014/179363; hydrophobic interaction of the antibody which may be measured by hydrophobic interaction chromatography (“HIC”) as measured as in e.g., Estep P, et al. MAbs. 2015 May-June; 7(3): 553-561.; solubility; production costs and/or time; stability; shelf life; in vivo half-life; and/or immunogenicity. Any of these or other properties may be used in addition to % heterodimer values when assessing a given variant CH3 domain set or a CH3 set. Therefore, a variant CH3 domain or CH3 set which gives relatively lower % heterodimer may just as ideal as another CH3 set with a relatively higher % heterodimer value, if the variant CH3 domain or CH3 set provides a good profile on one or more properties. For example, a CH3 set which gives 80% heterodimers with 3% aggregation (3% of the expression products are multimers of a full antibody) may be just as ideal as a CH3 set which gives 90% heterodimers with 10% aggregation.

There are five major classes of antibodies: IgA, IgD, IgE, IgG, and IgM, and several of these may be further divided into subclasses (isotypes), e.g., IgG1, IgG2, IgG3, IgG4, IgA1, and IgA2. The heavy chain constant domains that correspond to the different classes of immunoglobulins are called u, 6, E, y, and p, respectively. The constant domains according to the present disclosure may be of any antibody isotype, e.g., IgG1, IgG2, IgG3, IgG4, IgA1, IgA2, IgD, IgM, and IgE. The CH3 domain, as used herein, may be derived from CH3 of antibody isotypes, e.g., IgG1, IgG2, IgG3, IgG4, IgA1, IgA2, IgD, IgM, and IgE. The CH3 substitution(s) according to the present disclosure may be made to any CH3 domain sequences, such as but not limited to the CH3 reference sequence SEQ ID NO: 1. When CH1 and/or CH2 domain(s) are used with the variant CH3 domain of the present disclosure, the CH1 and/CH2 domain(s) may be derived from any antibody isotypes and the CH1 and/or CH2 domain isotype(s) does not necessarily need to be the same as the CH3 domain isotype.

A “library” is used herein to encompass any collections of biological materials such as nucleic acids, peptides, proteins, and sequence information thereof. For example, a “CH3 domain-encoding polynucleotide library” refers to a collection of polynucleotides encoding different CH3 domain polypeptides or of the polynucleotide sequences thereof; and a “CH3 domain polypeptide library” refers to a collection of different CH3 domain polypeptides or of the amino acid sequences thereof.

The term “linker” refers to a construct of variable length connecting two or more domains or portions of a polypeptide or connecting two or more polypeptides. In some cases, a linker is used to confer flexibility, improved spatial organization, proximity, etc and in such a case may be referred to as a flexible linker. Exemplary linkers may comprise one or more amino acids, optionally between 1-50 amino acids, such as one, two, three, four, five, six, seven, eight, nine, ten, eleven, twelve, thirteen, fourteen, fifteen, sixteen, seventeen, eighteen, nineteen, or twenty amino acids. In some embodiments, the linker may predominantly or entirely consist of of G, S, and/or A amino acid residues. In some embodiments, the linker may comprise an amino acid sequence which comprises or consists of the amino acid sequence selected from the group consisting of GGGGS (SEQ ID NO: 715, which may also be called “G4S”), GGGS (SEQ ID NO: 716, which may also be called “G3S”), GGGGGS (SEQ ID NO: 717, which may also be called “G5S”), G, GG, GGG, GS, SG, GGS (which may also be called “G2S”), GSG, SGG, GSS, SGS, and SSG. In some embodiments, the linker may comprise an amino acid sequence which comprises or consists of multiple repeats (e.g., two, three, four, five, or more repeats) of the amino acid sequence selected from the group consisting of SEQ ID NO: 101, SEQ ID NO: 102, SEQ ID NO: 103, G, GS, SG, GGS, GSG, SGG, GSS, SGS, and SSG. When the linker comprises or consists of multiple repeats of G5S, G4S, G3S, G2S, GS, or G, the linker may optionally called a (G5S)n linker, a (G4S)n linker, a (G3S)n linker, a (G2S)n linker, a (GS)n linker, or a (G)n linker, respectively (n is a natural number, optionally selected from 1-20, e.g., 2, 3, 4, 5, etc). In particular embodiments, the linker may comprise two or three repeats of SEQ ID NO: 101, i.e., have the sequence of GGGGSGGGGS (SEQ ID NO: 718) or GGGGSGGGGSGGGGS (SEQ ID NO: 719), respectively, and may optionally be called a (G4S)2 linker or a (G4S)3 linker, respectively.

A “pharmaceutical carrier”, as used herein, includes any and all solvents, dispersion media, coatings, antibacterial and antifungal agents, isotonic, and absorption delaying agents that are physiologically compatible. In one embodiment, the carrier is suitable for parenteral, intravenous, intraperitoneal, intramuscular, or sublingual administration. Pharmaceutically acceptable carriers include sterile aqueous solutions or dispersions and sterile powders for the extemporaneous preparation of sterile injectable solutions or dispersions. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions. In some embodiments, the carrier may be a liquid, in which an active therapeutic agent is formulated. The excipient generally does not provide any pharmacological activity to the formulation, though it may provide chemical and/or biological stability, and release characteristics. Exemplary formulations can be found, for example, in Remington's Pharmaceutical Sciences, Gennaro, A. editor, 19th edition, Philadelphia, PA: Williams and Wilkins (1995), which is incorporated by reference.

The term “scFv,” “single-chain Fv,” or “single-chain variable fragment” refers to a fusion protein comprising at least one heavy chain variable region (VH) and at least one light chain variable region (VL) of an antibody, wherein the VH and the VL are contiguously linked and wherein the scFv retains the specificity of the antibody from which it is derived (the antibody from which the VH and the VL are derived). Unless specified, as used herein an scFv may have the VH and the VL in either order, e.g., with respect to the N-terminal and C-terminal ends of the polypeptide. For example, the VH and the VL may be linked via a linker, such as a synthetic and/or flexible polypeptide linker, and the scFv may be capable of being expressed as a single chain polypeptide. When a linker connects the VH and the VL, a scFv may comprise the structure of VL-linker-VH or VH-linker-VL. When a linker connects the VH and the VL, the linker may be any appropriate linker such as but not limited to any of the linkers described herein. In some cases, the VH and the VL are additionally or alternatively connected by one or more disulfide bonds. In certain cases, the VH and/or the VL sequences may be modified (e.g., one or more amino acids may be substituted) to comprise a cysteine residue to allow for such a disulfide bond (e.g., see Weatherill et al., Protein Eng Des Sel. 2012 July; 25(7):321-9.).

“Conservative amino acid substitutions” are known in the art and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, Ile, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gln, Ser, Thr, Tyr, etc.), an amino acid with a β-branched side-chain substituted for another amino acid with a β-branched side-chain (e.g., Ile, Thr, and Val), an amino acid with an aromatic side-chain substituted for another amino acid with an aromatic side chain (e.g., His, Phe, Trp, and Tyr), etc.

### Variant CH3 Domains and CH3 Domain Sets

As described herein, certain positions within the CH3 domain and certain amino acid substitution(s) and substitution sets were found to influence the pairing of CH3 domains or formation of CH3-CH3 dimers (or Fc heterodimers).

In some embodiments, the variant CH3 domains described herein may contain an amino acid substitution(s) at one or more of the following amino acid positions: 364, 366, 368, 370, 399, 400, 405, 407, and 409, according to EU numbering. In some embodiments, the variant CH3 domains described herein may contain an amino acid substitution(s) at any of the positions listed in Table 7 or at any of the position combinations listed in Table 7.

The parent CH3 domain to which such an amino acid substitution(s) may be incorporated may comprise a wild-type or naturally occurring CH3 domain sequence or a variant or engineered version thereof. An exemplary sequence of such a parent polypeptide includes but is not limited to the reference CH3 sequence SEQ ID NO: 1, corresponding to amino acid positions 341 to 446 according to EU numbering.

The resultant variant CH3 domains preferentially form a CH3-CH3 heterodimer over a CH3-CH3 homodimer. Such variant CH3 domains may be useful in producing heterodimeric (or multimeric) polypeptides and molecules comprising such polypeptides. Such variant CH3 domains may be useful in producing multi-specific antibodies and antibody fragments, by improving the fidelity of heterologous Fc pairing while maintaining the native IgG structure of a bispecific antibody, which is favorable due to its well-established properties as a therapeutic molecule, including a long in vivo half-life and the ability to elicit effector functions. These variant CH3 domains may be used to solve, in whole or in part, chain mispairing when generating multispecific, e.g., bispecific, antibodies by promoting proper heavy chain-heavy chain pairing. More specifically, multispecific antibodies comprising these variant CH3 domains will form fewer unwanted product-related contaminants, i.e., molecules containing mis-paired domains or chains, whose elimination during manufacturing can be challenging.

In some embodiments, the amino acid substitution(s) in the variant CH3 domains may comprise or consist of an amino acid substitution(s) at: (i) position 366; (ii) position 368; (iii) position 407; (iv) positions 366 and 407; (v) positions 366 and 368; (vi) positions 366 and 409; (vii) positions 368 and 370; (viii) positions 368 and 407; (ix) positions 399 and 405; (x) positions 400 and 409; (xi) positions 364, 366, and 409; (xii) positions 364, 407, and 409; (xiii) positions 366, 368, and 370; (xiv) positions 366, 368, and 407; (xv) positions 366, 399, and 405; (xvi) positions 366, 400, and 409; (xvii) positions 366, 407, and 409; (xviii) positions 368, 400, and 409; (xix) positions 399, 405, and 407; (xx) positions 400, 407, and 409; (xxi) positions 366, 399, 405, and 407; (xxii) positions 366, 399, 405, and 409; (xxiii) positions 366, 400, 407, and 409; (xxiv) positions 366, 368, 399, 405, and 407; or (xxv) positions 366, 368, 400, 407, and 409.

In some embodiments, a variant CH3 domain may further comprise the Y349C or S354C substitution, which allows for a disulfide formation with another variant CH3 domain comprising the S354C or Y349C substitution, respectively. In some embodiments, a variant CH3 domain may comprise one or more of the following amino acid substitutions: S364D; S364L; T366Q; T366R; T366S; T366V; T366W; L368A; L368F; L368S; L368I; K370G; K370Y; D399Q; S400T; F405L; Y407V; Y407G; K409R; K409L; and/or K409G. In some embodiments, the variant CH3 domain may optionally further comprising Y349C or S354C.

In some embodiments, the amino acid substitution(s) in a variant CH3 domain may comprise or consist of any one of the following substitution combinations: T366W; T366S and Y407G; T366V; Y407V; T366Q and K409R; L368F; T366R and K409G; L368F and K370G; S400T and K409L; D399Q and F405L; S364D, Y407V, and K409G; T366V, L368S, and K370Y; S364L, T366W, and K409G; T366S, L368I, and Y407G; T366W, S400T, and K409L; T366S, L368A, Y407V, D399Q, and F405L; T366W, S400T, and K409L; T366S, Y407G, D399Q, and F405L; T366W, D399Q, and F405L; T366S, L368A, Y407V, S400T, and K409L; T366W, D399Q, and F405L; T366S, Y407G, S400T, and K409L; Y407V, S400T, and K409L; T366V, D399Q, and F405L; Y407V, D399Q, and F405L; T366V, S400T, and K409L; T366Q, K409R, D399Q, and F405L; L368F, S400T, and K409L; Y407V, T366Q, and K409R; T366V and L368F; S364L, T366W, and K409G; or L368I and Y407G. In some embodiments, the amino acid substitution(s) in a variant CH3 domain may comprise or consist of any one of the following substitution combinations: T366W; T366S and Y407G; T366V; Y407V; T366Q and K409R; L368F; T366R and K409G; L368F and K370G; S400T and K409L; D399Q and F405L; S364D, Y407V, and K409G; T366V, L368S, and K370Y; S364L, T366W, and K409G; T366S, L368I, and Y407G; or L368I and Y407G. In some embodiments, the S354C or Y349C substitution may be further added to any of the substitution combinations.

In some embodiments, these substitutions are made to the reference CH3 domain sequence of SEQ ID NO: 1. In such cases, the amino acid sequence of a variant CH3 domain according to the present disclosure may comprise or consist of the sequence in any one of SEQ ID NOS: 11-16, 21-26, 31-36, 41-46, 51-56, 61-66, 71-76, 81-86, 91-96, 101-106, 111-116, 121-126, 131-136, 141-146, 151-156, and 161-166. In some embodiments, a variant CH3 domain according to the present disclosure may comprise or consist of the sequence in any one of SEQ ID NOS: 11-16, 21-26, 31-36, 41-46, 51-56, 61-66, 71-76, and 161-166. In some preferred embodiments, the variant CH3 domain may comprise the amino acid sequence according to any one of SEQ ID NOS: 11-16 and 71-76.

In some embodiments, the variant CH3 domain according to the present disclosure may be paired with or form a heterodimer with another variant CH3 domain according to the CH3 domain disclosed herein.

The variant CH3 domain sets according to the present disclosure that preferentially form CH3-CH3 heterodimers are not identical to those previously identified as heterodimerization-preferring CH3 domain sets, such as the pre-existing CH3 technologies listed in Table 1. However, any of the inventive CH3 substitution sets described herein may be combined with the pre-existing CH3 technologies such as those in Table 1.

TABLE 1
Pre-existing CH3 heterodimerization technologies.

| CH3 Set Name | Alt. Name | CH3-1 | CH3-2 | Ref. patent |
|---|---|---|---|---|
| W-SAV | KiH | T366W | T366S, L368A, | U.S. Pat. No. 5,731,168 |
| | | | Y407V | (Genentech) |
| HA-TF | | S364H, F405A | Y349T, T394F | U.S. Pat. No. 10,472,427 |
| | | | | (Xencor) |
| VYAV-VLLW | ZW1 | T350V, L351Y, | T350V, T366L, | U.S. Pat. No. 9,499,634 |
| | | F405A, Y407V | K392L, T394W | (Zymeworks) |
| DMA-RRVV | 7.8.60 | K360D, D399M, | E345R, Q347R, | U.S. Pat. No. 10,774,156 |
| | | Y407A | T366V, K409V | (UNC/Eli Lilly) |
| SYMV-GDQA | 20.8.34 | Y349S, K370Y, | E356G, E357D, | U.S. Pat. No. 10,774,156 |
| | | T366M, K409V | S364Q, Y407A | (UNC/Eli Lilly) |
| DD-KK | | K392D, K409D | E356K, D399K | U.S. Pat. No. 8,592,562 |
| | | | | (Amgen) |
| EW-RVT | | K360E, K409W | Q347R, D399V, | U.S. Pat. No. 9,951,145 |
| | | | F405T | (Ajou University) |

* The CH3 set names as used herein are named by the amino acid positions (according to EU numbering) substituted in the CH3 domain of each chain, with a dash to separate chains. For example, the W-SAV set has W in the CH3 domain of the first heavy chain (at position 366) along with S, A, and V in the CH3 domain of the second heavy chain (at positions 366, 368, and 407).

In some embodiments, such a CH3 heterodimer may comprise any of the following CH3 sets: W-SG, V-V, QR-F, RG-FG, TL-QL, DVG-VSY, LWG-SIG, WTL-SAVQL, WTL-SGQL, WQL-SAVTL, WQL-SGTL, VTL-VQL, VQL-VTL, QRQL-FTL, VQR-VF, or LWG-IG. In some embodiments, such a CH3 heterodimer may comprise any of the following CH3 sets: W-SG, V-V, QR-F, RG-FG, TL-QL, DVG-VSY, LWG-SIG, or LWG-IG. In some embodiments, CH3 sets may be further added with the CH3 disulfide bond-allowing substitutions (“354/349” or “349/354” substitutions). In such embodiments, such a CH3 heterodimer may comprise any of the following CH3 sets: W-SG (349/354), V-V (349/354), QR-F (349/354), RG-FG (349/354), TL-QL (349/354), DVG-VSY (349/354), LWG-SIG (349/354), WTL-SAVQL (349/354), WTL-SGQL (349/354), WQL-SAVTL (349/354), WQL-SGTL (349/354), VTL-VQL (349/354), VQL-VTL (349/354), QRQL-FTL (349/354), VQR-VF (349/354), or LWG-IG (349/354), or W-SG (354/349), V-V (354/349), QR-F (354/349), RG-FG (354/349), TL-QL (354/349), DVG-VSY (354/349), LWG-SIG (354/349), WTL-SAVQL (354/349), WTL-SGQL (354/349), WQL-SAVTL (354/349), WQL-SGTL (354/349), VTL-VQL (354/349), VQL-VTL (354/349), QRQL-FTL (354/349), VQR-VF (354/349), or LWG-IG (354/349). In some embodiments, such a CH3 heterodimer may comprise any of the following CH3 sets: W-SG (349/354), V-V (349/354), QR-F (349/354), RG-FG (349/354), TL-QL (349/354), DVG-VSY (349/354), LWG-SIG (349/354), LWG-IG (349/354), W-SG (354/349), V-V (354/349), QR-F (354/349), RG-FG (354/349), TL-QL (354/349), DVG-VSY (354/349), LWG-SIG (354/349), or LWG-IG (354/349). Details of the amino acid positions and residues of the substitutions in these sets are shown in Appendix Tables E-G.

In some preferred embodiments, the CH3 set according to the present disclosure may be W-SG, LWG-SIG, W-SG (349/354), LWG-SIG (349/354), W-SG (354/349), or LWG-IG (354/349).

In further embodiments, any of the substitution sets described herein may be combined with another CH3 heterodimerization-preferring CH3 substitution or substitution set, such as any one of the inventive CH3 substitution or substitution set described herein, or any one of the pre-existing CH3 heterodimerization-preferring substitution or substitution set, such as those listed in Table 1, to further enhance or promote CH3 heterodimerization.

Furthermore, for each of the specific amino acid substitution in the CH3 domain that are provided herein to CH3 heterodimerization preference, the amino acid included as a result of substitution may be further substituted via a conservative amino acid substitution to obtain another variant CH3 domain that provide equivalent preference on CH3 heterodimerization. Alternatively, for each variant CH3 domain, one or more amino acid positions that were not affected in the variant CH3 domain relative to the wild-type sequence may be altered via a conservative substitution to obtain another variant CH3 domain that provide equivalent CH3 heterodimerization preference.

Provided below are a brief summary of some of the CH3 sets identified as shown in Examples and provide at least one superior property such as higher heterodimerization over a pre-existing CH3 heterodimerization variant CH3 domain set. For example, all of (1)-(7) sets show superior heterodimerization as measured by flow cytometry, when expressed as “modified Fc” on yeast cells, as shown in Examples. Some of the additional superior properties (non-exhaustive) for each of (1)-(7) are also provided below.

(1) The “W-SG” Set with or without the CH3 Disulfide Substitutions

The “W-SG” set comprises T366W in one CH3 domain and T366S and Y407G in another CH3 domain.

For example, the “W-SG” set shows higher % heterodimer values as measured by AlphaLISA® over tested controls (EW-RVT and KiH) (see FIG. 11B). The “W-SG” set with the CH3 disulfide substitutions further shows a very high % heterodimer value (%100) as measured by LC-MS, when expressed as a BsAb in HEK cells, over tested controls (EW-RVT and KiH with the CH3 disulfide substitutions) (see Table 13). Furthermore, the “W-SG” set with or without the CH3 disulfide substitutions shows less aggregation, as measured by SEC, relative to the respective controls pre-existing technologies tested (EW-RVT and KiH, with or without the CH3 disulfide substitutions) (see Table 6). Additionally, the “W-SG” set with the CH3 disulfide substitutions shown a higher yield over pre-existing technologies tested (EW-RVT and KiH with the CH3 disulfide substitutions) (see Table 6).

When such substitutions are made to the reference CH3 domain sequence of SEQ ID NO: 1, the set of the sequences of the variant CH3 domains comprises the amino acid sequences of SEQ ID NO: 11 and 12, respectively, 13 and 14 respectively, or 15 and 16 respectively.

(2) The “V-V” Set with or without the CH3 Disulfide Substitutions

The “V-V” set comprises T366V in one CH3 domain and Y407V in another CH3 domain.

For example, the “V-V” set shows higher % heterodimer values as measured by AlphaLISA® over pre-existing technologies tested (EW-RVT and KiH) (see FIG. 11B). The “V-V” set also shows a higher yield over tested controls (EW-RVT and KiH) when produced in HEK293 cells (see Table 6). Furthermore, the “V-V” set with or without the CH3 disulfide substitutions shows less aggregation, as measured by SEC, relative to the respective control pre-existing technologies tested (EW-RVT and KiH, with or without the CH3 disulfide substitutions, respectively) (see Table 6). Additionally, the “V-V” set provides less aggregation as measured by SEC and a much higher yield (207 mg/L), when expressed as a BsAb in HEK293 cells, over pre-existing technologies tested (EW-RVT and KiH) (see Table 13 and FIG. 18F).

Furthermore, when bsAbs comprising the “V-V” set were generated using the cFAE-mediated production method, 100% of the product was the intended bsAb and no mispairing was observed (see Table 18), which indicates that the “V-V” set is superior over the pre-existing “R-L” set in bsAb production efficiency. Additionally, bsAbs comprising the “V-V” set were further found to be resistant to glutathione challenge (see FIGS. 28A-28E).

When such substitutions are made to the reference CH3 domain sequence of SEQ ID NO: 1, the set of the sequences of the variant CH3 domains comprises the amino acid sequences of SEQ ID NO: 21 and 22, respectively, 23 and 24 respectively, or 25 and 26 respectively.

(3) The “QR-F” Set with or without the CH3 Disulfide Substitutions

The “QR-F” set comprises T366Q and K409R in one CH3 domain and L368F.

For example, the “QR-F” set shows (i) a higher % heterodimer value (100%) as measured by LC-MS and (ii) a higher Tm (64° C.), when expressed as a modified Fc, over the pre-existing technology tested (KiH) (see Table 8). The “QR-F” with the CH3 disulfide substitutions also show very high % heterodimers as measured by LC-MS (100%), which is higher than with the KiH control, when expressed as a BsAb in HEK293 cells (see Table 10). Additionally, the “QR-F” set with or without the CH3 disulfide substitutions provides less aggregation as measured by SEC over relevant pre-existing technology controls tested (EW-RVT and KiH with the CH3 disulfide substitutions) (see Table 13 and FIG. 18D).

When such substitutions are made to the reference CH3 domain sequence of SEQ ID NO: 1, the set of the sequences of the variant CH3 domains comprises the amino acid sequences of SEQ ID NO: 31 and 32, respectively, 33 and 34 respectively, or 35 and 36 respectively.

(4) The “RG-FG” Set with or without the CH3 Disulfide Substitutions

The “RG-FG” set comprises T366R and K409G in one CH3 domain and L368F and K370G.

For example, the “RG-FG” set shows (i) a higher % heterodimer value (90%) as measured by LC-MS and (ii) a higher Tm (64 deg C.), when expressed as a modified Fc, over the pre-existing technology tested (KiH) (see Table 8). The “RG-FG” set with the CH3 disulfide substitutions further shows a very high % heterodimer value (%100) as measured by LC-MS, when expressed as a BsAb in HEK cells, over tested controls (EW-RVT and KiH with the CH3 disulfide substitutions) (see Table 13).

When such substitutions are made to the reference CH3 domain sequence of SEQ ID NO: 1, the set of the sequences of the variant CH3 domains comprises the amino acid sequences of SEQ ID NO: 41 and 42, respectively, 43 and 44 respectively, or 45 and 46 respectively.

(5) The “TL-QL” Set with or without the CH3 Disulfide Substitutions

The “TL-QL” set comprises S400T and K409L in one CH3 domain and D399Q and F405L.

For example, the “TL-QL” set shows a higher Tm (65 deg C.), when expressed as a modified Fc, over the pre-existing technology tested (KiH) (see Table 8).

When such substitutions are made to the reference CH3 domain sequence of SEQ ID NO: 1, the set of the sequences of the variant CH3 domains comprises the amino acid sequences of SEQ ID NO: 51 and 52, respectively, 53 and 54 respectively, or 55 and 56 respectively.

(6) The “DVG-VSY” Set with or without the CH3 Disulfide Substitutions

The “DVG-VSY” set comprises S364D, Y407V, and K409G in one CH3 domain and T366V, L368S, and K370Y.

For example, the “DVG-VSY” set with the CH3 disulfide substitutions shows less aggregation as measured by SEC, when expressed as a BsAb in HEK293 cells, over the pre-existing technology tested (KiH) (see Table 11). Additionally, the “DVG-VSY” set with the CH3 disulfide substitutions provides less aggregation as measured by SEC over relevant pre-existing technology controls tested (EW-RVT and KiH with the CH3 disulfide substitutions) (see Table 13 and FIG. 18D).

When such substitutions are made to the reference CH3 domain sequence of SEQ ID NO: 1, the set of the sequences of the variant CH3 domains comprises the amino acid sequences of SEQ ID NO: 61 and 62, respectively, 63 and 64 respectively, or 65 and 66 respectively.

(7) The “LWG-SIG” Set with or without the CH3 Disulfide Substitutions

The “LWG-SIG” set comprises S364L, T366W, and K409G in one CH3 domain and T366S, L368I, and Y407G in the other CH3 domain of the set.

For example, the “LWG-SIG” set shows a higher Tm (62.5 deg C.), when expressed as a modified Fc, over the pre-existing technology tested (KiH) (see Table 8). Furthermore, the “LWG-SIG” sets with or without the CH3 disulfide bond substitutions show higher % heterodimers as measured by LC-MS or IEX when expressed as an anti-CD3/anti-HER2 BsAb, over the pre-existing technology tested (KiH) (see FIG. 18C and Table 13).

When such substitutions are made to the reference CH3 domain sequence of SEQ ID NO: 1, the set of the sequences of the variant CH3 domains comprises the amino acid sequences of SEQ ID NO: 71 and 72, respectively, 73 and 74 respectively, or 75 and 76 respectively.

Any of the variant CH3 domains described above or herein may be part of a polypeptide, such as a heavy chain polypeptide. Such polypeptides such as heavy chain polypeptides are also encompassed by the present invention.

### Polypeptides, Molecules, and Multi-Specific Antibodies

A variant CH3 domain according to the present disclosure may exist in a polypeptide such as an immunoglobulin polypeptide, a molecule, and/or a multi-specific antibody.

The “immunoglobulin polypeptide” as used herein refers to a polypeptide comprising at least one domain of an immunoglobulin (e.g., a CH3 domain). In certain instances, a first CH3 domain may exist in a first polypeptide. In certain instances, a second CH3 domain may be exist in a second polypeptide. When the first CH3 and second CH3 domains are CH3 domains that preferentially form a CH3-CH3 heterodimer, a heteromeric (e.g., dimeric) molecule may be formed between the first polypeptide and a second polypeptide. Such heteromeric molecule may be a multi-specific antibody having a structure such as but not limited to the structures disclosed in FIGS. 2-8.

In some embodiments, such a heterodimer-preferring CH3 domain sets may be any one of the following: W-SG, V-V, QR-F, RG-FG, TL-QL, DVG-VSY, LWG-SIG, WTL-SAVQL, WTL-SGQL, WQL-SAVTL, WQL-SGTL, VTL-VQL, VQL-VTL, QRQL-FTL, VQR-VF, or LWG-IG. In some embodiments, such a heterodimer-preferring CH3 domain sets may be any one of the following: W-SG, V-V, QR-F, RG-FG, TL-QL, DVG-VSY, LWG-SIG, or LWG-IG. In some embodiments, such a heterodimer-preferring CH3 domain sets may be any of the foregoing further comprising the CH3 disulfide bond substitutions (“345/354” or “354/345”), i.e., W-SG (349/354), V-V (349/354), QR-F (349/354), RG-FG (349/354), TL-QL (349/354), DVG-VSY (349/354), LWG-SIG (349/354), WTL-SAVQL (349/354), WTL-SGQL (349/354), WQL-SAVTL (349/354), WQL-SGTL (349/354), VTL-VQL (349/354), VQL-VTL (349/354), QRQL-FTL (349/354), VQR-VF (349/354), or LWG-IG (349/354), or W-SG (354/349), V-V (354/349), QR-F (354/349), RG-FG (354/349), TL-QL (354/349), DVG-VSY (354/349), LWG-SIG (354/349), WTL-SAVQL (354/349), WTL-SGQL (354/349), WQL-SAVTL (354/349), WQL-SGTL (354/349), VTL-VQL (354/349), VQL-VTL (354/349), QRQL-FTL (354/349), VQR-VF (354/349), or LWG-IG (354/349). In some embodiments, such a CH3 heterodimer may comprise any of the following CH3 sets: W-SG (349/354), V-V (349/354), QR-F (349/354), RG-FG (349/354), TL-QL (349/354), DVG-VSY (349/354), LWG-SIG (349/354), LWG-IG (349/354), W-SG (354/349), V-V (354/349), QR-F (354/349), RG-FG (354/349), TL-QL (354/349), DVG-VSY (354/349), LWG-SIG (354/349), or LWG-IG (354/349). Amino acid substitution positions in these CH3 sets are specified in Appendix Tables E-G).

Such an immunoglobulin polypeptide may further comprise one or more antigen-binding domains (such as VH, VL, scFv, or nanobody), CH1, and/or CH2 domain(s). An additional CH3 domain (with or without an amino acid substitution(s) may be further included. Such a polypeptide may be part of a multi-specific antibody molecule. In some embodiments, a peptide may comprise an antigen-binding domain (such as a VH, VL, scFv, or nanobody), and a variant CH3 domain. In some embodiments, a peptide may comprise an antigen-binding domain (such as a VH, VL, scFv, or nanobody), CH1, and a variant CH3 domain. In some embodiments, a peptide may comprise an antigen-binding domain (such as a VH, VL, scFv, or nanobody), CH2, and a variant CH3 domain. In some embodiments, a peptide may comprise an antigen-binding domain (such as a VH, VL, scFv, or nanobody), CH1, CH2, and a variant CH3 domain.

Alternatively, the immunoglobulin polypeptide may not comprise a VH, VL, CH1, or CH2 domains. For example, a first polypeptide may further comprise a first domain in addition to a first CH3. If a second polypeptide further comprises a second domain in addition to a second CH3 which preferentially forms a heterodimer with the first CH3, and if it is desired to form a heterodimer between the first and second domains, the preferential heterodimerization between the first and second CH3 domains will facilitate heterodimerization of the first and second domains.

In one embodiment, such a polypeptide may optionally utilize, in combination with the variant CH3 domains, other variants outside of the CH3 domain to further promote preferential pairing between two polypeptides that are different from each other.

In one embodiment, such a polypeptide may optionally utilize, in combination with the variant CH3 domains, a variant CH1 domain(s) that promote preferential lambda pairing or preferential kappa pairing. In another embodiment, such a polypeptide may optionally utilize, in combination with the variant CH3 domains, a variant CH1 domain(s) that preferentially pairs with a variant kappa CL domain over with another CL domain such a wild-type kappa CL domain. In another embodiment, such a polypeptide may optionally utilize, in combination with the variant CH3 domains, a variant CH1 domain(s) that preferentially pairs with a variant lambda CL domain over with another CL domain such a wild-type lambda CL domain. In yet another embodiment, such a polypeptide may optionally utilize, in combination with the variant CH3 domains, a variant kappa or lambda CL domain(s) that preferentially pairs with a variant CH1 domain over with another CH1 domain such a wild-type CH1 domain. Using such a polypeptide, generation of antibodies that are specific to more than two antibodies (e.g., tetraspecific antibodies) may be facilitated.

Any of such polypeptides may exist in a molecule which has a first polypeptide comprising a first variant CH3 domain and a second polypeptide comprising a second variant CH3 domain which preferentially forms a CH3-CH3 heterodimer with the first CH3. In some cases, the first and second polypeptide may be further linked, e.g., via one or more disulfide bond(s), linker(s), etc.

Such a molecule may be a multi-specific antibody or antigen-binding fragments having a structure such as but not limited to the structure disclosed in FIGS. 2-8. A multi-specific antibody according to the present disclosure may be bispecific, trispecific, tetraspecific, or specific to five, six, or more epitopes. A multi-specific antibody according to the present disclosure may be divalent, trivalent, or tetravalent or have valency of five, six, or higher.

### Polynucleotides, Vectors, Cells, and Compositions

Polypeptides, molecule, and/or multi-specific antibodies comprising variant CH3 domains described herein may be encoded by a polynucleotide or polynucleotides. Such polynucleotide or polynucleotides may be a DNA or RNA or a combination thereof.

Any of the polypeptide(s) described herein may be present in a vector.

Any of the CH3 domain(s), polypeptide(s), molecule(s), multi-specific antibody(ies), polynucleotide(s), and/or vector(s) may be present in a cell, e.g., a eukaryotic cell. In some embodiments, such polypeptides may be expressed in mammalian cells, such as HEK293 cells or Chinese hamster ovary (CHO) cells. In some embodiments, variant CH3 domains are expressed in yeast (e.g., Saccharomyces cerevisiae. In some embodiments, a yeast strain co-expresses one or more polypeptides, such as one or more light chains.

Any of the CH3 domain(s), polypeptide(s), molecule(s), multi-specific antibody(ies), polynucleotide(s), vector(s), and/or cells may be present in a composition. If the composition is a therapeutic composition, the composition may further comprise a pharmaceutically acceptable carrier.

### CH3 Domain Libraries and Variant CH3 Domain Screening/Selection

Also contemplated by the present disclosure are methods of generating a CH3 domain library. The library may be particularly used to screen for CH3 sequences and CH3 sets that preferentially form CH3 heterodimers.

In some embodiments, at least one nucleic acid position within the codon encoding any of the amino acid positions of CH3 at which an amino acid substitution is present in any of the inventive CH3 sets may be variegated. For example, such pre-determined amino acid position(s) may be position(s) 364, 366, 368, 370, 399, 400, 405, 407, and/or 409, or any combination thereof, according to EU numbering.

In some embodiments, any of the amino acid positions listed in Table 7 may be variegated.

In some embodiments, any of the amino acid positions considered as the CH3-CH3 “interface positions” may be variegated.

In certain embodiments, some of the CH3 domains expressed by the library may contain the CH3 disulfide bond substitutions (i.e., S354C/Y349C) in addition to the substitution(s) caused by variegation.

In some embodiments, a degenerate codon, optionally a degenerate RMW codon representing six naturally occurring amino acids (D, T, A, E, K, and N) or a degenerate NNK codon representing all 20 naturally occurring amino acid residues may be used, to induce variegation at a pre-determined position.

Also provided herein are methods of identifying one or more variant CH3 domains and CH3 sets that preferentially form CH3 heterodimers.

In some embodiments, the method may comprise at least three steps. The first step may be co-expressing in a cell (e.g. yeast cells, mammalian cells) or ex vivo (1) a first polypeptide comprising a first variant CH3 domain expressed from a first library, which is according to any of the libraries described herein and (2) a second polypeptide comprising a second variant CH3 domain expressed from a second library, which is according to any of the libraries described herein. The second step may be quantifying the amount of the CH3 heterodimers and homodimers. The third step may be selecting one or more CH3 sets which provides a desired % heterodimers.

In certain embodiments, the first library and the second library may differ by at least one pre-determined amino acid position.

In some embodiments, the predetermined position(s) in the first library and the predetermined position(s) in the second library may comprise or consist of any of the positions or position sets substituted in the CH3 sets identified herein as preferring heterodimerization.

The variegation may be made to any available CH3 sequence, i.e., wild-type or modified CH3 sequences. In some embodiments, the variegation may be made to the reference CH3 sequence of SEQ ID NO: 1.

In certain embodiments, the desired % heterodimers may be about >50%, about >55%, about >60%, about >65%, about >70%, about >75%, about >80%, about >85%, about >90%, about >95%, about >96%, about >97%, about >98%, about >99%, or about 100%.

In certain embodiments, the desired % heterodimers may be relative to a reference CH3 set, e.g., a pre-existing CH3 heterodimerization technology (e.g., in Table 1).

In some embodiments, the first polypeptide may contain or expressed with a first tag and the second polypeptide may contain or expressed with a second tag that is different from the first tag. This would allow specifically identifying CH3 heterodimers by techniques such as AlphaLISA®.

In some embodiments, the second step of quantifying heterodimers and homodimers may use, for example, liquid chromatography-mass spectrometry (LC-MS), AlphaLISA®, ion exchange chromatography (IEX), and/or flow cytometry.

In certain embodiments, the method of identifying may further comprise a step of selecting one or more sets of a first variant CH3 domain polypeptide and a second variant CH3 domain polypeptide based on one or more antibody characteristics. Exemplary characteristics may include but are not limited to: (i) (i-1) production yield, optionally assessed in one or more cell types, optionally mammalian cells such as CHO cells and HEK cells, yest cells, insect cells, and/or plant cells and/or (i-2) compatibility to one or more antibody purification methods, optionally comprising protein A affinity purification; (ii) degree of aggregation, optionally presence of multimers of a full-size antibody, optionally quantified using chromatography, optionally SEC or electrophoresis, optionally SDS-PAGE; (iii) the rate of correct pairing, optionally correct pairing between CH1 domains and/or between CH1 and CL domains, optionally assessed using LC-MS; (iv) Tm and/or Tagg, optionally Tagg266, optionally measured using DSF and/or DSC and/or using an instrument, optionally Uncle®; (v) pI; (vi) the level of interaction with PSR; (vii) hydrophobic interaction of the antibody optionally measured using HIC; (viii) self-interaction, optionally measured by (viii-1) AC-SINS or (viii-2) DLS; (ix) stability to high or low pH stress; (x) solubility; (xi) production costs and/or time; (xii) other stability parameters; (xiii) shelf life; (xiv) in vivo half-life; and/or (xv) immunogenicity.

Such characteristics may at least partly depend on (a) the particular structure of the molecule or multi-specific antibody or antigen-binding antibody fragment which incorporates a variant CH3 domain set and/or (b) the variable domains providing particular binding specificities. The suitability may be tested in the particular context of the antibody structure and antigen specificities of interest.

Therefore, also provided herein are methods of screening for a set of a first variant CH3 domain polypeptide and a second variant CH3 domain polypeptide suited for a multi-specific antibody or antigen-binding antibody fragment (e.g., having the any of the structures described herein) which has given antigen specificities.

In some embodiments, the method may comprise: (a) expressing the multiple multi-specific antibodies and/or antigen-binding antibody fragments, comprising different sets of a first variant CH3 domain polypeptide and a second variant CH3 domain polypeptide; and (b) selecting one or more sets of a first variant CH3 domain polypeptide and a second variant CH3 domain polypeptide based on one or more antibody characteristics of the multiple multi-specific antibodies and/or antigen-binding antibody fragments expressed in step (a).

In some embodiments, the one or more antibody characteristics may be selected from the characteristics (i)-(xv) described above.

cFAE-Mediated Multi-Specific Antibody or Antigen-Binding Antibody Fragment Production Methods; and Multi-Specific Antibodies and Antigen-Binding Antibody Fragments Produced by Such Methods

Also contemplated by the present disclosure are methods of producing a heteromeric molecule comprising a CH3 set that preferentially form CH3 heterodimers, which in some embodiments may be any of the CH3 sets described herein. The heteromeric molecule may be any of the heteromeric molecules or multi-specific antibodies and antigen-binding antibody fragments described herein, optionally having a structure depicted in any one of FIGS. 2-8.

In some embodiments, the heteromeric molecule of interest (one that the method intends to produce) may comprise (A) a first polypeptide (e.g., a first heavy chain) comprising a first variant CH3 domain polypeptide; and (B) a second polypeptide (e.g., a second heavy chain) comprising a second variant CH3 domain polypeptide, wherein the first and second polypeptides may be bound to or paired with each other optionally via at least one disulfide bond.

Examples herein demonstrated that the “V-V” set provided excellent bsAb production efficiency without causing any mispairing, as shown in Table 18, which is superior over the pre-existing “R-L” set in bsAb production efficiency. Additionally, bsAbs comprising the “V-V” set were further found to be resistant to glutathione challenge (see FIGS. 28A-28E).

Therefore, in particular embodiments, the CH3 set incorporated in the heteromeric molecule or multi-specific antibody or antigen-binding antibody fragment produced by the method may be the “V-V” set, including Y407V mutation in one CH3 domain of the set and T366V mutation in the second CH3 domain of the set. In certain embodiments, the CH3 set may comprises, in addition to the “V-V” set substitutions, additional substitutions, such as but not limited to, the disulfide modifications at position 349 and 354 as described herein (i.e., “V-V (349/354) or “V-V (354/349)” set).

In some embodiments, the method may comprise (i) incubating in a reducing environment (i-1) a first antibody (which may also be referred to as a first parent antibody or a first monospecific parent antibody) comprising at least two of the first polypeptides bound to or paired with each other optionally via at least one disulfide bond and (i-2) a second antibody (which may also be referred to as a second parent antibody or a second monospecific parent antibody) comprising at least two of the second polypeptides bound to or paired with each other optionally via at least one disulfide bond. The first and second parent antibodies may be IgGs (e.g., IgG1, IgG2, IgG3, and IgG4).

In some embodiments, the first polypeptide may further comprise a first antigen-binding domain. In some embodiments, the second polypeptide may further comprise a second antigen-binding domain. In some embodiments, the heteromeric molecule may further comprise a third polypeptide optionally comprising a third antigen-binding domain, optionally wherein the third polypeptide may be bound to or paired with the first polypeptide. In some embodiments, the heteromeric molecule may further comprise a fourth polypeptide optionally comprising a fourth antigen-binding domain, optionally wherein the fourth polypeptide may be bound to or paired with the second polypeptide.

In certain embodiments, the first polypeptide may comprise a first antigen-binding domain which forms a first antigen-binding site specific for a first epitope and/or the heteromeric molecule may comprise a third polypeptide comprising a third antigen-binding domain which forms a third antigen-binding site specific for a third epitope. Optionally, the first epitope may be same as or different from the third epitope. In alternative embodiments, the first polypeptide may comprise a first antigen-binding domain and the heteromeric molecule may comprise a third polypeptide comprising a third antigen-binding domain, wherein the first antigen-binding domain and the third antigen-binding domain form a first antigen-binding site specific for a first epitope.

In certain embodiments, the second polypeptide may comprise a second antigen-binding domain which forms a second antigen-binding site specific for a second epitope and/or the heteromeric molecule may comprise a fourth polypeptide comprising a fourth antigen-binding domain which forms a fourth antigen-binding site specific for a fourth epitope. Optionally, the second epitope may be same as or different from the fourth epitope. In alternative embodiments, the second polypeptide may comprise a second antigen-binding domain and the heteromeric molecule may comprise a fourth polypeptide comprising a fourth antigen-binding domain, wherein the second antigen-binding domain and the fourth antigen-binding domain form a second antigen-binding site specific for a second epitope.

The first and second antibodies may be produced in any appropriate cell types. Exemplary cells may include but not limited to: in a mammalian cell, a yeast cell, an insect cell, a plant cell, or a bacterial cell, and more specifically, a Chinese hamster ovary (CHO) cell or a Human embryonic kidney (HEK) cell.

In certain embodiments, the first and second antibodies may be incubated at a temperature between about 15° C. and about 40° C., between about 20° C. and about 40° C., between about 25° C. and about 35° C., between about 28° C. and about 32° C., or between about 29° C. and about 31° C., or at about 30° C. In certain embodiments, the first and second antibodies may be incubated for about 30 minutes to about 20 hours, for about 1 hour to about 15 hours, for about 2 hours to about 10 hours, for about 3 hours to about 7 hours, or for about 4 hours to about 6 hours, or for about 5 hours. In particular embodiments, the first and second antibodies may be incubated at about 30° C. for about 5 hours.

In certain embodiments, the first and second antibodies may be incubated in the presence of at least one reducing agent, optionally at least one mildly reducing agent. Ideally, the at least one reducing agent or the reducing environment is one that is capable of reducing the disulfide bond(s) between two heavy chains (or between the first and the second polypeptides) but not between heavy and light chains. Various reducing agents have been shown to provide this reducing function in the context of FAE (see e.g., van der Neut Kolfschoten et al. Science. 2007 Sep. 14; 317(5844):1554-1557). Exemplary reducing agents include but are not limited to 2-mercaptoethylamine (2-MEA), s-mercapto-ethanol (BME), L-cysteine, dithiothreitol (DTT), or dithionite.

In certain embodiments, the at least one reducing agent may be selected from: about 25 to about 125 mM, about 50 mM to about 100 mM, about 70 to about 80 mM, or about 75 mM of 2-MEA; about 20 to about 500 μM, about 40 to about 250 μM, about 80 to about 150 μM, about 90 to about 120 μM, or about 100 μM of BME; about 20 to about 500 μM, about 40 to about 250 μM, about 80 to about 150 μM, about 90 to about 120 μM, or about 100 μM of L-cysteine; about 15 to about 400 μM, about 20 to about 200 μM, about 25 to about 100 μM, about 30 to about 70 μM, or about 50 μM of DTT; or about 20 to about 500 μM, about 40 to about 250 μM, about 80 to about 150 μM, about 90 to about 120 μM, or about 100 μM of dithionite. In particular embodiments, the reducing environment may comprise at least 2-MEA at about 75 mM.

In some embodiments, the method may then comprise (ii) placing the incubation product of step (i) in a less reducing or non-reducing environment.

In certain embodiments, this step (ii) may allow for pairing between the first variant CH3 domain and the second variant CH3 domain, thus pairing between the first polypeptide and the second polypeptide.

In certain embodiments, the placing may be via buffer exchange, allowing removal of the reducing condition such as a reducing agent. For example, the buffer may be exchanged to PBS.

In particular embodiments, the buffer exchange may be performed via desalting or diafiltration.

In another embodiment, the placing may be performed by adding an oxidizing agent.

In some embodiments, the product of step (ii) may be incubated in the less reducing or non-reducing environment. In certain embodiments, the incubation may be carried out at a temperature between about PC and about 20° C., between about 2° C. and about 10° C., between about 3° C. and about 5° C., or at about 4° C. In certain embodiments, the incubation may be carried out for about 12 hour to about 154 hours, for about 24 hours to about 96 hours, for about 36 hours to about 72 hours, or for about 48 hours. In particular embodiments, the incubation may be carried out at about 4° C. for about 48 hours.

In some embodiments, the product of step (ii) and/or (iii) may be analyzed for the amount of the multi-specific antibody or antigen-binding antibody fragment of interest in the product of step (ii) and/or (iii). In some embodiments, the product of step (ii) and/or (iii) may be subjected to purification to obtain purified multi-specific antibody or antigen-binding antibody fragment. In certain embodiments, such analyses and/or purification may be performed by chromatography, such as but not limited to, LC-MS, IEX, and/or SEC. In particular embodiments, no mispairing may be observed by LC-MS.

In some embodiments, heteromeric molecules produced include multi-specific antibodies (e.g., bispecific, trispecific, and tetraspecific antibodies). Polypeptides of such multi-specific antibodies may include antibody heavy chains, which may be associated with antibody light chains, the antigen-binding domains of which may form antigen-binding sites. Multi-specific antibodies may include additional polypeptides, which may include additional antigen-binding domains and/or form additional antigen-binding sites. Such additional antigen-binding domains or sites may be associated with antibody heavy chains or light chains of multi-specific antibodies. Such associations may be via a linker. Such linkers may include flexible linkers that include polypeptides with multiple glycine and/or serine residues. In some embodiments, additional antigen-binding domains may include Fab antibody fragments or single chain Fv (scFv) fragments, which may be stabilized by disulfide bonds. In some embodiments, the multi-specific antibodies may be biparatopic antibodies.

In some production methods, a first IgG and a second IgG are used to prepare heteromeric molecules. First polypeptides of the first IgG may include a first antibody heavy chain including a first antigen-binding domain which forms a first antigen-binding site for a first epitope. Second polypeptides of the second IgG may include a second antibody heavy chain including a second antigen binding domain which forms a second antigen-binding site for a second epitope. The first epitope and the second epitope may be part of different antigens. The first epitope and the second epitope may be part of the same antigen. The heteromeric molecule may be an IgG that includes the first antibody heavy chain and the second antibody heavy chain.

Any multi-specific antibodies and antigen-binding antibody fragments produced by the methods of producing are further encompassed by the present disclosure.

Examples are provided below to illustrate the present invention. These examples are not meant to constrain the present invention to any particular application or theory of operation.

### EXAMPLES

In Examples described herein, the CH3 domain reference sequence (SEQ ID NO: 1) was used as a wild-type CH3 domain sequence of IgG1, and various amino acid substitutions were incorporated to the reference sequence for testing heterodimerization potential. Some of the sequences used in Examples are provided in Appendix Tables A-G and sequence listing. Although SEQ ID NO: 1 was used as the CH3 domain reference sequence in Examples, the present invention relating to a CH3 domain sequence modification(s) may also be applied to other CH3 domain reference sequences, such as but not limited to SEQ ID NO: 2, 3, or 4 (for IgG1) or another standard CH3 sequence of IgG1, IgG2, IgG3, or IgG4.

Unless otherwise noted, the CH1 and CH2 reference sequences (SEQ ID NOS: 6 and 7, respectively) were used in Examples, when applicable.

### Example 1: Evaluation of Flow Cytometry-Based Selection of Modified Fc-Presenting Yeast Library as a CH3 Domain Dimerization Readout Method Using Pre-Existing CH3 Heterodimerization Technologies (Proof of Concept Study)

For screening for heterodimer-preferring variant CH3 domains, a yeast library system in which each cell presents a “modified Fc” (a portion of the Fc encompassing positions D221-K447 (EU numbering) with a portion of the hinge (SPPS instead of CPPC), a modified CH2 domain (N297A) domain, and a CH3 domain having either wild-type or variant sequences) was designed, initially analyzed by flow cytometry to enrich cell populations comprising CH3 heterodimers.

First, to evaluate this system as a method for identifying variant CH3 domains that preferentially form CH3 heterodimers, the system was tested using the pre-existing variant CH3 domain sets, W-SAV (i.e., KiH) and EW-RVT (see Table 1) as controls. These two CH3 sets were confirmed to show high preference in forming CH3 heterodimers when used in bispecific antibodies, as measured by AlphaLISA® and liquid chromatography-mass spectrometry (LC-MS) (using the methods described later herein).

Libraries of variant CH3 domains were built and expressed in an engineered yeast strain. Specifically, two heavy chain expression plasmids (pAD6234 and pAD6233) were constructed, each of which contained “modified Fc”. pAD6234 included a FLAG tag and URA and pAD6233 included a HIS tag and TRP.

Three sets of pAD6234 and pAD6233 were generated. The first set encoded CH3 domains comprising the KiH substitution set (Knob_{HIS}Hole_{FLAG}), the second set encoded CH3 domains comprising the EW-RT set (EW_{HIS}RVT_{FLAG}), and the third set encoded CH3 domains comprising the WT-WT set (also referred to as “WT set”) (WT_{HIS}-WT_{FLAG}).

A yeast proof-of-concept (POC) library which is a 1:1:10,000 mix of yeast cells introduced with the first, second, and third plasmids set, respectively, was generated, propagated as described previously (see, e.g., WO2009036379; WO2010105256; WO2012009568; Xu et al., Protein Eng Des Sel. 2013 October; 26(10):663-70), and subjected to flow cytometry-based selection of high FLAG expressors (which indicates CH3 heterodimer expressors) (schematic in FIG. 9A).

Briefly, following expression of modified Fc, the engineered yeast cells (˜10⁷-10⁸) were stained for 15 minutes at 4° C. with anti-HIS FITC diluted 1:100 (Invitrogen, Carlsbad, California, Cat #MA1-81891) and anti-FLAG APC diluted 1:500 (BioLegend, San Diego, California, Cat #637308) in PBSF. After washing twice with ice-cold wash buffer, cell pellets were resuspended in 0.4 mL PBSF and transferred to strainer-capped sort tubes. Sorting was performed using a FACS ARIA sorter (BD Biosciences) and sort gates were determined in order to enrich for heterodimers. Libraries were selected over two rounds. Clones having both HIS and FLAG tags were sequenced out of the fourth and fifth rounds and analyzed for sequence uniqueness. Cells were plated on media lacking uracil and tryptophan to generate single isolates for sequence identification.

As expected, cells expressing the CH3 heterodimers (KiH or EW-RVT) were significantly enriched after two rounds of selection (FIG. 9B), indicating that the system in which the modified Fc library coupled with the flow cytometry-based selection indeed can be used to identify CH3 heterodimer-preferring variant CH3 domains.

### Example 2: Cycle 1 Selection Using a Variant CH3 Domain Library Saturating One or More of the Four KiH Positions

In this example, the variant CH3 domain selection system described in Example 1 was used to identify novel variant CH3 domains that have an amino acid substitution(s) at one or more of the KiH substitution positions (KiH has W at position 366 (“Knob” position) in one CH3 and S, A, and V at positions 366, 368, and 407 (“Hole” positions) in another CH3, see Table 1).

Specifically, two pools of variant CH3 domain DNA fragments were generated for insertion into the expression plasmids (FIG. 10A). The first pool was generated by allowing for substitution of all twenty amino acids at position T366 of the first CH3 domain and positions T366, L368, and Y407 of the second CH3 domain. Two variations were made within the first pool: one in which the Hole position variegation is made in a strand with the Flag tag (Knob^{SSM}-HIS Hole^{SSM}-FLAG) (Library 1 in FIG. 10A); and one in which the Knob position variegation is made in a strand with the Flag tag (Hole^{SSM}-HIS Knob^{SSM}-FLAG) (Library 2 in FIG. 10A). The second pool was generated by fixing position T336W of the first CH3 domain constant and variegating positions T366, L368, and Y407 of the second CH3 domain with all twenty amino acids (Knob-HIS Hole^{SSM}-FLAG) (Library 3 in FIG. 10A). Libraries were built using DNA shuffling methods, as described previously (Stemmer, Proc. Natl. Acad. Sci., 91 (1994), pp. 10747-10751). The libraries were propagated and subjected to six rounds of selection, and the selection products were sequenced, as described in Example 1 (FIGS. 10B-10D).

As shown in Table 2, the selection did not result in a collapse in sequence.

TABLE 2
Sequencing of selections did not show a collapse
in sequence and revealed reversion mutations.

| Round | LAD | WT | ½ WT | 0 WT (Uniques) |
|---|---|---|---|---|
| R3 | Knob^{SSM}_{HIS} Hole^{SSM}_{FLAG} | 4 | 11 | 11 (11) |
| | Hole^{SSM}_{HIS} Knob^{SSM}_{FLAG} | 9 | 14 | 6 (6) |
| | Knob_{HIS} Hole^{SSM}_{FLAG} | 1 | — | 4 (4) |
| R4 | Knob^{SSM}_{HIS} Hole^{SSM}_{FLAG} | 1 | 6 | 12 (11) |
| | Hole^{SSM}_{HIS} Knob^{SSM}_{FLAG} | 6 | 14 | 9 (8) |
| | Knob_{HIS} Hole^{SSM}_{FLAG} | 0 | — | 9 (3) |
| R5 | Knob^{SSM}_{HIS} Hole^{SSM}_{FLAG} | 0 | 0 | 6 (3) |
| | Hole^{SSM}_{HIS} Knob^{SSM}_{FLAG} | 4 | 11 | 13 (13) |
| | Knob_{HIS} Hole^{SSM}_{FLAG} | 1 | — | 20 (11) |
| R6 | Knob^{SSM}_{HIS} Hole^{SSM}_{FLAG} | 0 | 7 | 8 (8) |
| | Hole^{SSM}_{HIS} Knob^{SSM}_{FLAG} | 1 | 6 | 6 (6) |
| | Knob_{HIS} Hole^{SSM}_{FLAG} | 1 | — | 4 (3) |

At the end of the 6^{th} round, 86 unique CH3 heterodimers were found. The top substitution combinations at the Knobs-into-Holes (KiH) positions are provided with occurrence frequencies (“repeats”) in Table 3.

TABLE 3
Top KiH position substitutions identified after the 6^{th} round.

| “Knob” | “Hole” | Repeats |
|---|---|---|
| T366W | T366S L368Y Y407 | 23 |
| T366L | T366 L386 Y407F | 9 |
| T366M | T366 L368 Y407A | 6 |
| T366W | T366S L386W Y407V | 5 |
| T366L | T366 L368 Y407A | 4 |
| T366W | T366A L368V Y407V | 4 |
| T366I | T366 L386 Y407G | 3 |
| T366N | T366 L368 Y407V | 3 |
| T366Q | T366 L368 Y407F | 3 |
| T366C | T366 L368 Y407T | 3 |
| T366V | T366 L368 Y407L | 3 |
| T366V | T366 L368 Y407V | 3 |

* Substitutions relative to the reference sequence are in bold.

The 86 unique CH3 heterodimer sequences were produced in yeast and characterized by AlphaLISA®, ion exchange chromatography (IEX), and size exclusion chromatography (SEC) in the following Examples. Melting temperatures were also determined.

### Example 3: AlphaLISA® Analyses on CH3 Sets Identified in Example 2

The 86 unique CH3 heterodimers identified in Example 2 were analyzed by AlphaLISA® (FIG. 11A left). AlphaLISA® was used to determine the relative degree of heterodimerization of Fc fragments. Briefly, 5 μl of fragment was added as a 0.5 nM final testing concentration solution to the Perkin Elmer AlphaLISA immunoassay buffer (10×), together with 10× biotin-a-Flag (5 μl, 20 nM final testing concentration) and put into a 384-well AlphaPlate (Perkin Elmer). Then, 10× acceptor bead solution (5 μl) having a-His was added, and the plates were covered with a black cover and incubated at RT for 1 hour. Next, 10× (5 μl) donor beads (SA coated) solution was added to the assay in a dark room and incubated for 30 minutes at room temperature. Plates were read using the EnSpire Alpha program (Perkin Elmer).

Data on the pre-existing CH3 heterodimerizing substitution sets demonstrated that AlphaLISA® can be used to measure CH3 heterodimerization (FIG. 11A right). These results are provided in FIG. 11B. Five CH3 sets showed an AlphaLISA® value higher than the positive controls (KiH and EW-RT) (Table 4). AlphaLISA® values (as fold-over-background) were further plotted against the a-Flag APC values (as fold-over-background) on the x axis FIG. 11C. The “V-V” set was found in both orientations (366V with FLAG and 407V with FLAG).

TABLE 4
Substitution combinations of samples with
AlphaLISA FOB higher than EW-RVT.

| AlphaLISA FOB | Knob | Hole | Repeats |
|---|---|---|---|
| 87 | T366V | T366 L368 Y407V | 3 |
| 68 | T366L | T366 L368 Y407V | 2 |
| 64 | T366L | T366 L368 Y407M | 1 |
| 61 | T366I | T366 L368 Y407F | 1 |
| 61 | T366W | T366S L368 Y407G | 1 |

### Example 4: Size Exclusion Chromatography (SEC) Analyses on CH3 Sets Selected in Example 3

Expression and quality of the purified antibodies comprising one of the five CH3 sets identified in Example 3 (“V-V”) was tested in both orientations, so total of 6 variations) were assessed by size exclusion chromatography (SEC). High main peak % represents high quality. Briefly, an Agilent 1 100 HPLC was employed to monitor the column chromatography (TSKgel Super SW3000 column). The column was pre-conditioned with highly glycosylated and aggregated IgG in order to minimize potential for antibody-column interactions and equilibrated with wash buffer (200 mM Sodium Phosphate, 250 mM Sodium Chloride pH 6.8) prior to use. Approximately 2-5 μg of protein sample was injected onto column and flow rate adjusted to 0.400 mL/min. Protein migration was monitored at wavelength 280 nm. Total assay time was approximately 11 minutes. Data was analyzed using ChemStation software.

SEC chromatographs for the WT and control CH3 sets (W-SAV (i.e., KiH) and EW-RVT) are shown in FIG. 12A, and SEC chromatographs for the CH3 sets selected in Example 3 are shown in FIG. 12B.

### Example 5: Ion Exchange (IEX) Chromatography Analyses on Cycle 1 Outputs

Ion exchange chromatography (IEX) was performed on a subset of clones. All chromatographic separations were performed on a computer controlled AKTA Avant 150 preparative chromatography system equipped with an integrated pH electrode, enabling in-line pH monitoring, and a Mono S 5/50 GL column. The cation exchange buffer was composed of 15.6 mM CAPS, 9.4 mM CHES, 4.6 mM TAPS, 9.9 mM HEPPSO, 8.7 mM MOPSO, 11.0 mM MES, 13.0 mM Acetate, 9.9 mM Formate, 10 mM NaCl, and the pH was adjusted up to 4.0 (buffer A) or 11.0 (buffer B) using NaOH. 500 ug of protein was buffer exchanged into 25% buffer B and filtered through a 0.2 mm filter. Before each separation, the column was equilibrated with 10 column volumes of 25% buffer B. The protein was then loaded onto the column via a capillary loop, followed by a 10 column volume wash with 25% buffer B, a 20 column volume linear pH gradient from 25% to 100% buffer B, and a 10 column volume hold at 100% B.

IEX chromatographs for the WT and control CH3 sets (W-SAV (i.e., KiH) and EW-RVT) are shown in FIG. 13A, and SEC chromatographs for the CH3 sets selected in Example 3 are shown in FIG. 14B. Some CH3 sets with low AlphaLISA® values showed poor quality as measured by SEC and IEX.

### Example 6: Production of Cycle 1 Outputs in HEK293

The impact of the identified variant CH3 domains on a control bispecific common light chain antibody in an IgG-like format (2 Fab regions attached N-terminally to a dimeric Fc molecule) was also assessed. The W-SG substitution set (comprising T366W in one CH3 and T366S and Y407G in the other CH3) and the V-V substitution set (comprising T366V in one CH3 and Y407V in the other CH3) were selected as exemplary test sets for production in HEK293 cells as anti-Her2/anti-CD3 bispecific antibodies. The VH-CH1 sequences derived from two antibodies, ADI-29235 (anti-HER2) and ADI-26908 (anti-CD3), were used. A wild-type CH3 set, the W-SAV set (i.e., KiH), and the EW-RVT set were included as controls. Additionally, CH3 domain substitutions (S354C/Y349C) were introduced to promote desired heterodimeric pairing of the heavy chains. Tested CH3 sets are summarized in Table 5.

DNA plasmids were confirmed via Sanger sequencing prior to transfection into HEK293 cells via standard protocols. Transfected HEK cells were cultured in CD optiCHO media (Invitrogen), and on day 6 post transfection the supernatants were collected and subjected to Protein A-based affinity purification.

TABLE 5
Substitution combinations produced in HEK293.

| | CH3 substitutions of HC1 (anti-Her2) | | CH3 substitutions of HC2 (anti-CD3) | |
|---|---|---|---|---|
| CH3 Set Name | CH3 S═S | | CH3 S═S | |
| WT | | | | |
| W-SAV | | T366W | | T366S L368A Y407V |
| W-SAV (354/349) | S354C | T366W | Y349C | T366S L368A Y407V |
| EW-RVT | | K360E K409W | | Q347R D399V F405T |
| EW-RVT (354/349) | S354C | K360E K409W | Y349C | Q347R D399V F405T |
| V-V | | T366V | | T366 Y407V |
| V-V (354/349) | S354C | T366V | Y349C | T366 Y407V |
| W-SG | | T366W | | T366S Y407G |
| W-SG (354/349) | S354C | T366W | Y349C | T366S Y407G |
| Nivo | | | | |

Anti-Her2/anti-CD3 bispecific antibodies including control antibodies (containing CH3 sets that are WT or comprise the W-SAV (KiH) or the EW-RVT substitution) produced in HEK293 cells are summarized in FIG. 14A.

The percentage of heterodimers (HC1/HC2 hetero) and homodimers (HC1 homo and HC2 homo) among the full-size antibodies and the presence of half antibodies (“1/2 Ab”, i.e. only comprising one heavy chain (HC)) were analyzed by LC-MS. To assess CH3 heterodimerization using LC-MS, antibody samples were digested with PNGaseF glycosidase to remove N-linked glycans and subsequently injected onto an Acquity Ultra Performance liquid chromatography (UPLC) system (Waters), equipped with a with a Thermo Scientific MabPac RP® 4 μm Column, (2.1×100 mm) maintained at 80° C. After injection, samples were eluted from the column using a 13 minute gradient from 20-55% acetonitrile at a flow rate of 0.3 mL/min (mobile phase A: 0.1% formic acid in H2O; mobile phase B: 0.1% formic acid in acetonitrile). Species eluted from the column were detected by a Q Exactive mass spectrometer (Thermo) in positive electrospray ionization mode. The instrument parameters were set as spray voltage of 3.5 kV, capillary temperature of 350° C., sheath gas flow rate at 35 and aux gas flow rate at 10 and S-lens RF level at 90. MS spectra were acquired at the scan range of 750-4000 m/z. Acquired MS data were analyzed using Biopharma Finder software (Thermo Scientific) followed by manual inspection to ensure correct assignment and relative quantification accuracy. Relative quantitation for each of the heterodimer and homodimer species were calculated based on the intensities of the peaks with respect to the sum of all the heterodimer and homodimer peak intensities.

The HEK production products were also analyzed by protein A-based size exclusion chromatograph (SEC) and ion exchange (IEX) chromatography and chromatography profiles by SEC and IEX are shown in FIGS. 14B and 14C. LC-MS results, SEC results, and titers obtained are summarized in Table 6. “(354/349)” means that HC1 contained Y349C and HC2 contained S354C.

TABLE 6
% CH3 heterodimers by LC-MS, % monomer by SEC, and titers of Cycle 1 outputs.

| | | | | | | SEC % | |
|---|---|---|---|---|---|---|---|
| | Sample | HC1 | HC1/HC2 | HC2 | | monomer | Titer |
| CH3 Set Name | ID | homo | hetero | Homo | ½ Ab | full Ab | (mg/mL) |
| WT | A01 | 14% | 45% | 41% | | 96% | 115.1 |
| W-SAV | B01 | 0% | 84% | 16% | detected | 91% | 111.9 |
| W-SAV (354/349) | C01 | 2% | 90% | 8% | | 96% | 109.7 |
| EW-RVT | D01 | 2% | 93% | 5% | detected | 84% | 84.4 |
| EW-RVT (354/349) | E01 | 6% | 87% | 7% | | 92% | 96.9 |
| V-V | F01 | 5% | 80% | 15% | | 97% | 148.7 |
| V-V (354/349) | G01 | 8% | 80% | 12% | detected | 95% | 62.3 |
| W-SG | D02 | 0% | 91% | 9% | detected | 94% | 94.7 |
| W-SG (354/349) | E02 | 1% | 93% | 6% | | 97% | 125.9 |
| Nivo | F02 | 100% | N/A | N/A | | 99% | 60.2 |

### Example 7: Cycle 2 Library Generation Based on CH3-CH3 Interface Positions and Cycle 1 Outputs

To further explore variant CH3 domains that preferentially form CH3-CH3 heterodimers, new CH3 domain libraries were designed based on amino acid positions in the CH3-CH3 interface (“interface positions”) and KiH positions (including Cycle 1 output substitutions). First, to identify interface positions, a set of 32 high-resolution, aligned, wild-type CH3 crystal structures was assembled from the Protein Data Bank (PDB) and used for a structure-guided approach to identify CH3 interface residues for diversification. Interface residues to variegate were defined as residues with: 1) side-chain SASA (Solvent Accessible Surface Area) in monomer equal to or greater than 15%; 2) contact distance neighbor atoms are less than or equal to 8.2 Å (distance set to capture distance between known knob-in-hole mutations); and 3) residues do not point away from partner chain or into solvent (determined by manual inspection). Applying these rules resulted in the identification of 24 positions to variegate in the CH3 interface.

Next, a library was designed to test one or two “anchor” mutations on one side of the interface (chain A) against one, two, or three “neighbor” mutations on the opposing side of the interface (chain B). For each anchor position A on chain A, a set of neighbor positions B was identified as the subset of interface positions on chain B that are in contact with position A (interchain Cb-Cb distance <=8.2 Å (Cb=beta carbon). For glycine, the C-alpha atom was used, as glycine has no Cb atom). Then, combinations of all possible singlets, doublet, and triplet mutations within the set of neighbors (B) were generated. Dimers and trimers containing pairs of residues not within the intrachain Cb-Cb distance cutoff of <=8.0 Å were filtered out. The resulting set identified the following neighbor mutations to test: 24 singlets, 39 doublets, and 16 triplets.

The sets of neighbor/anchor paired positions were split into 14 library pools for screening via the following steps: 1) neighbor/anchor paired positions were sorted by increasing diversity (singlets, doublets, triplets) and by general position in the protein; and 2) neighbor/anchor pairs were combined into pools (choosing the closest pool as measured by interchain contact distance) until the diversity limit was reached. Each individual library pool contained ˜10⁶ diversity. Additionally, two pools were built on outputs obtained in Example 1 (T366V/Y407V (“V-V”) and T366W/T366S Y407G (“W-SG”)). The anchor and neighbor positions and position combinations to be variegated and DNA sequence and amino acid sequence diversity possible by the variegation in some of the pools are summarized in Table 7. The DNA sequence diversity was calculated as:

$$diversity = ( ( {N}_{anchor monomers} * {32}^{1} + {N}_{anchor dimers} * {32}^{2} ) * ( {N}_{neighbor monomers} * {32}^{1} + {N}_{neighbor dimers} * {32}^{2} + {N}_{neighbor trimers} * {32}^{2} ) ) .$$

TABLE 7
CH3 domain library pools - anchor/neighbor positions and diversity

| | Anchor positions | | DNA | Amino acid |
|---|---|---|---|---|
| Pool | and combinations | Neighbor positions and combinations | diversity | diversity |
| 1 | (399); (400); (364); | (400); (364); (349, 350); (354); (356); | 9.69e+06 | 2.42e+06 |
| | (349); (350); (351); | (394); (405); (349, 439); (407); (349, 351); | | |
| | (390); (354); (392); | (354, 357); (409); (357); (351, 368); (411); | | |
| | (366); (393); (356); | (397); (399); (439); (354, 356); (357, 364); | | |
| | (394); (405); (368); | (350, 351); (351); (357, 360); (366); (354, | | |
| | (407); (370); (357); | 364); (368); (350, 368); (347, 349); (370); | | |
| | (347) | (351, 366); (360); (349, 368); (356, 357) | | |
| 2 | (399); (394); (405); | (364, 409); (390, 411); (397, 399); (366, | 9.83e+06 | 2.40e+06 |
| | (368); (400); (364); | 368); (368, 407); (392, 393); (370, 399); | | |
| | (407); (370); (409); | (368, 370); (368, 405); (354, 364); (399, | | |
| | (397); (392); (366) | 400); (366, 409); (393, 394); (399, 405); | | |
| | | (394, 407); (392, 409); (351, 366); (392, | | |
| | | 394); (351, 368); (349, 370); (366, 407); | | |
| | | (397, 405); (407, 409); (394, 397); (364, | | |
| | | 366) | | |
| 3 | (354); (349); (409) | (349, 350, 351); (354, 357, 364); (399, | 9.73e+06 | 1.51e+06 |
| | | 405); (354, 356, 357); (405, 407); (370, | | |
| | | 405) | | |
| 4 | (364); (354) | (349, 351, 368); (349, 350, 368); (349, | 8.39e+06 | 1.28e+06 |
| | | 368, 370); (350, 351, 368) | | |
| 5 | (349, 368); (366, | (366, 407); (354, 364); (351, 366) | 9.44e+06 | 1.44e+06 |
| | 368); (351, 366) | | | |
| 6 | (368); (366) | (366, 368, 407); (364, 366, 409); (351, | 8.39e+06 | 1.28e+06 |
| | | 366, 368); (366, 407, 409) | | |
| 7 | (364, 409); (366, | (366, 407); (368, 407); (368, 370) | 9.44e+06 | 1.44e+06 |
| | 407); (366, 409) | | | |
| 8 | (392, 409); (368, | (399, 405); (394, 407); (407, 409) | 9.44e+06 | 1.44e+06 |
| | 407); (394, 407) | | | |
| 9 | (407); (397); (392) | (397, 399, 405); (392, 393, 394); (366, | 9.44e+06 | 1.44e+06 |
| | | 368, 407) | | |
| 10 | (407); (409) | (368, 405, 407); (370, 399, 405); (366, | 8.39e+06 | 1.28e+06 |
| | | 407, 409); (368, 370, 405) | | |

### Example 8: Cycle 2 Selection Step1: Selection Using Modified Fc Displayed on Yeast

Library DNA was synthesized on a BioXp system and transformed into yeast as previously described. Clones were selected using anti-His and anti-Flag reagents for the high His and high Flag signals. Libraries were selected over five rounds (FIG. 15A). Clones were sequenced out of rounds 4 and 5 and analyzed for sequence uniqueness. 430 unique variant CH3 domain sets were obtained.

The 430 CH3 sets were characterized by IEX (subset) and AlphaLISA as previously described. The 430 CH3 sets were also characterized using Rosetta Scoring. ΔΔG, defined as the change in interface binding energy (as predicted by Rosetta) was determined as described in Barlow et al, J Phys Chem B (2018), pp. 5389-5399. Briefly, ΔΔG was calculated for the input PDB crystal structures and averaged (l16x, 2iwg, 4wi2, 5gsq). The heterodimer state ΔΔG score was next calculated along with the ΔΔG score for both possible homodimer states. Lastly, the Rosetta heterodimerization score (RHS) was calculated, where RHS=ΔΔG_{heterodimer}−min(ΔΔG_{homodimerA}, ΔΔG_{homodimerB}).

Based on uniqueness, IEX, AlphaLISA®, and Rosetta characterization data, 48 CH3 sets were selected for further production in HEK293 cells in Example 9. Comparison of the three variables, IEX (percent inter-chain contact), AlphaLISA® values, and Rosetta scores, between the selected CH3 sets and unselected CH3 sets are provided in FIG. 15B.

Sets were further characterized by sequence uniqueness using t-SNE (t-distributed stochastic neighbor embedding) visualization. A t-SNE plot was constructed to visualize position space in a 2d plot, where each point represented a set of mutated positions, and points closer together on the plot contained similar mutated positions. Clones selected to be carried forward spanned a broad range of the design space visualized in the t-SNE plot (FIGS. 15C and 15D). Clones selected to be carried forward span a broad range of the design space as visualized in the t-SNE plot.

### Example 9: Cycle 2 Selection Step 2: Selection Using Modified Fc Production in HEK293 Cells

The 48 variant CH3 domain sets selected in Example 8 were cloned as CH2-CH3 constructs, produced in HEK293 cells, and further characterized using LCMS (as previously described), melting temperature, and 14-day stability.

Melting temperature (Tm) was measured by differential scanning fluorometry (DSF). Twenty microliters of sample, at 0.1-1 mg/ml, was mixed with 10 μl of 20× Sypro orange (Sigma-Aldrich) before being subjected to a controlled temperature increase from 40 to 95° C., at 0.5° C. intervals in a C1000 thermocycler (BioRad) to collect Fret signal. Melting temperature was obtained by taking the negative of first derivative of the raw signal.

For accelerated stability testing, samples were incubated at 40° C. for 14 days in HBS, and samples from day 0, 1, 2, 7, and 14 were taken. Samples were then analyzed for aggregation by SEC. For SEC analysis, the running buffer composition was 200 mM sodium phosphate, 250 mM sodium chloride, pH 7.0. Accelerated stability slope was calculated from the percent aggregated, measured on the SEC.

Based on the additional characterization data, five CH3 sets were selected as Cycle 2 outputs for further production as an IgG-like bispecific antibody (BsAb) in Example 10. FIG. 16 provides plots showing % heterodimer values measured by LC-MS and stability measured by SEC of the tested CH3 sets, with filled circle data points representing the variant CH3 domain sets nominated for bispecific antibody production in HEK293 cells. Table 8 summarizes the five Cycle 2 outputs along with controls (wild-type and W-SAV (i.e., KiH”)) with respective % heterodimer values measured by LC-MS and melting temperature Tm measured by Differential scanning fluorimetry (DSF).

TABLE 8
Summary of Cycle 2 outputs

| CH3 Set | Chain A | Chain B | % heterodimer | Tm by |
|---|---|---|---|---|
| Name | substitutions | substitutions | modified Fc (by LC-MS) | DSF (° C.) |
| WT | No substitution | No substitution | — | 66.5 |
| W-SAV | T366W | T366S L368A Y407V | 86% | 60.0 |
| QR-F | T366Q K409R | L368F | 100% | 64.0 |
| RG-FG | T366R K409G | L368F K370G | 90% | 64.0 |
| TL-QL | S400T K409L | D399Q F405L | 83% | 65.0 |
| DVG-VSY | S364D Y407V K409G | T366V L368S K370Y | 80% | 58.0 |
| LWG-SIG | S364L T366W K409G | T366S L368I Y407G | 90% | 62.5 |

### Example 10: Characterization of Cycle 2 Outputs Produced as Bispecific Antibodies (BsAbs) in HEK293 Cells

The five Cycle 2 output variant CH3 domain sets selected in Example 9 were produced as BsAbs with three different Fv sets to evaluate heterodimerization efficiency in an IgG-like format. The following three Fv sets were used: anti-CD3/anti-HER2 (Adimab), anti-CD20/anti-CD3 (Regeneron), or anti-HEL/anti-BCMA (Nanjing Legend Bio/Janssen), in different orientations (Orientation 1 or Orientation 2) with a total of five different structures per output variant CH3 domain set, as shown in FIG. 17A. The anti-BCMA antigen-binding domain is a nanobody (VHH).

The wild-type CH3 domain set (i.e. dimer of the reference sequence SEQ ID NO: 1), either in bispecifics or monospecifics, were used as a negative control, and the W-SAV (i.e., KiH”) substitution set, used in an anti-CD3/anti-HER2 bispecific, was used as a positive control. Furthermore, anti-CD3/anti-HER2 bispecific antibodies comprising a wild-type CH3 set or a Cycle 2 output variant CH3 domain set in Orientation 1 additionally incorporated with the S354C/Y349C substitutions were also produced.

In total, 41 different antibodies, as summarized in Table 9, were produced. The heavy and light chain sequences of the antibodies of Table 9 are provided in Appendix Tables A-D.

TABLE 9
Bispecific antibodies comprising a Cycle 2 output CH3 set with or without CH3
disulfide bond substitutions or a control CH3 set produced in HEK293 cells.

| | | | Chain A | | Chain B | |
|---|---|---|---|---|---|---|
| | Antibody | CH3 | Variable | Chain A CH3 | Variable | Chain B CH3 |
| Index | Name | Set Name | Region | Substitutions | Region | Substitutions |
| 1 | ADI-59166 | WT | Anti-CD3 | WT | Anti-HER2 | WT |
| 2 | ADI-59167 | QR-F | | T366Q K409R | | L368F |
| 3 | ADI-59168 | RG-FG | | T366R K409G | | L368F K370G |
| 4 | ADI-59169 | TL-QL | | S400T K409L | | D399Q F405L |
| 5 | ADI-59170 | DVG-VSY | | S364D Y407V K409G | | T366V L368S K370Y |
| 6 | ADI-59171 | LWG-SIG | | S364L T366W K409G | | T366S L368I Y407G |
| 7 | ADI-59172 | QR-F | | L368F | | T366Q K409R |
| 8 | ADI-59173 | RG-FG | | L368F K370G | | T366R K409G |
| 9 | ADI-59174 | TL-QL | | D399Q F405L | | S400T K409L |
| 10 | ADI-59175 | DVG-VSY | | T366V L368S K370Y | | S364D Y407V K409G |
| 11 | ADI-59176 | LWG-SIG | | T366S L368I Y407G | | S364L T366W K409G |
| 12 | ADI-59177 | WT | | S354C | | Y349C |
| | | (354/349) | | | | |
| 13 | ADI-59178 | QR-F | | S354C T366Q K409R | | Y349C L368F |
| | | (354/349) | | | | |
| 14 | ADI-59179 | RG-FG | | S354C T366R K409G | | Y349C L368F K370G |
| | | (354/349) | | | | |
| 15 | ADI-59180 | TL-QL | | S354C S400T K409L | | Y349C D399Q F405L |
| | | (354/349) | | | | |
| 16 | ADI-59181 | DVG-VSY | | S354C S364D Y407V | | Y349C T366V L368S |
| | | (354/349) | | K409G | | K370Y |
| 17 | ADI-59182 | LWG-SIG | | S354C S364L T366W | | Y349C T366S L368I |
| | | (354/349) | | K409G | | Y407G |
| 18 | ADI-59183 | WT | Anti-CD20 | WT | Anti-CD3 | WT |
| 19 | ADI-59184 | QR-F | | T366Q K409R | | L368F |
| 20 | ADI-59185 | RG-FG | | T366R K409G | | L368F K370G |
| 21 | ADI-59186 | TL-QL | | S400T K409L | | D399Q F405L |
| 22 | ADI-59187 | DVG-VSY | | S364D Y407V K409G | | T366V L368S K370Y |
| 23 | ADI-59188 | LWG-SIG | | S364L T366W K409G | | T366S L368I Y407G |
| 24 | ADI-59189 | QR-F | | L368F | | T366Q K409R |
| 25 | ADI-59190 | RG-FG | | L368F K370G | | T366R K409G |
| 26 | ADI-59191 | TL-QL | | D399Q F405L | | S400T K409L |
| 27 | ADI-59192 | DVG-VSY | | T366V L368S K370Y | | S364D Y407V K409G |
| 28 | ADI-59193 | LWG-SIG | | T366S L368I Y407G | | S364L T366W K409G |
| 29 | ADI-59194 | WT | Anti-HEL | WT | Anti-BCMA | WT |
| 30 | ADI-59195 | QR-F | | T366Q K409R | | L368F |
| 31 | ADI-59196 | RG-FG | | T366R K409G | | L368F K370G |
| 32 | ADI-59197 | TL-QL | | S400T K409L | | D399Q F405L |
| 33 | ADI-59198 | DVG-VSY | | S364D Y407V K409G | | T366V L368S K370Y |
| 34 | ADI-59199 | LWG-SIG | | S364L T366W K409G | | T366S L368I Y407G |
| 35 | ADI-59200 | KiH | Anti-CD3 | T366S L368A Y407V | Anti-HER2 | T366W |
| 36 | ADI-59201 | WT | Anti-CD3 | WT | N.A. | N.A. |
| 37 | ADI-59202 | WT | Anti-HER2 | WT | N.A. | N.A. |
| 38 | ADI-59203 | WT | Anti-CD20 | WT | N.A. | N.A. |
| 39 | ADI-59204 | WT | Anti-CD3 | WT | N.A. | N.A. |
| 40 | ADI-59205 | WT | Anti-HEL | WT | N.A. | N.A. |
| 41 | ADI-59206 | WT | Anti-BCMA | WT | N.A. | N.A. |

The antibodies in Table 9 which were produced in HEK293 cells were analyzed by LC-MS (% heterodimers and % homodimers among the full-size antibodies), IEX (% heterodimers), and AlphaLISA®, as described above. The results are summarized in Table 10.

TABLE 10
% Heterodimer analyses on HEK293-produced bispecific antibodies of Table 9.

| | | | Specificity | LC-MS | | | | |
|---|---|---|---|---|---|---|---|---|
| | Antibody | CH3 Set | (Chain A/ | | % Homo | % Homo | IEX | AlphaLISA ® |
| Index | Name | Name | Chain B) | % Hetero | A | B | % Hetero | % Hetero |
| 1 | ADI-59166 | WT | CD3/HER2 | 46.0 | 31.0 | 23.0 | 45.6 | 24.0 |
| 2 | ADI-59167 | QR-F | | 78.0 | 14.0 | 8.0 | 75.0 | 50.1 |
| 3 | ADI-59168 | RG-FG | | 85.0 | 0.0 | 11.0 | 81.9 | 66.9 |
| 4 | ADI-59169 | TL-QL | | 83.0 | 14.0 | 3.0 | 81.3 | 63.3 |
| 5 | ADI-59170 | DVG-VSY | | 83.0 | 7.0 | 10.0 | 80.6 | 65.3 |
| 6 | ADI-59171 | LWG-SIG | | 95.0 | 3.0 | 0.0 | 90.9 | 82.4 |
| 7 | ADI-59172 | QR-F | | 77.0 | 18.0 | 5.0 | 73.4 | 50.4 |
| 8 | ADI-59173 | RG-FG | | 78.0 | 20.0 | 0.0 | 75.7 | 66.9 |
| 9 | ADI-59174 | TL-QL | | 82.0 | 6.0 | 12.0 | 76.7 | 97.1 |
| 10 | ADI-59175 | DVG-VSY | | 79.0 | 18.0 | 3.0 | 76.0 | 80.5 |
| 11 | ADI-59176 | LWG-SIG | | 90.0 | 0.0 | 2.0 | 90.2 | 129.7 |
| 12 | ADI-59177 | WT | | 67.0 | 15.0 | 12.0 | 47.7 | 38.7 |
| | | (354/349) | | | | | | |
| 13 | ADI-59178 | QR-F | | 100.0 | 0.0 | 0.0 | 77.6 | 95.0 |
| | | (354/349) | | | | | | |
| 14 | ADI-59179 | RG-FG | | 97.0 | 3% unknown peaks | | 85.5 | 119.3 |
| | | (354/349) | | | | | | |
| 15 | ADI-59180 | TL-QL | | 53.0 | 47% various | | 80.6 | 103.7 |
| | | (354/349) | | | incomplete | | | |
| | | | | | antibodies | | | |
| 16 | ADI-59181 | DVG-VSY | | 95.0 | 5% unknown peaks | | 75.6 | 115.1 |
| | | (354/349) | | | | | | |
| 17 | ADI-59182 | LWG-SIG | | 96.0 | 4.0 | 0.0 | 91.5 | 122.5 |
| | | (354/349) | | | | | | |
| 18 | ADI-59183 | WT | CD20/CD3 | 44.0 | 23.0 | 33.0 | 45.0 | N.D. |
| 19 | ADI-59184 | QR-F | | 73.0 | 4.0 | 23.0 | 62.1 | N.D. |
| 20 | ADI-59185 | RG-FG | | 81.0 | 0.0 | 19.0 | 73.1 | N.D. |
| 21 | ADI-59186 | TL-QL | | 83.0 | 8.0 | 9.0 | 73.5 | N.D. |
| 22 | ADI-59187 | DVG-VSY | | 75.0 | 4.0 | 21.0 | 63.5 | N.D. |
| 23 | ADI-59188 | LWG-SIG | | 83.0 | 2.0 | 0.0 | 84.7 | N.D. |
| 24 | ADI-59189 | QR-F | | 77.0 | 11.0 | 12.0 | 68.1 | N.D. |
| 25 | ADI-59190 | RG-FG | | 84.0 | 10.0 | 0.0 | 81.0 | N.D. |
| 26 | ADI-59191 | TL-QL | | 80.0 | 3.0 | 17.0 | 93.4 | N.D. |
| 27 | ADI-59192 | DVG-VSY | | 82.0 | 11.0 | 7.0 | 82.8 | N.D. |
| 28 | ADI-59193 | LWG-SIG | | 94.0 | 0.0 | 4.0 | 88.1 | N.D. |
| 29 | ADI-59194 | WT | HEL/BCMA | 54.0 | 23.0 | 23.0 | 40.5 | 17.3 |
| 30 | ADI-59195 | QR-F | | 79.0 | 0.0 | 21.0 | N.D. | 26.7 |
| 31 | ADI-59196 | RG-FG | | 80.0 | 0.0 | 20.0 | N.D. | 38.0 |
| 32 | ADI-59197 | TL-QL | | 82.0 | 3.0 | 14.0 | N.D. | 36.7 |
| 33 | ADI-59198 | DVG-VSY | | 83.0 | 2.0 | 15.0 | N.D. | 72.9 |
| 34 | ADI-59199 | LWG-SIG | | 89.0 | 0.0 | 5.0 | N.D. | 110.6 |
| 35 | ADI-59200 | KiH | CD3/HER2 | 90.0 | 5.0 | 0.0 | 90.5 | 132.0 |
| 36 | ADI-59201 | WT | CD3/None | 0.0 | 100.0 | 0.0 | N.D. | N.D. |
| 37 | ADI-59202 | WT | HER2/None | 0.0 | 100.0 | 0.0 | N.D. | N.D. |
| 38 | ADI-59203 | WT | CD20/None | 0.0 | 100.0 | 0.0 | N.D. | N.D. |
| 39 | ADI-59204 | WT | CD3/None | 0.0 | 100.0 | 0.0 | N.D. | N.D. |
| 40 | ADI-59205 | WT | HEL/None | 0.0 | 100.0 | 0.0 | N.D. | N.D. |
| 41 | ADI-59206 | WT | BCMA/None | 0.0 | 100.0 | 0.0 | N.D. | N.D. |

The antibodies of Table 9 produced in HEK293 cells were further analyzed for accelerated stability by SEC, i.e., % full antibodies (“% monomer full Ab”) on Day 0 (on the day of production) and changes in % full antibodies (“Δ % monomer full Ab”) by Day 14 on protein A-purified samples. SEC results and process yield from production in HEK293 cells are summarized in Table 11.

TABLE 11
% Full antibody analyses on HEK293-produced bispecific antibodies of Table 9 and process yields.

| | | | | Pro A SEC | | |
|---|---|---|---|---|---|---|
| | | | | % | Δ % | |
| | | | Specificity | monomer | monomer full | |
| | Antibody | | (Chain A/ | full Ab | Ab | |
| Index | Name | CH3 Set Name | Chain B) | on Day 0 | by Day 14 | Process Yield (mg/L) |
| 1 | ADI-59166 | WT | CD3/HER2 | 99.0 | 2.2 | 203 |
| 2 | ADI-59167 | QR-F | | 97.2 | 1.1 | 160 |
| 3 | ADI-59168 | RG-FG | | 93.3 | 3.2 | 158 |
| 4 | ADI-59169 | TL-QL | | 96.4 | 1.8 | 243 |
| 5 | ADI-59170 | DVG-VSY | | 97.6 | 1.2 | 214 |
| 6 | ADI-59171 | LWG-SIG | | 93.6 | 1.5 | 171 |
| 7 | ADI-59172 | QR-F | | 96.9 | 0.8 | 148 |
| 8 | ADI-59173 | RG-FG | | 94.7 | 1.2 | 163 |
| 9 | ADI-59174 | TL-QL | | 94.6 | 2.9 | 166 |
| 10 | ADI-59175 | DVG-VSY | | 97.2 | 1.3 | 196 |
| 11 | ADI-59176 | LWG-SIG | | 88.4 | 1.1 | 124 |
| 12 | ADI-59177 | WT (354/349) | | 98.1 | 0.9 | 200 |
| 13 | ADI-59178 | QR-F (354/349) | | 97.3 | 1.4 | 182 |
| 14 | ADI-59179 | RG-FG (354/349) | | 96.5 | 2.0 | 148 |
| 15 | ADI-59180 | TL-QL (354/349) | | 91.1 | 1.1 | 196 |
| 16 | ADI-59181 | DVG-VSY (354/349) | | 96.9 | 1.8 | 86 |
| 17 | ADI-59182 | LWG-SIG (354/349) | | 96.5 | 1.6 | 142 |
| 18 | ADI-59183 | WT | CD20/CD3 | 98.2 | 1.3 | 213 |
| 19 | ADI-59184 | QR-F | | 97.1 | 1.8 | 174 |
| 20 | ADI-59185 | RG-FG | | 95.5 | 0.0 | 165 |
| 21 | ADI-59186 | TL-QL | | 93.2 | 3.4 | 198 |
| 22 | ADI-59187 | DVG-VSY | | 96.3 | 1.0 | 164 |
| 23 | ADI-59188 | LWG-SIG | | 85.2 | 3.4 | 146 |
| 24 | ADI-59189 | QR-F | | 97.0 | 1.6 | 189 |
| 25 | ADI-59190 | RG-FG | | 93.1 | 4.4 | 203 |
| 26 | ADI-59191 | TL-QL | | 96.3 | 0.1 | 218 |
| 27 | ADI-59192 | DVG-VSY | | 96.1 | 0.6 | 214 |
| 28 | ADI-59193 | LWG-SIG | | 92.8 | 1.7 | 185 |
| 29 | ADI-59194 | WT | HEL/BCMA | 61.0 | 9.4 | 182 |
| 30 | ADI-59195 | QR-F | | 77.7 | 8.8 | 328 |
| 31 | ADI-59196 | RG-FG | | 82.0 | 9.3 | 244 |
| 32 | ADI-59197 | TL-QL | | 84.1 | 8.5 | 227 |
| 33 | ADI-59198 | DVG-VSY | | 86.0 | 11.4 | 247 |
| 34 | ADI-59199 | LWG-SIG | | 85.9 | 4.5 | 232 |
| 35 | ADI-59200 | KiH | CD3/HER2 | 92.3 | 0.3 | 176 |
| 36 | ADI-59201 | WT | CD3/None | 96.7 | 1.5 | 207 |
| 37 | ADI-59202 | WT | HER2/None | 98.5 | 1.4 | 214 |
| 38 | ADI-59203 | WT | CD20/None | 98.3 | 1.2 | 198 |
| 39 | ADI-59204 | WT | CD3/None | 97.0 | 1.3 | 215 |
| 40 | ADI-59205 | WT | HEL/None | 99.2 | 1.5 | 198 |
| 41 | ADI-59206 | WT | BCMA/None | 98.6 | −0.3 | 313 |

The % heterodimer data, % monomer full-size antibody data, and yields were further compared among specific sets of antibodies among the 41 antibodies (FIGS. 17B-17J). In these comparisons when “anti-CD3/anti-HER2 Orientation 1”, “anti-CD3/anti-HER2 Orientation 2”, “anti-CD20/anti-CD3 Orientation 1”, “anti-CD20/anti-CD3 Orientation 2”, and “anti-HEL/anti-BCMA Orientation 1” were compared, % heterodimers as measured by LC-MS were comparable among the BsAbs having the same CH3 substitution set (FIG. 17B). When % heterodimers, as measured by LC-MS, and % heterodimers, as measured by IEX on a BsAB, were compared, the LC-MS and IEX provided different % heterodimer values (e.g., 54% and 41%, respectively) for the anti-HEL/anti-BCMA antibodies (BsAbs containing a nanobody in one Fab arm), in which the anti-BCMA binding moiety is a nanobody (“VHH”) instead of a VH/VL pair (FIG. 17C). By contrast, the LC-MS and IEX % heterodimer values correlated well for other antibodies having two VH/VL pairs (FIG. 17D). The correlation between LC-MS and IEX % heterodimer values were not as clear in some of the anti-CD3/anti-HER2 BsAbs having the 354/349 disulfide bond (FIG. 17E).

When % heterodimer values as measured by LC-MS on CH3 sets with and without the 354/349 substitutions were compared, most CH3 sets (except for “TL-QL”)) with the 354/349 substitutions showed higher % heterodimer values than the corresponding CH3 sets without 354/349 substitutions (FIG. 17F), indicating that the disulfide bond between S354C and Y349C enhances heterodimerization in most sets (except for “TL-QL”).

The results show that when % heterodimer values, as measured by AlphaLISA®, were compared with % heterodimer values, as measured by LC-MS or IEX, even if there were some discrepancies in the % values, the order (i.e., rank) of CH3 sets in terms of the heterodimerizing potential were maintained (FIG. 17G).

Overall, regardless of the method used to determine % heterodimers, the “LWG-SIG”, in Orientation 1 or Orientation 2, with or without the 354/349 substitutions, appears to provide the highest % heterodimers among the different CH3 sets tested (FIG. 17H).

The stability of BsAbs were also compared based on the % monomer full Ab on Day 0 and the change in % monomer full Ab (Δ % monomer full Ab) by Day 14, as measured by SEC. As shown in FIG. 17I, regardless of whether the antibody is an “anti-CD3/anti-HER2 Orientation 1”, “anti-CD3/anti-HER2 Orientation 2”, “anti-CD20/anti-CD3 Orientation 1”, “anti-CD20/anti-CD3 Orientation 2”, and “anti-HEL/anti-BCMA Orientation 1”, the % monomer full Ab values on Day 0 were low (i.e., low aggregation) and little aggregation occurs by Day 14.

Finally, the production yields of BsAbs in HEK293 cells were compared. As shown in FIG. 17J, regardless of whether the antibody is an “anti-CD3/anti-HER2 Orientation 1”, “anti-CD3/anti-HER2 Orientation 2”, “anti-CD3/anti-HER2 Orientation 1 with the 354/349 substitutions”, “anti-CD20/anti-CD3 Orientation 1”, “anti-CD20/anti-CD3 Orientation 2”, and “anti-HEL/anti-BCMA Orientation 1”, the production yields were similar.

### Example 11: Simultaneous Characterization of BsAbs Comprising Cycle 1 Output Substitutions, Cycle 2 Output Substitutions, or a Combination of Cycle 1 and Cycle 2 Output Substitutions

In Example 11, anti-CD3/anti-HER2 BsAbs comprising Cycle 1 output substitutions (W-SG or V-V) and BsAbs comprising Cycle 2 output substitutions (QR-F, RG-FG, TL-QL, DVG-VSY, or LWG-SIG) were compared side-by side, with or without the 354/349 substitutions. Additionally, BsAbs comprising some of the combinations of Cycle 2 output substitutions with Cycle 1 output substitutions or with KiH substitutions (WTL-SAVQL, WTL-SGQL, WQL-SAVTL, WQL-SGTL, VTL-VQL, VQL-VTL, QRQL-FTL, or VQR-VF), along with a modified version of a Cycle 2 output substitution (LWG-IG), were also tested in parallel.

The BsAbs used in Example 11 are summarized in Table 12. All BsAbs were produced in HEK293 cells.

TABLE 12
Anti-CD3/anti-HER2 bispecific antibodies (BsAbs) comprising a Cycle 1 or Cycle
2 output CH3 set, with or without CH3 disulfide bond substitutions, or comprising
a combination of Cycle 2 with Cycle 1 output or with KiH CH3 substitutions.

| | | Chain A | | Chain B | |
|---|---|---|---|---|---|
| | | Variable | Chain A CH3 | Variable | Chain B CH3 |
| Index | CH3 Set Name | Region | Substitutions | Region | Substitutions |
| 101 | WT | Anti-CD3 | WT | Anti-HER2 | WT |
| 102 | W-SAV | Anti-CD3 | T366W | Anti-HER2 | T366S L368A Y407V |
| 103 | EW-RVT | Anti-CD3 | K360E K409W | Anti-HER2 | Q347R D399V F405T |
| 104 | VYAV-VLLW | Anti-CD3 | T350V Y351Y F405A | Anti-HER2 | T350V T366L K392L |
| | | | Y407V | | T394W |
| 105 | W-SG | Anti-CD3 | T366W | Anti-HER2 | T366S Y407G |
| 106 | V-V | Anti-CD3 | Y407V | Anti-HER2 | T366V |
| 107 | QR-F | Anti-CD3 | T366Q K409R | Anti-HER2 | L368F |
| 108 | RG-FG | Anti-CD3 | T366R K409G | Anti-HER2 | L368F K370G |
| 109 | TL-QL | Anti-CD3 | S400T K409L | Anti-HER2 | D399Q F405L |
| 110 | DVG-VSY | Anti-CD3 | S364D Y407V K409G | Anti-HER2 | T366V L368S K370Y |
| 111 | LWG-SIG | Anti-CD3 | S364L T366W K409G | Anti-HER2 | T366S L368I Y407G |
| 112 | WT | Anti-CD3 | S354C | Anti-HER2 | Y349C |
| | (354/349) | | | | |
| 113 | W-SAV | Anti-CD3 | S354C T366W | Anti-HER2 | Y349C T366S L368A |
| | (354/349) | | | | Y407V |
| 114 | EW-RVT | Anti-CD3 | S354C K360E K409W | Anti-HER2 | Y349C Q347R D399V |
| | (354/349) | | | | F405T |
| 115 | W-SG | Anti-CD3 | S354C T366W | Anti-HER2 | Y349C T366S Y407G |
| | (354/349) | | | | |
| 116 | QR-F | Anti-CD3 | S354C T366Q K409R | Anti-HER2 | Y349C L368F |
| | (354/349) | | | | |
| 117 | RG-FG | Anti-CD3 | S354C T366R K409G | Anti-HER2 | Y349C L368F K370G |
| | (354/349) | | | | |
| 118 | TL-QL | Anti-CD3 | S354C S400T K409L | Anti-HER2 | Y349C D399Q F405L |
| | (354/349) | | | | |
| 119 | DVG-VSY | Anti-CD3 | S354C S364D Y407V | Anti-HER2 | Y349C T366V L368S |
| | (354/349) | | K409G | | K370Y |
| 120 | LWG-SIG | Anti-CD3 | S354C S364L T366W | Anti-HER2 | Y349C T366S L368I |
| | (354/349) | | K409G | | Y407G |
| 121 | WTL-SAVQL | Anti-CD3 | T366W S400T K409L | Anti-HER2 | T366S L368A Y407V |
| | | | | | D399Q F405L |
| 122 | WTL-SGQL | Anti-CD3 | T366W S400T K409L | Anti-HER2 | T366S Y407G D399Q |
| | | | | | F405L |
| 123 | WQL-SAVTL | Anti-CD3 | T366W D399Q F405L | Anti-HER2 | T366S L368A Y407V |
| | | | | | S400T K409L |
| 124 | WQL-SGTL | Anti-CD3 | T366W D399Q F405L | Anti-HER2 | T366S Y407G S400T |
| | | | | | K409L |
| 125 | VTL-VQL | Anti-CD3 | Y407V S400T K409L | Anti-HER2 | T366V D399Q F405L |
| 126 | VQL-VTL | Anti-CD3 | Y407V D399Q F405L | Anti-HER2 | T366V S400T K409L |
| 127 | QRQL-FTL | Anti-CD3 | T366Q K409R D399Q | Anti-HER2 | L368F S400T K409L |
| | | | F405L | | |
| 128 | VQR-VF | Anti-CD3 | Y407V T366Q K409R | Anti-HER2 | T366V L368F |
| 129 | LWG-IG | Anti-CD3 | S364L T366W K409G | Anti-HER2 | L368I Y407G |
| 130 | WT | Anti-CD3 | WT | N/A | N/A |
| 131 | WT | Anti-HER2 | WT | N/A | N/A |

The BsAbs in Table 12 which were produced in HEK293 cells were analyzed for % heterodimers by LC-MS and IEX. The results are summarized in Table 13.

TABLE 13
% Heterodimer analyses on HEK293-produced bispecific antibodies of Table 12.

| | | Specificity | | | | |
|---|---|---|---|---|---|---|
| | | (Chain A/ | LC-MS | | | IEX |
| Index | CH3 Set Name | Chain B) | % Hetero | % Homo A | % Homo B | % Hetero |
| 101 | WT | CD3/HER2 | 46.0 | 34.0 | 20.0 | 44.6 |
| 102 | W-SAV | | 92.0 | 0.0 | 3.0 | |
| 103 | EW-RVT | | 78.0 | 18.0 | 0.0 | |
| 104 | VYAV-VLLW | | 86.0 | 3.0 | 0.0 | |
| 105 | W-SG | | 75.0 | 0.0 | 0.0 | |
| 106 | V-V | | 72.0 | 23.0 | 5.0 | |
| 107 | QR-F | | 79.0 | 11.0 | 10.0 | |
| 108 | RG-FG | | 79.0 | 0.0 | 16.0 | |
| 109 | TL-QL | | 85.0 | 3.0 | 12.0 | |
| 110 | DVG-VSY | | 82.0 | 10.0 | 8.0 | |
| 111 | LWG-SIG | | 94.0 | 3.0 | 0.0 | |
| 112 | WT (354/349) | | 60.0 | 24.0 | 16.0 | 43.8 |
| 113 | W-SAV (354/349) | | 96.0 | 0.0 | 4.0 | 79.6 |
| 114 | EW-RVT (354/349) | | 96.0 | 4.0 | 0.0 | 85.1 |
| 115 | W-SG (354/349) | | 100.0 | 0.0 | 0.0 | 88.8 |
| 116 | QR-F (354/349) | | 90.0 | 0.0 | 10.0 | 64.6 |
| 117 | RG-FG (354/349) | | 100.0 | 0.0 | 0.0 | 84.6 |
| 118 | TL-QL (354/349) | | 93.0 | 7.0 | 0.0 | 74.3 |
| 119 | DVG-VSY (354/349) | | 96.0 | 2.0 | 2.0 | 79.4 |
| 120 | LWG-SIG (354/349) | | 92.0 | 4.0 | 0.0 | 89.5 |
| 121 | WTL-SAVQL | | 86.0 | 2.0 | 0.0 | |
| 122 | WTL-SGQL | | 88.0 | 0.0 | 0.0 | |
| 123 | WQL-SAVTL | | 14.0 | 7.0 | 42.0 | |
| 124 | WQL-SGTL | | 93.0 | 0.0 | 4.0 | |
| 125 | VTL-VQL | | 79.0 | 8.0 | 2.0 | |
| 126 | VQL-VTL | | 75.0 | 2.0 | 18.0 | |
| 127 | QRQL-FTL | | 72.0 | 0.0 | 22.0 | |
| 128 | VQR-VF | | 76.0 | 0.0 | 20.0 | |
| 129 | LWG-IG | | 94.0 | 3.0 | 0.0 | |
| 130 | WT | CD3/none | 100.0 | 0.0 | 0.0 | |
| 131 | WT | HER2/none | 100.0 | 0.0 | 0.0 | |

The bsAbs of Table 12 which were produced in HEK293 cells and protein A-purified were further analyzed for 0% monomer full Ab by SEC. The SEC results and process yields from production in HEK 293 cells are summarized in Table 14.

TABLE 14
% Heterodimer analyses on HEK293-produced bispecific antibodies of Table 12.

| | | Specificity | Pro A SEC | Process Yield |
|---|---|---|---|---|
| Index | CH3 Set Name | (Chain A/Chain B) | % monomer full Ab | (mg/L) |
| 101 | WT | CD3/HER2 | 99 | 170 |
| 102 | W-SAV | | 93 | 147 |
| 103 | EW-RVT | | 92 | 191 |
| 104 | VYAV-VLLW | | 88 | 184 |
| 105 | W-SG | | 84 | 130 |
| 106 | V-V | | 95 | 207 |
| 107 | QR-F | | 95 | 144 |
| 108 | RG-FG | | 91 | 151 |
| 109 | TL-QL | | 87 | 139 |
| 110 | DVG-VSY | | 93 | 165 |
| 111 | LWG-SIG | | 94 | 139 |
| 112 | WT (354/349) | | 98 | 168 |
| 113 | W-SAV (354/349) | | 91 | 118 |
| 114 | EW-RVT (354/349) | | 89 | 140 |
| 115 | W-SG (354/349) | | 93 | 172 |
| 116 | QR-F (354/349) | | 98 | 93 |
| 117 | RG-FG (354/349) | | 88 | 127 |
| 118 | TL-QL (354/349) | | 92 | 141 |
| 119 | DVG-VSY (354/349) | | 94 | 164 |
| 120 | LWG-SIG (354/349) | | 92 | 174 |
| 121 | WTL-SAVQL | | 91 | 221 |
| 122 | WTL-SGQL | | 92 | 238 |
| 123 | WQL-SAVTL | | 58 | 183 |
| 124 | WQL-SGTL | | 92 | 210 |
| 125 | VTL-VQL | | 85 | 134 |
| 126 | VQL-VTL | | 91 | 222 |
| 127 | QRQL-FTL | | 92 | 176 |
| 128 | VQR-VF | | 93 | 229 |
| 129 | LWG-IG | | 93 | 217 |
| 130 | WT | CD3/none | 96 | 144 |
| 131 | WT | HER2/none | 96 | 123 |

The % heterodimer data, % monomer full-size antibody data, and yields were further compared among specific sets of antibodies among the 31 antibodies. This comparison revealed that when % heterodimer values, measured by LC-MS and IEX, were compared on BsAbs comprising wild-type or Cycle 1 or 2 output CH3 domains without the 354/349 substitutions, good correlation was observed between LC-MS and IEX values (FIG. 18A). Moreover, when the same comparison was made on BsAbs comprising wild-type or Cycle 1 or 2 output CH3 domains further comprising the 354/349 substitutions, even if there were some discrepancies in the % values, the order (i.e., rank) of CH3 sets in terms of the heterodimerizing potential were maintained (FIG. 18B).

As was observed in Example 10, among the CH3 sets identified in Cycle 1 and Cycle 2 selections (i.e., Cycle 1 outputs (W-SG and V-V) and Cycle 2 outputs (QR-F, RG-FG, TL-QL, DVG-VSY, and LWG-SIG)) and the pre-existing heterodimerization technologies tested (i.e., KiH, EW-RVT, and ZW1), with or without the 354/349 substitutions, LWG-SIG consistently provided highest rates of heterodimerization, with or without the 354/349 substitutions (FIG. 18C). W-SG, RG-FG, and QR-F, when combined with the 354/349 substitutions, also exceeded the rate of heterodimerization of the pre-existing heterodimerization technologies tested (i.e., KiH, EW-RVT, and ZW1).

Interestingly, several from Cycle 1 and Cycle 2 output CH3 sets, such as V-V and QR-F (with or without the 354/349 substitutions), showed less aggregation as measured by SEC on protein A-purified production products, compared to the pre-existing heterodimerization technologies tested (i.e., KiH, EW-RVT, and ZW1) (FIG. 18D).

When CH3 sets were compared between those with and without the 354/349 substitutions, the CH3 sets with the 354/349 substitutions overall showed higher % heterodimer values measured by LC-MS and higher % monomer full Ab values measured by SEC (FIG. 18E). This indicates that addition of the 354/349 substitutions facilitated CH3 heterodimerization and improved stability.

Production yields were compared across different CH3 sets, with or without the 354/349 substitutions. As shown in FIG. 18F, no CH3 substitution sets appeared to reduce production yields. Furthermore, it was observed that certain CH3 sets, e.g., V-V, seem to have higher yields over the pre-existing heterodimerization technologies tested (i.e., KiH, EW-RVT, and ZW1). Additionally, as evident from Table 13, many of the combination substitution sets (combination between Cycle 1 and Cycle 2 substitutions or between Cycle 2 and KiH substitutions), e.g., WTL-SAVQL, WTL-SGQL, WQL-SGTL, and VQL-VTL, and particularly the LWG-IG substitution provided superior production yields over the pre-existing heterodimerization technologies tested (i.e., KiH, EW-RVT, and ZW1).

The new CH3 set “LWG-IG”, a variant of Cycle 2 output LWG-SIG, showed overall similar characteristics with LWG-SIG, except that LWG-IG provided even higher production yield over LWG-SIG, as shown in FIG. 18G. This similarity between LWG-SIG and LWG-IG is in agreement with the Rosetta heterodimer scores of LWG-SIG and LWG-IG.

Many of the inventive CH3 domain substitution sets described in Examples, such as Cycle 1 and Cycle 2 outputs and additional variant CH3 domains tested in Example 11 (i.e., combinations between Cycle 1 and Cycle 2 output substitutions, combinations between Cycle 2 and Cycle 2 output substitutions, combinations between Cycle 2 output and KiH, substitutions, and LWG-IG), with or without the CH3 disulfide bond substitutions (which may be the 354/349 or 349/354 substitutions), and related variations thereof are provided in Appendix Tables E-G. Exemplary variant CH3 domain sequences, in which such CH3 substitution sets are incorporated to the reference CH3 domain sequence of SEQ ID NO: 1 are also provided in Appendix Tables E-G. The exemplary sequences are those used in Examples herein. It is to be noted, however, that those sequences are exemplary, and the same CH3 substitution sets may be incorporated into any CH3 domain sequences, i.e., not limited to SEQ ID NO: 1. A summary of SEQ ID NOs assigned to those exemplary variant CH3 domain sequences is provided in FIG. 19.

### Example 12: Simultaneous Evaluation of Effects of Cycle 1 Output Sets and Cycle 2 Output Sets on Tm

In Example 12, two variant CH3 domain sets from Cycle 1 (W-SG and V-V) and four variant CH3 domain sets from Cycle 2 (QR-F, RG-FG, DVG-VSY, and LWG-SIG) along with pre-existing CH3 sets (W-SAV (also referred to as KiH), VYAV-VLLW (also referred to as ZW1), and EW-RVT), with or without the 354/349 substitutions, were produced in HEK293 cells as CH2-CH3 constructs (i.e., Fc-only constructs) and effects of the CH3 substitutions on melting temperatures (Tm) measured by differential scanning calorimetry (DSC) were analyzed.

### Method

### Protein Expression:

Heterodimer fc-only constructs of CH3 mutation sets were expressed as HIS tag and FLAG purification tags such that the heterodimer would contain both an HIS and FLAG tag. Proteins were transiently transfected in HEK cells as previously described.

### Primary Capture:

Transiently transfected HEK cultures were harvested by centrifugation for 5 min at 2400G. The supernatant was decanted off the cell pellet and spun a second time for 5 min at 2400G before being loaded onto Ni Sepharose 6 Fast Flow resin (Cytiva 1753180) that had been equilibrated with 10 column volumes of 20 mM sodium phosphate, 500 mM NaCl, pH 7.4 buffer. The bound protein was then washed with 5 column volumes of equilibration buffer containing 2 mM imidazole and eluted with 5 column volumes of equilibration buffer containing 250 mM imidazole. The eluate was immediately desalted into 25 mM HEPES, 150 mM sodium chloride, pH 7.2 using Sephadex G25 medium (Cytiva 1700330).

### Secondary Purification:

The protein was treated with 10× binding buffer (0.5M tris, 1.5M sodium chloride, 100 mM calcium chloride, pH 7.4) prior to being loaded onto anti-FLAG M1 resin (Sigma Aldrich A4596) that had been equilibrated with 15 column volumes of 50 mM tris, 150 mM sodium chloride, pH 7.4. The bound protein was washed with 36 column volumes of equilibration buffer containing 1 mM calcium chloride and eluted with 4 column volumes of equilibration buffer containing 2 mM EDTA. The eluate was buffer exchanged into 25 mM HEPES, 150 mM sodium chloride, pH 7.2 over 3×5 diafiltration volumes through Amicon™ Ultra-15 Centrifugal Filter Units. The protein was normalized to a final target concentration of 1 mg/mL and 0.2 um filtered.

### Tm Measurement by DSC:

DSC measurements were carried out by using MicroCal VP-capillary DSC (now Malvern Panalytical). Data were collected typically over a range of 15-100° C. at 120° C./hr with HBS buffer as the reference. 400 μL of sample were used for the DSC study. The running software was VPViewer2000. Analysis software was Microcal, LLC Cap DSC Version Origin70-L3 and was used to convert the raw data into molar heat capacity (MHC).

### Results

The 1st and 2nd Tm values (Tm1 and Tm2) obtained are provided in Table 15 with the substitutions in each CH3 domain. Tm2 values are further visualized in FIG. 20.

TABLE 15
Tm analyses on Fc constructs with Cycle 1 or Cycle 2 output CH3
sets with or without the CH3 disulfide bond substitutions.

| CH3 Set | Chain A | Chain B | Tm1 by DSC | Tm2 by DSC |
|---|---|---|---|---|
| Name | substitutions | substitutions | (° C.) | (° C.) |
| WT | No substitution | No substitution | 71.4 | 85.7 |
| W-SAV | T366W | T366S L368A Y407V | 72.5 | 72.5* |
| VYAV-VLLW | T350V Y351Y F405A Y407V | T350V T366L K392L T394W | 71.9 | 84.9 |
| W-SG | T366W | T366S Y407G | 69.9 | 71.9 |
| V-V | Y407V | T366V | 72.2 | 76.0 |
| QR-F | T366Q K409R | L368F | 72.5 | 72.5* |
| RG-FG | T366R K409G | L368F K370G | 72.4 | 72.4* |
| DVG-VSY | S364D Y407V K409G | T366V L368S K370Y | 70.3 | 72.1 |
| LWG-SIG | S364L T366W K409G | T366S L368I Y407G | 69.5 | 71.5 |
| W-SAV | S354C T366W | Y349C T366S L368A Y407V | 71.5 | 82.7 |
| (354/349) | | | | |
| EW-RVT | S354C K360E K409W | Y349C Q347R D399V F405T | 71.8 | 77.6 |
| (354/349) | | | | |
| W-SG | S354C T366W | Y349C T366S Y407G | 71.6 | 79.9 |
| (354/349) | | | | |
| QR-F | S354C T366Q K409R | Y349C L368F | 71.5 | 80.2 |
| (354/349) | | | | |
| RG-FG | S354C T366R K409G | Y349C L368F K370G | 72.4 | 76.4 |
| (354/349) | | | | |
| V-V | S354C Y407V | Y349C T366V | 71.3 | 79.8 |
| (354/349) | | | | |
| DVG-VSY | S354C S364D Y407V K409G | Y349C T366V L368S K370Y | 70.6 | 72.7 |
| (354/349) | | | | |
| LWG-SIG | S354C S364L T366W K409G | Y349C T366S L368I Y407G | 72.3 | 76.6 |
| (354/349) | | | | |

*In these antibodies, Tm2 values were equal to Tm1 values. Generally, Tm1 is associated with CH2 dissociation and Tm2 is associated with CH3 dissociation. Therefore, when Tm1 and Tm2 values are same, it indicates that CH2 and CH3 dissociations occur at the same time.

As shown in Table 15, all variant CH3 sets presented largely similar Tm1 values. Tm1 values for V-V, QR-F, RG-FG, RG-FG (354/349), and LWG-SIG (354/349) were slightly higher than the Tm1 of WT CH3 set. As shown in Table 15 and in FIG. 20, CH3 sets with the 354/349 substitutions presented higher Tm2 values than the corresponding CH3 sets without the 354/349 substitutions. In CH3 sets without the 354/349 substitutions which did not provide a distinct Tm2 peak (W-SAV, QR-F, and RG-FG), a more distinct Tm2 peak was obtained when the 354/349 substitutions were added.

### Example 13: Structural Analysis of IgG1 Fc with LWG-SIG

To analyze the effect of substitutions in CH3 on CH3-CH3 interaction, a Fc-only construct comprising the LWG-SIG set, named ADI-64950, was produced in CHO-K1 cells and the crystal structure was analyzed.

### Method

### Crystallization and Structure Determination of ADI-64950 IgG1 Fc:

ADI-64950, which is a human IgG1 Fc dimer comprising a variant CH3 (of IgG1) domain comprising T366S, L368I, and Y407G on Chain A and a variant CH3 (of IgG1) domain comprising S364L, T366W, K409G on Chain B, where Chain B also contains the Fc-III knockout substitutions (M252E, I253A, and Y436A), was concentrated to 10.9 mg/mL into a buffer containing 2 mM Tris-HCl pH 8.0 and 150 mM NaCl. ADI-64950 at 10.9 mg/ml was mixed with 1 mM Fc-III dissolved in DMSO to 25 mM. JCSG+, PACT, BCS and ProPlex screens were set up using 100+100 nl sitting drops in MRC plates over reservoir. The crystal used for data collection was grown in the JCSG+ screen, well B9, over reservoir: 0.1 M citrate pH 5.0 and 20% (w/v) PEG (polyethylene glycol) 6000. Crystals were flash-frozen in liquid nitrogen after addition of cryo-solution containing: 0.1 M citrate pH 5.0, 20% (w/v) PEG (polyethylene glycol) 6000 and 25% glycerol. Data were collected at 100 K at station BioMAX, MAX IV, Lund, Sweden (k=0.9763 Å). 3600 images were collected with an oscillation range of 0.1° per image. The beamline was equipped with an Eiger 16M hybrid-pixel detector. Data extending to 2.7 Å were processed using autoPROC (Vonrhein, C., Flensburg, C., Keller, P., Sharff, A., Smart, O., Paciorek, W., Womack, T. & Bricogne, G. (2011). Data processing and analysis with the autoPROC toolbox. Acta Crystallogr. D Biol. Crystallogr. 67, 293-302), which includes the software XDS (Kabsch W. (2010) “XDS” Acta. Crystallogr. D Biol. Crystallogr. 66, 125-132) and Aimless (Evans P. R. and Murshudov, G. N. (2013) “How good are my data and what is the resolution” Acta Crystallogr D Biol. Crystallogr. 69, 1204-1214). Crystals consisted of a single molecule in the asymmetric unit (ASU) in P21 space group. A molecular replacement solution for ADI-64950 was obtained by PHASER (McCoy, A. J., Grosse-Kunstleve, R. W., Adams, P. D., Winn, M. D., Storoni, L. C., & Read, R. J. (2007). Phaser crystallographic software. Journal of Applied Crystallography, 40(4), 658-674) using the PDB entry 5DJ6 (Leaver-Fay et al. (2016). Computationally Designed Bispecific Antibodies using Negative State Repertoires. Structure. 24(4):641-651). The structures were built manually in COOT (Emsley P., Lohkamp, B., Scott, W. G. and Cowtan K. (2010) “Features and development of Coot” Acta Crystallogr. D Biol. Crystallogr. 66, 486-501) and refined using Refmac5 (Murshudov, G. N., Skubak, P., Lebedev, A. A., Pannu, N. S., Steiner, R. A., Nicholls, R. A., Winn, M. D. Long, F. and Vagin, A. A. (2011) REFMAC5 for the refinement of macromolecular crystal structures, Acta Crystallogr. D Biol. Crystallogr. 67, 355-367) to a final R and Rfree of 20.4% and 25.6%, respectively (FIG. 21).

PDB ID: 5JII as the WT reference was used for comparison.

### Results

### CH3-CH3 Pairing Mediated by Substitutions Present in ADI-64950:

ADI-64950 was found to have a stronger CH3-CH3 interaction than a human IgG1 Fe dimer comprising WT CH3 domains, based on the free energy gain upon formation of the CH3-CH3 interface calculated via PISA (Proteins, Interfaces, Structures and Assemblies) (FIG. 21). Pairing between Chain A (T366S, L368I, and Y407G) and Chain B (S364L, T366W, K409G) (A-B heterodimer) was found to be mediated by several novel polar contacts at the CH3-CH3 interface (FIG. 22). These contacts include: a salt-bridge formed between Chain A Lys409 and Chain B Asp399; and hydrogen bonds between Chain A Lys409 and Chain B Asp399, between Chain A Glu357 and Chain B Lys370, between Chain A Ser364 and Chain B Lys370, between Chain A Leu398 and Chain B Lys392, between Chain A T366S and Chain B Tyr407, between Chain A Lys360 and Chain B Tyr349, and between Chain A Ser354 and Chain B Thr350 (FIG. 22).

Steric clashes at the CH3-CH3 interface of ADI-64950 off-products A-A and B-B homodimers are predicted to reduce propensity for mispairing:

Potential off-product homodimers “A-A” (i.e., dimer of two Chain A) and “B-B” (i.e., dimer of two Chain B) were generated by aligning the Chain A to Chain B and vice-versa and then probed for clashes in PyMol. Several substantial clashes were observed (FIG. 23) including (a) Chain A Phe405 and Chain A Lys409 (b) Chain B Tyr349 and Chain B Asp356 (c) Chain B T366W and Chain B Tyr407 as well as the orthogonal (d) Chain B T366W and Chain B Tyr407. These clashes are predicted to reduce the propensity of the formation of the ADI-64950 mispaired homodimers constructs A-A and B-B.

### Example 14: cFAE Compatibility Test, Part 1—Production of Antibodies Comprising Two Identical Variant CH3 Domains

Some of many different methods of making bispecific antibodies rely on FAE. Some of the Cycle 1 and 2 output CH3 domain sets were tested for their applicability to cFAE-based manufacturing methods, using the WT CH3 set as a negative control and the pre-existing CH3 domain set “R-L” known to mediate FEA between IgG1 molecules (Labrijn et al. Proc Natl Acad Sci USA. 2013 Mar. 26; 110(13):5145-50) as a positive control, (see Table 16).

TABLE 16
CH3 sets tested for cFAE-based manufacturing

| | Chain A | Chain B |
|---|---|---|
| CH3 Set Name | Substitutions | substitutions |
| WT | No substitution | No substitution |
| R-L | K409R | F405L |
| V-V | Y407V | T366V |
| QR-F | T366Q K409R | L368F |
| RG-FG | T366R K409G | L368F K370G |

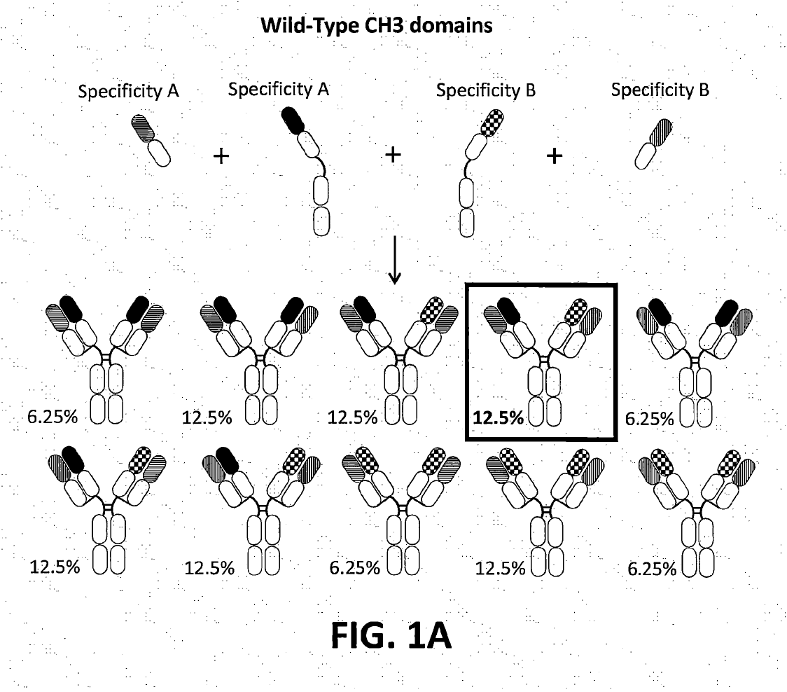

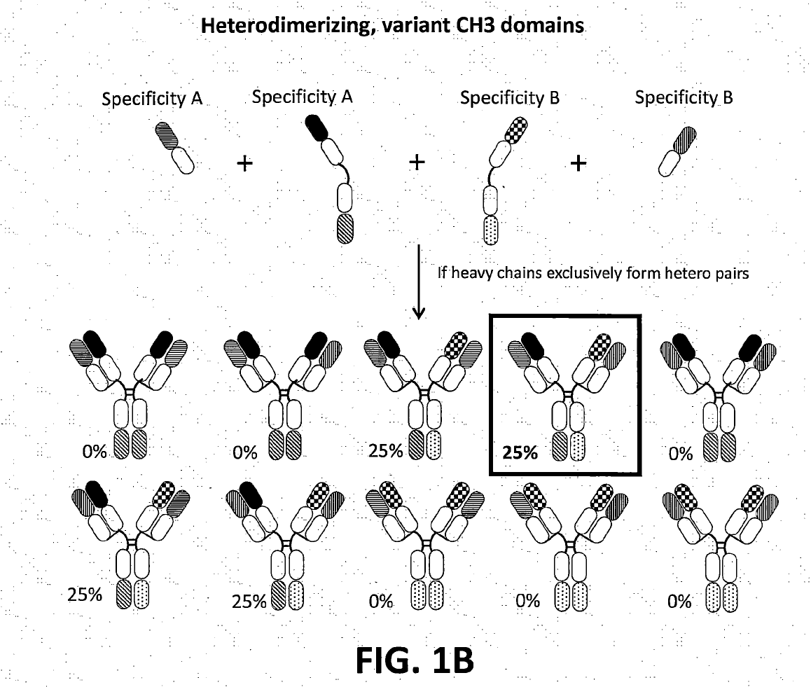

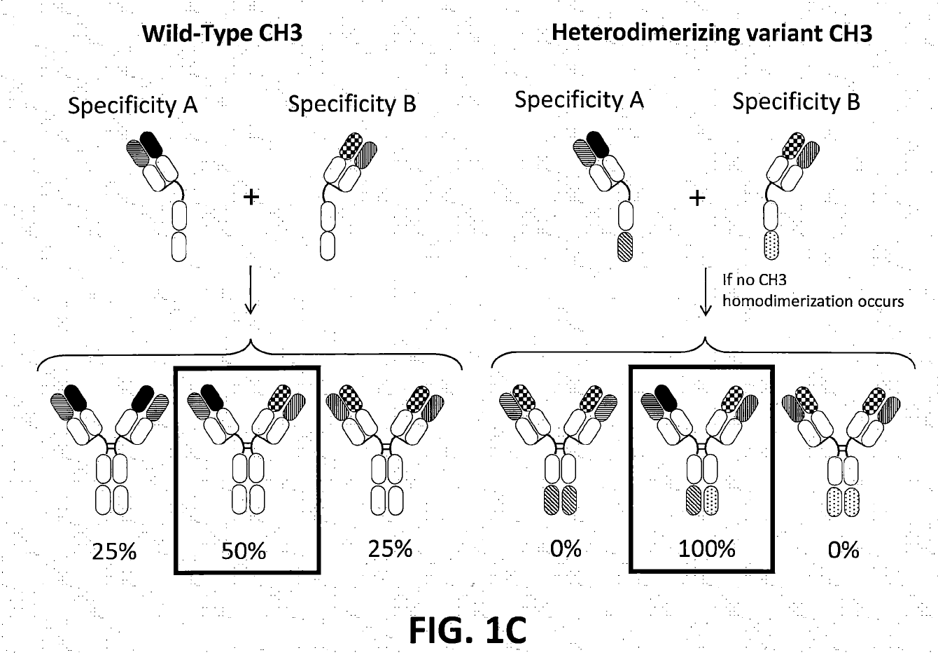

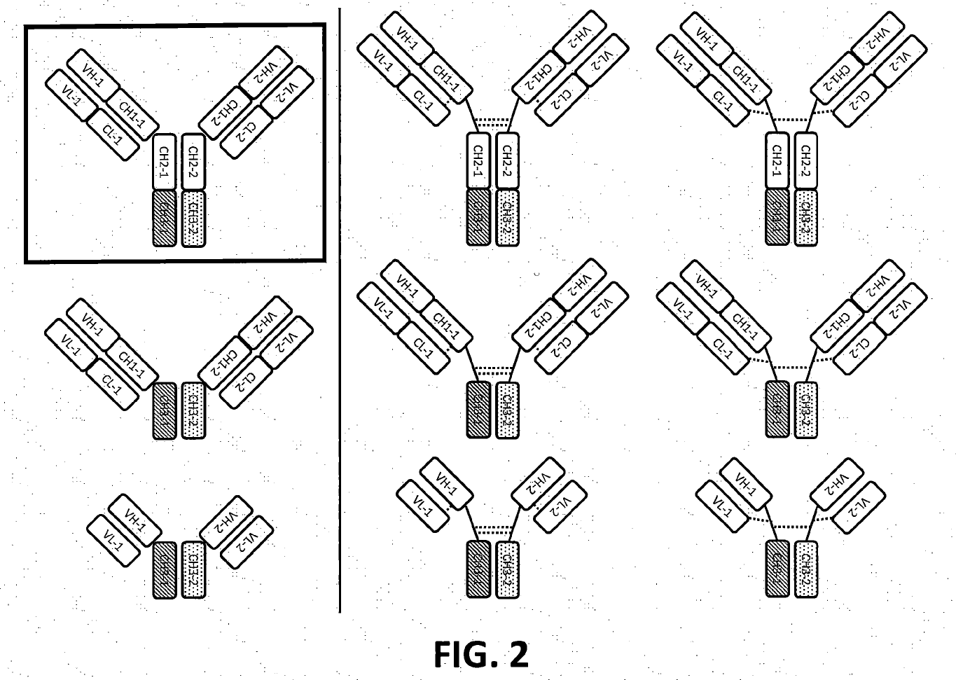

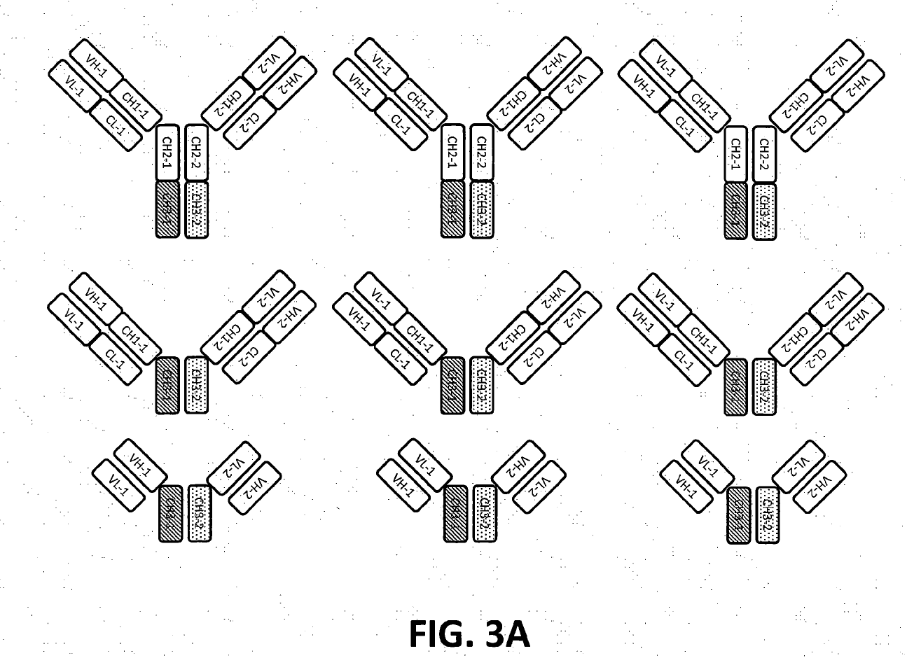

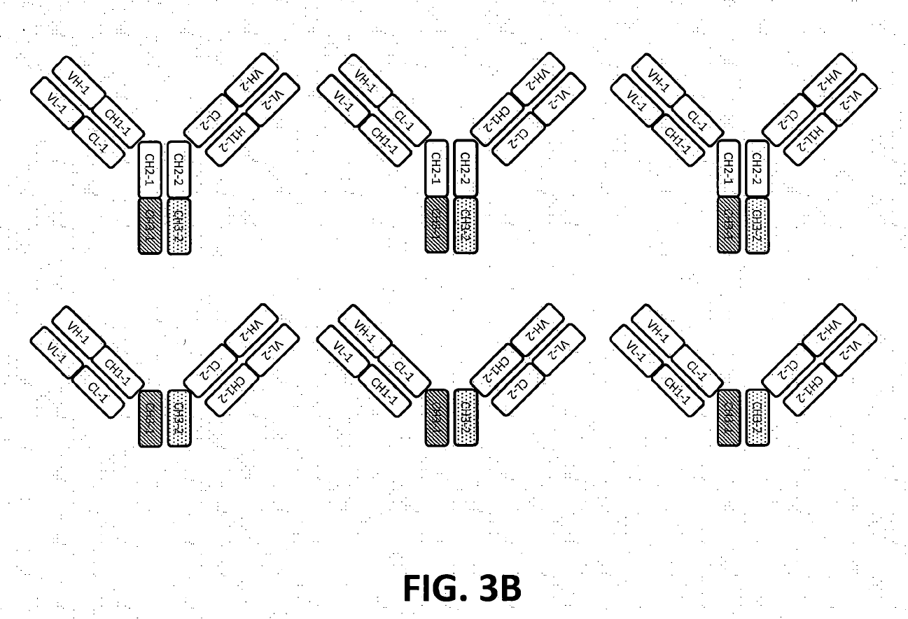

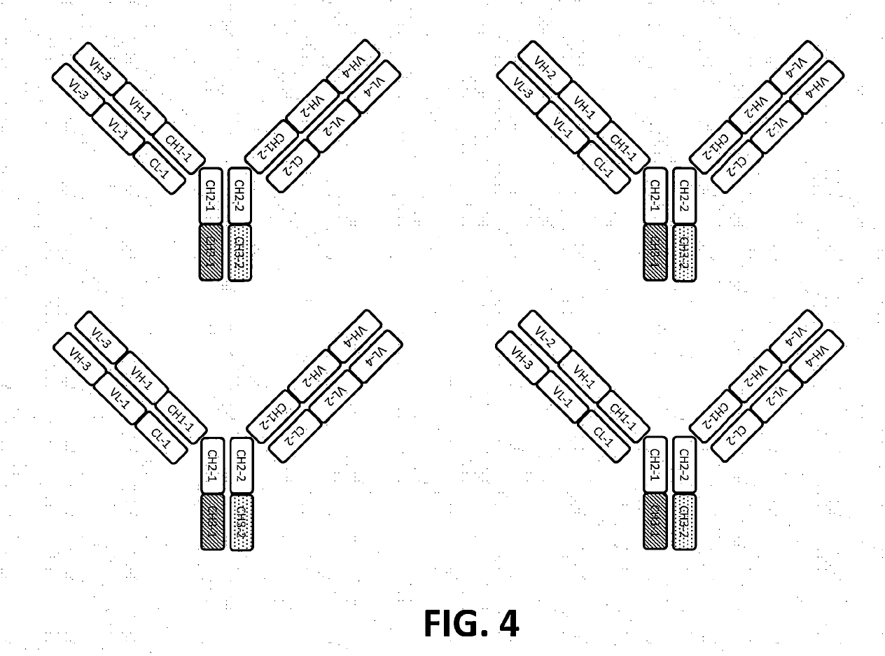

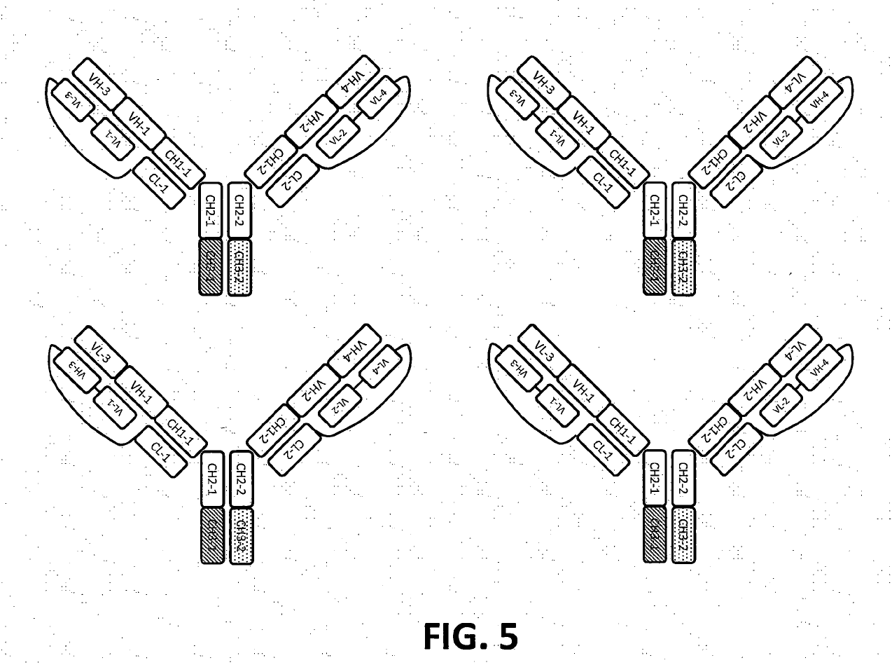

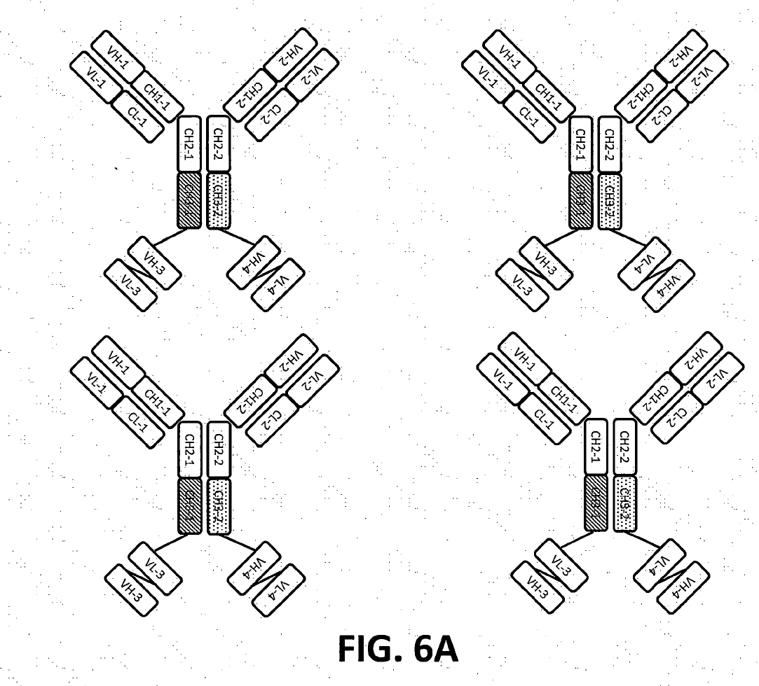

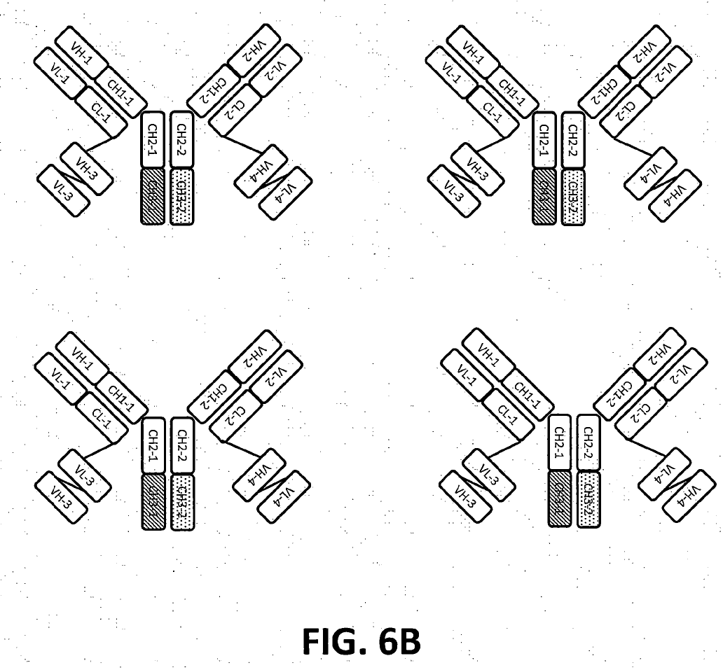

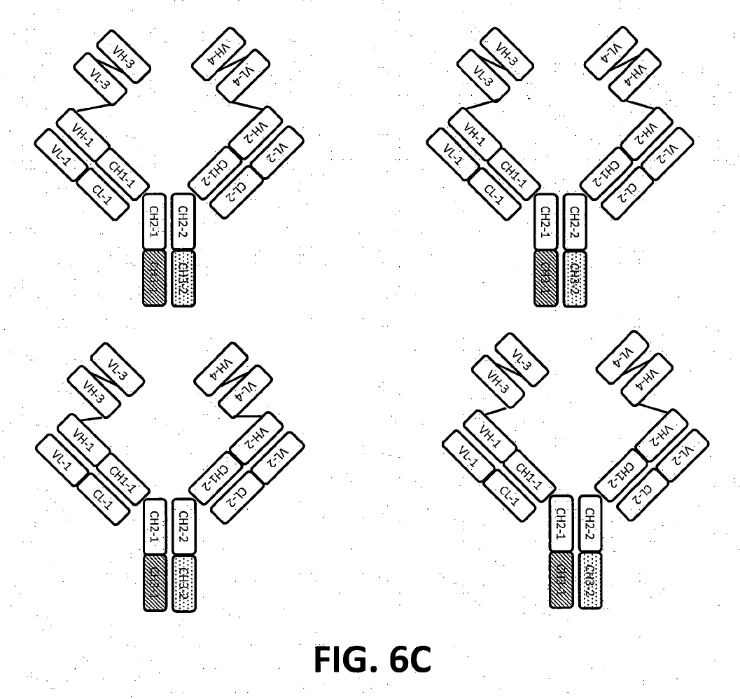

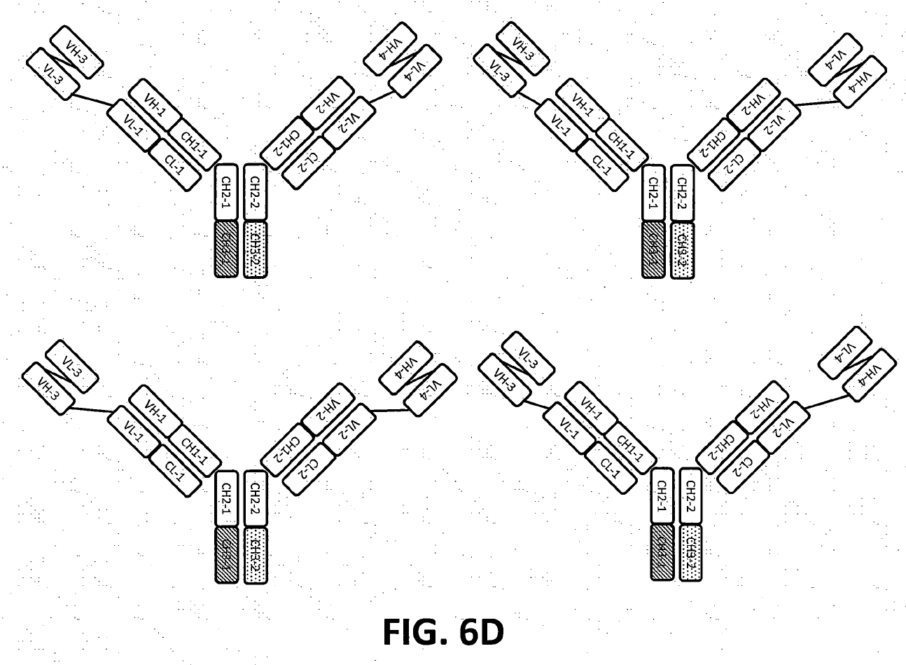

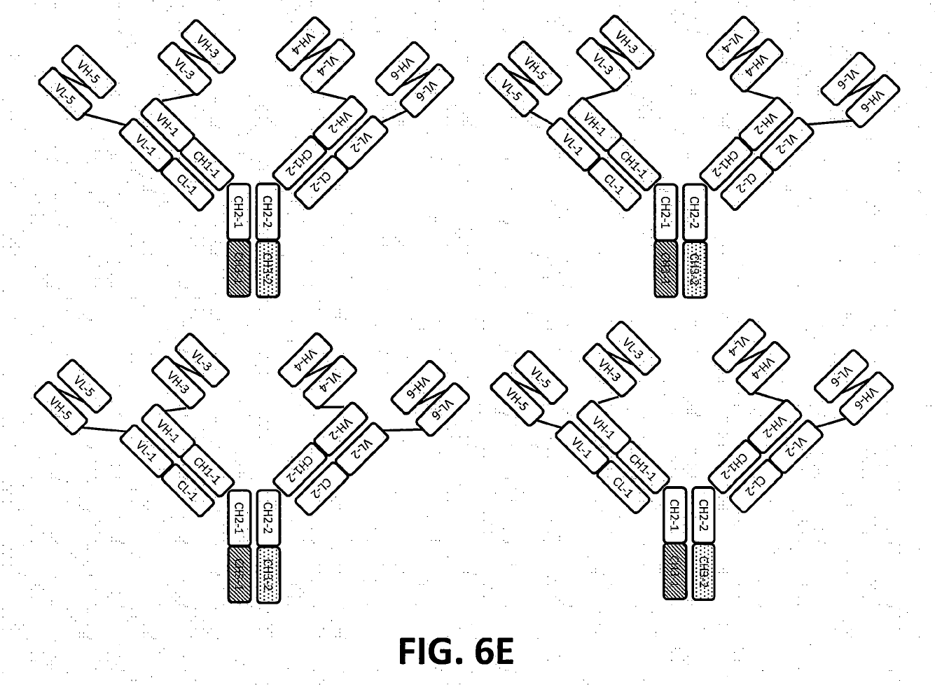

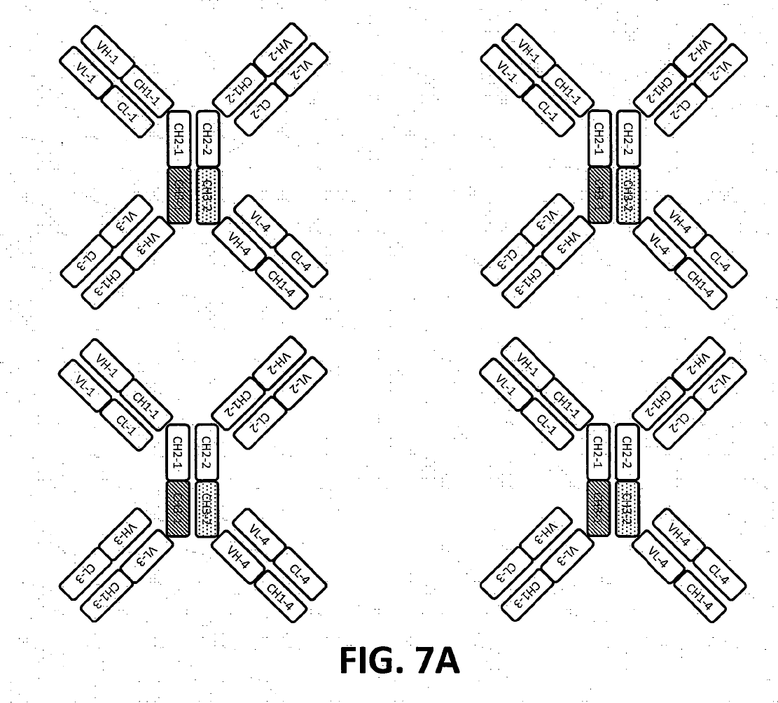

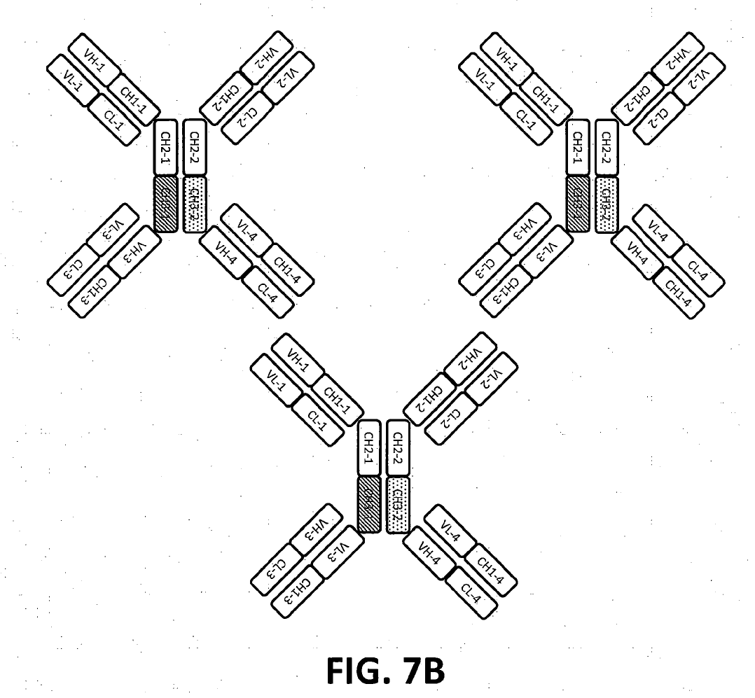

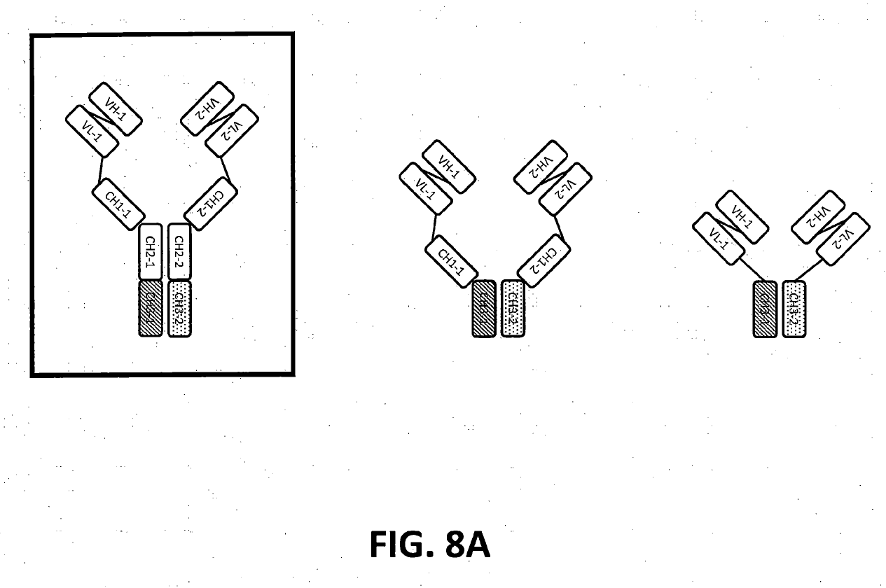

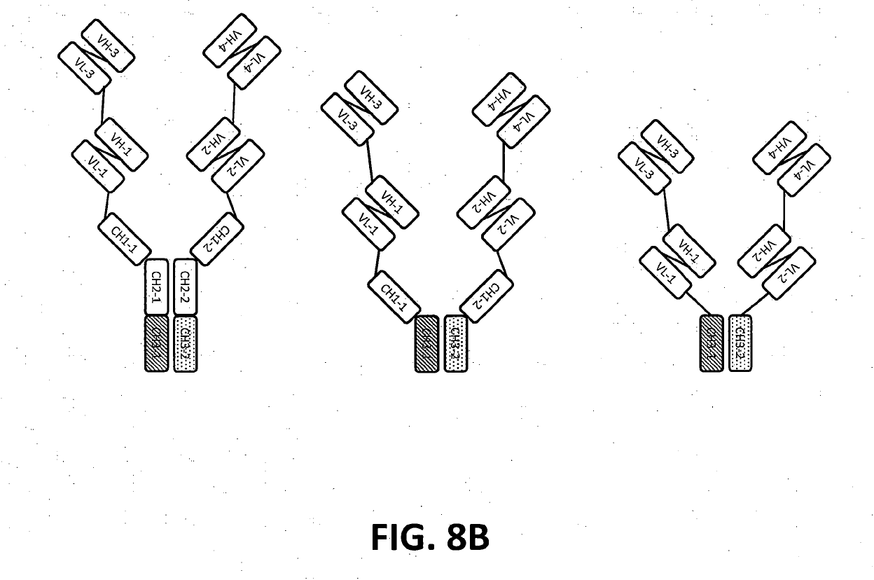

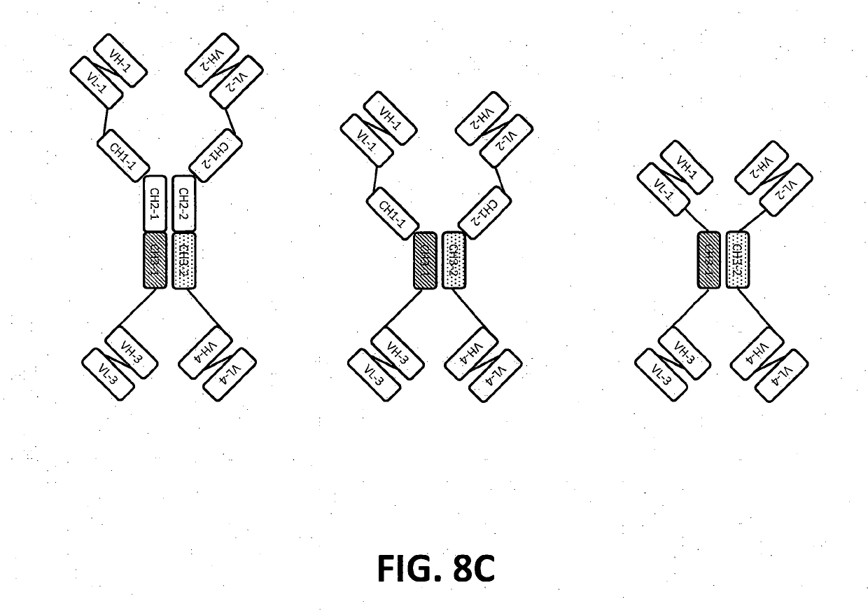

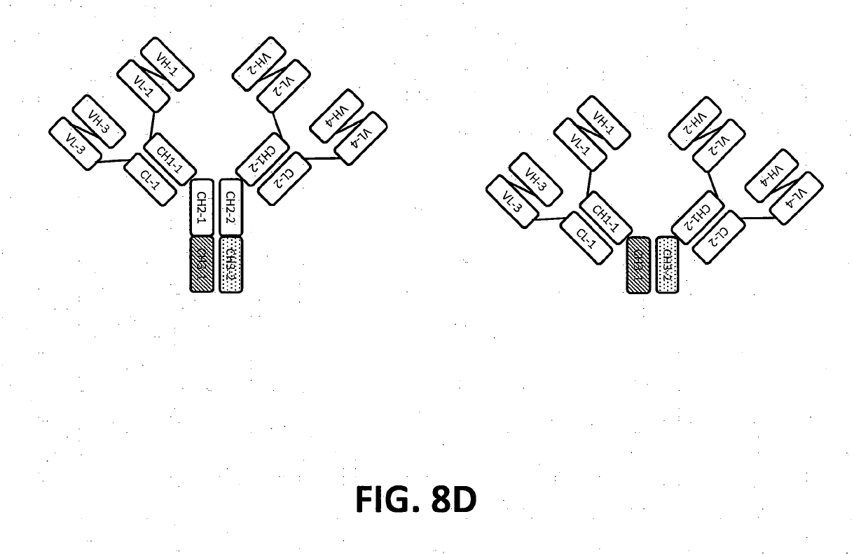

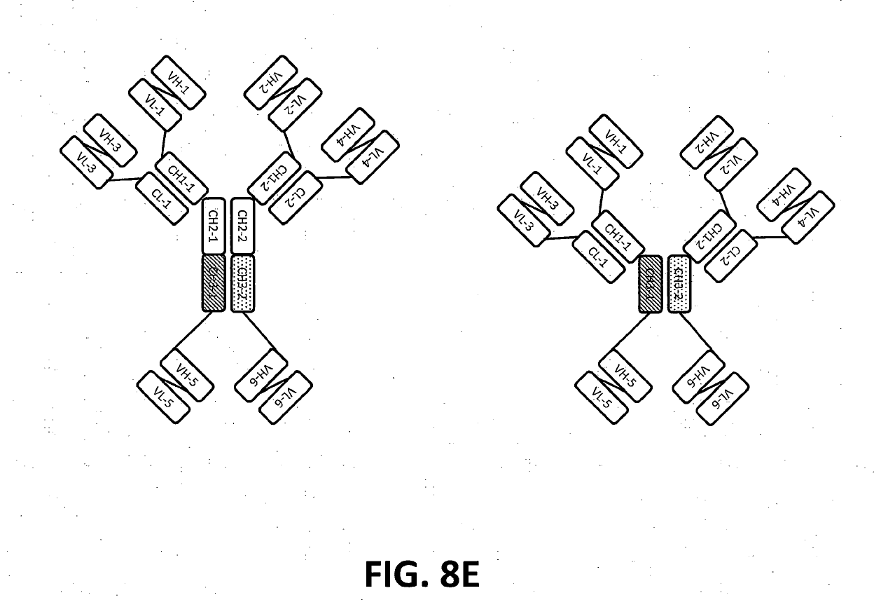

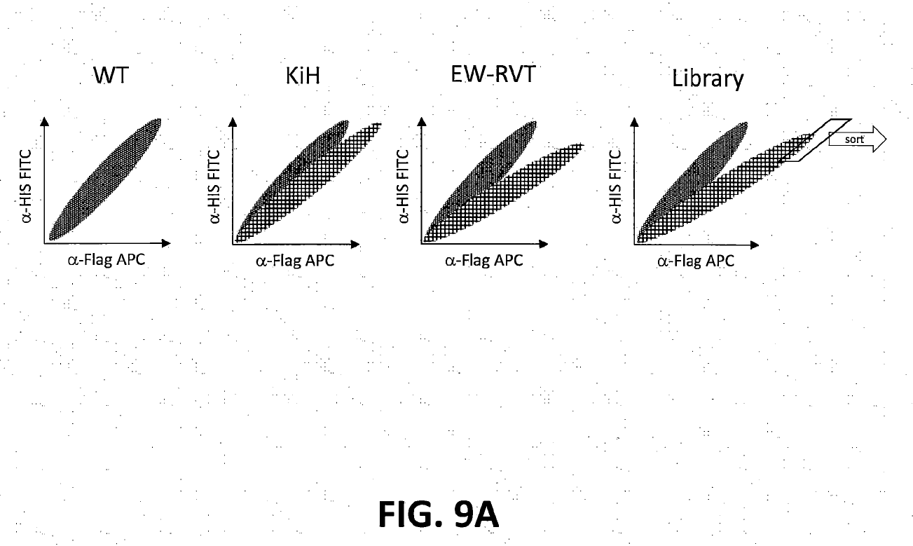

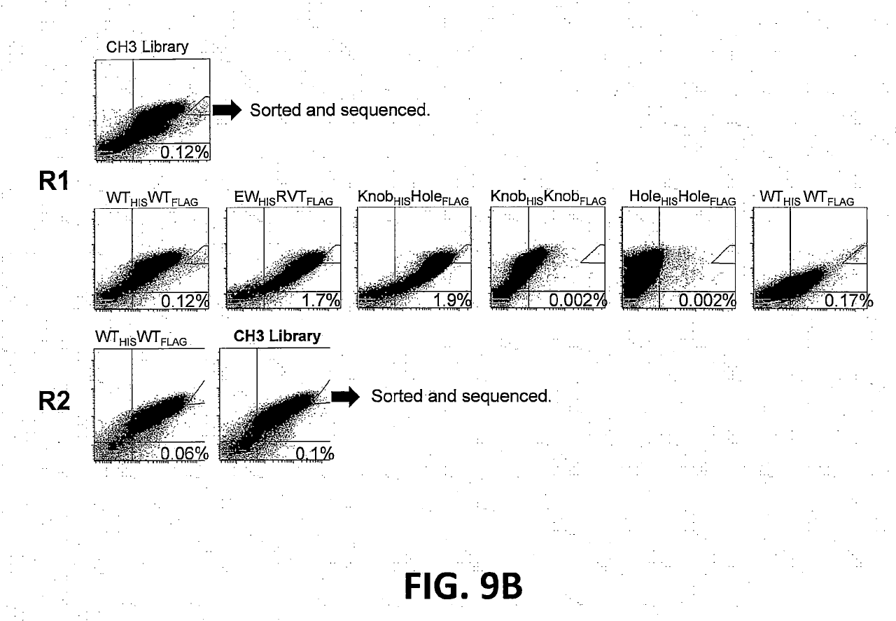

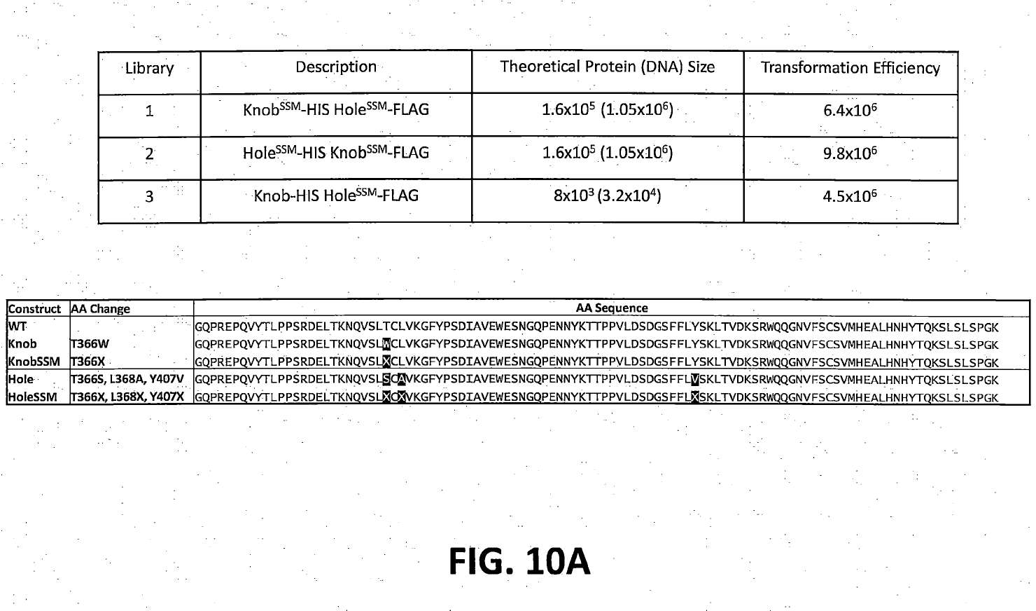

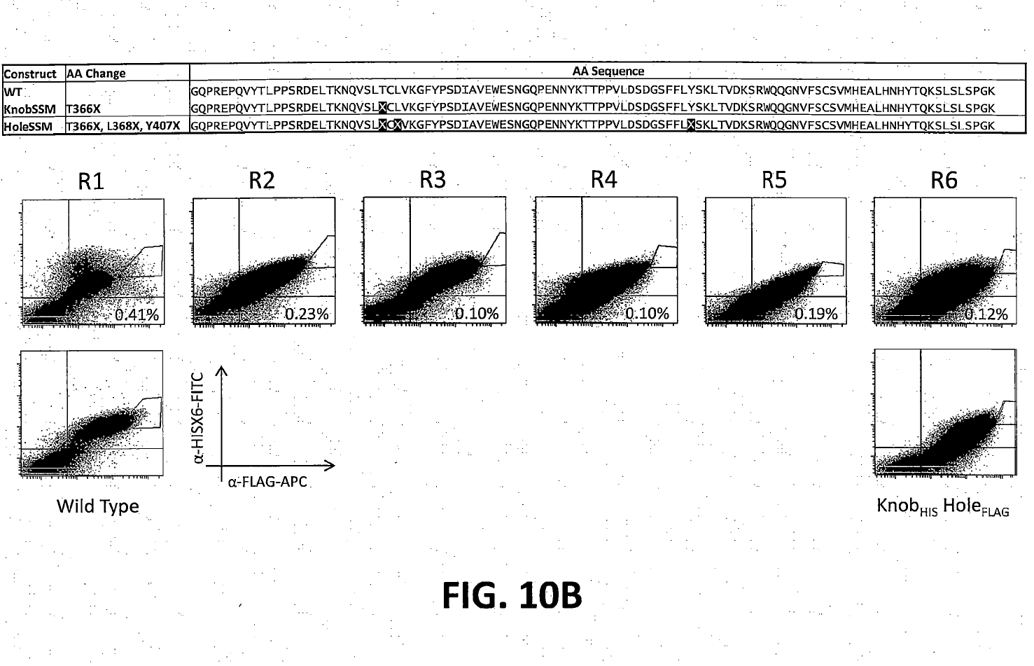

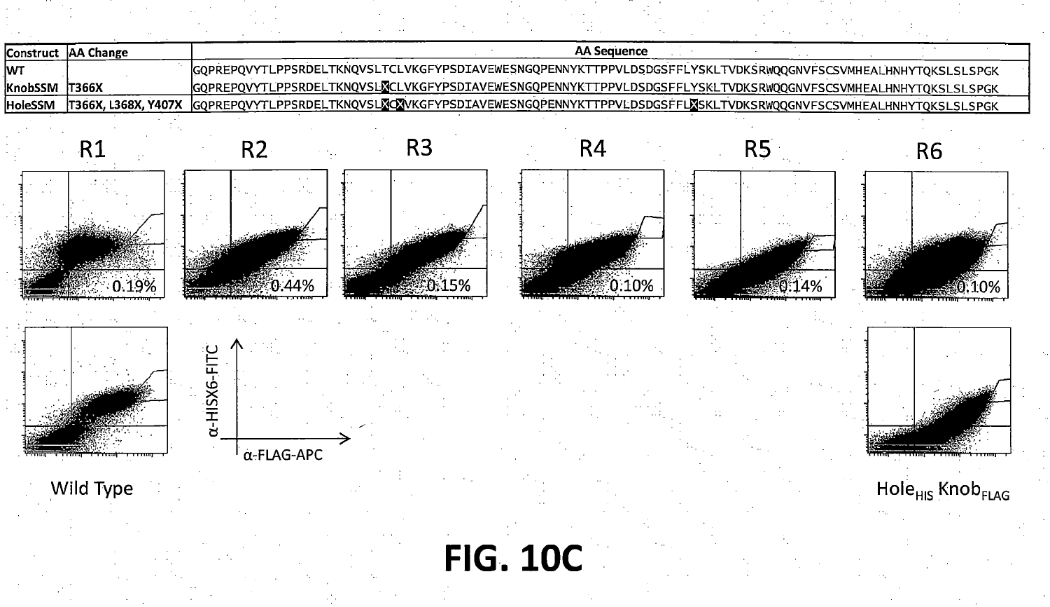

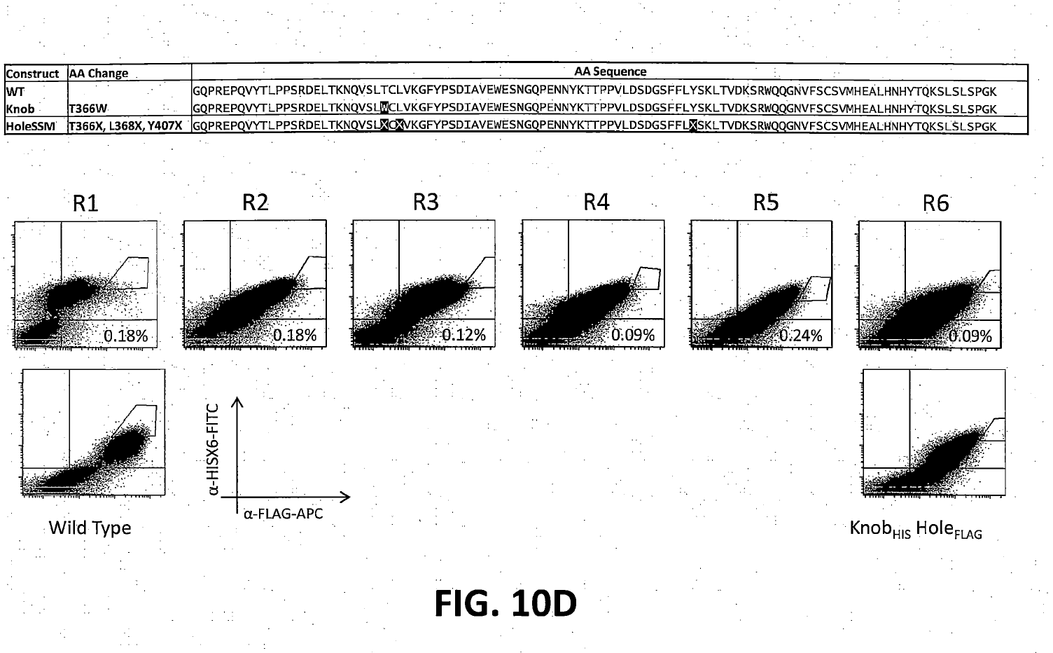

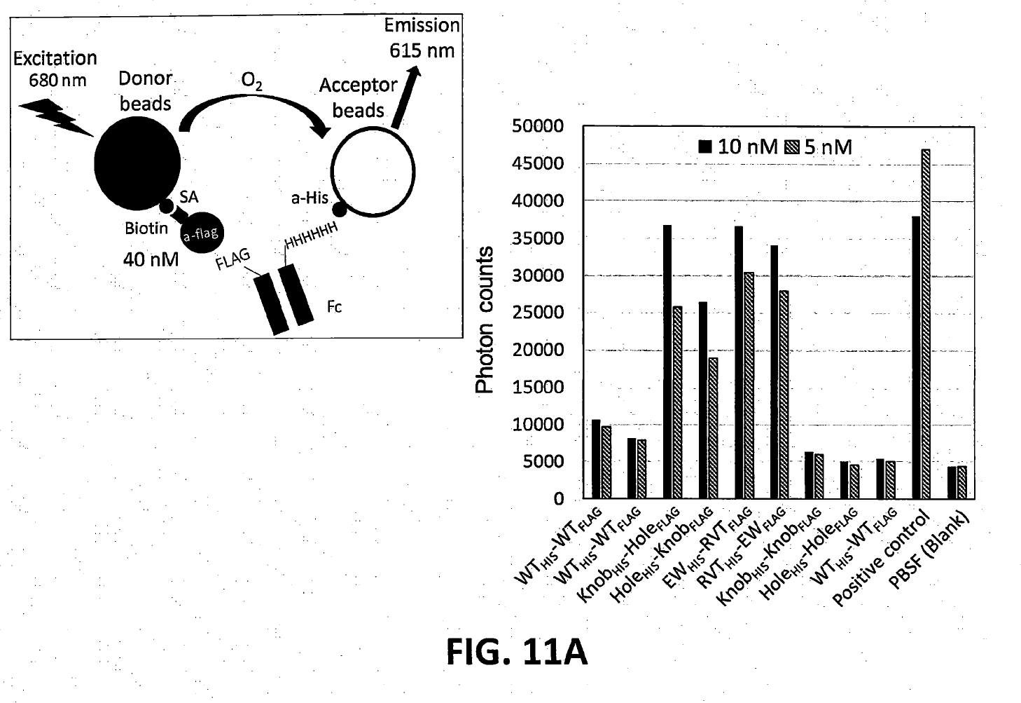

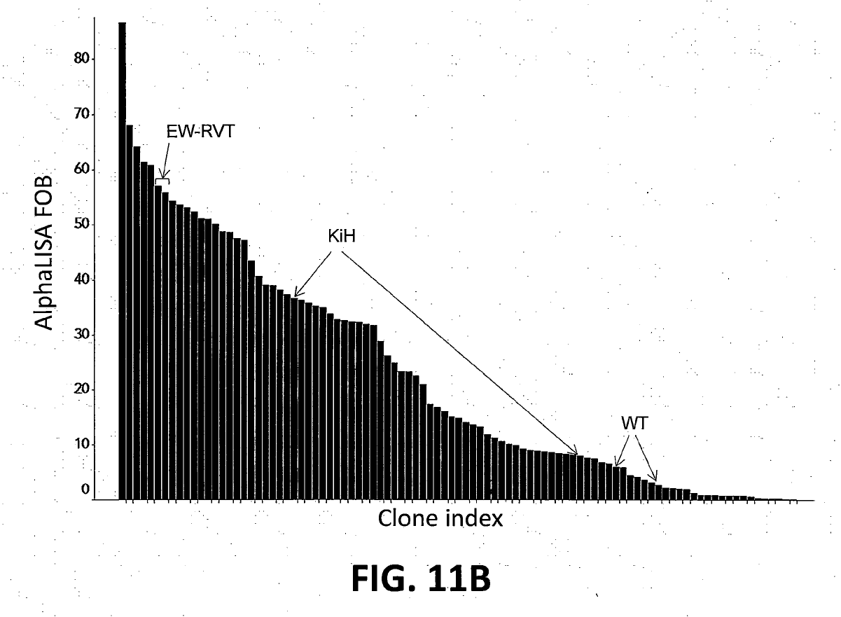

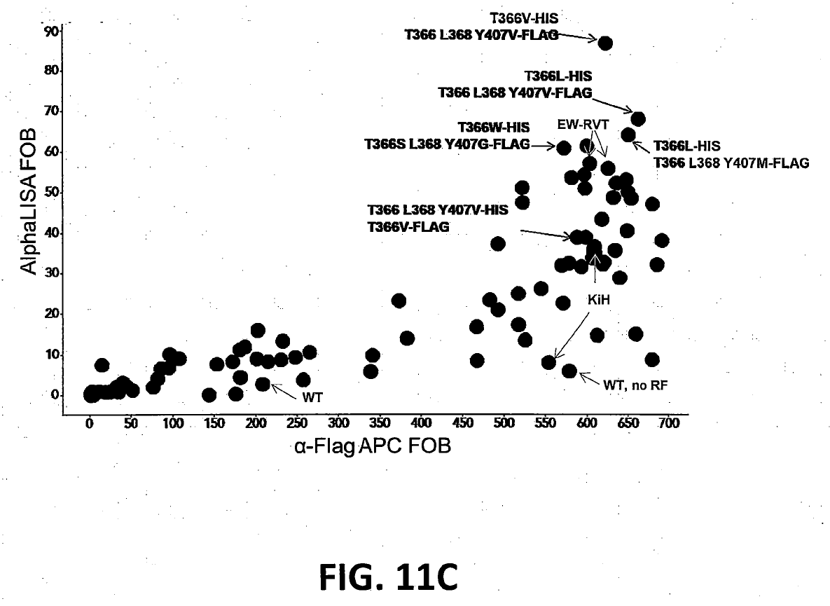

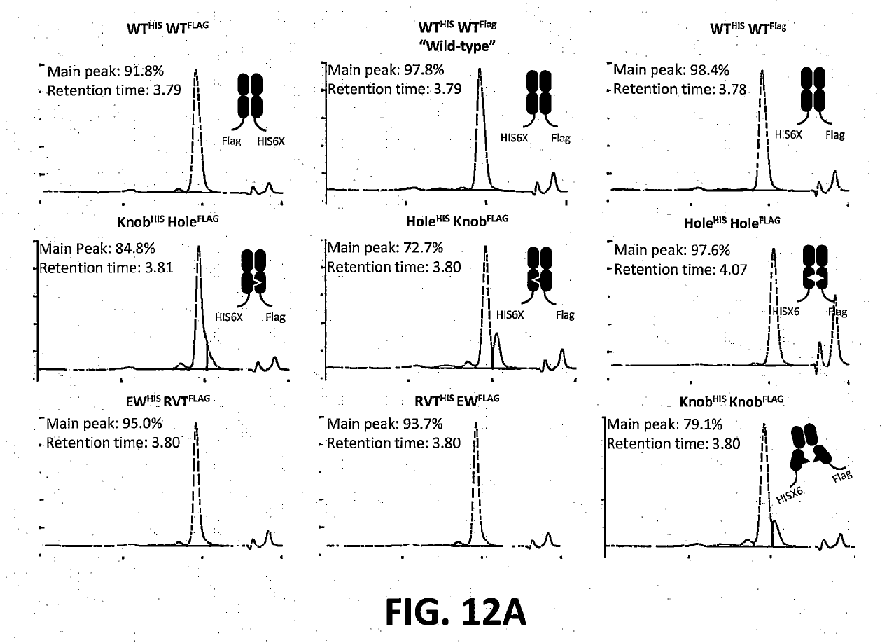

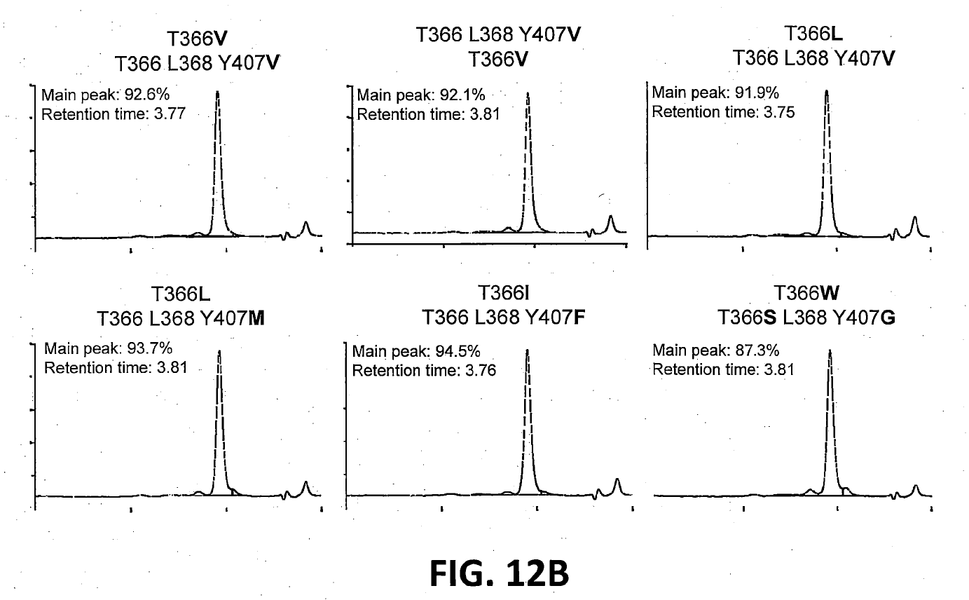

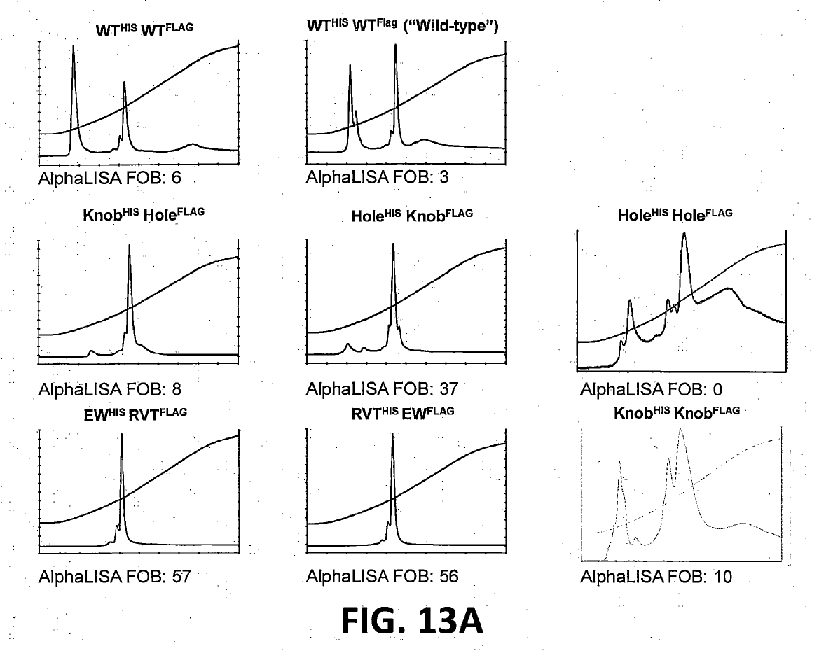

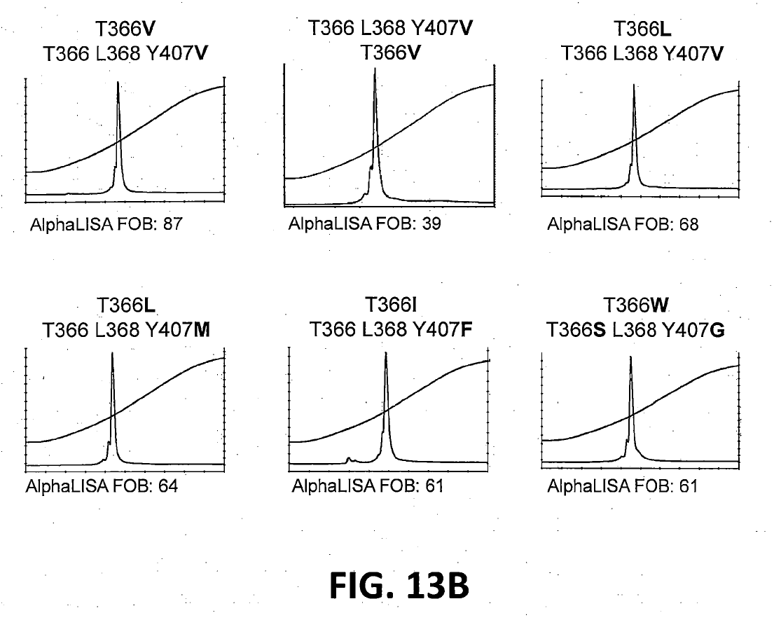

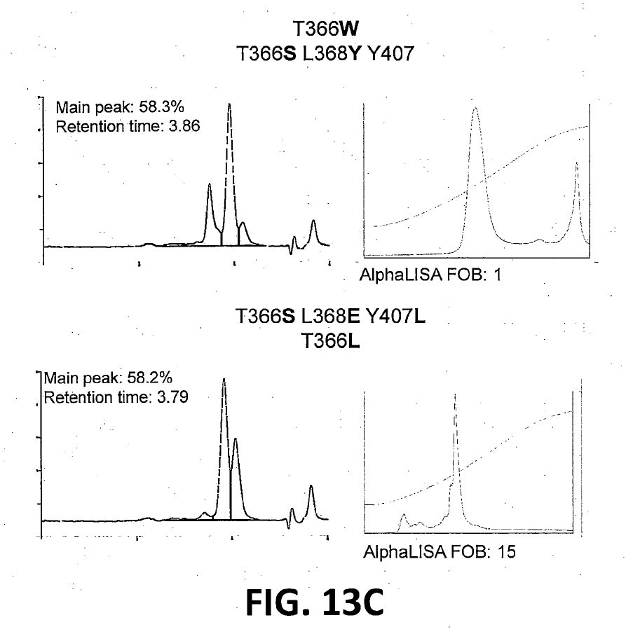

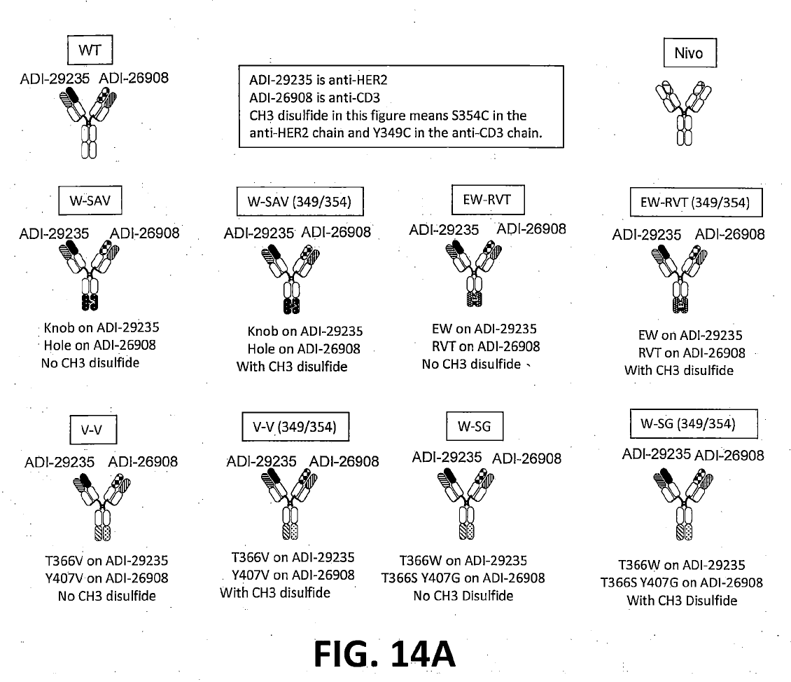

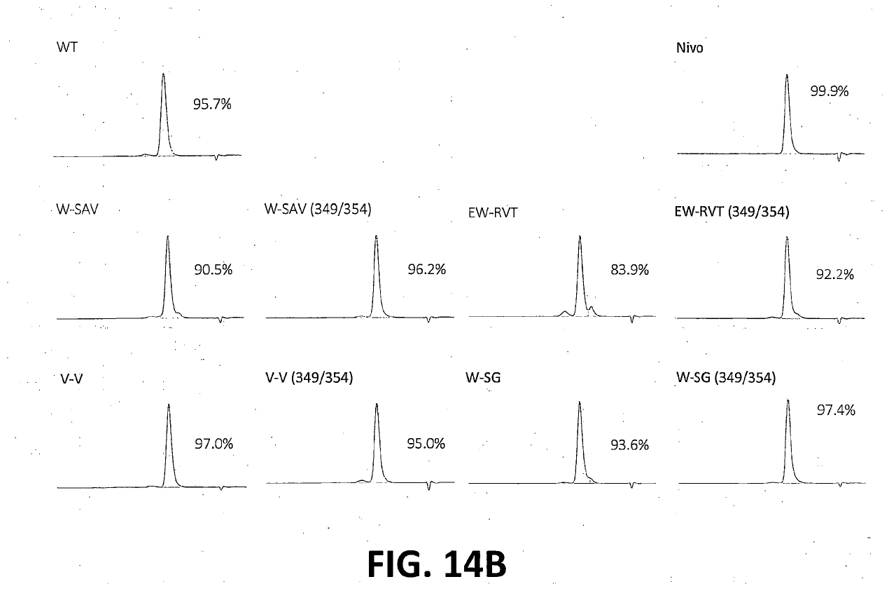

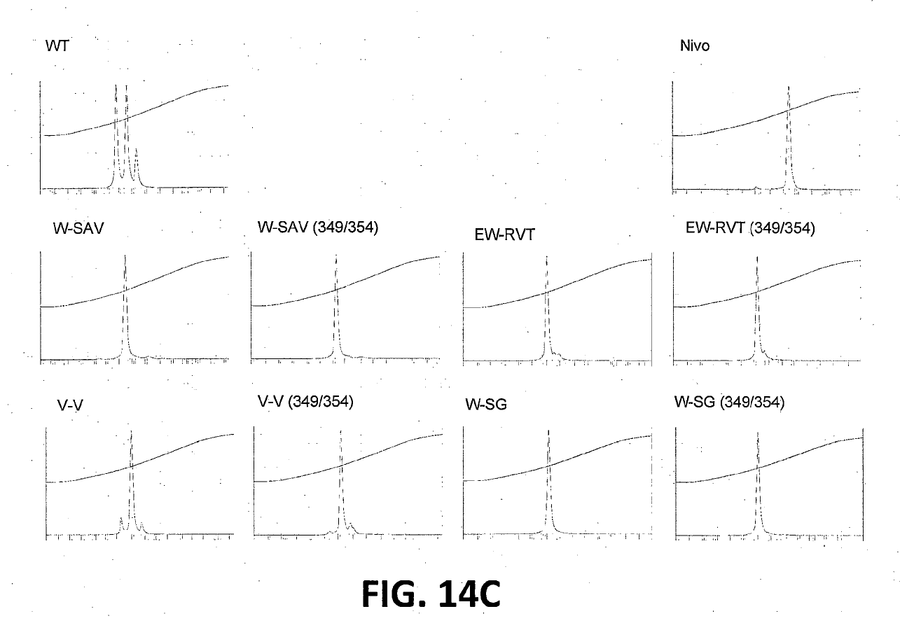

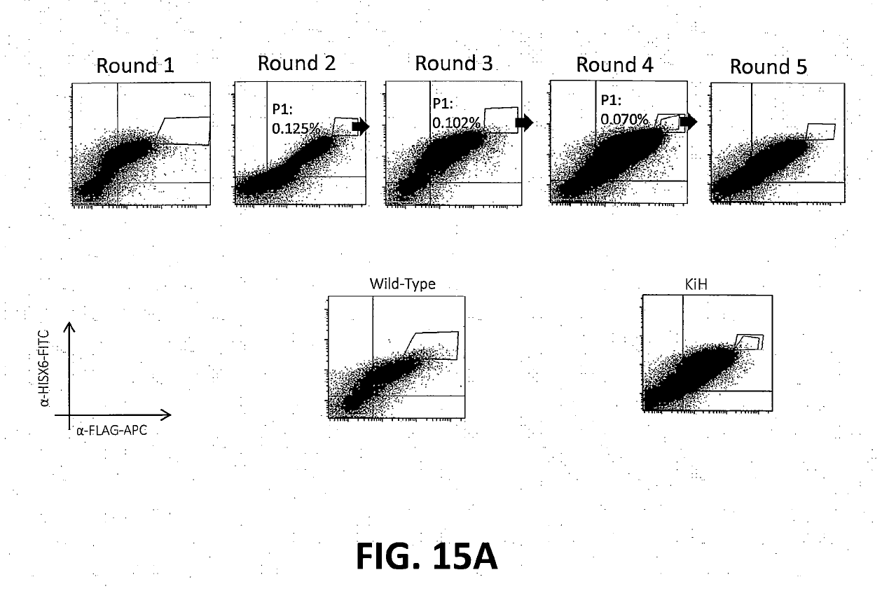

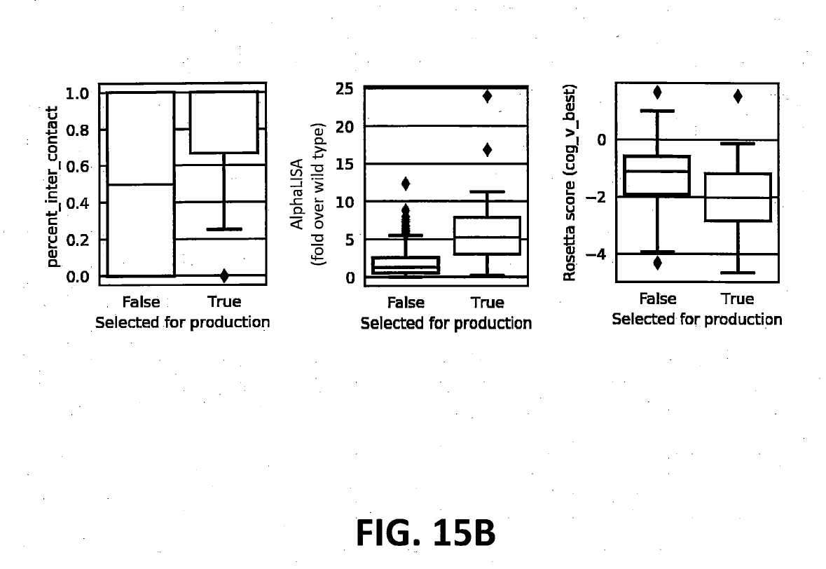

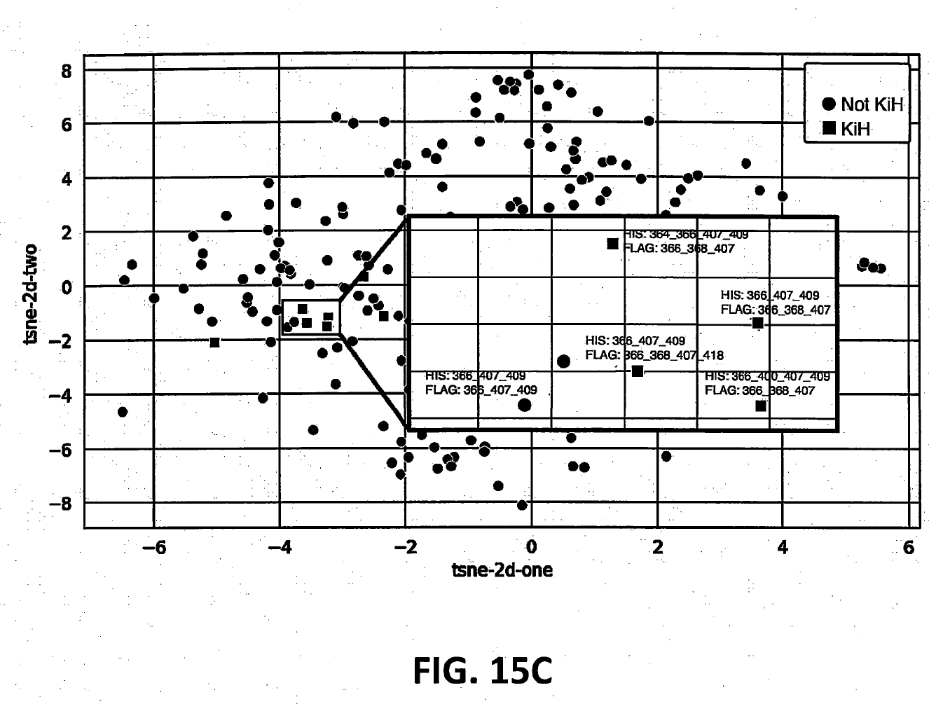

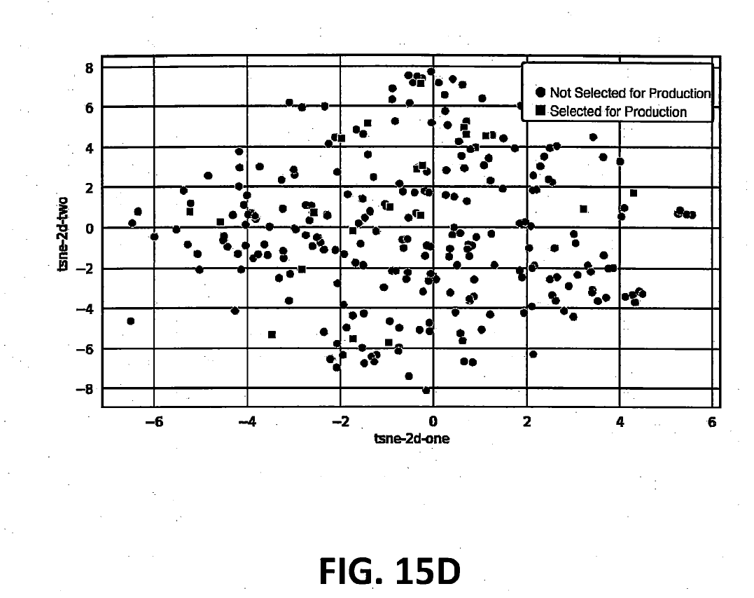

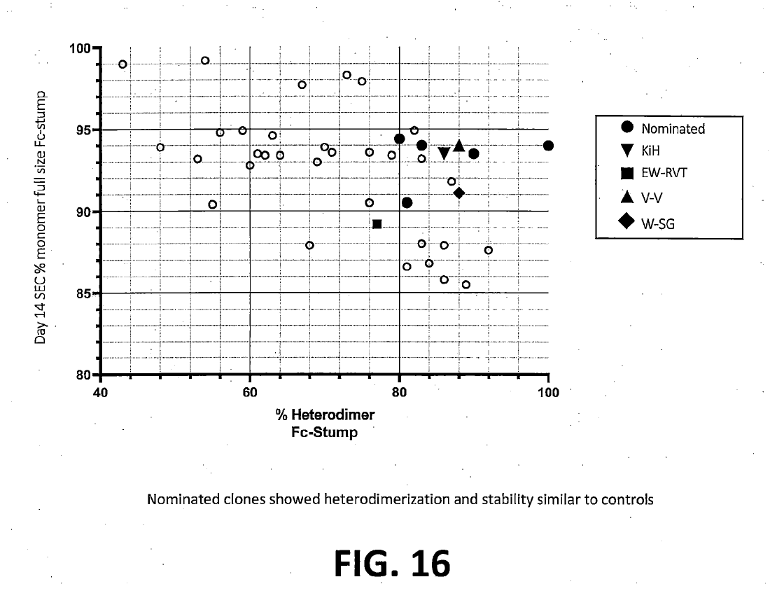

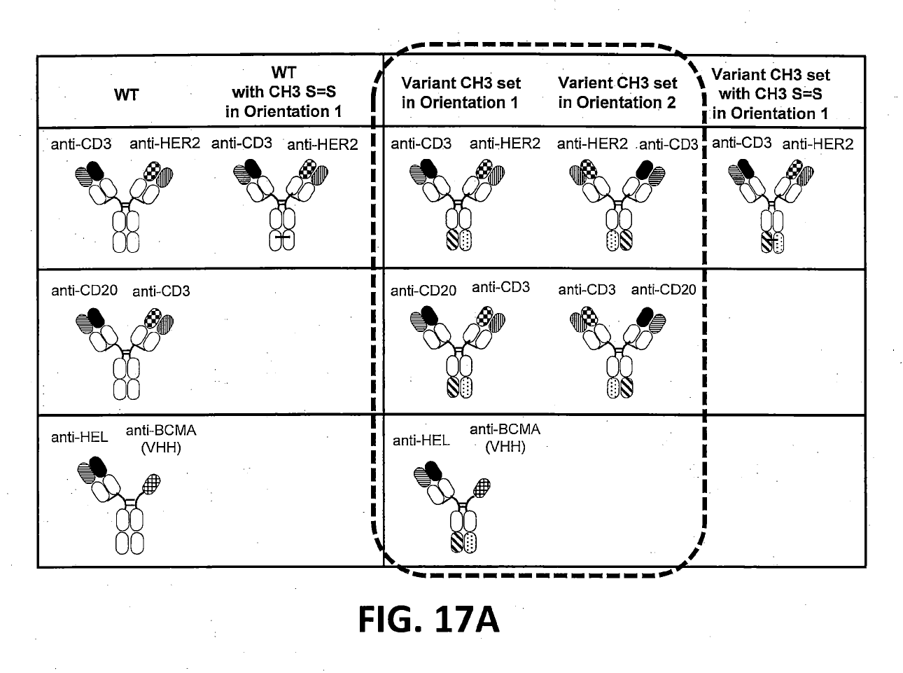

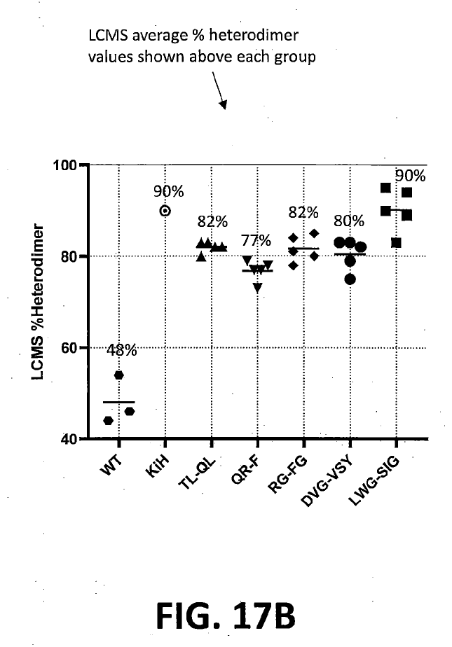

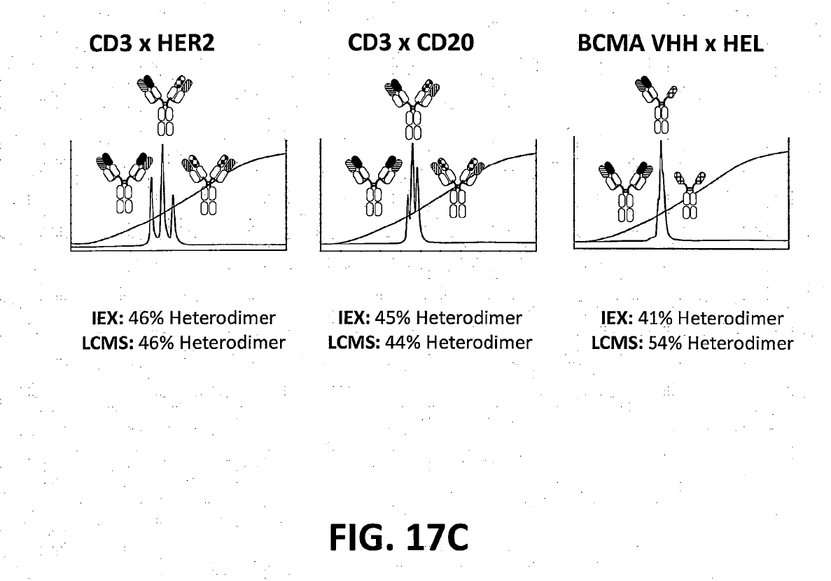

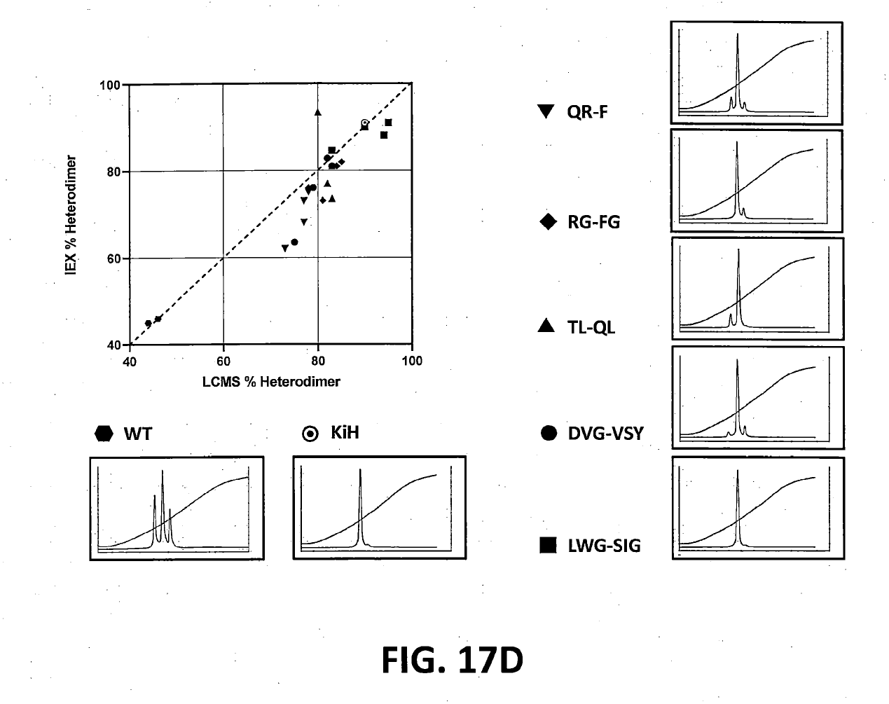

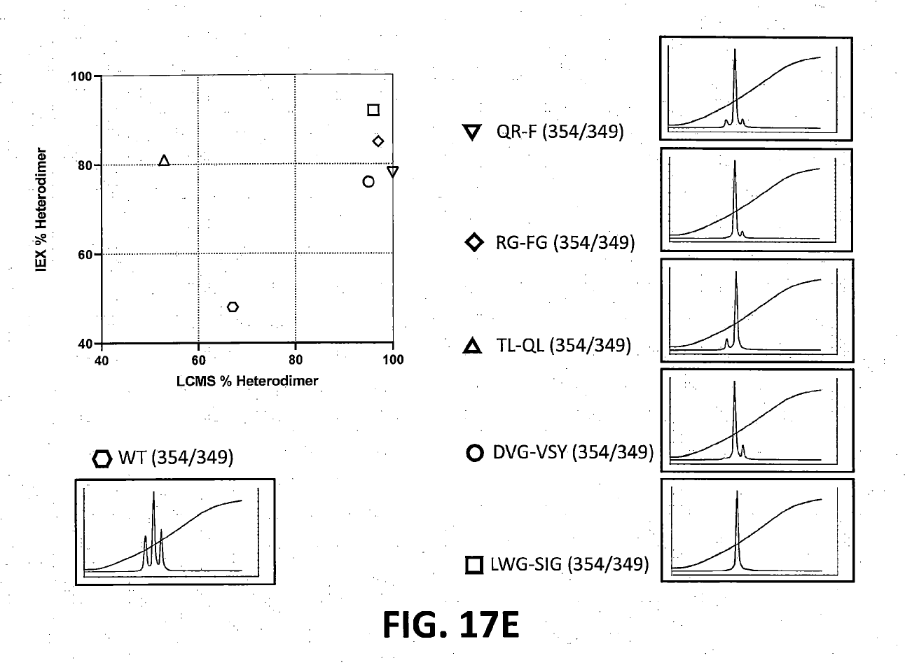

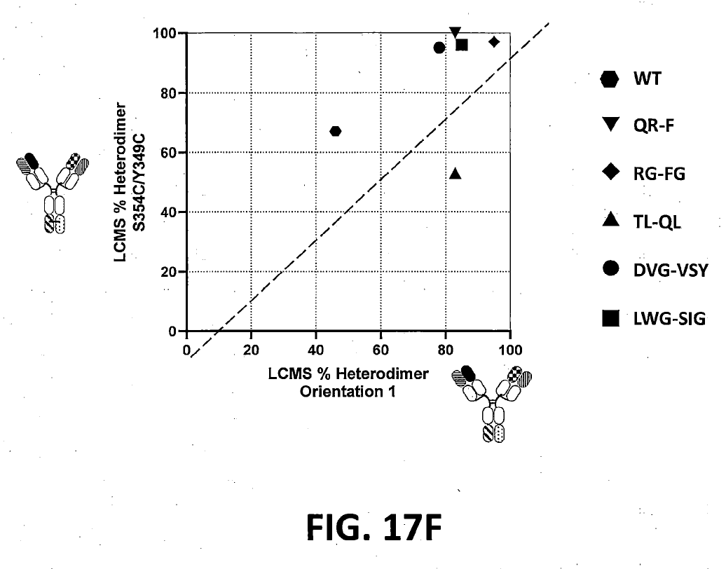

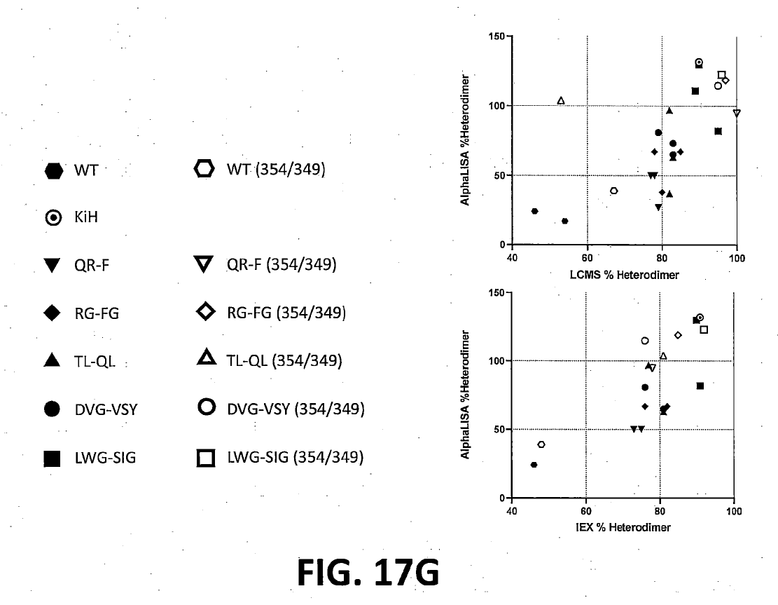

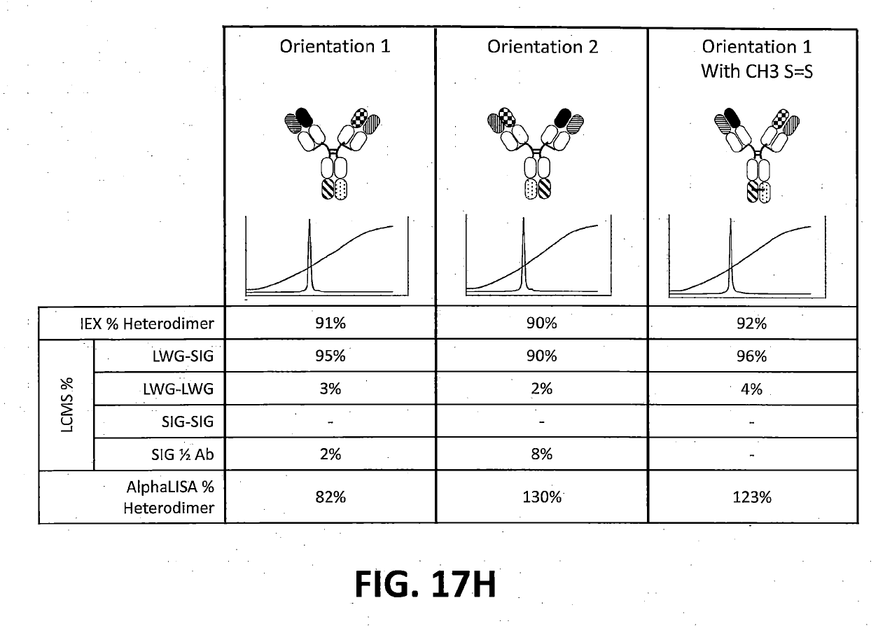

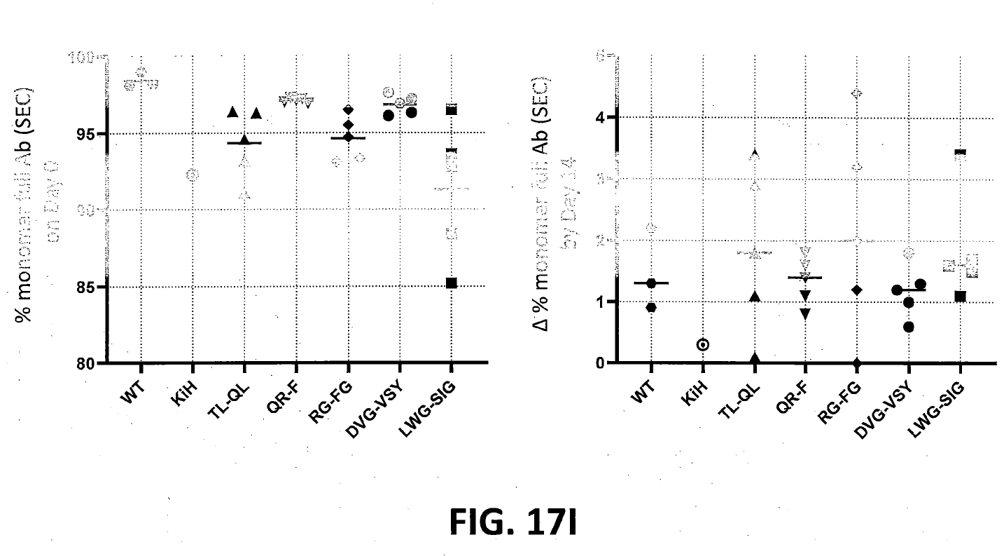

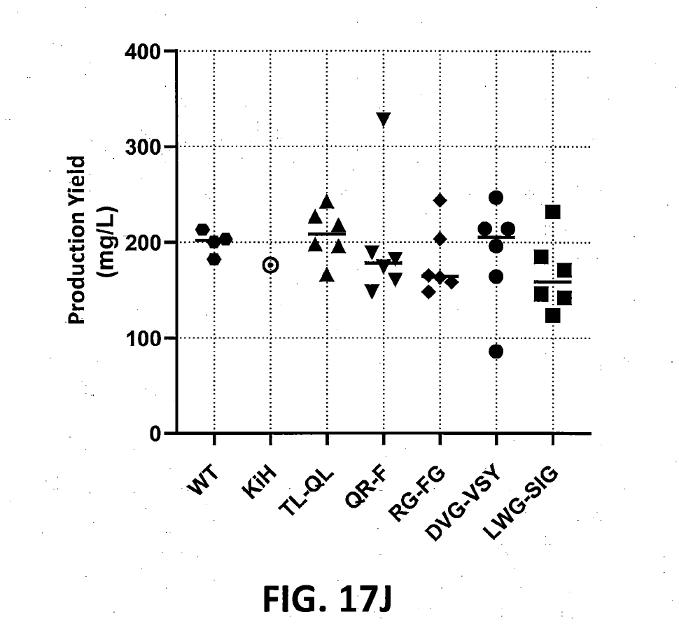

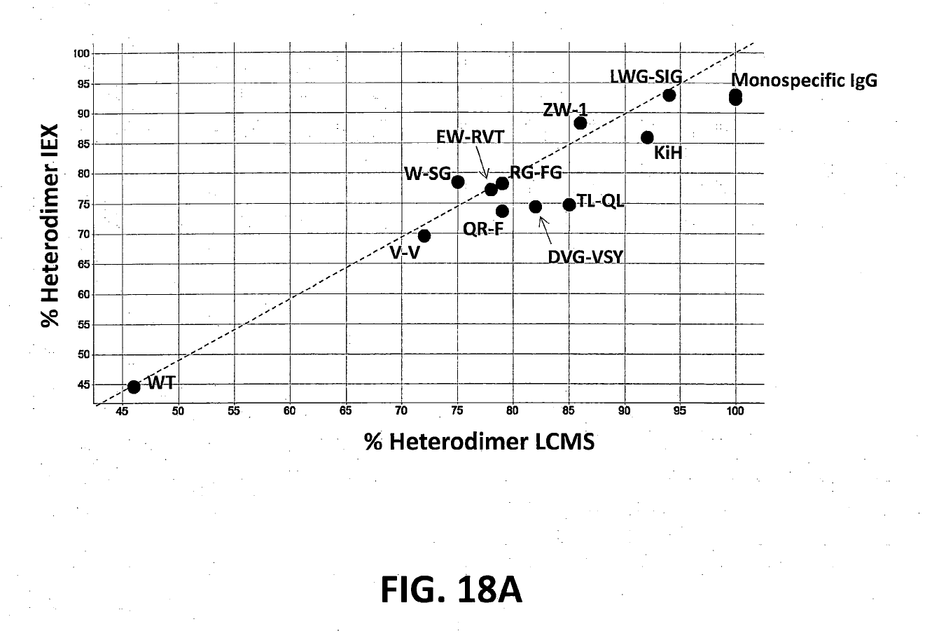

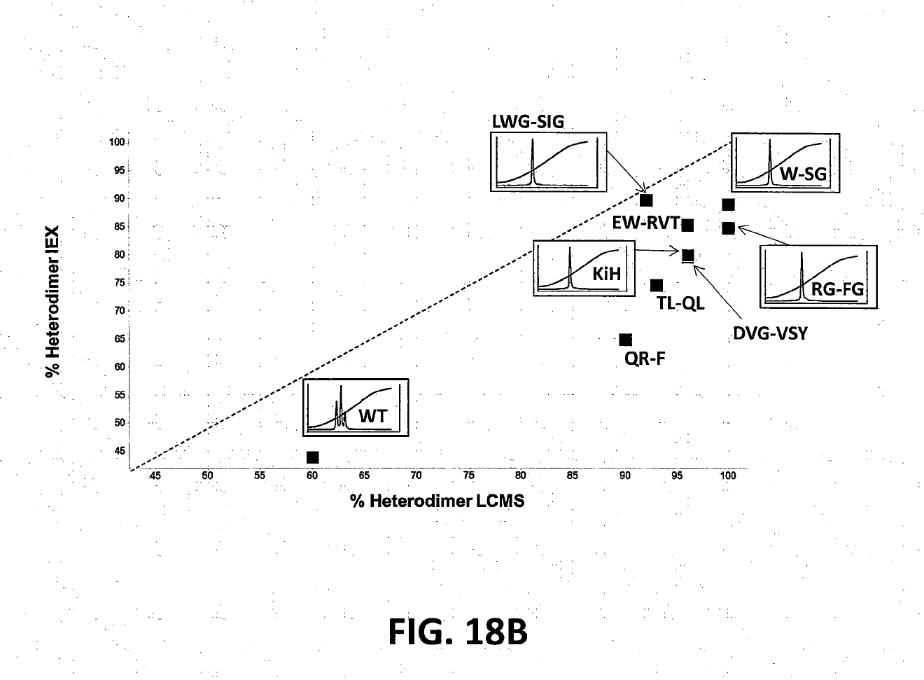

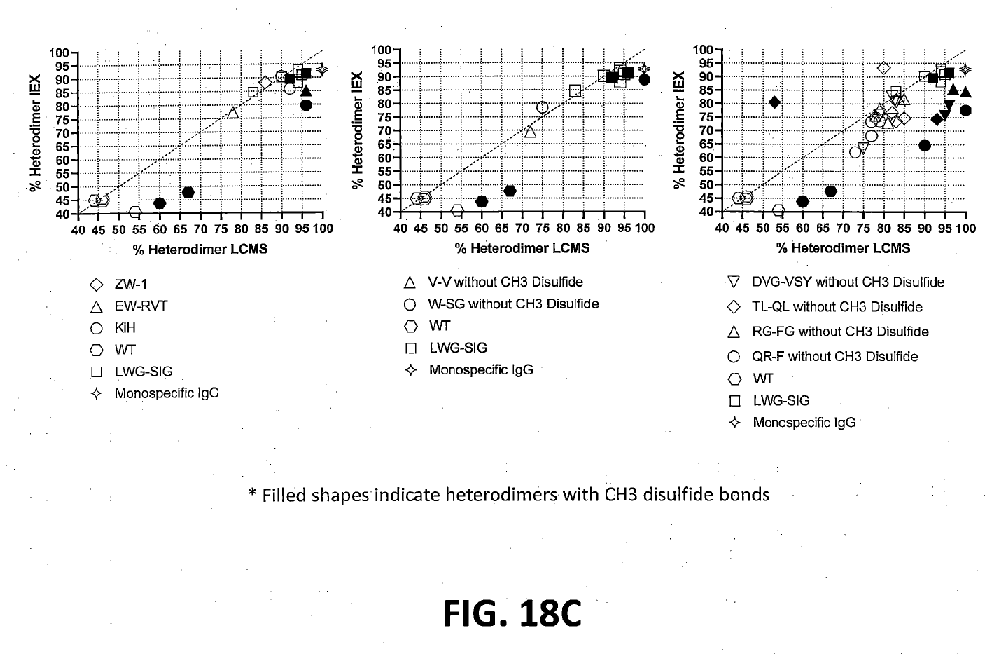

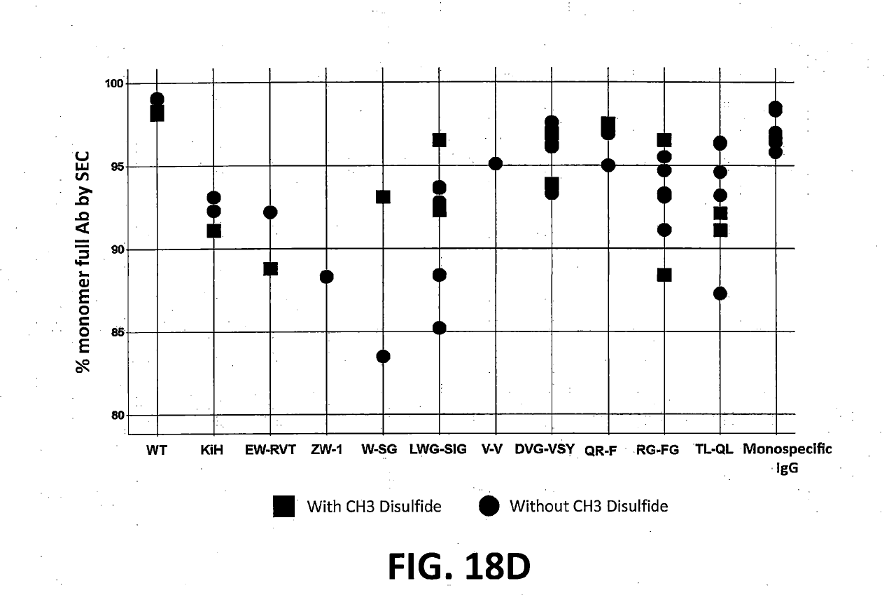

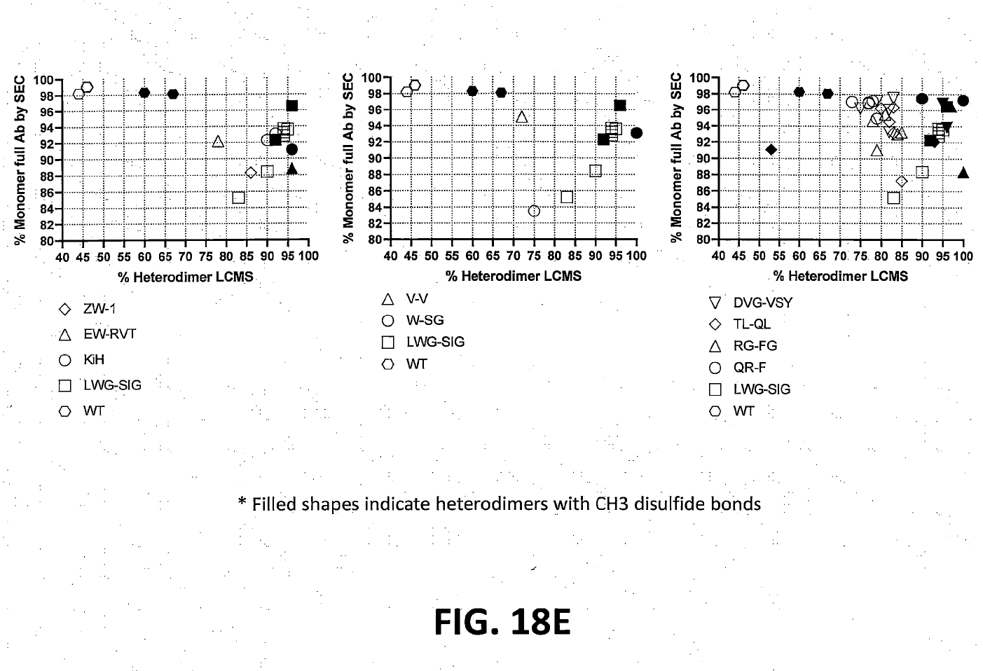

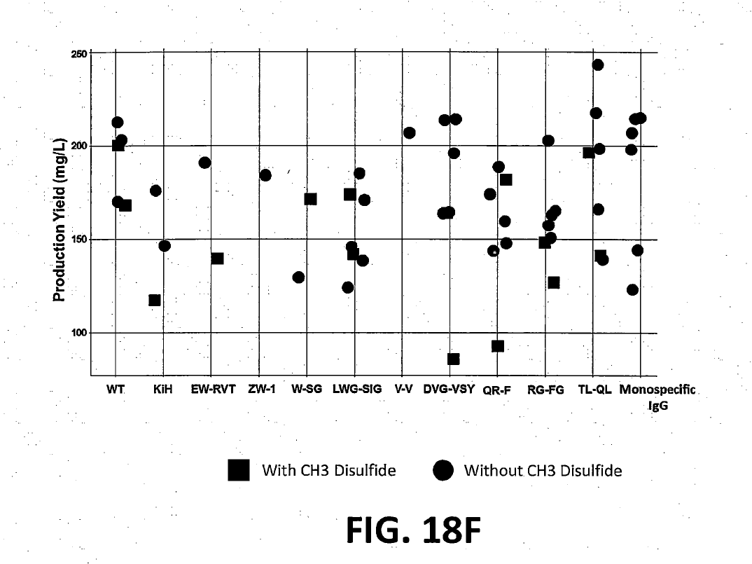

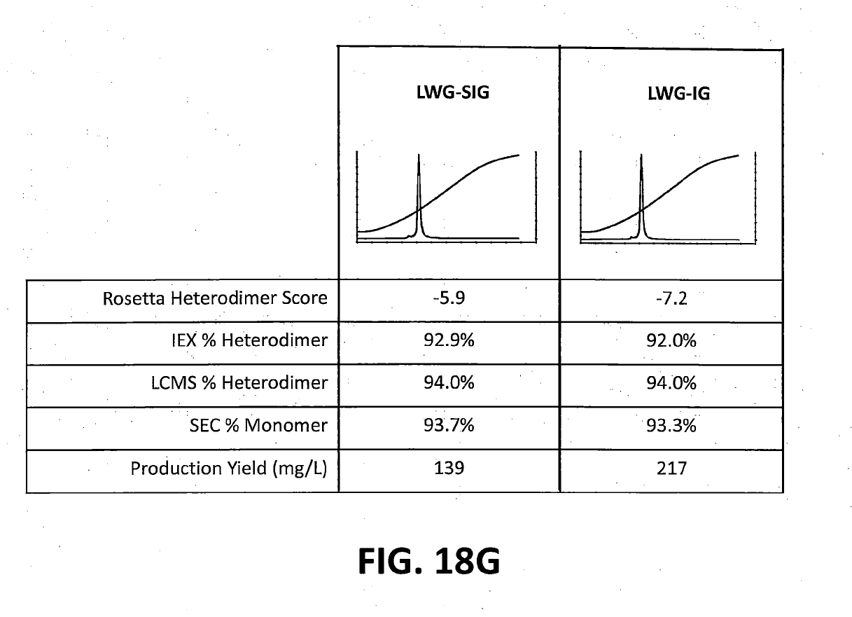

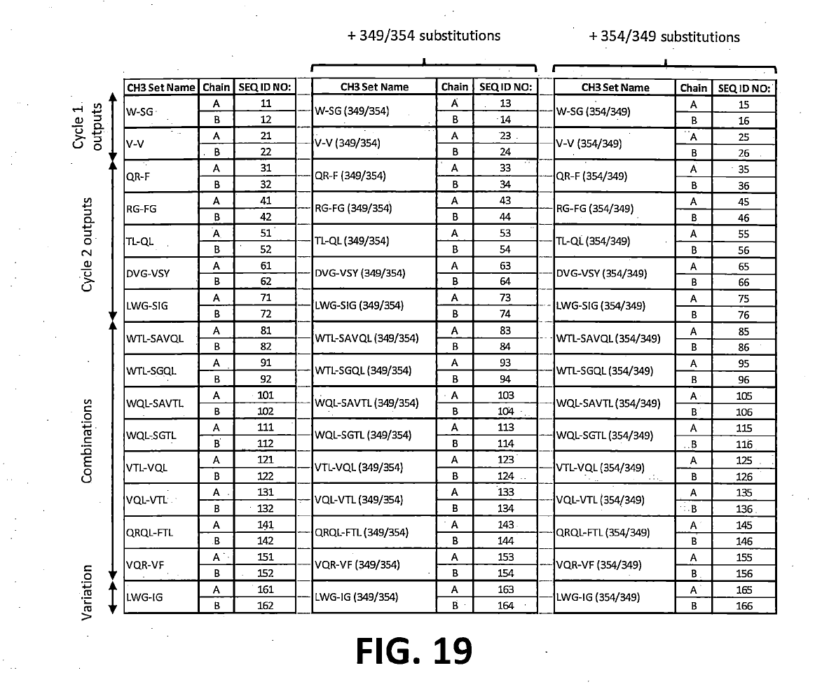

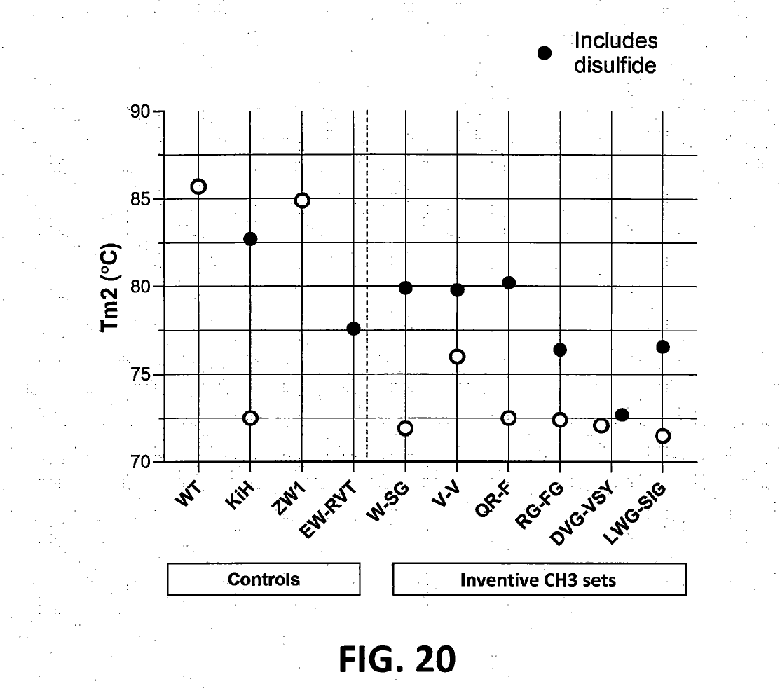

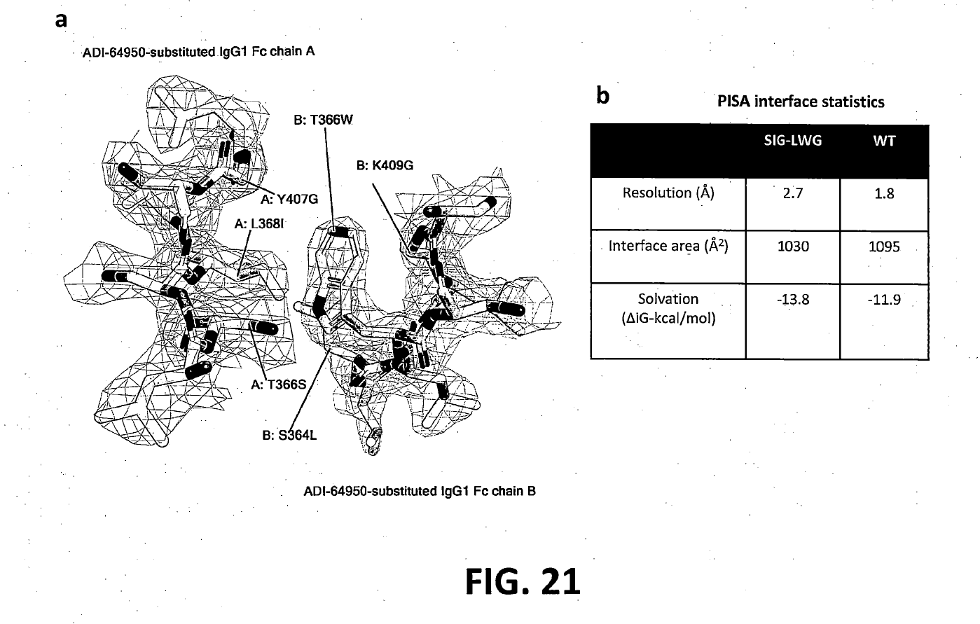

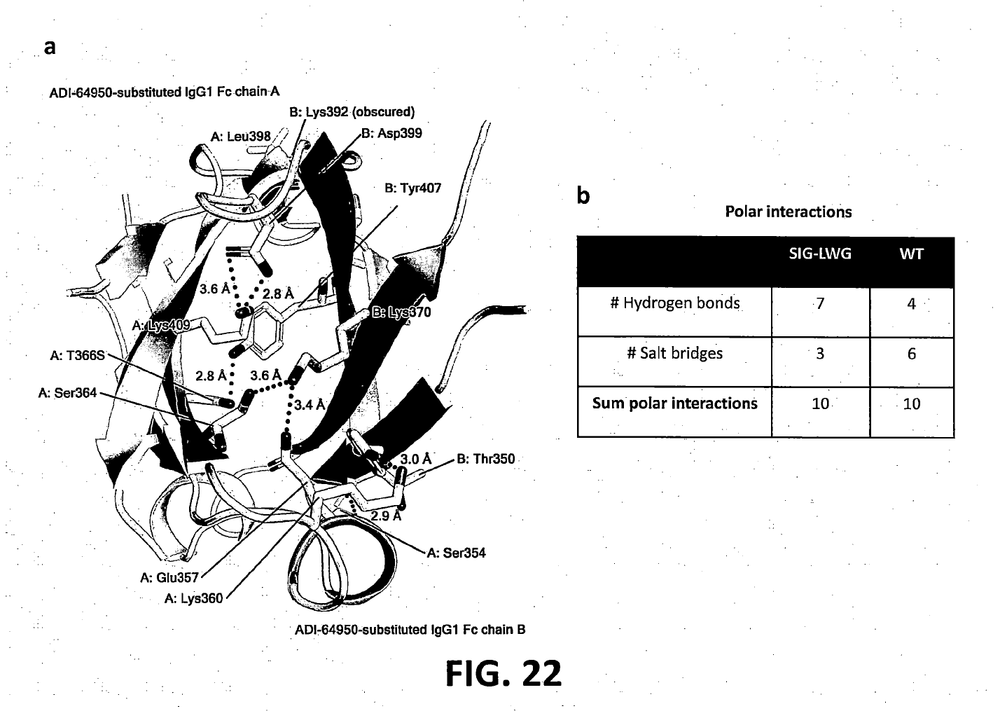

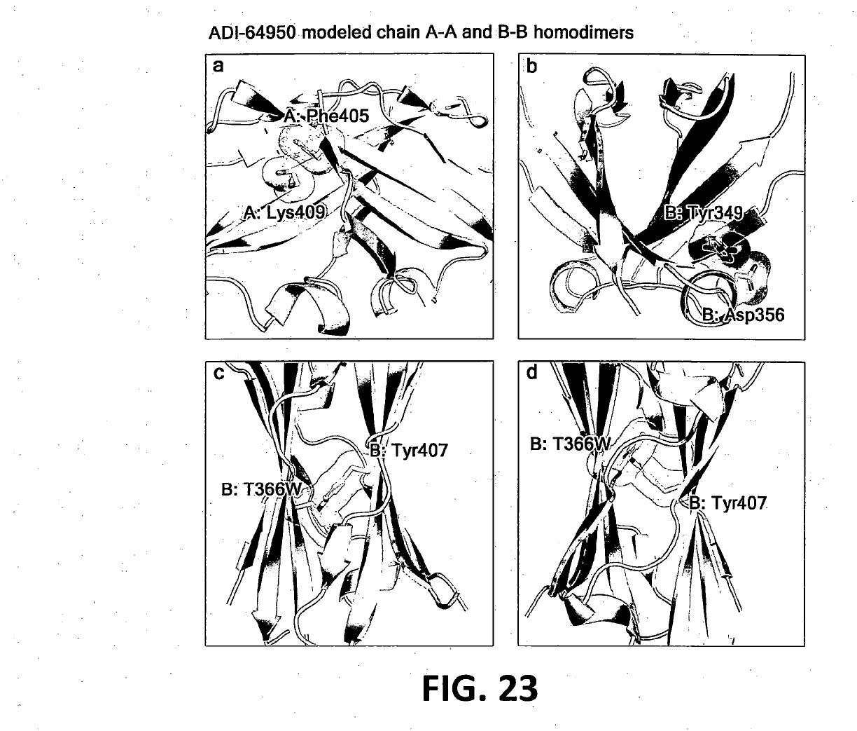

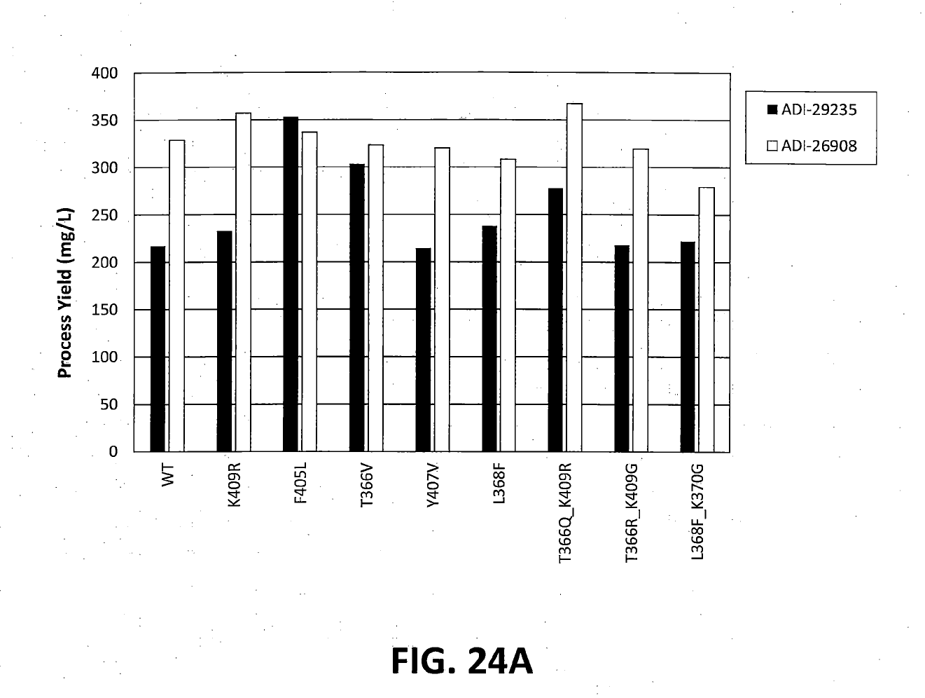

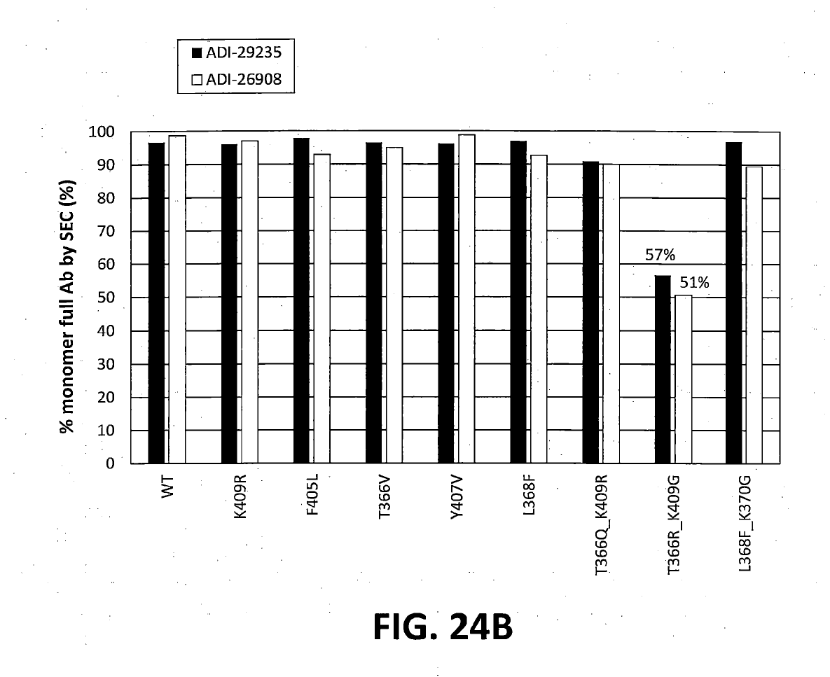

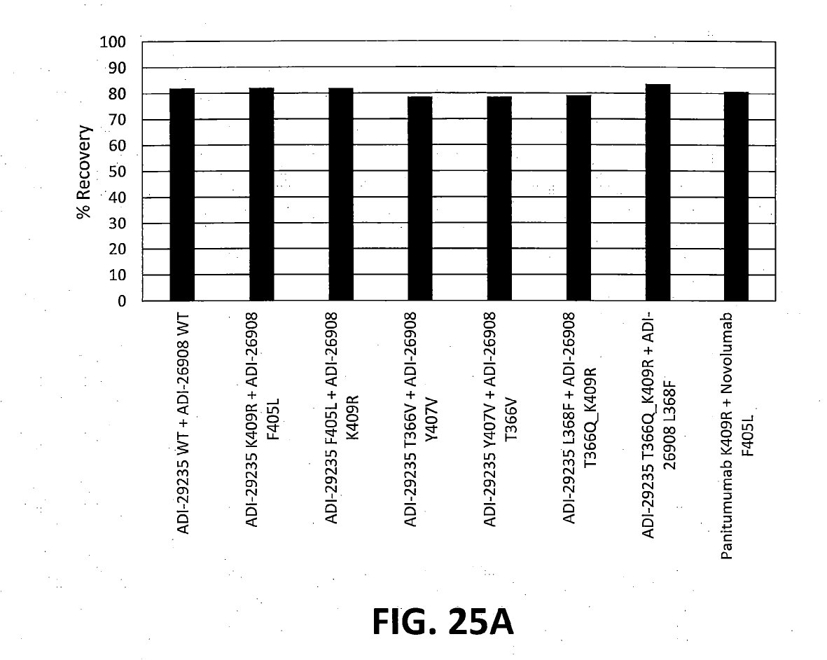

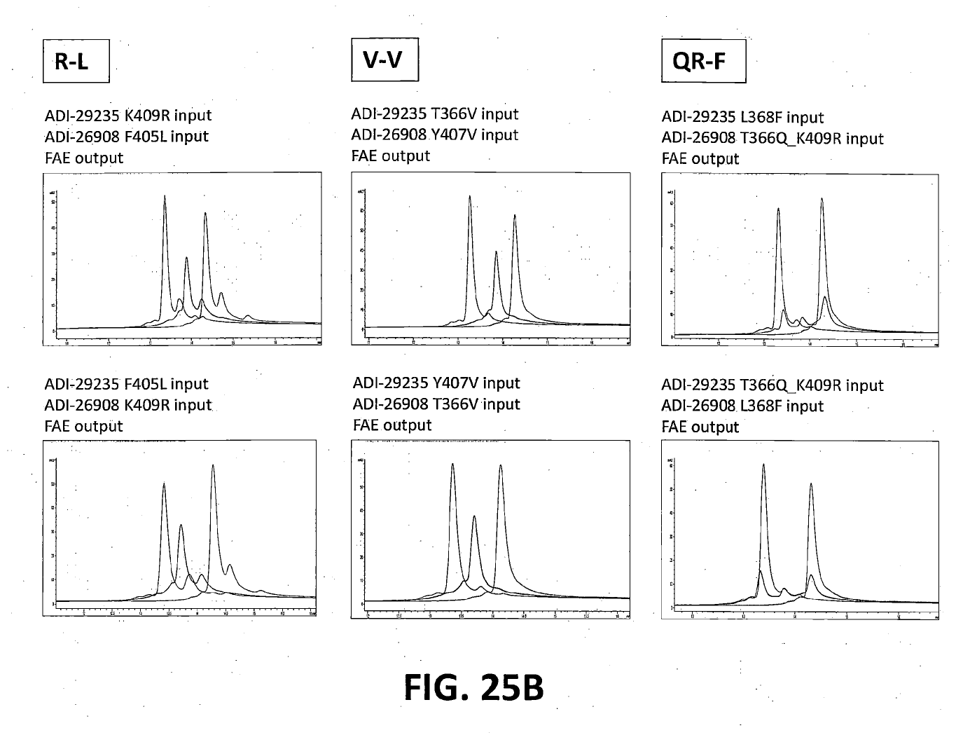

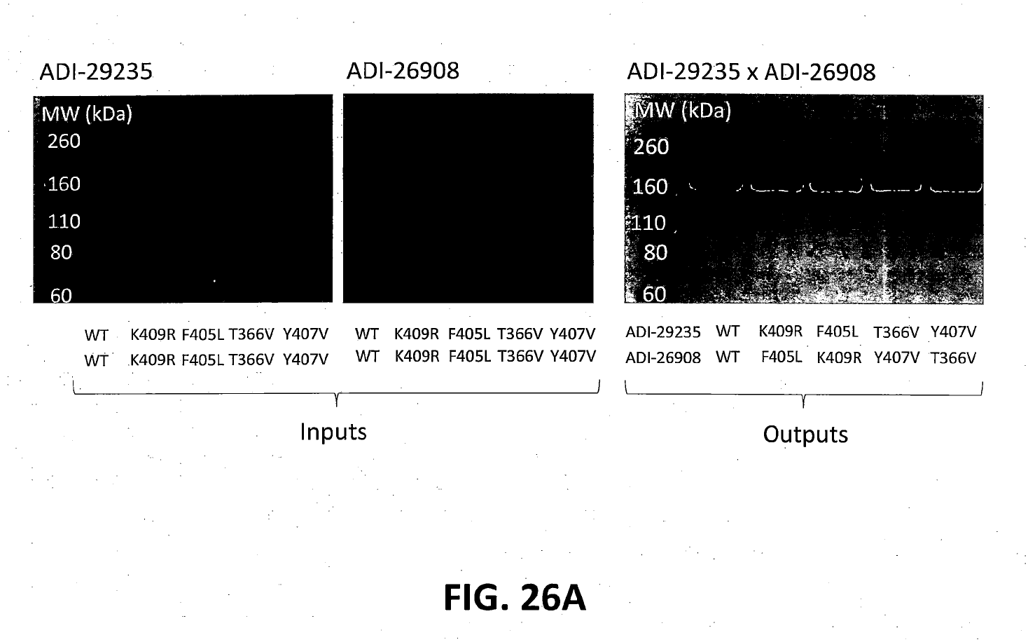

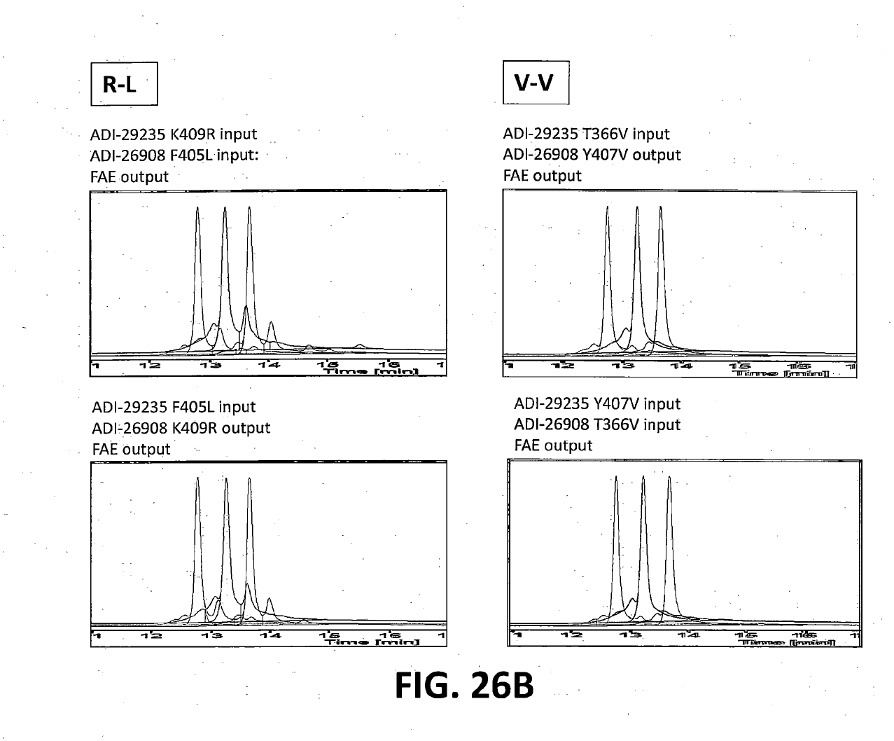

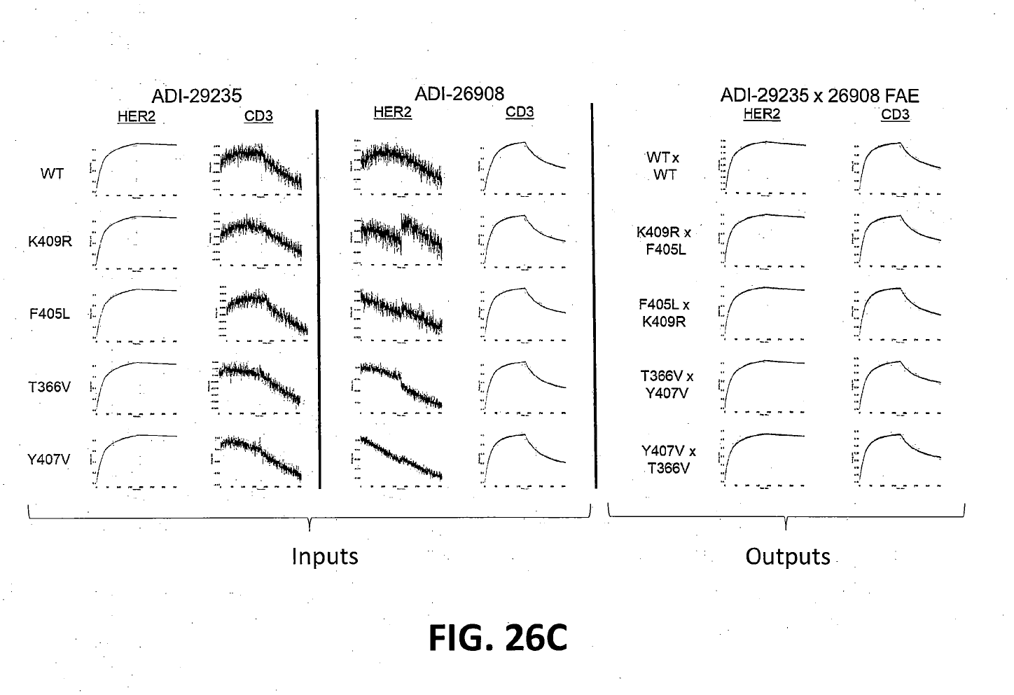

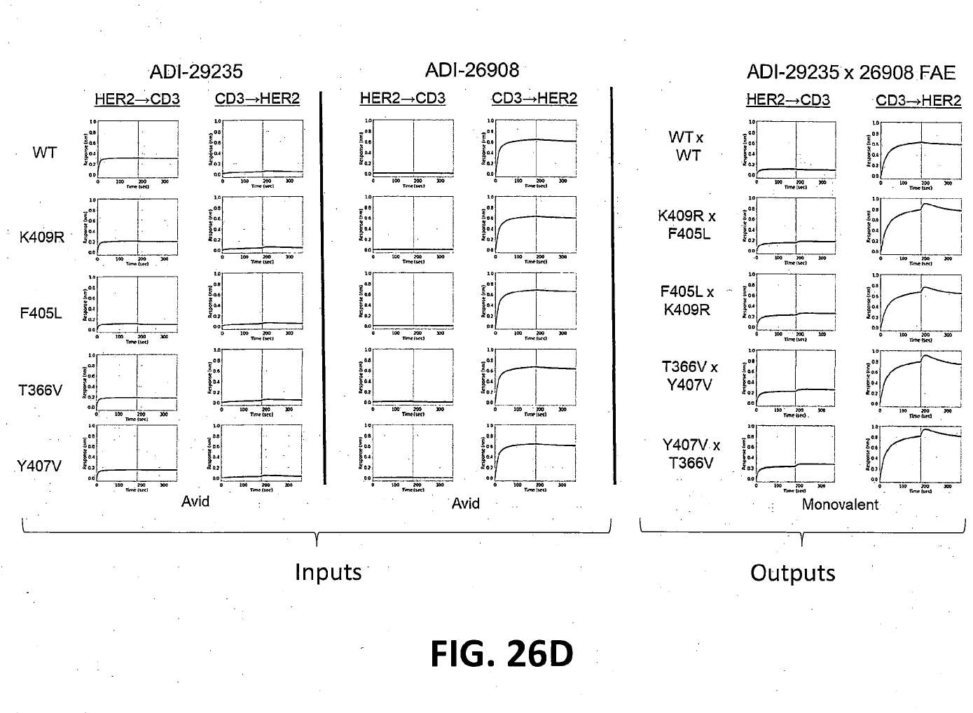

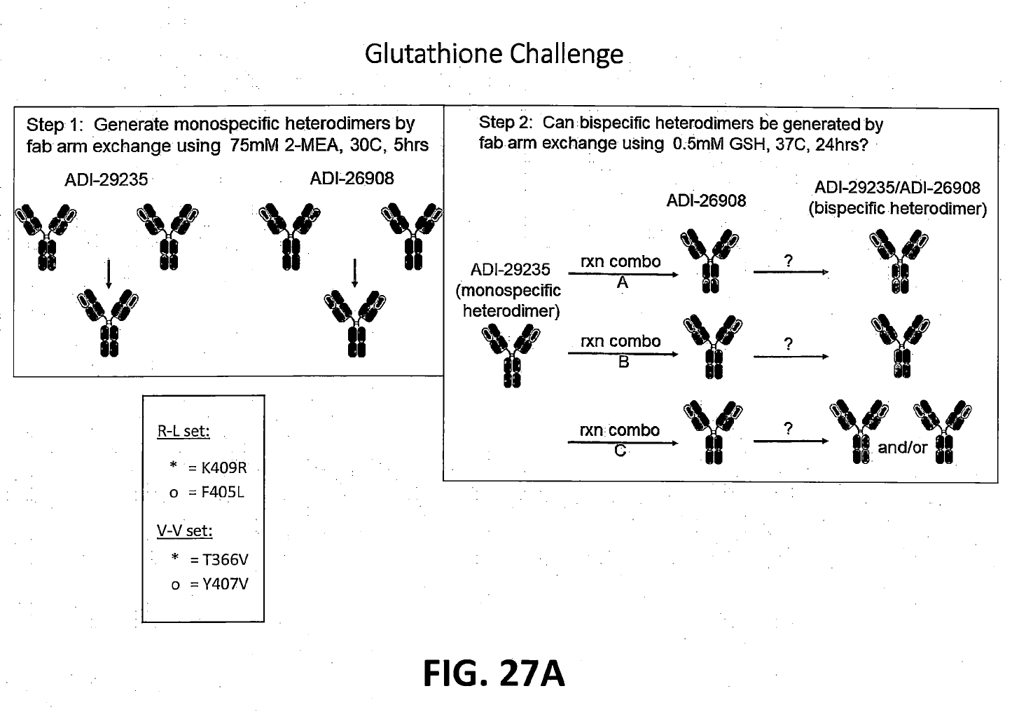

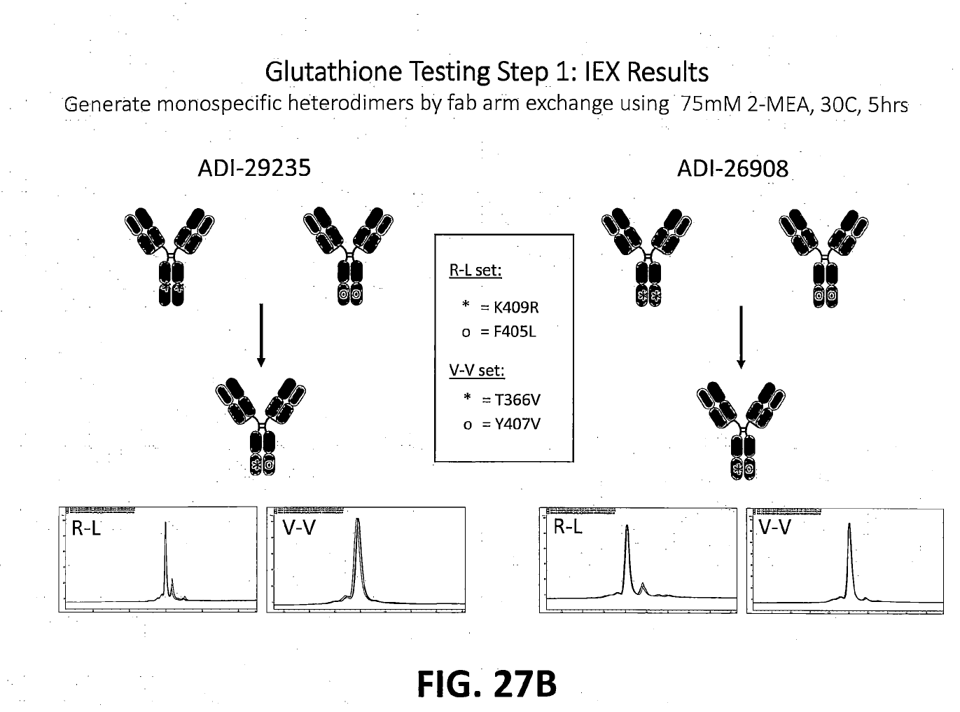

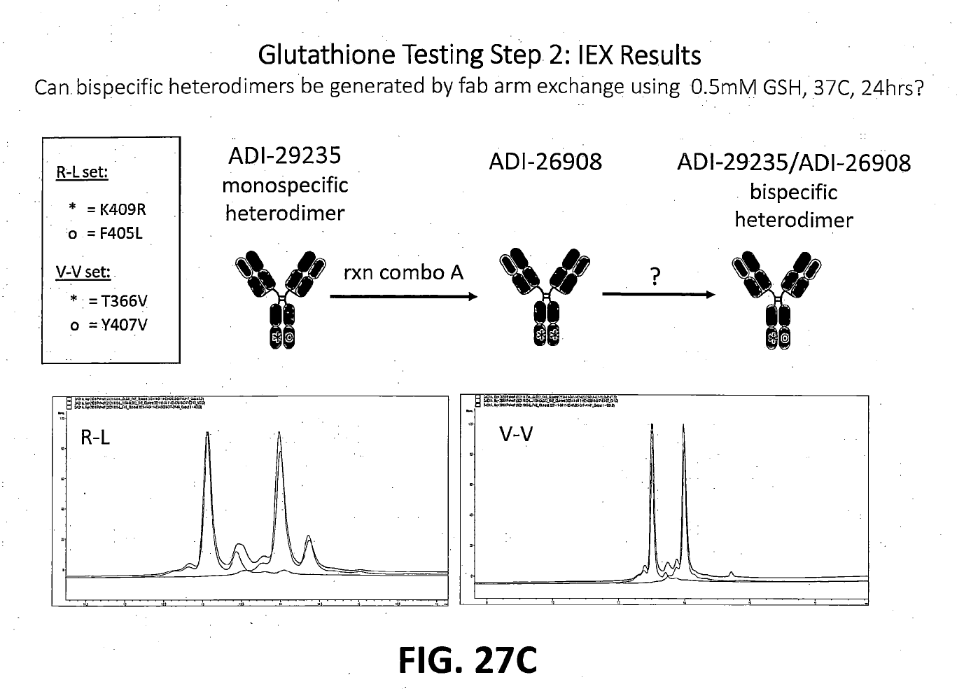

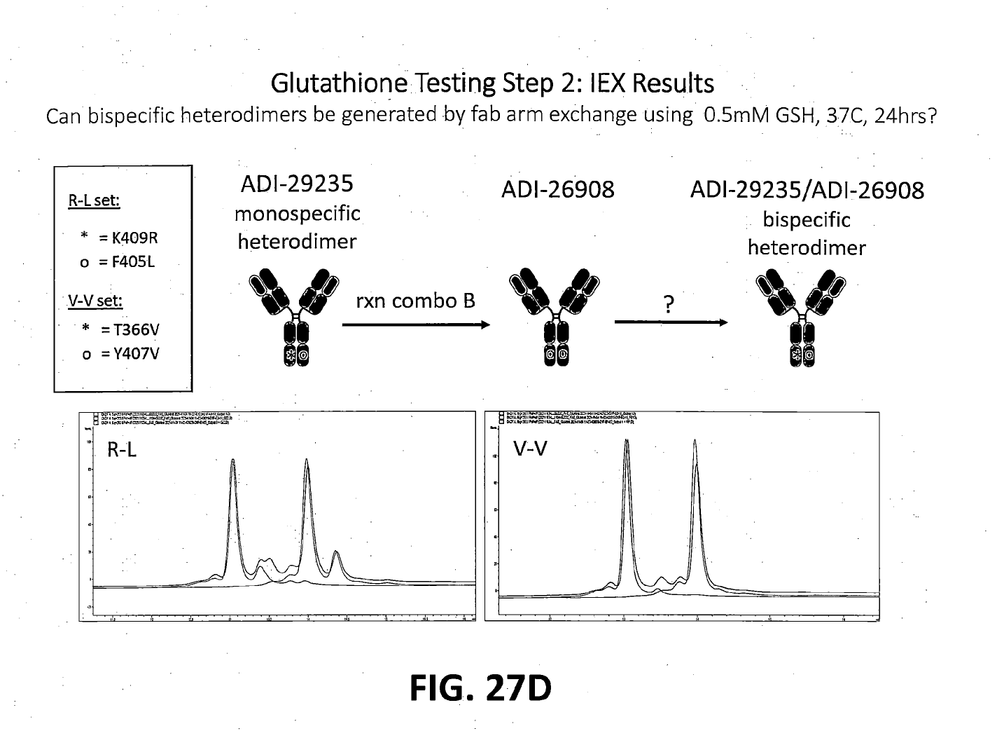

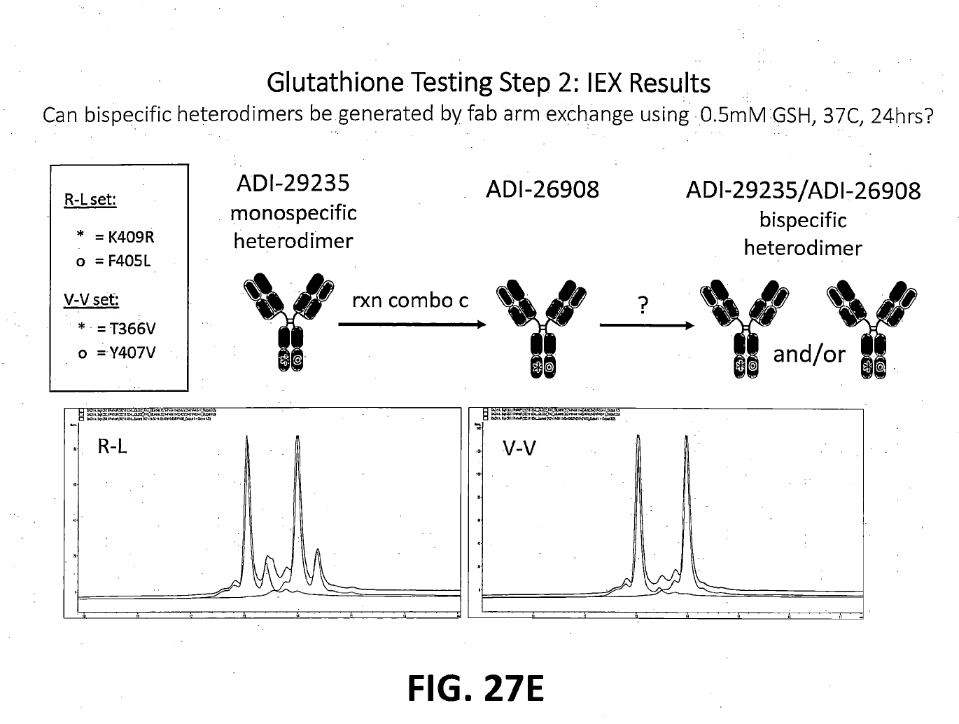

For example, when an antibody of interest comprises: (a) a half antibody specific to epitope A, comprising a heavy chain A (comprising a VH) and a light chain A (comprising a VL); and (b) a half antibody specific to epitope B, comprising a heavy chain B (comprising a VH) and a light chain B (comprising a VL), one may produce (a) an antibody A comprising two of the half antibody specific to epitope A (antibody A) and (b) an antibody B comprising two of the half antibody specific to epitope B (antibody B). Antibodies A and B may be then placed together under a mildly reducing condition, which allows for reduction of disulfide bonds between the heavy chains, resulting in respective half antibody molecules. If heavy chain A comprises a variant CH3 domain (CH3 domain A) and heavy chain B comprises a variant CH3 domain (CH3 domain B) and CH3 domains A and B preferentially form CH3-CH3 heterodimers, upon removal of the mildly reducing condition, heterodimers between heavy chains A and B may be formed preferentially over heavy chain A homodimers and heavy chain B homodimers due to cFAE, resulting in more of the bispecific antibody of interest than monospecific antibodies A and B (see FIG. 1C).

Since monospecific antibodies A and B are produced first, if the production of an antibody comprising two of a given variant CH3 domain (two of CH3 domains A or two of CH3 domains B) is poor, such bispecific antibody manufacturing methods may not be performed efficiently. This means that not all CH3 sets that prefer heterodimerization may be useful for generating bispecific antibodies via cFAE. For example, based on Applicant's experience, the pre-existing CH3 set, KiH (Table 1), is not compatible with cFAE-mediated production because the monospecific parent antibodies do not produce well. To this end, antibodies having two of the same CH3 domains belonging to the CH3 sets listed Table 16, i.e., monospecific parent antibodies, were first tested for their production yield and purity.

### Method

Monospecific IgG1 antibodies comprising (i) the variable region sequences of the anti-HER2 antibody named ADI-29235 or the anti-CD3 antibody named ADI-26908 and (ii) CH3 domains (i.e., two CH3 domains identical to each other) of WT, K409R, F405L, Y407V, T366V, T366Q_K409R, L368F, T366R_K409G, or L368F_K370G were produced in CHO cells and subjected to Protein A-based affinity purification. The production yields (mg/L) were compared. The purity of the purification products in terms of % full-size, monomer IgG molecules was also analyzed by SEC as described above.

### Results

Production yields obtained are summarize in FIG. 24A. As shown in FIG. 24A, all variant CH3 domains resulted in sufficient production yields. Some variant CH3 domains (such as ADI-29235 with the T366V CH3 domains; ADI-29235 with the L368F CH3 domains; and ADI-29235 and ADI-26908 with the T366Q_K409R CH3 domains) provided higher yields compared to the WT CH3.

Purity values after Protein A-based purification in terms of % full-size, monomer Ab are summarized in FIG. 24B. As shown in FIG. 24B, all variant CH3 domains except for the T366R_K409G CH3 domain resulted in high purity. Based on this result, “V-V” and “QR-F” sets were further tested for cFAE-based manufacturing in the following Examples, along with the control CH3 sets. However, it is noted that even though the purity of antibodies comprising the T366R_K409G CH3 domains was relatively low when produced in CHO cells, it is still possible that such antibodies may achieve high purity when produced and/or purified using different conditions such as using a different cell type.

These results overall highlight that even if a bsAb comprising a variant CH3 set is efficiently produced when all four chains of the bsAb are expressed in the same cell, it does not mean that the parent antibodies each comprising only one of the variant CH3 domains of the set and not the other variant CH3 domain of the set are efficiently produced. I.e., applicability of a variant CH3 set to FAE-based bsAb manufacturing methods is not necessarily predictable based on how preferentially the variant CH3 set forms a heterodimer.

### Example 15: cFAE Compatibility Test, Part 2—cFAE-Based bsAb Production

This Example tested if the “V-V” set and/or the “QR-F” set can mediate cFAE to produce bsAbs. The bsAbs of interest in Example 15 comprises (a) an anti-HER2 half antibody, comprising a heavy chain A (comprising the VH of ADI-29235, WT CH1 domain through CH2 domain, and a CH3 domain of a test CH3 set listed in Table 17) and a light chain A (comprising the VL of ADI-29235 and WT CL domain); and (b) an anti-CD3 half antibody, comprising a heavy chain B (comprising the VH of ADI-26908, WT CH1 domain through CH2 domain, and the other CH3 domain of said test CH3 set) and a light chain B (comprising the VL of ADI-26908 and WT CL domain). ADI-29235 and ADI-26908 share a common light chain, so the light chain A and the light chain B are identical to each other.

### Method

Anti-HER2 full-size antibodies comprising two of the anti-HER2 half antibodies and anti-CD3 full-size antibodies comprising two of the anti-CD3 antibodies (for producing bsAbs in Table 17) were produced in CHO cells and subjected to Protein A-based affinity purification. Additionally, panitumumab comprising two K409R CH3 domains and nivolumumab comprising two F405L CH3 domains (for producing bsAb Index #3 of Table 17) were also produced and purified.

The purification products were then subjected to the following FAE reaction steps and the reaction products were analyzed based on protein recovery rates (protein contents in the FAE products per 1 mg protein reaction) and bsAb formation evaluated by IEX.

### FAE Reaction:

10× 2-mercaptoethylamine-HCl (2-MEA) stock solution (750 mM) was prepared by dissolving 1.70 g of 2-MEA in 20 mL PBS. The pH was adjust to 7.4 using about 600-700 μL 2N NaOH.

For each bsAb to be tested, 500 μg of the corresponding anti-HER2 full-size antibody (250 μL of 2 mg/mL in PBS) and 500 μg of the corresponding anti-CD3 full-size antibody (250 μL of 2 mg/mL in PBS) were placed in a well of a deep well plate. In case of bsAb Index #3, 500 μg of panitumumab with K409R and 500 μg of nivolumab with F405L were used. 400 μL of PBS followed by 100 μL of 10× 2-MEA stock solution was then added to each well to give a final 2-MEA concentration of 75 mM. Samples were incubated stationary for 5 hours at 30° C.

Desalting with Sephadex® G25 plate (1 mL split over three wells) was performed for each sample to exchange buffer into PBS to remove 2-MEA (to achieve about <50 μM). Samples were then incubated for additional 48 hours at 4° C.

TABLE 17
BsAbs tested for cFAE-based production

| bsAb Index | CH3 Set Name | Chain | CH3 substitutions | Variable region |
|---|---|---|---|---|
| 1 | WT | A | None | ADI-29235 (anti-HER2) |
| | | B | None | ADI-26908 (anti-CD3) |
| 2 | R-L | A | K409R | ADI-29235 (anti-HER2) |
| | | B | F405L | ADI-26908 (anti-CD3) |
| 3 | R-L | A | K409R | Panitumumab |
| | | B | F405L | Nivolumab |
| 4 | R-L | A | F405L | ADI-29235 (anti-HER2) |
| | | B | K409R | ADI-26908 (anti-CD3) |
| 5 | V-V | A | T366V | ADI-29235 (anti-HER2) |
| | | B | Y407V | ADI-26908 (anti-CD3) |
| 6 | V-V | A | Y407V | ADI-29235 (anti-HER2) |
| | | B | T366V | ADI-26908 (anti-CD3) |
| 7 | QR-F | A | T366Q K409R | ADI-29235 (anti-HER2) |
| | | B | L368F | ADI-26908 (anti-CD3) |
| 8 | QR-F | A | L368F | ADI-29235 (anti-HER2) |
| | | B | T366Q K409R | ADI-26908 (anti-CD3) |

### Results

### 1. Protein Recovery

The protein recovery rates are provided in FIG. 25A. As shown in FIG. 25A, the recovery rates were about 80% and similar among different bsAb samples.

### 2. BsAb Formation Evaluated by IEX

Exemplary IEX results for the three variant CH3 sets tested, R-L, V-V, and QR-F (corresponding to BsAb ID #2 and 4-8), each panel showing an overlay of a chromatogram of a FAE reaction product (shown as “output”) and chromatograms of purification products of the corresponding parent antibodies (shown as “input”) are provided in FIG. 25B. As shown in FIG. 25B, the R-L set (bsAb Index #2 and 4) and the V-V set (bsAb Index #5 and 6) successfully resulted in the intended bsAbs upon FAE reaction steps. In contrast, much smaller amounts of the intended bsAbs were obtained using the QR-F set.

These results particularly highlight that even if a variant CH3 domain set preferentially form heterodimers over homodimers and allows for preferential production of bsAbs when all four chains are expressed in the same cell, it does not mean that the variant CH3 domain set mediates FAE. I.e., applicability to FAE-based bsAb manufacturing methods is not predictable.

### Example 16: cFAE Compatibility Test, Part 3—Further Analyses on the V-V Set

This Example further analyzed the FAE products for bsAb production using the “V-V” set (bsAb Index #5-6), along with the negative control (WT) (bsAb Index #1) and positive control (“R-L”) (bsAb Index #3-4). Specifically, the product quality analyzed by SDS-PAGE, bsAb formation efficiency analyzed by LC-MS, and separate or simultaneous binding to cognate antigens analyzed by biolayer interferometry (BLI) were compared between the FAE reaction products (“output”) and their monodpecific parent antibody (“input”).

### 1. FAE Product Quality by SDS-PAGE:

The protein contents in the FAE products and the purification products (FAE output) of the monospecific parent antibodies (FAE input) were visualized by SDS-PAGE (non-reduced) and compared.

The SDS-PAGE results are provided in FIG. 26A. As shown in FIG. 26A, similar band patterns were observed between the input and output samples in all tested CH3 sets. No prominent bands of <60 kDa were observed. I.e., the protein quality was consistent between inputs and outputs.

### 2. BsAb Formation Evaluated by LC-MS:

The FAE products for bsAbs comprising the R-L or V-V set (bsAb Index #2 and 4-6) and their parent antibodies were analyzed by LC-MS in a non-reducing condition.

Exemplary LC-MS results, each panel showing an overlay of a chromatogram of a FAE reaction product (shown as “output”) and chromatograms of purification products of the corresponding parent antibodies (shown as “input”) are provided in FIG. 26B. As shown in FIG. 26B, both the R-L and V-V sets provided successful production of the intended bsAbs. % of each species (“aAAa”, “aABa”, or “aBBa”) obtained among the total full-size antibody products, calculated based on the LC-MS results are provided in Table 18. In Table 18, “aABa” represents an antibody having one heavy chain A (“A”) and one heavy chain B (“B”), each paired with the common light chain (“a”), i.e., the intended bsAb; “aAAa” represents the parent antibody A comprising two heavy chains A each paired with the common light chain, i.e., ADI-29235 comprising the indicated variant CH3; and “aBBa” represents the parent antibody B comprising two heavy chains B, each paired with the common light chain, i.e., ADI-26908 comprising the indicated variant CH3. The % values are % of all full-size IgG molecules obtained. As shown in Table 18, the V-V set achieved excellent bsAb production providing 100% of the intended bsAb, which is even higher than what was achieved using the positive control (the R-L set).

TABLE 18
BsAbs tested for cFAE-based production

| | CH3 | | | | | | |
|---|---|---|---|---|---|---|---|
| bsAb | Set | | CH3 | | aAAa | aABa | aBBa |
| Index | Name | Chain | substitutions | Variable region | (%) | (%) | (%) |
| 2 | R-L | A | K409R | ADI-29235 (anti-HER2) | 4 | 96 | 0 |
| | | B | F405L | ADI-26908 (anti-CD3) | | | |
| 4 | R-L | A | F405L | ADI-29235 (anti-HER2) | 3 | 94 | 3 |
| | | B | K409R | ADI-26908 (anti-CD3) | | | |
| 5 | V-V | A | T366V | ADI-29235 (anti-HER2) | 0 | 100 | 0 |
| | | B | Y407V | ADI-26908 (anti-CD3) | | | |
| 6 | V-V | A | Y407V | ADI-29235 (anti-HER2) | 0 | 100 | 0 |
| | | B | T366V | ADI-26908 (anti-CD3) | | | |

### 3. Binding Kinetics to Cognate Antigens:

Binding kinetics of the FAE products (bsAb Index #1-2 and 4-6) and their monospecific parent antibodies to the cognate antigen(s) were compared.

### Method

Binding to a cognate antigen (HER2 or CD3) was measured by BLI using a FortéBio Octet HTX instrument (Molecular Devices). The IgGs were captured (1.5 nm) to anti-human IgG capture (AHC) biosensors Molecular Devices) and allowed to stand in PBSF (PBS with 0.1% w/v BSA) for a minimum of 30 min. After a short (60 s) baseline step in PBSF, the IgG-loaded biosensor tips were exposed (180 s, 1000 rpm of orbital shaking) to HER2 or CD3 (100 nM in PBSF) and then dipped (180 s, 1000 rpm of orbital shaking) into PBSF to measure any dissociation of the antigen from the biosensor tip surface. Data for which binding responses were >0.1 nm were aligned, inter-step corrected (to the association step) and fit to a 1:1 binding model using the FortéBio Data Analysis Software, version 11.1.

### Results

Exemplary binding kinetic curves are provided in FIG. 26C. The binding kinetics of bsAbs to cognate antigens matched those of their corresponding, monospecific parent antibodies. The binding kinetics were not significantly affected by the CH3 substitutions.

### 4. Simultaneous Antigen Binding:

Finally, the FAE products (bsAb Index #1-2 and 4-6) were tested whether they are able to bind simultaneously to two cognate antigens and the binding kinetics were compared with those of their parent antibodies.

### Method

Simultaneous antigen binding was tested at 25° C. on a ForteBio Octet HTX instrument (Sartorius, Gottingen, Germany). Binding kinetics of individual bsAbs and the respective monospecific parent antibodies to HER2 and then CD3 or to CD3 and then HER2 were analyzed All reagents were formulated into phosphate buffered saline with 0.1% (w/w) BSA (PBSF).

To test binding to HER2 and then CD3, monomeric HER2-moFc (100 nM) was first loaded to anti-mouse Fc IgG capture sensor tips (Sartorius, Gottingen, Germany) and then allowed to stand in PBSF for a minimum of 15 minutes. These loaded sensor tips were initially exposed (60 s) to wells containing PBSF to establish a stable baseline for the assay before exposure (180 s) to the bsAb (100 nM) and then finally (600 s) to CD3 (100 nM).

To test binding to CD3 and then HER2, monomeric CD3-moFc (100 nM) was first loaded to anti-mouse Fc IgG capture sensor tips (Sartorius, Gottingen, Germany) and then allowed to stand in PBSF for a minimum of 15 minutes. These loaded sensor tips were initially exposed (60 s) to wells containing PBSF to establish a stable baseline for the assay before exposure (180 s) to the bsAb (100 nM) and then finally (600 s) to HER2 (100 nM).

BsAbs with sufficient binding responses in the final two steps of the assay were considered as dual binders.

### Results

Exemplary binding kinetic curves are provided in FIG. 26D. As shown in FIG. 26D, the FAE products from the V-V set and the R-L set showed simultaneous binding to HER2 and CD3, regardless of whether the FAE products were exposed to HER2 first or to CD3 first.

### Example 17: cFAE Compatibility Test, Part 4—Glutathione Challenge

This Example tested whether antibodies comprising the V-V set produced by the FAE-based method are stable in the presence of glutathione (GSH). Specifically, Example 17 tested whether, when exposed to GSH, a CH3 heterodimer generated by FAE under 2-MEA would dissociate and recombine with another CH3 domain generated from another (homo or hetero) CH3 set. The stability was compared with that of the R-L set.

### Method

For each of R-L and V-V sets, the following steps 1 and 2 were performed.

Step 1: First, a first anti-HER2 IgG1 comprising (i) the ADI-29235 variable domains and (ii) one variant CH3 of a test CH3 set (i.e., two same CH3 domains), was produced and purified. A second anti-HER2 IgG1 comprising (i) the ADI-29235 variable domains and (ii) the other variant CH3 of said test CH3 set (i.e., two same CH3 domains), was also produced and purified. Next, FAE reactions were performed on the mixture the first and second anti-HER2 IgG1s, essentially as described in Example 15 using 75 mM 2-MEA and incubation of 5 hours at 30° C., to obtain an anti-HER2, CH3 hetero IgG1 comprising (i) ADI-29235 variable domains and (ii) said test CH3 set.

Similarly, a first anti-CD3 IgG1 comprising (i) the ADI-26908 variable domains and (ii) one variant CH3 of said test CH3 set (i.e., two same CH3 domains), was produced and purified. A second anti-CD3 IgG1 comprising (i) the ADI-26908 variable domains and (ii) the other variant CH3 of said test CH3 set (i.e., two same CH3 domains), was also produced and purified. Next, FAE reactions were performed on the mixture the first and second anti-CD3 IgG1s, essentially as described in Example 15 using 75 mM 2-MEA and incubation of 5 hours at 30° C., to obtain a an anti-CD3, CH3 hetero IgG1 comprising (i) ADI-26908 variable domains and (ii) said test CH3 set.

Step 2: The anti-HER2, CH3 hetero antibody was mixed with (I) the first anti-CD3 IgG1, (II) the second anti-CD3 IgG1, or (III) the anti-CD3, CH3 hetero IgG1 and was placed in a mildly reducing environment comprising 0.5 mM GSH and incubated for 24 hours at 37° C. (this process of incubation with GSH is referred to as “GDH challenge” herein). The GSH challenge products were analyzed by IEX to determine whether further FAE occurred.

The experimental scheme of Example 17 is summarized in FIG. 27A.

### Results

FIG. 27B show exemplary IEX results for R-L and V-V sets in FAE using 2-MEA in Step 1. FIG. 27C-27E provide exemplary IEX results for R-L and V-V sets in GSH challenge in Step 2. Each graph panel shows an overlay of a chromatogram of a GSH challenge product and chromatograms of the two GSH challenge input antibodies (i.e., the anti-HER2, CH3 hetero antibody; and (I) the first anti-CD3 IgG1 in case of FIG. 27C, (II) the second anti-CD3 IgG1 in case of FIG. 27D, or (III) the anti-CD3, CH3 hetero in case of FIG. 27E). As shown in FIG. 27C-27E, GSH challenge did not result in a new IEX peak, indicating that chain recombination between the GSH challenge input antibodies did not occur. I.e., CH3 heterodimers generated by FAE under 2-MEA is stable and do not recombine with another CH3 domain generated from another (homo or hetero) CH3 set in the presence of GSH. The stability of the V-V set under the GSH stress was comparable to that of the R-L set.

### Example 18: FAE Under 75 mM 2-MEA at 30° C. for 5 Hours does not Cause Dissociation Between Heavy and Light Chains

This Example tested whether the cFAE reaction condition used in Example 15-17 would cause dissociation between heavy and light chains. BsAbs as shown in Table 19 each comprising (i) a half antibody specific for a first antigen, comprising a heavy chain A and a light chain A and (ii) a half antibody specific for a second antigen, comprising a heavy chain B and a light chain B, were the bsAbs of interest in this Example. The variable sequences used were those from panitumumab (anti-EGFR), nivolumab (anti-PD-1), or imgatuzumab (anti-EGFR).

TABLE 19
BsAbs tested for cFAE-based production

| bsAb Index | CH3 Set Name | Chain | CH3 substitutions | Variable region |
|---|---|---|---|---|
| 9 | R-L | A | K409R | Panitumumab |
| | | B | F405L | Nivolumab |
| 10 | R-L | A | F405L | Panitumumab |
| | | B | K409R | Nivolumab |
| 11 | WT | A | None | Panitumumab |
| | | B | None | Nivolumab |
| 12 | R-L | A | K409R | Panitumumab |
| | | B | F405L | Imgatuzumab |
| 13 | R-L | A | F405L | Panitumumab |
| | | B | K409R | Imgatuzumab |
| 14 | WT | A | None | Panitumumab |
| | | B | None | Imgatuzumab |
| 15 | R-L | A | K409R | Nivolumab |
| | | B | F405L | Imgatuzumab |
| 16 | R-L | A | F405L | Nivolumab |
| | | B | K409R | Imgatuzumab |
| 17 | WT | A | None | Nivolumab |
| | | B | None | Imgatuzumab |

### Method

For each of the bsAbs listed in Table 19, the respective monospecific parent antibodies (i.e., antibody A specific for the first antigen and antibody B specific for the second antigen with the indicated CH3 modifications) were produced in CHO cells and subjected to Protein A-based affinity purification. The purification products were then subjected to the following FAE reaction steps. The FAE reaction products were digested by GingisKHAN® enzyme to obtain Fab fragments, which were analyzed by LC-MS.

### FAE Reaction:

10× 2-mercaptoethylamine-HCl (2-MEA) stock solution (750 mM) was prepared by dissolving 1.70 g of 2-MEA in 20 mL PBS. The pH was adjust to 7.4 using about 600-700 μL 2N NaOH.

For each bsAb to be tested, 500 μg of the corresponding anti-HER2 full-size antibody (250 μL of 2 mg/mL in PBS) and 500 μg of the corresponding anti-CD3 full-size antibody (250 μL of 2 mg/mL in PBS) were placed in a well of a deep well plate. In case of bsAb Index #3, 500 μg of panitumumab with K409R and 500 μg of nivolumab with F405L were used. 400 μL of PBS followed by 100 μL of 10× 2-MEA stock solution was then added to each well to give a final 2-MEA concentration of 75 mM. Samples were incubated stationary for 5 hours at 30° C.

2-MEA was removed via buffer exchanged by diafiltration using 10 kDa MWCO spin tubes (<500 μL+3 mL×6). Samples were then incubated for additional 48 hours at 4° C.

### Results

Fabs species identified by LC-MS are provided in Table 20. In Table 20: “aA” represents the Fab derived from a half antibody comprising one heavy chain A (“A”) and one light chain A (“a”); “bA” represents the Fab derived from a half antibody comprising one heavy chain A (“A”) and one light chain B (“b”); “aB” represents the Fab derived from a half antibody comprising one heavy chain B (“B”) and one light chain A (“a”); and “bB” represents the Fab derived from a half antibody comprising one heavy chain B (“B”) and one light chain B (“b”). I.e., “aA” and “bB” are cognate pairs and “bA” and “aB” are non-cognate pairs. The % values are % of all Fabs obtained from digestion of the FAE products. As shown in Table 20, no non-cognate pairs were found in any specificity combinations tested. I.e., the cFAE reaction condition does not break the disulfide bond between heavy and light chains.

TABLE 20
Fab species pairing percentages

| bsAb | CH3 Set | | CH3 | Variable | | | | |
|---|---|---|---|---|---|---|---|---|
| Index | Name | Chain | substitutions | region | aA (%) | bA (%) | aB (%) | bB (%) |
| 9 | R-L | A | K409R | pani | 56 | 0 | 0 | 44 |
| | | B | F405L | nivo | | | | |
| 10 | R-L | A | F405L | pani | 54 | 0 | 0 | 46 |
| | | B | K409R | nivo | | | | |
| 11 | WT | A | None | pani | 55 | 0 | 0 | 45 |
| | | B | None | nivo | | | | |
| 12 | R-L | A | K409R | pani | 61 | 0 | 0 | 39 |
| | | B | F405L | imga | | | | |
| 13 | R-L | A | F405L | pani | 61 | 0 | 0 | 39 |
| | | B | K409R | imga | | | | |
| 14 | WT | A | None | pani | 64 | 0 | 0 | 36 |
| | | B | None | imga | | | | |
| 15 | R-L | A | K409R | nivo | 60 | 0 | 0 | 39 |
| | | B | F405L | imga | | | | |
| 16 | R-L | A | F405L | nivo | 58 | 0 | 0 | 42 |
| | | B | K409R | imga | | | | |
| 17 | WT | A | None | nivo | 61 | 0 | 0 | 38 |
| | | B | None | imga | | | | |

### Example 19: FAE with Alternative Variable Domains

To confirm broad applicability of FAE for use with various parental antibody combinations, additional FAE reactions were carried out using human IgG1 antibodies directed to different targets, each with different variable domain pairs. Parental antibody constructs were prepared using sequences encoding antibody variable domains derived from mosunetuzumab, panitumumab, or nivolumab; CH2 domain Fc-silencing mutations (L234A, L235A, and P329A, according to EU numbering); and either Y407V or T366V CH3 mutations. Specific pairs tested are shown in the Table below.

TABLE 21
Parental antibody pairs

| Pair | Parental | | Parental | |
|---|---|---|---|---|
| Number | Antibody 1 | Description | Antibody 2 | Description |
| 1 | Mosun CD20 | IgG1 antibody with | Mosun CD3 | IgG1 antibody with |
| | (Y407V) | both arms having anti- | (T366V) | both arms having anti- |
| | | CD20 variable domains | | CD3 variable domains |
| | | of mosunetuzumab | | of mosunetuzumab |
| | | and Y407V CH3 | | and T366V CH3 |
| | | mutations | | mutations |
| 2 | Mosun CD20 | IgG1 antibody with | Mosun CD3 | IgG1 antibody with |
| | (T366V) | both arms having anti- | (Y407V) | both arms having anti- |
| | | CD20 variable domains | | CD3 variable domains |
| | | of mosunetuzumab | | of mosunetuzumab |
| | | and T366V CH3 | | and Y407V CH3 |
| | | mutations | | mutations |
| 3 | Panitumumab | IgG1 antibody with | Nivolumab | IgG1 antibody with |
| | (Y407V) | anti-EGFR variable | (T366V) | anti-PD-1 variable |
| | | domains of | | domains of nivolumab |
| | | panitumumab and | | and T366V CH3 |
| | | Y407V CH3 mutations | | mutations |
| 4 | Panitumumab | IgG1 antibody with | Nivolumab | IgG1 antibody with |
| | (T366V) | anti-EGFR variable | (Y407V) | anti-PD-1 variable |
| | | domains of | | domains of nivolumab |
| | | panitumumab and | | and Y407V CH3 |
| | | T366V CH3 mutations | | mutations |

Parental antibody constructs were expressed in CHO cells and isolated by Protein A-based affinity purification. For FAE reactions, 10× 2-mercaptoethylamine-HCl (2-MEA) stock solution (750 mM) was prepared by dissolving 1.70 g of 2-MEA in 20 mL PBS. The pH was adjusted to 7.4 using about 600-700 μL 2N NaOH. 500 μg of each parental antibody (250 μL of 2 mg/mL in PBS) was placed in a well of a deep well plate. 400 μL of PBS followed by 100 μL of 10× 2-MEA stock solution was then added to each well to give a final 2-MEA concentration of 75 mM. Samples were incubated for 5 hours at 30° C.

Desalting with SEPHADEX® G25 plate (1 mL split over three wells) was performed for each sample to exchange buffer into PBS to remove 2-MEA (to achieve about <50 μM). Samples were then incubated for 48 hours at 4° C.

Resulting antibody outputs were examined for: (1) percentage of protein recovered; (2) percentage of full antibody monomer by size-exclusion chromatography (SEC), and (3) formation of desired heterodimeric antibody species by comparison of retention time obtained by analytical ion exchange chromatography (IEX) with parental antibody retention times (see Table below).

TABLE 22
FAE output analysis

| | | | IEX, | IEX, | IEX, parental | |
|---|---|---|---|---|---|---|
| | | | parental | parental | antibody | |
| | | SEC, % full | antibody 1 | antibody 2 | retention | IEX, output |
| Pair | % | antibody | retention | retention | time, average | retention time |
| Number | Recovery | monomer | time (min) | time (min) | (min) | (min) |
| 1 | 80 | 95.6 | 15.19 | 17.27 | 16.23 | 16.22 |
| 2 | 81 | 95.7 | 15.15 | 17.31 | 16.23 | 16.24 |
| 3 | 74 | 96.5 | 11.07 | 16.16 | 13.62 | 13.37 |
| 4 | 73 | 96.9 | 11.06 | 16.21 | 13.64 | 13.37 |

The results demonstrated greater than 70% protein recovery with greater than 95% representing full, unaggregated antibody. Further, output retention times were closer to average parental antibody retention times than individual parental antibody retention times indicating successful heterodimeric chain pairing.

### Example 20: FAE Generation of Biparatopic Antibodies

Biparatopic antibodies are bispecific antibodies where each paratope is directed to a different epitope of the same antigen. To assess suitability of FAE for generating biparatopic antibodies, FAE was carried out using two parental antibody groups, Group 1 and Group 2. Group 1 antibodies each targeted a different epitope of the same viral antigen. Group 2 antibodies each targeted a different epitope of the same cell surface antigen. Each Group 1 and 2 antibody was expressed in CHO cells as human IgG1 antibodies with either Y407V (parental antibody 1) or T366V (parental antibody 2) CH3 mutations.

Parental antibody constructs were expressed in CHO cells and isolated by Protein A-based affinity purification. For FAE reactions, 10× 2-mercaptoethylamine-HCl (2-MEA) stock solution (750 mM) was prepared by dissolving 1.70 g of 2-MEA in 20 mL PBS. The pH was adjusted to 7.4 using about 600-700 μL 2N NaOH. 500 μg of each parental antibody (250 μL of 2 mg/mL in PBS) was placed in a well of a deep well plate. 400 μL of PBS followed by 100 μL of 10× 2-MEA stock solution was then added to each well to give a final 2-MEA concentration of 75 mM. Samples were incubated for 5 hours at 30° C.

Desalting with SEPHADEX® G25 plate (1 mL split over three wells) was performed for each sample to exchange buffer into PBS to remove 2-MEA (to achieve about <50 μM). Samples were then incubated for 48 hours at 4° C.

Resulting antibody outputs were examined for percentage of protein recovered and formation of desired heterodimeric antibody species by comparison of retention time obtained by analytical ion exchange chromatography (IEX) with parental antibody retention times (see Table below).

TABLE 23
Parental antibody pairs

| | | | | | IEX | | | |
|---|---|---|---|---|---|---|---|---|
| | | | | | | | Avg Parent | |
| | | Parent | Parent | % | Parent Ab 1 | Parent Ab 2 | Ab ret. | Output ret. |
| Group # | Pair # | Ab 1 | Ab 2 | Recovery | ret. time (min) | ret. time (min) | time (min) | Time (min) |
| 1 | A01 | 003 | 003 | 86 | 12.04 | 12.04 | 12.04 | 12.07 |
| 1 | A02 | 001 | 003 | 75 | 13.27 | 12.04 | 12.66 | 12.54 |
| 1 | A03 | 002 | 003 | 72 | 14.68 | 12.04 | 13.36 | 13.31 |
| 1 | A04 | 005 | 003 | 73 | 12.30 | 12.04 | 12.17 | 12.13 |
| 1 | A05 | 011 | 003 | 73 | 14.36 | 12.04 | 13.20 | 13.23 |
| 1 | A06 | 006 | 003 | 75 | 14.09 | 12.04 | 13.07 | 13.00 |
| 1 | A07 | 007 | 003 | 79 | 14.68 | 12.04 | 13.36 | 13.35 |
| 1 | A08 | 009 | 003 | 78 | 12.70 | 12.04 | 12.37 | 12.31 |
| 1 | A09 | 008 | 003 | 79 | 15.40 | 12.04 | 13.72 | 14.01 |
| 1 | A10 | 004 | 003 | 73 | 10.87 | 12.04 | 11.46 | 11.54 |
| 1 | A11 | 012 | 003 | 73 | 12.71 | 12.04 | 12.38 | 12.37 |
| 1 | A12 | 010 | 003 | 74 | 14.24 | 12.04 | 13.14 | 13.11 |
| 1 | A13 | 003 | 001 | 74 | 12.04 | 13.27 | 12.66 | 12.55 |
| 1 | A14 | 001 | 001 | 66 | 13.27 | 13.27 | 13.27 | 13.33 |
| 1 | A15 | 002 | 001 | 67 | 14.68 | 13.27 | 13.98 | 13.99 |
| 1 | A16 | 005 | 001 | 72 | 12.30 | 13.27 | 12.79 | 12.80 |
| 1 | A17 | 011 | 001 | 66 | 14.36 | 13.27 | 13.82 | 13.86 |
| 1 | A18 | 006 | 001 | 65 | 14.09 | 13.27 | 13.68 | 13.71 |
| 1 | A19 | 007 | 001 | 65 | 14.68 | 13.27 | 13.98 | 14.05 |
| 1 | A20 | 009 | 001 | 66 | 12.70 | 13.27 | 12.99 | 13.00 |
| 1 | A21 | 008 | 001 | 69 | 15.40 | 13.27 | 14.34 | 14.62 |
| 1 | A22 | 004 | 001 | 66 | 10.87 | 13.27 | 12.07 | 12.02 |
| 1 | A23 | 012 | 001 | 67 | 12.71 | 13.27 | 12.99 | 12.90 |
| 1 | A24 | 010 | 001 | 69 | 14.24 | 13.27 | 13.76 | 13.66 |
| 1 | A25 | 003 | 002 | 79 | 12.04 | 14.68 | 13.36 | 13.32 |
| 1 | A26 | 001 | 002 | 67 | 13.27 | 14.68 | 13.98 | 13.99 |
| 1 | A27 | 002 | 002 | 64 | 14.68 | 14.68 | 14.68 | 14.76 |
| 1 | A28 | 005 | 002 | 67 | 12.30 | 14.68 | 13.49 | 13.54 |
| 1 | A29 | 011 | 002 | 65 | 14.36 | 14.68 | 14.52 | 14.58 |
| 1 | A30 | 006 | 002 | 64 | 14.09 | 14.68 | 14.39 | 14.46 |
| 1 | A31 | 007 | 002 | 66 | 14.68 | 14.68 | 14.68 | 14.75 |
| 1 | A32 | 009 | 002 | 74 | 12.70 | 14.68 | 13.69 | 13.76 |
| 1 | A33 | 008 | 002 | 69 | 15.40 | 14.68 | 15.04 | 15.18 |
| 1 | A34 | 004 | 002 | 63 | 10.87 | 14.68 | 12.78 | 12.86 |
| 1 | A35 | 012 | 002 | 65 | 12.71 | 14.68 | 13.70 | 13.78 |
| 1 | A36 | 010 | 002 | 64 | 14.24 | 14.68 | 14.46 | 14.48 |
| 1 | A37 | 003 | 005 | 74 | 12.04 | 12.30 | 12.17 | 12.14 |
| 1 | A38 | 001 | 005 | 67 | 13.27 | 12.30 | 12.79 | 12.81 |
| 1 | A39 | 002 | 005 | 68 | 14.68 | 12.30 | 13.49 | 13.54 |
| 1 | A40 | 005 | 005 | 74 | 12.30 | 12.30 | 12.30 | 12.36 |
| 1 | A41 | 011 | 005 | 67 | 14.36 | 12.30 | 13.33 | 13.44 |
| 1 | A42 | 006 | 005 | 66 | 14.09 | 12.30 | 13.20 | 13.25 |
| 1 | A43 | 007 | 005 | 64 | 14.68 | 12.30 | 13.49 | 13.58 |
| 1 | A44 | 009 | 005 | 65 | 12.70 | 12.30 | 12.50 | 12.55 |
| 1 | A45 | 008 | 005 | 67 | 15.40 | 12.30 | 13.85 | 14.22 |
| 1 | A46 | 004 | 005 | 70 | 10.87 | 12.30 | 11.59 | 11.60 |
| 1 | A47 | 012 | 005 | 69 | 12.71 | 12.30 | 12.51 | 12.45 |
| 1 | A48 | 010 | 005 | 69 | 14.24 | 12.30 | 13.27 | 13.20 |
| 1 | A49 | 003 | 011 | 77 | 12.04 | 14.36 | 13.20 | 13.24 |
| 1 | A50 | 001 | 011 | 66 | 13.27 | 14.36 | 13.82 | 13.87 |
| 1 | A51 | 002 | 011 | 63 | 14.68 | 14.36 | 14.52 | 14.59 |
| 1 | A52 | 005 | 011 | 68 | 12.30 | 14.36 | 13.33 | 13.45 |
| 1 | A53 | 011 | 011 | 67 | 14.36 | 14.36 | 14.36 | 14.43 |
| 1 | A54 | 006 | 011 | 66 | 14.09 | 14.36 | 14.23 | 14.30 |
| 1 | A55 | 007 | 011 | 68 | 14.68 | 14.36 | 14.52 | 14.59 |
| 1 | A56 | 009 | 011 | 68 | 12.70 | 14.36 | 13.53 | 13.65 |
| 1 | A57 | 008 | 011 | 69 | 15.40 | 14.36 | 14.88 | 15.03 |
| 1 | A58 | 004 | 011 | 68 | 10.87 | 14.36 | 12.62 | 12.97 |
| 1 | A59 | 012 | 011 | 64 | 12.71 | 14.36 | 13.54 | 13.66 |
| 1 | A60 | 010 | 011 | 64 | 14.24 | 14.36 | 14.30 | 14.31 |
| 1 | A61 | 003 | 006 | 75 | 12.04 | 14.09 | 13.07 | 13.02 |
| 1 | A62 | 001 | 006 | 64 | 13.27 | 14.09 | 13.68 | 13.73 |
| 1 | A63 | 002 | 006 | 68 | 14.68 | 14.09 | 14.39 | 14.47 |
| 1 | A64 | 005 | 006 | 73 | 12.30 | 14.09 | 13.20 | 13.26 |
| 1 | A65 | 011 | 006 | 67 | 14.36 | 14.09 | 14.23 | 14.31 |
| 1 | A66 | 006 | 006 | 67 | 14.09 | 14.09 | 14.09 | 14.18 |
| 1 | A67 | 007 | 006 | 67 | 14.68 | 14.09 | 14.39 | 14.48 |
| 1 | A68 | 009 | 006 | 67 | 12.70 | 14.09 | 13.40 | 13.47 |
| 1 | A69 | 008 | 006 | 68 | 15.40 | 14.09 | 14.75 | 14.96 |
| 1 | A70 | 004 | 006 | 70 | 10.87 | 14.09 | 12.48 | 12.52 |
| 1 | A71 | 012 | 006 | 69 | 12.71 | 14.09 | 13.40 | 13.43 |
| 1 | A72 | 010 | 006 | 70 | 14.24 | 14.09 | 14.17 | 14.15 |
| 1 | A73 | 003 | 007 | 80 | 12.04 | 14.68 | 13.36 | 13.36 |
| 1 | A74 | 001 | 007 | 68 | 13.27 | 14.68 | 13.98 | 14.07 |
| 1 | A75 | 002 | 007 | 66 | 14.68 | 14.68 | 14.68 | 14.76 |
| 1 | A76 | 005 | 007 | 68 | 12.30 | 14.68 | 13.49 | 13.60 |
| 1 | A77 | 011 | 007 | 63 | 14.36 | 14.68 | 14.52 | 14.59 |
| 1 | A78 | 006 | 007 | 66 | 14.09 | 14.68 | 14.39 | 14.48 |
| 1 | A79 | 007 | 007 | 73 | 14.68 | 14.68 | 14.68 | 14.76 |
| 1 | A80 | 009 | 007 | 72 | 12.70 | 14.68 | 13.69 | 13.80 |
| 1 | A81 | 008 | 007 | 73 | 15.40 | 14.68 | 15.04 | 15.16 |
| 1 | A82 | 004 | 007 | 68 | 10.87 | 14.68 | 12.78 | 13.06 |
| 1 | A83 | 012 | 007 | 66 | 12.71 | 14.68 | 13.70 | 13.79 |
| 1 | A84 | 010 | 007 | 66 | 14.24 | 14.68 | 14.46 | 14.48 |
| 1 | A85 | 003 | 009 | 77 | 12.04 | 12.70 | 12.37 | 12.31 |
| 1 | A86 | 001 | 009 | 69 | 13.27 | 12.70 | 12.99 | 13.00 |
| 1 | A87 | 002 | 009 | 72 | 14.68 | 12.70 | 13.69 | 13.77 |
| 1 | A88 | 005 | 009 | 70 | 12.30 | 12.70 | 12.50 | 12.54 |
| 1 | A89 | 011 | 009 | 72 | 14.36 | 12.70 | 13.53 | 13.65 |
| 1 | A90 | 006 | 009 | 74 | 14.09 | 12.70 | 13.40 | 13.46 |
| 1 | A91 | 007 | 009 | 71 | 14.68 | 12.70 | 13.69 | 13.79 |
| 1 | A92 | 009 | 009 | 71 | 12.70 | 12.70 | 12.70 | 12.73 |
| 1 | A93 | 008 | 009 | 72 | 15.40 | 12.70 | 14.05 | 14.41 |
| 1 | A94 | 004 | 009 | 74 | 10.87 | 12.70 | 11.79 | 11.74 |
| 1 | A95 | 012 | 009 | 71 | 12.71 | 12.70 | 12.71 | 12.65 |
| 1 | A96 | 010 | 009 | 73 | 14.24 | 12.70 | 13.47 | 13.42 |
| 1 | A97 | 003 | 008 | 78 | 12.04 | 15.40 | 13.72 | 14.02 |
| 1 | A98 | 001 | 008 | 67 | 13.27 | 15.40 | 14.34 | 14.64 |
| 1 | A99 | 002 | 008 | 66 | 14.68 | 15.40 | 15.04 | 15.19 |
| 1 | A100 | 005 | 008 | 66 | 12.30 | 15.40 | 13.85 | 14.23 |
| 1 | A101 | 011 | 008 | 64 | 14.36 | 15.40 | 14.88 | 15.03 |
| 1 | A102 | 006 | 008 | 67 | 14.09 | 15.40 | 14.75 | 14.96 |
| 1 | A103 | 007 | 008 | 69 | 14.68 | 15.40 | 15.04 | 15.16 |
| 1 | A104 | 009 | 008 | 69 | 12.70 | 15.40 | 14.05 | 14.42 |
| 1 | A105 | 008 | 008 | 69 | 15.40 | 15.40 | 15.40 | 15.46 |
| 1 | A106 | 004 | 008 | 65 | 10.87 | 15.40 | 13.14 | 13.68 |
| 1 | A107 | 012 | 008 | 67 | 12.71 | 15.40 | 14.06 | 14.48 |
| 1 | A108 | 010 | 008 | 67 | 14.24 | 15.40 | 14.82 | 15.01 |
| 1 | A109 | 003 | 004 | 73 | 12.04 | 10.87 | 11.46 | 11.53 |
| 1 | A110 | 001 | 004 | 66 | 13.27 | 10.87 | 12.07 | 12.03 |
| 1 | A111 | 002 | 004 | 67 | 14.68 | 10.87 | 12.78 | 13.03 |
| 1 | A112 | 005 | 004 | 76 | 12.30 | 10.87 | 11.59 | 11.75 |
| 1 | A113 | 011 | 004 | 67 | 14.36 | 10.87 | 12.62 | 12.96 |
| 1 | A114 | 006 | 004 | 65 | 14.09 | 10.87 | 12.48 | 12.68 |
| 1 | A115 | 007 | 004 | 66 | 14.68 | 10.87 | 12.78 | 12.90 |
| 1 | A116 | 009 | 004 | 66 | 12.70 | 10.87 | 11.79 | 11.74 |
| 1 | A117 | 008 | 004 | 67 | 15.40 | 10.87 | 13.14 | 13.67 |
| 1 | A118 | 004 | 004 | 67 | 10.87 | 10.87 | 10.87 | 11.05 |
| 1 | A119 | 012 | 004 | 66 | 12.71 | 10.87 | 11.79 | 11.94 |
| 1 | A120 | 010 | 004 | 74 | 14.24 | 10.87 | 12.56 | 12.80 |
| 1 | A121 | 003 | 012 | 82 | 12.04 | 12.71 | 12.38 | 12.36 |
| 1 | A122 | 001 | 012 | 69 | 13.27 | 12.71 | 12.99 | 12.89 |
| 1 | A123 | 002 | 012 | 67 | 14.68 | 12.71 | 13.70 | 13.78 |
| 1 | A124 | 005 | 012 | 71 | 12.30 | 12.71 | 12.51 | 12.44 |
| 1 | A125 | 011 | 012 | 66 | 14.36 | 12.71 | 13.54 | 13.65 |
| 1 | A126 | 006 | 012 | 67 | 14.09 | 12.71 | 13.40 | 13.42 |
| 1 | A127 | 007 | 012 | 73 | 14.68 | 12.71 | 13.70 | 13.79 |
| 1 | A128 | 009 | 012 | 72 | 12.70 | 12.71 | 12.71 | 12.64 |
| 1 | A129 | 008 | 012 | 69 | 15.40 | 12.71 | 14.06 | 14.48 |
| 1 | A130 | 004 | 012 | 67 | 10.87 | 12.71 | 11.79 | 11.94 |
| 1 | A131 | 012 | 012 | 68 | 12.71 | 12.71 | 12.71 | 12.75 |
| 1 | A132 | 010 | 012 | 65 | 14.24 | 12.71 | 13.48 | 13.56 |
| 1 | A133 | 003 | 010 | 74 | 12.04 | 14.24 | 13.14 | 13.12 |
| 1 | A134 | 001 | 010 | 68 | 13.27 | 14.24 | 13.76 | 13.66 |
| 1 | A135 | 002 | 010 | 67 | 14.68 | 14.24 | 14.46 | 14.49 |
| 1 | A136 | 005 | 010 | 70 | 12.30 | 14.24 | 13.27 | 13.20 |
| 1 | A137 | 011 | 010 | 67 | 14.36 | 14.24 | 14.30 | 14.30 |
| 1 | A138 | 006 | 010 | 73 | 14.09 | 14.24 | 14.17 | 14.14 |
| 1 | A139 | 007 | 010 | 67 | 14.68 | 14.24 | 14.46 | 14.47 |
| 1 | A140 | 009 | 010 | 71 | 12.70 | 14.24 | 13.47 | 13.41 |
| 1 | A141 | 008 | 010 | 67 | 15.40 | 14.24 | 14.82 | 15.01 |
| 1 | A142 | 004 | 010 | 65 | 10.87 | 14.24 | 12.56 | 12.62 |
| 1 | A143 | 012 | 010 | 69 | 12.71 | 14.24 | 13.48 | 13.56 |
| 1 | A144 | 010 | 010 | 72 | 14.24 | 14.24 | 14.24 | 14.29 |
| 2 | B01 | 215 | 212 | 69 | 12.17 | 15.93 | 14.05 | 14.21 |
| 2 | B02 | 215 | 208 | 69 | 12.17 | 20.66 | 16.42 | 16.80 |
| 2 | B03 | 215 | 211 | 63 | 12.17 | 13.27 | 12.72 | 12.66 |
| 2 | B04 | 215 | 213 | 66 | 12.17 | 15.47 | 13.82 | 13.54 |
| 2 | B05 | 215 | 216 | 65 | 12.17 | 15.06 | 13.62 | 13.49 |
| 2 | B06 | 215 | 206 | 70 | 12.17 | 14.21 | 13.19 | 13.12 |
| 2 | B07 | 215 | 214 | 67 | 12.17 | 12.49 | 12.33 | 12.23 |
| 2 | B08 | 215 | 209 | 70 | 12.17 | 12.12 | 12.15 | 12.05 |
| 2 | B09 | 215 | 204 | 66 | 12.17 | 12.89 | 12.53 | 12.43 |
| 2 | B10 | 215 | 202 | 64 | 12.17 | 11.24 | 11.71 | 11.59 |
| 2 | B11 | 215 | 210 | 69 | 12.17 | 12.27 | 12.22 | 12.12 |
| 2 | B12 | 215 | 217 | 67 | 12.17 | 12.19 | 12.18 | 12.08 |
| 2 | B13 | 215 | 218 | 70 | 12.17 | 11.74 | 11.96 | 11.90 |
| 2 | B14 | 215 | 201 | 65 | 12.17 | 11.40 | 11.79 | 11.68 |
| 2 | B15 | 203 | 212 | 72 | 16.74 | 15.93 | 16.34 | 16.24 |
| 2 | B16 | 203 | 208 | 75 | 16.74 | 20.66 | 18.70 | 18.91 |
| 2 | B17 | 203 | 211 | 66 | 16.74 | 13.27 | 15.01 | 14.89 |
| 2 | B18 | 203 | 213 | 70 | 16.74 | 15.47 | 16.11 | 16.02 |
| 2 | B19 | 203 | 216 | 68 | 16.74 | 15.06 | 15.90 | 15.87 |
| 2 | B20 | 203 | 206 | 71 | 16.74 | 14.21 | 15.48 | 15.41 |
| 2 | B21 | 203 | 214 | 68 | 16.74 | 12.49 | 14.62 | 14.51 |
| 2 | B22 | 203 | 209 | 68 | 16.74 | 12.12 | 14.43 | 14.34 |
| 2 | B23 | 203 | 204 | 67 | 16.74 | 12.89 | 14.82 | 14.77 |
| 2 | B24 | 203 | 202 | 69 | 16.74 | 11.24 | 13.99 | 13.76 |
| 2 | B25 | 203 | 210 | 72 | 16.74 | 12.27 | 14.51 | 14.22 |
| 2 | B26 | 203 | 217 | 72 | 16.74 | 12.19 | 14.47 | 14.32 |
| 2 | B27 | 203 | 218 | 71 | 16.74 | 11.74 | 14.24 | 14.13 |
| 2 | B28 | 203 | 201 | 66 | 16.74 | 11.40 | 14.07 | 13.89 |
| 2 | B29 | 207 | 212 | 66 | 15.19 | 15.93 | 15.56 | 15.57 |
| 2 | B30 | 207 | 208 | 68 | 15.19 | 20.66 | 17.93 | 18.20 |
| 2 | B31 | 207 | 211 | 66 | 15.19 | 13.27 | 14.23 | 14.21 |
| 2 | B32 | 207 | 213 | 71 | 15.19 | 15.47 | 15.33 | 15.20 |
| 2 | B33 | 207 | 216 | 68 | 15.19 | 15.06 | 15.13 | 15.12 |
| 2 | B34 | 207 | 206 | 72 | 15.19 | 14.21 | 14.70 | 14.69 |
| 2 | B35 | 207 | 214 | 66 | 15.19 | 12.49 | 13.84 | 13.82 |
| 2 | B36 | 207 | 209 | 66 | 15.19 | 12.12 | 13.66 | 13.68 |
| 2 | B37 | 207 | 204 | 65 | 15.19 | 12.89 | 14.04 | 14.05 |
| 2 | B38 | 207 | 202 | 63 | 15.19 | 11.24 | 13.22 | 13.12 |
| 2 | B39 | 207 | 210 | 71 | 15.19 | 12.27 | 13.73 | 13.63 |
| 2 | B40 | 207 | 217 | 73 | 15.19 | 12.19 | 13.69 | 13.64 |
| 2 | B41 | 207 | 218 | 72 | 15.19 | 11.74 | 13.47 | 13.48 |
| 2 | B42 | 207 | 201 | 67 | 15.19 | 11.40 | 13.30 | 13.25 |
| 2 | B43 | 205 | 212 | 72 | 17.68 | 15.93 | 16.81 | 16.81 |
| 2 | B44 | 205 | 208 | 74 | 17.68 | 20.66 | 19.17 | 19.22 |
| 2 | B45 | 205 | 211 | 70 | 17.68 | 13.27 | 15.48 | 15.68 |
| 2 | B46 | 205 | 213 | 73 | 17.68 | 15.47 | 16.58 | 16.79 |
| 2 | B47 | 205 | 216 | 75 | 17.68 | 15.06 | 16.37 | 16.56 |
| 2 | B48 | 205 | 206 | 81 | 17.68 | 14.21 | 15.95 | 16.13 |
| 2 | B49 | 205 | 214 | 74 | 17.68 | 12.49 | 15.09 | 15.46 |
| 2 | B50 | 205 | 209 | 74 | 17.68 | 12.12 | 14.90 | 15.31 |
| 2 | B51 | 205 | 204 | 72 | 17.68 | 12.89 | 15.29 | 15.69 |
| 2 | B52 | 205 | 202 | 69 | 17.68 | 11.24 | 14.46 | 14.78 |
| 2 | B53 | 205 | 210 | 76 | 17.68 | 12.27 | 14.98 | 15.15 |
| 2 | B54 | 205 | 217 | 76 | 17.68 | 12.19 | 14.94 | 15.28 |
| 2 | B55 | 205 | 218 | 78 | 17.68 | 11.74 | 14.71 | 15.10 |
| 2 | B56 | 205 | 201 | 78 | 17.68 | 11.40 | 14.54 | 14.91 |
| 2 | B57 | 212 | 211 | 67 | 15.97 | 13.27 | 14.62 | 14.74 |
| 2 | B58 | 212 | 213 | 67 | 15.97 | 15.47 | 15.72 | 15.71 |
| 2 | B59 | 212 | 216 | 67 | 15.97 | 15.06 | 15.52 | 15.58 |
| 2 | B60 | 212 | 206 | 71 | 15.97 | 14.21 | 15.09 | 15.17 |
| 2 | B61 | 212 | 214 | 68 | 15.97 | 12.49 | 14.23 | 14.44 |
| 2 | B62 | 212 | 209 | 72 | 15.97 | 12.12 | 14.05 | 14.29 |
| 2 | B63 | 212 | 204 | 69 | 15.97 | 12.89 | 14.43 | 14.65 |
| 2 | B64 | 212 | 202 | 71 | 15.97 | 11.24 | 13.61 | 13.81 |
| 2 | B65 | 212 | 210 | 72 | 15.97 | 12.27 | 14.12 | 14.20 |
| 2 | B66 | 212 | 217 | 72 | 15.97 | 12.19 | 14.08 | 14.27 |
| 2 | B67 | 212 | 218 | 72 | 15.97 | 11.74 | 13.86 | 14.11 |
| 2 | B68 | 212 | 201 | 67 | 15.97 | 11.40 | 13.69 | 13.93 |
| 2 | B69 | 208 | 211 | 67 | 20.70 | 13.27 | 16.99 | 17.39 |
| 2 | B70 | 208 | 213 | 71 | 20.70 | 15.47 | 18.09 | 18.74 |
| 2 | B71 | 208 | 216 | 71 | 20.70 | 15.06 | 17.88 | 18.39 |
| 2 | B72 | 208 | 206 | 78 | 20.70 | 14.21 | 17.46 | 17.84 |
| 2 | B73 | 208 | 214 | 71 | 20.70 | 12.49 | 16.60 | 17.31 |
| 2 | B74 | 208 | 209 | 71 | 20.70 | 12.12 | 16.41 | 17.12 |
| 2 | B75 | 208 | 204 | 68 | 20.70 | 12.89 | 16.80 | 17.60 |
| 2 | B76 | 208 | 202 | 66 | 20.70 | 11.24 | 15.97 | 16.48 |
| 2 | B77 | 208 | 210 | 75 | 20.70 | 12.27 | 16.49 | 16.79 |
| 2 | B78 | 208 | 217 | 73 | 20.70 | 12.19 | 16.45 | 17.09 |
| 2 | B79 | 208 | 218 | 73 | 20.70 | 11.74 | 16.22 | 16.79 |
| 2 | B80 | 208 | 201 | 73 | 20.70 | 11.40 | 16.05 | 16.61 |
| 2 | B81 | 211 | 214 | 67 | 13.26 | 12.49 | 12.88 | 12.85 |
| 2 | B82 | 211 | 209 | 67 | 13.26 | 12.12 | 12.69 | 12.68 |
| 2 | B83 | 211 | 204 | 63 | 13.26 | 12.89 | 13.08 | 13.08 |
| 2 | B84 | 211 | 202 | 62 | 13.26 | 11.24 | 12.25 | 12.21 |
| 2 | B85 | 211 | 210 | 67 | 13.26 | 12.27 | 12.77 | 12.73 |
| 2 | B86 | 21: | 217 | 67 | 13.26 | 12.19 | 12.73 | 12.71 |
| 2 | B87 | 211 | 218 | 70 | 13.26 | 11.74 | 12.50 | 12.53 |
| 2 | B88 | 211 | 201 | 68 | 13.26 | 11.40 | 12.33 | 12.31 |
| 2 | B89 | 213 | 214 | 70 | 15.49 | 12.49 | 13.99 | 13.88 |
| 2 | B90 | 213 | 209 | 69 | 15.49 | 12.12 | 13.81 | 13.68 |
| 2 | B91 | 213 | 204 | 66 | 15.49 | 12.89 | 14.19 | 14.12 |
| 2 | B92 | 213 | 202 | 64 | 15.49 | 11.24 | 13.37 | 13.16 |
| 2 | B93 | 213 | 210 | 69 | 15.49 | 12.27 | 13.88 | 13.66 |
| 2 | B94 | 213 | 217 | 69 | 15.49 | 12.19 | 13.84 | 13.68 |
| 2 | B95 | 213 | 218 | 70 | 15.49 | 11.74 | 13.62 | 13.41 |
| 2 | B96 | 213 | 201 | 70 | 15.49 | 11.40 | 13.45 | 13.27 |
| 2 | B97 | 216 | 214 | 71 | 15.08 | 12.49 | 13.79 | 13.74 |
| 2 | B98 | 216 | 209 | 72 | 15.08 | 12.12 | 13.60 | 13.55 |
| 2 | B99 | 216 | 204 | 68 | 15.08 | 12.89 | 13.99 | 13.97 |
| 2 | B100 | 216 | 202 | 68 | 15.08 | 11.24 | 13.16 | 13.03 |
| 2 | B101 | 216 | 210 | 71 | 15.08 | 12.27 | 13.68 | 13.52 |
| 2 | B102 | 216 | 217 | 73 | 15.08 | 12.19 | 13.64 | 13.54 |
| 2 | B103 | 216 | 218 | 75 | 15.08 | 11.74 | 13.41 | 13.34 |
| 2 | B104 | 216 | 201 | 73 | 15.08 | 11.40 | 13.24 | 13.14 |
| 2 | B105 | 206 | 214 | 73 | 14.24 | 12.49 | 13.37 | 13.33 |
| 2 | B106 | 206 | 209 | 74 | 14.24 | 12.12 | 13.18 | 13.16 |
| 2 | B107 | 206 | 204 | 71 | 14.24 | 12.89 | 13.57 | 13.54 |
| 2 | B108 | 206 | 202 | 69 | 14.24 | 11.24 | 12.74 | 12.65 |
| 2 | B109 | 206 | 210 | 76 | 14.24 | 12.27 | 13.26 | 13.15 |
| 2 | B110 | 206 | 217 | 75 | 14.24 | 12.19 | 13.22 | 13.16 |
| 2 | B111 | 206 | 218 | 79 | 14.24 | 11.74 | 12.99 | 12.98 |
| 2 | B112 | 206 | 201 | 74 | 14.24 | 11.40 | 12.82 | 12.74 |
| 2 | B113 | 214 | 202 | 66 | 12.51 | 11.24 | 11.88 | 11.84 |
| 2 | B114 | 214 | 210 | 70 | 12.51 | 12.27 | 12.39 | 12.38 |
| 2 | B115 | 214 | 217 | 70 | 12.51 | 12.19 | 12.35 | 12.33 |
| 2 | B116 | 209 | 202 | 67 | 12.13 | 11.24 | 11.69 | 11.68 |
| 2 | B117 | 209 | 210 | 70 | 12.13 | 12.27 | 12.20 | 12.21 |
| 2 | B118 | 209 | 217 | 71 | 12.13 | 12.19 | 12.16 | 12.17 |
| 2 | B119 | 204 | 202 | 68 | 12.90 | 11.24 | 12.07 | 12.01 |
| 2 | B120 | 204 | 210 | 72 | 12.90 | 12.27 | 12.59 | 12.57 |
| 2 | B121 | 204 | 217 | 73 | 12.90 | 12.19 | 12.55 | 12.54 |

The results demonstrated greater than 60% protein recovery with all FAE reactions. Further, output retention times were closer to average parental antibody retention times than individual parental antibody retention times indicating successful heterodimeric chain pairing and biparatopic antibody formation.

### Example 21: FAE with Conjugated Antibody Fragments

To examine compatibility of FAE with antibodies having conjugated antibody fragments, FAE was carried out using IgG1 antibodies with tethered scFv fragments. Parental antibodies were produced by expression of constructs encoding antibody heavy and light chains in CHO cells. Encoded light chains included anti-CD3 scFv fragments tethered to light chain C-termini via a flexible linker (GGGGSGGGGS (SEQ ID NO: 718)). Anti-CD3 scFv variable domains were also joined by a flexible linker (GGGGSGGGGSGGGGS (SEQ ID NO: 719)). CH3 domains of corresponding heavy chains included either Y407V or T366V mutations to facilitate preferred pairing.

For FAE reactions, 10× 2-mercaptoethylamine-HCl (2-MEA) stock solution (750 mM) was prepared by dissolving 1.70 g of 2-MEA in 20 mL PBS. The pH was adjusted to 7.4 using about 600-700 μL 2N NaOH. 500 μg of each parental antibody (250 μL of 2 mg/mL in PBS) was placed in a well of a deep well plate. 400 μL of PBS followed by 100 μL of 10× 2-MEA stock solution was then added to each well to give a final 2-MEA concentration of 75 mM. Samples were incubated for 5 hours at 30° C.

Desalting with SEPHADEX® G25 plate (1 mL split over three wells) was performed for each sample to exchange buffer into PBS to remove 2-MEA (to achieve about <50 μM). Samples were then incubated for 48 hours at 4° C.

Resulting antibody outputs were examined by SEC, IEX, and LC-MS for formation of desired heterodimeric antibody species. Heterodimeric species with correct chain pairing were evident from the analysis indicating suitability of FAE for bispecific antibody formation from parental antibodies with conjugated antibody fragments.

### Exemplary Embodiments

Described herein below are some exemplary embodiments according to the present disclosure.

Embodiment 1. A first immunoglobulin heavy chain constant region 3 (“CH3”) domain variant polypeptide comprising an amino acid substitution(s) at one or more of the following amino acid positions: 364, 366, 368, 370, 399, 400, 405, 407, and 409, according to EU numbering,
optionally such that the CH3 domain variant polypeptide preferentially forms a heterodimer with a second CH3 domain variant polypeptide, wherein the second CH3 domain variant polypeptide:
   (a) differs from the first CH3 domain variant polypeptide by at least one amino acid; and
   (b) comprises an amino acid substitution(s) at one or more of the following positions: 364, 366, 368, 370, 399, 400, 405, 407, and 409, according to EU numbering,
and optionally wherein:
   (i) the first CH3 domain variant polypeptide further comprises the amino acid substitution S354C and the second CH3 domain variant polypeptide further comprises the amino acid substitution Y349C; or
   (ii) the first CH3 domain variant polypeptide further comprises the amino acid substitution Y349C and the second CH3 domain variant polypeptide further comprises the amino acid substitution S354C,
further optionally wherein:
   (i) when the amino acid substitution(s) in the first CH3 domain variant polypeptide consists of T366Y, the amino acid substitution(s) in the second CH3 domain variant polypeptide does not consist of Y407T;
   (ii) when the amino acid substitution(s) in the first CH3 domain variant polypeptide consists of Y407T, the amino acid substitution(s) in the second CH3 domain variant polypeptide does not consist of T366Y;
   (iii) when the amino acid substitution(s) in the first CH3 domain variant polypeptide consists of T366W, the amino acid substitution(s) in the second CH3 domain variant polypeptide does not consist of T366S, L368A, and Y407V;
   (iv) when the amino acid substitution(s) in the first CH3 domain variant polypeptide consists of T366S, L368A, and Y407V, the amino acid substitution(s) in the second CH3 domain variant polypeptide does not consist of T366W;
   (v) when the amino acid substitution(s) in the first CH3 domain variant polypeptide consists of S354C and T366W, the amino acid substitution(s) in the second CH3 domain variant polypeptide does not consist of Y349C, T366S, L368A, and Y407V;
   (vi) when the amino acid substitution(s) in the first CH3 domain variant polypeptide consists of Y349C, T366S, L368A, and Y407V, the amino acid substitution(s) in the second CH3 domain variant polypeptide does not consist of S354C and T366W;
   (vii) when the amino acid substitution(s) in the first CH3 domain variant polypeptide consists of S364H and F405A, the amino acid substitution(s) in the second CH3 domain variant polypeptide does not consist of Y349T and T394F;
   (viii) when the amino acid substitution(s) in the first CH3 domain variant polypeptide consists of Y349T and T394F, the amino acid substitution(s) in the second CH3 domain variant polypeptide does not consist of S364H and F405A;
   (ix) when the amino acid substitution(s) in the first CH3 domain variant polypeptide consists of T350V, L351Y, F405A, and Y407V, the amino acid substitution(s) in the second CH3 domain variant polypeptide does not consist of T350V, T366L, K392L, and T394W;
   (x) when the amino acid substitution(s) in the first CH3 domain variant polypeptide consists of T350V, T366L, K392L, and T394W, the amino acid substitution(s) in the second CH3 domain variant polypeptide does not consist of T350V, L351Y, F405A, and Y407V;
   (xi) when the amino acid substitution(s) in the first CH3 domain variant polypeptide consists of K392D and K409D, the amino acid substitution(s) in the second CH3 domain variant polypeptide does not consist of E356K and D399K;
   (xii) when the amino acid substitution(s) in the first CH3 domain variant polypeptide consists of E356K and D399K, the amino acid substitution(s) in the second CH3 domain variant polypeptide does not consist of K392D and K409D;
   (xiii) when the amino acid substitution(s) in the first CH3 domain variant polypeptide consists of D221E, P228E, and L368E, the amino acid substitution(s) in the second CH3 domain variant polypeptide does not consist of D221R, P228R, and K409R, wherein the first CH3 domain and the second CH3 domain are derived from a human IgG1 CH3 domain;
   (xiv) when the amino acid substitution(s) in the first CH3 domain variant polypeptide consists of D221R, P228R, and K409R, the amino acid substitution(s) in the second CH3 domain variant polypeptide does not consist of D221E, P228E, and L368E, wherein the first CH3 domain and the second CH3 domain are derived from a human IgG1 CH3 domain;
   ((xv) when the amino acid substitution(s) in the first CH3 domain variant polypeptide consists of C223E, P228E, and L368E, the amino acid substitution(s) in the second CH3 domain variant polypeptide does not consist of C223R, E225R, P228R, and K409R, wherein the first CH3 domain and the second CH3 domain are derived from a human IgG2 CH3 domain;
   (xvi) when the amino acid substitution(s) in the first CH3 domain variant polypeptide consists of C223R, E225R, P228R, and K409R, the amino acid substitution(s) in the second CH3 domain variant polypeptide does not consist of C223E, P228E, and L368E, wherein the first CH3 domain and the second CH3 domain are derived from a human IgG2 CH3 domain;
   (xvii) when the amino acid substitution(s) in the first CH3 domain variant polypeptide consists of K360E and K409W, the amino acid substitution(s) in the second CH3 domain variant polypeptide does not consist of Q347R, D399V, and F405T; (xviii) when the amino acid substitution(s) in the first CH3 domain variant polypeptide consists of Q347R, D399V, and F405T, the amino acid substitution(s) in the second CH3 domain variant polypeptide does not consist of K360E and K409W;
   (xix) when the amino acid substitution(s) in the first CH3 domain variant polypeptide consists of K360E, K409W, and Y349C, the amino acid substitution(s) in the second CH3 domain variant polypeptide does not consist of Q347R, D399V, F405T, and S354C;
   (xx) when the amino acid substitution(s) in the first CH3 domain variant polypeptide consists of Q347R, D399V, F405T, and S354C, the amino acid substitution(s) in the second CH3 domain variant polypeptide does not consist of K360E, K409W, and Y349C;
   (xxi) when the amino acid substitution(s) in the first CH3 domain variant polypeptide consists of 366K or of 366K and 351K, the amino acid substitution(s) in the second CH3 domain variant polypeptide does not consist of 351D, of 349E, of 349D, of 368E, of 368D, of 349E and 355E, of 349E and 355D, of 349D and 355E, or of 349D and 355D;
   (xxii) when the amino acid substitution(s) in the first CH3 domain variant polypeptide consists 351D, of 349E, of 349D, of 368E, of 368D, of 349E and 355E, of 349E and 355D, of 349D and 355E, or of 349D and 355D, the amino acid substitution(s) in the second CH3 domain variant polypeptide does not consist of 366K or of 366K and 351K;
   (xxiii) when the amino acid substitution(s) in the first CH3 domain variant polypeptide consists of F405L, the amino acid substitution(s) in the second CH3 domain variant polypeptide does not consist of K409R;
   (xxiv) when the amino acid substitution(s) in the first CH3 domain variant polypeptide consists of K409R, the amino acid substitution(s) in the second CH3 domain variant polypeptide does not consist of F405L;
   (xxv) when the amino acid substitution(s) in the first CH3 domain variant polypeptide consists of K360D, D399M, and Y407A, the amino acid substitution(s) in the second CH3 domain variant polypeptide does not consist of E345R, Q347R, T366V, and K409V;
   (xxvi) when the amino acid substitution(s) in the first CH3 domain variant polypeptide consists of E345R, Q347R, T366V, and K409V, the amino acid substitution(s) in the second CH3 domain variant polypeptide does not consist of K360D, D399M, and Y407A;
   (xxvii) when the amino acid substitution(s) in the first CH3 domain variant polypeptide consists of Y349S, K370Y, T366M, and K409V, the amino acid substitution(s) in the second CH3 domain variant polypeptide does not consist of E356G, E357D, S364Q, and Y407A; and
   (xxviii) when the amino acid substitution(s) in the first CH3 domain variant polypeptide consists of E356G, E357D, S364Q, and Y407A, the amino acid substitution(s) in the second CH3 domain variant polypeptide does not consist of Y349S, K370Y, T366M, and K409V.

Embodiment 2. The first CH3 domain variant polypeptide of embodiment 1, which:
(I) comprises an amino acid substitution(s) at one or more of the following amino acid positions 364, 366, 400, 407, and 409 and which optionally preferentially forms a heterodimer with a second CH3 domain variant polypeptide comprising one or more of the following amino acid positions: 366, 368, 370, 399, 405, and 407; or
(II) comprises an amino acid substitution(s) at one or more of the following amino acid positions: 366, 368, 370, 399, 405, and 407 and which optionally preferentially forms a heterodimer with a second CH3 domain variant polypeptide comprising one or more of the following amino acid positions: 364, 366, 400, 407, and 409.

Embodiment 3. The first CH3 domain variant polypeptide of embodiment 1 or 2, wherein the first CH3 domain only comprises an amino acid substitution(s) at:
(I) one or more of the following amino acid positions: 364, 366, 400, 407, and 409; or
(II) one or more of the following amino acid positions: 366, 368, 370, 399, 405, and 407.

Embodiment 4. The first CH3 domain variant polypeptide of any one of embodiments 1-2, wherein the amino acid substitution(s) in the first CH3 domain variant polypeptide comprise(s) or consist(s) of an amino acid substitution(s) at:
(i) position 366; (ii) positions 366 and 407; (iii) positions 364, 366, and 409; (iv) positions 366, 368, and 407; (v) position 368; (vi) position 407; (vii) positions 366 and 368; (viii) positions 366 and 409; (ix) positions 368 and 370; (x) positions 368 and 407; (xi) positions 399 and 405; (xii) positions 400 and 409; (xiii) positions 364, 407, and 409; (xiv) positions 366, 368, and 370; (xv) positions 366, 399, and 405; (xvi) positions 366, 400, and 409; (xvii) positions 366, 407, and 409; (xviii) positions 368, 400, and 409; (xix) positions 399, 405, and 407; (xx) positions 400, 407, and 409; (xxi) positions 366, 399, 405, and 407; (xxii) positions 366, 399, 405, and 409; (xxiii) positions 366, 400, 407, and 409; (xxiv) positions 366, 368, 399, 405, and 407; or (xxv) positions 366, 368, 400, 407, and 409.

Embodiment 5. The first CH3 domain variant polypeptide of any one of embodiments 1-4, wherein:
(i) the amino acid substitution(s) in the first CH3 domain variant polypeptide comprise(s) or consist(s) of an amino acid substitution at position 366, and optionally the amino acid substitutions in the second CH3 domain variant polypeptide comprise or consist of amino acid substitutions at positions 366 and 407;
(ii) the amino acid substitutions in the first CH3 domain variant polypeptide comprise or consist of amino acid substitutions at positions 366 and 407, and optionally the amino acid substitution(s) in the second CH3 domain variant polypeptide comprise(s) or consist(s) of an amino acid substitution at position 366;
(iii) the amino acid substitutions in the first CH3 domain variant polypeptide comprise or consist of amino acid substitutions at positions 364, 366, and 409, and optionally the amino acid substitutions in the second CH3 domain variant polypeptide comprise or consist of amino acid substitutions at positions 366, 368, and 407;
(iv) the amino acid substitutions in the first CH3 domain variant polypeptide comprise or consist of amino acid substitutions at positions 366, 368, and 407, and optionally the amino acid substitutions in the second CH3 domain variant polypeptide comprise or consist of amino acid substitutions at positions 364, 366, and 409;
(v) the amino acid substitution(s) in the first CH3 domain variant polypeptide comprise(s) or consist(s) of an amino acid substitution at position 366, and optionally the amino acid substitution(s) in the second CH3 domain variant polypeptide comprise(s) or consist(s) of an amino acid substitution at position 407;
(vi) the amino acid substitution(s) in the first CH3 domain variant polypeptide comprise(s) or consist(s) of an amino acid substitution at position 368, and optionally the amino acid substitutions in the second CH3 domain variant polypeptide comprise or consist of amino acid substitutions at positions 366 and 409;
(vii) the amino acid substitution(s) in the first CH3 domain variant polypeptide comprise(s) or consist(s) of an amino acid substitution at position 407, and optionally the amino acid substitution(s) in the second CH3 domain variant polypeptide comprise(s) or consist(s) of an amino acid substitution at position 366;
(viii) the amino acid substitutions in the first CH3 domain variant polypeptide comprise or consist of amino acid substitutions at positions 366 and 409, and optionally the amino acid substitution(s) in the second CH3 domain variant polypeptide comprise(s) or consist(s) of an amino acid substitution(s) at position 368 or at positions 368 and 370; (ix) the amino acid substitutions in the first CH3 domain variant polypeptide comprise or consist of amino acid substitutions at positions 368 and 370, and optionally the amino acid substitutions in the second CH3 domain variant polypeptide comprise or consist of amino acid substitutions at positions 366 and 409;
(x) the amino acid substitutions in the first CH3 domain variant polypeptide comprise or consist of amino acid substitutions at positions 399 and 405, and optionally the amino acid substitutions in the second CH3 domain variant polypeptide comprise or consist of amino acid substitutions at positions 400 and 409;
(xi) the amino acid substitutions in the first CH3 domain variant polypeptide comprise or consist of amino acid substitutions at positions 400 and 409, and optionally the amino acid substitutions in the second CH3 domain variant polypeptide comprise or consist of amino acid substitutions at positions 399 and 405;
(xii) the amino acid substitutions in the first CH3 domain variant polypeptide comprise or consist of amino acid substitutions at positions 364, 407, and 409, and optionally the amino acid substitutions in the second CH3 domain variant polypeptide comprise or consist of amino acid substitutions at positions 366, 368, and 370;
(xiii) the amino acid substitutions in the first CH3 domain variant polypeptide comprise or consist of amino acid substitutions at positions 366, 368, and 370, and optionally the amino acid substitutions in the second CH3 domain variant polypeptide comprise or consist of amino acid substitutions at positions 364, 407, and 409;
(xiv) the amino acid substitutions in the first CH3 domain variant polypeptide comprise or consist of amino acid substitutions at positions 366 and 368, and optionally the amino acid substitutions in the second CH3 domain variant polypeptide comprise or consist of amino acid substitutions at positions 366, 407, and 409;
(xv) the amino acid substitutions in the first CH3 domain variant polypeptide comprise or consist of amino acid substitutions at positions 366, 407, and 409, and optionally the amino acid substitutions in the second CH3 domain variant polypeptide comprise or consist of amino acid substitutions at positions 366 and 368;
(xvi) the amino acid substitutions in the first CH3 domain variant polypeptide comprise or consist of amino acid substitutions at positions 368 and 407, and optionally the amino acid substitutions in the second CH3 domain variant polypeptide comprise or consist of amino acid substitutions at positions 364, 366, and 409;
(xvii) the amino acid substitutions in the first CH3 domain variant polypeptide comprise or consist of amino acid substitutions at positions 364, 366, and 409, and optionally the amino acid substitutions in the second CH3 domain variant polypeptide comprise or consist of amino acid substitutions at positions 368 and 407;
(xviii) the amino acid substitutions in the first CH3 domain variant polypeptide comprise or consist of amino acid substitutions at positions 366, 399, and 405, and optionally the amino acid substitutions in the second CH3 domain variant polypeptide comprise or consist of amino acid substitutions at positions 400, 407, and 409, at positions 366, 400, 407, and 409, or at positions 366, 368, 400, 407, and 409;
(xix) the amino acid substitutions in the first CH3 domain variant polypeptide comprise or consist of amino acid substitutions at positions 400, 407, and 409, at positions 366, 400, 407, and 409, or at positions 366, 368, 400, 407, and 409, and optionally the amino acid substitutions in the second CH3 domain variant polypeptide comprise or consist of amino acid substitutions at positions 366, 399, and 405;
(xx) the amino acid substitutions in the first CH3 domain variant polypeptide comprise or consist of amino acid substitutions at positions 366, 400, and 409, and optionally the amino acid substitutions in the second CH3 domain variant polypeptide comprise or consist of amino acid substitutions at positions 399, 405, and 407, at positions 366, 399, 405, and 407, or at positions 366, 368, 399, 405, and 407;
(xxi) the amino acid substitutions in the first CH3 domain variant polypeptide comprise or consist of amino acid substitutions at positions 399, 405, and 407, at positions 366, 399, 405, and 407, or at positions 366, 368, 399, 405, and 407, and optionally the amino acid substitutions in the second CH3 domain variant polypeptide comprise or consist of amino acid substitutions at positions 366, 400, and 409;
(xxii) the amino acid substitutions in the first CH3 domain variant polypeptide comprise or consist of amino acid substitutions at positions 368, 400, and 409, and optionally the amino acid substitutions in the second CH3 domain variant polypeptide comprise or consist of amino acid substitutions at positions 366, 399, 405, and 409;
(xxiii) the amino acid substitutions in the first CH3 domain variant polypeptide comprise or consist of amino acid substitution at positions 366, 399, 405, and 409 and optionally the amino acid substitutions in the second CH3 domain variant polypeptide comprise or consist of amino acid substitutions at positions 368, 400, and 409;
(xxiv) the amino acid substitutions in the first CH3 domain variant comprise or consist of amino acid substitutions at positions 366 and 368, and optionally the amino acid substitutions in the second CH3 domain variant comprise or consist of amino acid substitution at positions 366, 407, and 409;
(xxv) the amino acid substitutions in the first CH3 domain variant comprise or consist of amino acid substitutions at positions 366, 407, and 409, and optionally the amino acid substitutions in the second CH3 domain variant comprise or consist of amino acid substitutions at positions 366 and 368;
(xxvi) the amino acid substitutions in the first CH3 domain variant comprise or consist of amino acid substitutions at positions 368, and 407, and optionally the amino acid substitutions in the second CH3 domain variant comprise or consist of amino acid substitutions at positions 364, 366, and 409;
(xxvii) the amino acid substitutions in the first CH3 domain variant comprise or consist of amino acid substitutions at positions 364, 366, and 409, and optionally the amino acid substitutions in the second CH3 domain variant comprise or consist of amino acid substitutions at positions 368 and 407;
(xxviii) the amino acid substitutions in the first CH3 domain variant comprise or consist of amino acid substitutions at positions 366, 399, and 405, and optionally the amino acid substitutions in the second CH3 domain variant comprise or consist of amino acid substitutions at positions 400, 407, and 409, at positions 366, 400, 407, and 409, or at positions 366, 368, 400, 407, and 409;
(xxix) the amino acid substitutions in the first CH3 domain variant comprise or consist of amino acid substitutions at positions 400, 407, and 409, at positions 366, 400, 407, and 409, or at positions 366, 368, 400, 407, and 409, and optionally the amino acid substitutions in the second CH3 domain variant comprise or consist of amino acid substitutions at positions 366, 399, and 405;
(xxx) the amino acid substitutions in the first CH3 domain variant comprise or consist of amino acid substitutions at positions 366, 400, and 409, and optionally the amino acid substitutions in the second CH3 domain variant comprise or consist of amino acid substitutions at positions 399, 405, and 407, at positions 366, 399, 405, and 407, or at positions 366, 368, 399, 405, and 407;
(xxxi) the amino acid substitutions in the first CH3 domain variant comprise or consist of amino acid substitutions at positions 399, 405, and 407, at positions 366, 399, 405, and 407, or at positions 366, 368, 399, 405, and 407, and optionally the amino acid substitutions in the second CH3 domain variant comprise or consist of amino acid substitutions at positions 366, 400, and 409;
(xxxii) the amino acid substitutions in the first CH3 domain variant comprise or consist of amino acid substitutions at positions 368, 400, and 409, and optionally the amino acid substitutions in the second CH3 domain variant comprise or consist of amino acid substitutions at positions 366, 399, 405, and 409; or
(xxxiii) the amino acid substitutions in the first CH3 domain variant consist of amino acid substitutions at positions 366, 399, 405, and 409, and optionally the amino acid substitutions in the second CH3 domain variant comprise or consist of amino acid substitutions at positions 368, 400, and 409.

Embodiment 6. The first CH3 domain variant polypeptide of any one of embodiments 1-2 or 4-5, wherein:
(i) the amino acid substitutions in the first CH3 domain variant polypeptide consist of amino acid substitutions at positions 349 and 366, and optionally the amino acid substitutions in the second CH3 domain variant polypeptide consist of amino acid substitution at positions 354, 366, and 407;
(ii) the amino acid substitutions in the first CH3 domain variant polypeptide consist of amino acid substitutions at positions 349, 366, and 407, and optionally the amino acid substitutions in the second CH3 domain variant polypeptide consist of amino acid substitutions at positions 354 and 366;
(iii) the amino acid substitutions in the first CH3 domain variant polypeptide consist of amino acid substitutions at positions 349, 364, 366, and 409, and optionally the amino acid substitutions in the second CH3 domain variant polypeptide consist of amino acid substitutions at positions 354, 366, 368, and 407;
(iv) the amino acid substitutions in the first CH3 domain variant polypeptide consist of amino acid substitutions at positions 349, 366, 368, and 407, and optionally the amino acid substitutions in the second CH3 domain variant polypeptide consist of amino acid substitutions at positions 354, 364, 366, and 409,
(v) the amino acid substitutions in the first CH3 domain variant polypeptide consist of amino acid substitutions at positions 349 and 366, and optionally the amino acid substitutions in the second CH3 domain variant polypeptide consist of amino acid substitution at positions 354 and 407;
(vi) the amino acid substitutions in the first CH3 domain variant polypeptide consist of amino acid substitutions at positions 349 and 368, and optionally the amino acid substitutions in the second CH3 domain variant polypeptide consist of amino acid substitutions at positions 354, 366, and 409;
(vii) the amino acid substitutions in the first CH3 domain variant polypeptide consist of amino acid substitutions at positions 349 and 407, and optionally the amino acid substitutions in the second CH3 domain variant polypeptide consist of amino acid substitutions at positions 354 and 366;
(viii) the amino acid substitutions in the first CH3 domain variant polypeptide consist of amino acid substitutions at positions 349, 366, and 409, and optionally the amino acid substitutions in the second CH3 domain variant polypeptide consist of amino acid substitutions at positions 354 and 368 or at positions 354, 368, and 370;
(ix) the amino acid substitutions in the first CH3 domain variant polypeptide consist of amino acid substitutions at positions 349, 368, and 370, and optionally the amino acid substitutions in the second CH3 domain variant polypeptide consist of amino acid substitutions at positions 354, 366, and 409;
(x) the amino acid substitutions in the first CH3 domain variant polypeptide consist of amino acid substitutions at positions 349, 399, and 405, and optionally the amino acid substitutions in the second CH3 domain variant polypeptide consist of amino acid substitutions at positions 354, 400, and 409;
(xi) the amino acid substitutions in the first CH3 domain variant polypeptide consist of amino acid substitutions at positions 349, 400, and 409, and optionally the amino acid substitutions in the second CH3 domain variant polypeptide consist of amino acid substitutions at positions 354, 399, and 405;
(xii) the amino acid substitutions in the first CH3 domain variant polypeptide consist of amino acid substitutions at positions 349, 364, 407, and 409, and optionally the amino acid substitutions in the second CH3 domain variant polypeptide consist of amino acid substitutions at positions 354, 366, 368, and 370;
(xiii) the amino acid substitutions in the first CH3 domain variant polypeptide consist of amino acid substitutions at positions 349, 366, 368, and 370, and optionally the amino acid substitutions in the second CH3 domain variant polypeptide consist of amino acid substitutions at positions 354, 364, 407, and 409;
(xiv) the amino acid substitutions in the first CH3 domain variant polypeptide consist of amino acid substitutions at positions 349, 366, and 368, and optionally the amino acid substitutions in the second CH3 domain variant polypeptide consist of amino acid substitutions at positions 354, 366, 407, and 409;
(xv) the amino acid substitutions in the first CH3 domain variant polypeptide consist of amino acid substitutions at positions 349, 366, 407, and 409, and optionally the amino acid substitutions in the second CH3 domain variant polypeptide consist of amino acid substitutions at positions 354, 366, and 368;
(xvi) the amino acid substitutions in the first CH3 domain variant polypeptide consist of amino acid substitutions at positions 349, 368, and 407, and optionally the amino acid substitutions in the second CH3 domain variant polypeptide consist of amino acid substitutions at positions 354, 364, 366, and 409;
(xvii) the amino acid substitutions in the first CH3 domain variant polypeptide consist of amino acid substitutions at positions 349, 364, 366, and 409, and optionally the amino acid substitutions in the second CH3 domain variant polypeptide consist of amino acid substitutions at positions 354, 368, and 407;
(xviii) the amino acid substitutions in the first CH3 domain variant polypeptide consist of amino acid substitutions at positions 349, 366, 399, and 405, and optionally the amino acid substitutions in the second CH3 domain variant polypeptide consist of amino acid substitutions at positions 354, 400, 407, and 409, at positions 354, 366, 400, 407, and 409, or at positions 354, 366, 368, 400, 407, and 409;
(xix) the amino acid substitutions in the first CH3 domain variant polypeptide consist of amino acid substitutions at positions 349, 400, 407, and 409, at positions 349, 366, 400, 407, and 409, or at positions 349, 366, 368, 400, 407, and 409, and optionally the amino acid substitutions in the second CH3 domain variant polypeptide consist of amino acid substitutions at positions 354, 366, 399, and 405;
(xx) the amino acid substitutions in the first CH3 domain variant polypeptide consist of amino acid substitutions at positions 349, 366, 400, and 409, and optionally the amino acid substitutions in the second CH3 domain variant polypeptide consist of amino acid substitutions at positions 354, 399, 405, and 407, at positions 354, 366, 399, 405, and 407, or at positions 354, 366, 368, 399, 405, and 407;
(xxi) the amino acid substitutions in the first CH3 domain variant polypeptide consist of amino acid substitutions at positions 349, 399, 405, and 407, at positions 349, 366, 399, 405, and 407, or at positions 349, 366, 368, 399, 405, and 407, and optionally the amino acid substitutions in the second CH3 domain variant polypeptide consist of amino acid substitution at positions 354, 366, 400, and 409;
(xxii) the amino acid substitutions in the first CH3 domain variant polypeptide consist of amino acid substitutions at positions 349, 368, 400, and 409, and optionally the amino acid substitutions in the second CH3 domain variant polypeptide consist of amino acid substitutions at positions 354, 366, 399, 405, and 409; or
(xxiii) the amino acid substitutions in the first CH3 domain variant polypeptide consist of amino acid substitutions at positions 349, 366, 399, 405, and 409, and optionally the amino acid substitutions in the second CH3 domain variant polypeptide consist of amino acid substitutions at positions 354, 368, 400, and 409.
(xxiv) the amino acid substitutions in the first CH3 domain variant polypeptide consist of amino acid substitutions at positions 349, 349, 366 and 368, and optionally the amino acid substitutions in the second CH3 domain variant polypeptide consist of amino acid substitution at positions 354, 366, 407, and 409;
(xxv) the amino acid substitutions in the first CH3 domain variant polypeptide consist of amino acid substitutions at positions 349, 366, 407, and 409, and optionally the amino acid substitutions in the second CH3 domain variant polypeptide consist of amino acid substitutions at positions 354, 366 and 368;
(xxvi) the amino acid substitutions in the first CH3 domain variant polypeptide consist of amino acid substitutions at positions 349, 368, and 407, and optionally the amino acid substitutions in the second CH3 domain variant polypeptide consist of amino acid substitutions at positions 354, 364, 366, and 409;
(xxvii) the amino acid substitutions in the first CH3 domain variant polypeptide consist of amino acid substitutions at positions 349, 364, 366, and 409, and optionally the amino acid substitutions in the second CH3 domain variant polypeptide consist of amino acid substitutions at positions 354, 368 and 407;
(xxviii) the amino acid substitutions in the first CH3 domain variant polypeptide consist of amino acid substitutions at positions 349, 366, 399, and 405, and optionally the amino acid substitutions in the second CH3 domain variant polypeptide consist of amino acid substitutions at positions 354, 400, 407, and 409, at positions 354, 366, 400, 407, and 409, or at positions 354, 366, 368, 400, 407, and 409;
(xxix) the amino acid substitutions in the first CH3 domain variant polypeptide consist of amino acid substitutions at positions 349, 400, 407, and 409, at positions 349, 366, 400, 407, and 409, or at positions 349, 366, 368, 400, 407, and 409, and optionally the amino acid substitutions in the second CH3 domain variant polypeptide consist of amino acid substitutions at positions 354, 366, 399, and 405;
(xxx) the amino acid substitutions in the first CH3 domain variant polypeptide consist of amino acid substitutions at positions 349, 366, 400, and 409, and optionally the amino acid substitutions in the second CH3 domain variant polypeptide consist of amino acid substitutions at positions 354, 399, 405, and 407, at positions 354, 366, 399, 405, and 407, or at positions 354, 366, 368, 399, 405, and 407;
(xxxi) the amino acid substitutions in the first CH3 domain variant polypeptide consist of amino acid substitutions at positions 349, 399, 405, and 407, at positions 349, 366, 399, 405, and 407, or at positions 349, 366, 368, 399, 405, and 407, and optionally the amino acid substitutions in the second CH3 domain variant polypeptide consist of amino acid substitutions at positions 354, 366, 400, and 409;
(xxxii) the amino acid substitutions in the first CH3 domain variant polypeptide consist of amino acid substitutions at positions 349, 368, 400, and 409, and optionally the amino acid substitutions in the second CH3 domain variant polypeptide consist of amino acid substitutions at positions 354, 366, 399, 405, and 409; or
(xxxiii) the amino acid substitutions in the first CH3 domain variant polypeptide consist of amino acid substitutions at positions 349, 366, 399, 405, and 409, and optionally the amino acid substitutions in the second CH3 domain variant polypeptide consist of amino acid substitutions at positions 354, 368, 400, and 409, wherein the amino acid substitution at position 349 in the first CH3 domain variant polypeptide is Y349C and the amino acid substitution at position 354 in the second CH3 domain variant polypeptide is S354C.

Embodiment 7. The first CH3 domain variant polypeptide of any one of embodiments 1-2 or 4-5, wherein:
(i) the amino acid substitutions in the first CH3 domain variant polypeptide consist of amino acid substitutions at positions 354 and 366, and optionally the amino acid substitutions in the second CH3 domain variant polypeptide consist of amino acid substitutions at positions 349, 366, and 407;
(ii) the amino acid substitutions in the first CH3 domain variant polypeptide consist of amino acid substitutions at positions 354, 366, and 407, and optionally the amino acid substitutions in the second CH3 domain variant polypeptide consist of amino acid substitutions at positions 349 and 366;
(iii) the amino acid substitutions in the first CH3 domain variant polypeptide consist of amino acid substitutions at positions 354, 364, 366, and 409, and optionally the amino acid substitutions in the second CH3 domain variant polypeptide consist of amino acid substitutions at positions 349, 366, 368, and 407;
(iv) the amino acid substitutions in the first CH3 domain variant polypeptide consist of amino acid substitutions at positions 354, 366, 368, and 407, and optionally the amino acid substitutions in the second CH3 domain variant polypeptide consist of amino acid substitutions at positions 349, 364, 366, and 409,
(v) the amino acid substitutions in the first CH3 domain variant polypeptide consist of amino acid substitutions at positions 354 and 366, and optionally the amino acid substitutions in the second CH3 domain variant polypeptide consist of amino acid substitutions at positions 349 and 407;
(vi) the amino acid substitutions in the first CH3 domain variant polypeptide consist of amino acid substitutions at positions 354 and 368, and optionally the amino acid substitutions in the second CH3 domain variant polypeptide consist of amino acid substitutions at positions 349, 366, and 409;
(vii) the amino acid substitutions in the first CH3 domain variant polypeptide consist of amino acid substitutions at positions 354 and 407, and optionally the amino acid substitutions in the second CH3 domain variant polypeptide consist of amino acid substitutions at positions 349 and 366;
(viii) the amino acid substitutions in the first CH3 domain variant polypeptide consist of amino acid substitutions at positions 354, 366, and 409, and optionally the amino acid substitutions in the second CH3 domain variant polypeptide consist of amino acid substitutions at positions 349 and 368 or of positions 349, 368, and 370;
(ix) the amino acid substitutions in the first CH3 domain variant polypeptide consist of amino acid substitutions at positions 354, 368, and 370, and optionally the amino acid substitutions in the second CH3 domain variant polypeptide consist of amino acid substitutions at positions 349, 366, and 409;
(x) the amino acid substitutions in the first CH3 domain variant polypeptide consist of amino acid substitutions at positions 354, 399, and 405, and optionally the amino acid substitutions in the second CH3 domain variant polypeptide consist of amino acid substitutions at positions 349, 400, and 409;
(xi) the amino acid substitutions in the first CH3 domain variant polypeptide consist of amino acid substitutions at positions 354, 400, and 409, and optionally the amino acid substitutions in the second CH3 domain variant polypeptide consist of amino acid substitutions at positions 349, 399, and 405;
(xii) the amino acid substitutions in the first CH3 domain variant polypeptide consist of amino acid substitutions at positions 354, 364, 407, and 409, and optionally the amino acid substitutions in the second CH3 domain variant polypeptide consist of amino acid substitutions at positions 349, 366, 368, and 370;
(xiii) the amino acid substitutions in the first CH3 domain variant polypeptide consist of amino acid substitutions at positions 354, 366, 368, and 370, and optionally the amino acid substitutions in the second CH3 domain variant polypeptide consist of amino acid substitutions at positions 349, 364, 407, and 409;
(xiv) the amino acid substitutions in the first CH3 domain variant polypeptide consist of amino acid substitutions at positions 354, 366, and 368, and optionally the amino acid substitutions in the second CH3 domain variant polypeptide consist of amino acid substitutions at positions 349, 366, 407, and 409;
(xv) the amino acid substitutions in the first CH3 domain variant polypeptide consist of amino acid substitutions at positions 354, 366, 407, and 409, and optionally the amino acid substitutions in the second CH3 domain variant polypeptide consist of amino acid substitutions at positions 349, 366, and 368;
(xvi) the amino acid substitutions in the first CH3 domain variant polypeptide consist of amino acid substitutions at positions 354, 368, and 407, and optionally the amino acid substitutions in the second CH3 domain variant polypeptide consist of amino acid substitutions at positions 349, 364, 366, and 409;
(xvii) the amino acid substitutions in the first CH3 domain variant polypeptide consist of amino acid substitutions at positions 354, 364, 366, and 409, and optionally the amino acid substitutions in the second CH3 domain variant polypeptide consist of amino acid substitutions at positions 349, 368, and 407;
(xviii) the amino acid substitutions in the first CH3 domain variant polypeptide consist of amino acid substitutions at positions 354, 366, 399, and 405, and optionally the amino acid substitutions in the second CH3 domain variant polypeptide consist of amino acid substitutions at positions 349, 400, 407, and 409, at positions 349, 366, 400, 407, and 409, or at positions 349, 366, 368, 400, 407, and 409;
(xix) the amino acid substitutions in the first CH3 domain variant polypeptide consist of amino acid substitutions at positions 354, 400, 407, and 409, at positions 354, 366, 400, 407, and 409, or at positions 354, 366, 368, 400, 407, and 409, and optionally the amino acid substitutions in the second CH3 domain variant polypeptide consist of amino acid substitutions at positions 349, 366, 399, and 405;
(xx) the amino acid substitutions in the first CH3 domain variant polypeptide consist of amino acid substitutions at positions 354, 366, 400, and 409, and optionally the amino acid substitutions in the second CH3 domain variant polypeptide consist of amino acid substitutions at positions 349, 399, 405, and 407, at positions 349, 366, 399, 405, and 407, or at positions 349, 366, 368, 399, 405, and 407;
(xxi) the amino acid substitutions in the first CH3 domain variant polypeptide consist of amino acid substitutions at positions 354, 399, 405, and 407, at positions 354, 366, 399, 405, and 407, or at positions 354, 366, 368, 399, 405, and 407, and optionally the amino acid substitutions in the second CH3 domain variant polypeptide consist of amino acid substitutions at positions 349, 366, 400, and 409;
(xxii) the amino acid substitutions in the first CH3 domain variant polypeptide consist of amino acid substitutions at positions 354, 368, 400, and 409, and optionally the amino acid substitutions in the second CH3 domain variant polypeptide consist of amino acid substitutions at positions 349, 366, 399, 405, and 409; or
(xxiii) the amino acid substitutions in the first CH3 domain variant polypeptide consist of amino acid substitutions at positions 354, 366, 399, 405, and 409 and optionally the amino acid substitutions in the second CH3 domain variant polypeptide consist of amino acid substitutions at positions 349, 368, 400, and 409. (xxiv) the amino acid substitutions in the first CH3 domain variant polypeptide consist of amino acid substitutions at positions 354, 366 and 368, and optionally the amino acid substitutions in the second CH3 domain variant polypeptide consist of amino acid substitutions at positions 349, 366, 407, and 409;
(xxv) the amino acid substitutions in the first CH3 domain variant polypeptide consist of amino acid substitutions at positions 354, 366, 407, and 409, and optionally the amino acid substitutions in the second CH3 domain variant polypeptide consist of amino acid substitutions at positions 349, 366 and 368;
(xxvi) the amino acid substitutions in the first CH3 domain variant polypeptide consist of amino acid substitutions at positions 354, 368 and 407, and optionally the amino acid substitutions in the second CH3 domain variant polypeptide consist of amino acid substitutions at positions 349, 364, 366, and 409;
(xxvii) the amino acid substitutions in the first CH3 domain variant polypeptide consist of amino acid substitutions at positions 354, 364, 366, and 409, and optionally the amino acid substitutions in the second CH3 domain variant polypeptide consist of amino acid substitutions at positions 349, 368, and 407;
(xxviii) the amino acid substitutions in the first CH3 domain variant polypeptide consist of amino acid substitutions at positions 354, 366, 399, and 405, and optionally the amino acid substitutions in the second CH3 domain variant polypeptide consist of amino acid substitutions at positions 349, 400, 407, and 409, at positions 349, 366, 400, 407, and 409, or at positions 349, 366, 368, 400, 407, and 409;
(xxix) the amino acid substitutions in the first CH3 domain variant polypeptide consist of amino acid substitutions at positions 354, 400, 407, and 409, at positions 354, 366, 400, 407, and 409, or at positions 354, 366, 368, 400, 407, and 409, and optionally the amino acid substitutions in the second CH3 domain variant polypeptide consist of amino acid substitutions at positions 349, 366, 399, and 405;
(xxx) the amino acid substitutions in the first CH3 domain variant polypeptide consist of amino acid substitutions at positions 354, 366, 400, and 409, and optionally the amino acid substitutions in the second CH3 domain variant polypeptide consist of amino acid substitutions at positions 349, 399, 405, and 407, at positions 349, 366, 399, 405, and 407, or at positions 349, 366, 368, 399, 405, and 407;
(xxxi) the amino acid substitutions in the first CH3 domain variant polypeptide consist of amino acid substitutions at positions 354, 399, 405, and 407, at positions 354, 366, 399, 405, and 407, or at positions 354, 366, 368, 399, 405, and 407, and optionally the amino acid substitutions in the second CH3 domain variant polypeptide consist of amino acid substitutions at positions 349, 366, 400, and 409;
(xxxii) the amino acid substitutions in the first CH3 domain variant polypeptide consist of amino acid substitutions at positions 354, 368, 400, and 409, and optionally the amino acid substitutions in the second CH3 domain variant polypeptide consist of amino acid substitutions at positions 349, 366, 399, 405, and 409; or
(xxxiii) the amino acid substitutions in the first CH3 domain variant polypeptide consist of amino acid substitutions at positions 354, 366, 399, 405, and 409, and optionally the amino acid substitutions in the second CH3 domain variant polypeptide consist of amino acid substitutions at positions 349, 368, 400, and 409,
wherein the amino acid substitution at position 354 in the first CH3 domain variant polypeptide is S354C, and optionally the amino acid substitution at position 349 in the second CH3 domain variant polypeptide is Y349C.

Embodiment 8. The first CH3 domain variant polypeptide of any one of embodiments 1-2 or 4-7, comprising one or more of the following amino acid substitutions: S364D; S364L; T366Q; T366R; T366S; T366V; T366W; L368A; L368F; L368S; L368I; K370G; K370Y; D399Q; S400T; F405L; Y407V; Y407G; K409R; K409L; and/or K409G, optionally further comprising Y349C or S354C.

Embodiment 9. The first CH3 domain variant polypeptide of any one of embodiments 1-2 or 4-8, comprising the following amino acid substitution(s):
(i) T366W; (ii) T366S and Y407G; (iii) S364L, T366W, and K409G; (iv) T366S, L368I, and Y407G; (v) T366V; (vi) L368F; (vii) Y407V; (viii) T366V and L368F; (ix) T366Q and K409R; (x) T366R and K409G; (xi) L368F and K370G; (xii) L368I and Y407G; (xiii) S400T and K409L; (xiv) D399Q and F405L; (xv) S364D, Y407V, and K409G; (xvi) T366V, L368S, and K370Y; (xvii) T366V, D399Q, and F405L; (xviii) T366W, D399Q, and F405L; (xix) T366V, S400T, and K409L; (xx) T366W, S400T, and K409L; (xxi) T366Q, Y407V, and K409R; (xxii) L368F, S400T, and K409L; (xxiii) D399Q, Y407V, and F405L; (xxiv) S400T, Y407V, and K409L; (xxv) T366S, D399Q, F405L, and Y407G; (xxvi) T366Q, D399Q, F405L, and K409R; (xxvii) T366S, S400T, Y407G, and K409L; (xxviii) T366S, L368A, D399Q, Y407V, and F405L; or (xxiv) T366S, L368A, S400T, Y407V, and K409L;
optionally further comprising Y349C or S354C.

Embodiment 10. The first CH3 domain variant polypeptide of any one of embodiments 1-2 or 4-9, wherein:
(i) the amino acid substitution(s) in the first CH3 domain variant polypeptide comprise(s) or consist(s) of T366W, and optionally the amino acid substitutions in the second CH3 domain variant polypeptide comprise or consist of T366S and Y407G;
(ii) the amino acid substitutions in the first CH3 domain variant polypeptide comprise or consist of T366S and Y407G, and optionally the amino acid substitution(s) in the second CH3 domain variant polypeptide comprise(s) or consist(s) of T366W;
(iii) the amino acid substitutions in the first CH3 domain variant polypeptide comprise or consist of S364L, T366W, and K409G, and optionally the amino acid substitutions in the second CH3 domain variant polypeptide comprise or consist of T366S, L368I, and Y407G;
(iv) the amino acid substitutions in the first CH3 domain variant polypeptide comprise or consist of T366S, L368I, and Y407G, and optionally the amino acid substitutions in the second CH3 domain variant polypeptide comprise or consist of S364L, T366W, and K409G;
(v) the amino acid substitution(s) in the first CH3 domain variant polypeptide comprise(s) or consist(s) of T366V, and optionally the amino acid substitution(s) in the second CH3 domain variant polypeptide comprise(s) or consist(s) of Y407V;
(vi) the amino acid substitution(s) in the first CH3 domain variant polypeptide comprise(s) or consist(s) of L368F, and optionally the amino acid substitutions in the second CH3 domain variant polypeptide comprise or consist of T366Q and K409R;
(vii) the amino acid substitution(s) in the first CH3 domain variant polypeptide comprise(s) or consist(s) of Y407V, and optionally the amino acid substitution(s) in the second CH3 domain variant polypeptide comprise(s) or consist(s) of T366V;
(viii) the amino acid substitutions in the first CH3 domain variant polypeptide comprise or consist of T366Q and K409R, and optionally the amino acid substitution in the second CH3 domain variant polypeptide comprises or consists of L368F;
(ix) the amino acid substitutions in the first CH3 domain variant polypeptide comprise or consist of T366R and K409G, and optionally the amino acid substitutions in the second CH3 domain variant polypeptide comprise or consist of L368F and K370G;
(x) the amino acid substitutions in the first CH3 domain variant polypeptide comprise or consist of L368F and K370G, and optionally the amino acid substitutions in the second CH3 domain variant polypeptide comprise or consist of T366R and K409G;
(xi) the amino acid substitutions in the first CH3 domain variant polypeptide comprise or consist of S400T and K409L, and optionally the amino acid substitutions in the second CH3 domain variant polypeptide comprise or consist of D399Q and F405L;
(xii) the amino acid substitutions in the first CH3 domain variant polypeptide comprise or consist of D399Q and F405L, and optionally the amino acid substitutions in the second CH3 domain variant polypeptide comprise or consist of S400T and K409L;
(xiii) the amino acid substitutions in the first CH3 domain variant polypeptide comprise or consist of S364D, Y407V, and K409G, and optionally the amino acid substitutions in the second CH3 domain variant polypeptide comprise or consist of T366V, L368S, and K370Y;
(xiv) the amino acid substitutions in the first CH3 domain variant polypeptide comprise or consist of T366V, L368S, and K370Y, and optionally the amino acid substitutions in the second CH3 domain variant polypeptide comprise or consist of S364D, Y407V, and K409G;
(xv) the amino acid substitutions in the first CH3 domain variant polypeptide comprise or consist of T366W, S400T, and K409L, and optionally the amino acid substitutions in the second CH3 domain variant polypeptide comprise or consist of T366S, L368A, Y407V, D399Q, and F405L;
(xvi) the amino acid substitutions in the first CH3 domain variant polypeptide comprise or consist of T366S, L368A, Y407V, D399Q, and F405L, and optionally the amino acid substitutions in the second CH3 domain variant polypeptide comprise or consist of T366W, S400T, and K409L;
(xvii) the amino acid substitutions in the first CH3 domain variant polypeptide comprise or consist of T366W, S400T, and K409L, and optionally the amino acid substitutions in the second CH3 domain variant polypeptide comprise or consist of T366S, Y407G, D399Q, and F405L;
(xviii) the amino acid substitutions in the first CH3 domain variant polypeptide comprise or consist of T366S, Y407G, D399Q, and F405L, and optionally the amino acid substitutions in the second CH3 domain variant polypeptide comprise or consist of T366W, S400T, and K409L;
(xix) the amino acid substitutions in the first CH3 domain variant polypeptide comprise or consist of T366W, D399Q, and F405L, and optionally the amino acid substitutions in the second CH3 domain variant polypeptide comprise or consist of T366S, L368A, Y407V, S400T, and K409L;
(xx) the amino acid substitutions in the first CH3 domain variant polypeptide comprise or consist of T366S, L368A, Y407V, S400T, and K409L, and optionally the amino acid substitutions in the second CH3 domain variant polypeptide comprise or consist of T366W, D399Q, and F405L;
(xxi) the amino acid substitutions in the first CH3 domain variant polypeptide comprise or consist of T366W, D399Q, and F405L, and optionally the amino acid substitutions in the second CH3 domain variant polypeptide comprise or consist of T366S, Y407G, S400T, and K409L;
(xxii) the amino acid substitutions in the first CH3 domain variant polypeptide comprise or consist of T366S, Y407G, S400T, and K409L, and optionally the amino acid substitutions in the second CH3 domain variant polypeptide comprise or consist of T366W, D399Q, and F405L;
(xxiii) the amino acid substitutions in the first CH3 domain variant polypeptide comprise or consist of Y407V, S400T, and K409L, and optionally the amino acid substitutions in the second CH3 domain variant polypeptide comprise or consist of T366V, D399Q, and F405L;
(xxiv) the amino acid substitutions in the first CH3 domain variant polypeptide comprise or consist of T366V, D399Q, and F405L, and optionally the amino acid substitutions in the second CH3 domain variant polypeptide comprise or consist of Y407V, S400T, and K409L;
(xxv) the amino acid substitutions in the first CH3 domain variant polypeptide comprise or consist of Y407V, D399Q, and F405L, and optionally the amino acid substitutions in the second CH3 domain variant polypeptide comprise or consist of T366V, S400T, and K409L;
(xxvi) the amino acid substitutions in the first CH3 domain variant polypeptide comprise or consist of T366V, S400T, and K409L, and optionally the amino acid substitutions in the second CH3 domain variant polypeptide comprise or consist of Y407V, D399Q, and F405L;
(xxvii) the amino acid substitutions in the first CH3 domain variant polypeptide comprise or consist of T366Q, K409R, D399Q, and F405L, and optionally the amino acid substitutions in the second CH3 domain variant polypeptide comprise or consist of L368F, S400T, and K409L;
(xxviii) the amino acid substitutions in the first CH3 domain variant polypeptide comprise or consist of L368F, S400T, and K409L, and optionally the amino acid substitutions in the second CH3 domain variant polypeptide comprise or consist of T366Q, K409R, D399Q, and F405L;
(xxix) the amino acid substitutions in the first CH3 domain variant polypeptide comprise or consist of Y407V, T366Q, and K409R, and optionally the amino acid substitutions in the second CH3 domain variant polypeptide comprise or consist of T366V and L368F;
(xxx) the amino acid substitutions in the first CH3 domain variant polypeptide comprise or consist of T366V and L368F, and optionally the amino acid substitutions in the second CH3 domain variant polypeptide comprise or consist of Y407V, T366Q, and K409R;
(xxxi) the amino acid substitutions in the first CH3 domain variant polypeptide comprise or consist of S364L, T366W, and K409G, and optionally the amino acid substitutions in the second CH3 domain variant polypeptide comprise or consist of L368I and Y407G; or
(xxxii) the amino acid substitutions in the first CH3 domain variant polypeptide comprise or consist of L368I and Y407G, and optionally the amino acid substitutions in the second CH3 domain variant polypeptide comprise or consist of S364L, T366W, and K409G.

Embodiment 11. The first CH3 domain variant polypeptide of any one of embodiments 1-2 or 4-9, wherein:
(i) the amino acid substitutions in the first CH3 domain variant polypeptide consist of Y349C and T366W, and optionally the amino acid substitutions in the second CH3 domain variant polypeptide consist of S354C, T366S, and Y407G;
(ii) the amino acid substitutions in the first CH3 domain variant polypeptide consist of Y349C, T366S, and Y407G, and optionally the amino acid substitutions in the second CH3 domain variant polypeptide consist of S354C, S354C, and T366W;
(iii) the amino acid substitutions in the first CH3 domain variant polypeptide consist of Y349C, S364L, T366W, and K409G, and optionally the amino acid substitutions in the second CH3 domain variant polypeptide consist of S354C, T366S, L368I, and Y407G;
(iv) the amino acid substitutions in the first CH3 domain variant polypeptide consist of Y349C, T366S, L368I, and Y407G, and optionally the amino acid substitutions in the second CH3 domain variant polypeptide consist of S354C, S364L, T366W, and K409G;
(v) the amino acid substitutions in the first CH3 domain variant polypeptide consist of Y349C and T366V, and optionally the amino acid substitutions in the second CH3 domain variant polypeptide consist of S354C and Y407V;
(vi) the amino acid substitutions in the first CH3 domain variant polypeptide consist of Y349C and L368F, and optionally the amino acid substitutions in the second CH3 domain variant polypeptide consist of S354C, T366Q, and K409R;
(vii) the amino acid substitutions in the first CH3 domain variant polypeptide consist of Y349C and Y407V, and optionally the amino acid substitutions in the second CH3 domain variant polypeptide consist of S354C, S354C, and T366V;
(viii) the amino acid substitutions in the first CH3 domain variant polypeptide consist of Y349C, T366Q, and K409R, and optionally the amino acid substitutions in the second CH3 domain variant polypeptide consist of S354C and L368F;
(ix) the amino acid substitutions in the first CH3 domain variant polypeptide consist of Y349C, T366R, and K409G, and optionally the amino acid substitutions in the second CH3 domain variant polypeptide consist of S354C, L368F, and K370G;
(x) the amino acid substitutions in the first CH3 domain variant polypeptide consist of Y349C, L368F, and K370G, and optionally the amino acid substitutions in the second CH3 domain variant polypeptide consist of S354C, T366R, and K409G;
(xi) the amino acid substitutions in the first CH3 domain variant polypeptide consist of Y349C, S400T, and K409L, and optionally the amino acid substitutions in the second CH3 domain variant polypeptide consist of S354C, D399Q, and F405L;
(xii) the amino acid substitutions in the first CH3 domain variant polypeptide consist of Y349C, D399Q, and F405L, and optionally the amino acid substitutions in the second CH3 domain variant polypeptide consist of S354C, S400T, and K409L;
(xiii) the amino acid substitutions in the first CH3 domain variant polypeptide consist of Y349C, S364D, Y407V, and K409G, and optionally the amino acid substitutions in the second CH3 domain variant polypeptide consist of S354C, T366V, L368S, and K370Y;
(xiv) the amino acid substitutions in the first CH3 domain variant polypeptide consist of Y349C, T366V, L368S, and K370Y, and optionally the amino acid substitutions in the second CH3 domain variant polypeptide consist of S354C, S364D, Y407V, and K409G;
(xv) the amino acid substitutions in the first CH3 domain variant polypeptide consist of Y349C, T366W, S400T, and K409L, and optionally the amino acid substitutions in the second CH3 domain variant polypeptide consist of S354C, T366S, L368A, Y407V, D399Q, and F405L;
(xvi) the amino acid substitutions in the first CH3 domain variant polypeptide consist of Y349C, T366S, L368A, Y407V, D399Q, and F405L, and optionally the amino acid substitutions in the second CH3 domain variant polypeptide consist of S354C, T366W, S400T, and K409L;
(xvii) the amino acid substitutions in the first CH3 domain variant polypeptide consist of Y349C, T366W, S400T, and K409L, and optionally the amino acid substitutions in the second CH3 domain variant polypeptide consist of S354C, T366S, Y407G, D399Q, and F405L;
(xviii) the amino acid substitutions in the first CH3 domain variant polypeptide consist of Y349C, T366S, Y407G, D399Q, and F405L, and optionally the amino acid substitutions in the second CH3 domain variant polypeptide consist of S354C, T366W, S400T, and K409L;
(xix) the amino acid substitutions in the first CH3 domain variant polypeptide consist of Y349C, T366W, D399Q, and F405L, and optionally the amino acid substitutions in the second CH3 domain variant polypeptide consist of S354C, T366S, L368A, Y407V, S400T, and K409L;
(xx) the amino acid substitutions in the first CH3 domain variant polypeptide consist of Y349C, T366S, L368A, Y407V, S400T, and K409L, and optionally the amino acid substitutions in the second CH3 domain variant polypeptide consist of S354C, T366W, D399Q, and F405L;
(xxi) the amino acid substitutions in the first CH3 domain variant polypeptide consist of Y349C, T366W, D399Q, and F405L, and optionally the amino acid substitutions in the second CH3 domain variant polypeptide consist of S354C, T366S, Y407G, S400T, and K409L;
(xxii) the amino acid substitutions in the first CH3 domain variant polypeptide consist of Y349C, T366S, Y407G, S400T, and K409L, and optionally the amino acid substitutions in the second CH3 domain variant polypeptide consist of S354C, T366W, D399Q, and F405L;
(xxiii) the amino acid substitutions in the first CH3 domain variant polypeptide consist of Y349C, Y407V, S400T, and K409L, and optionally the amino acid substitutions in the second CH3 domain variant polypeptide consist of S354C, T366V, D399Q, and F405L;
(xxiv) the amino acid substitutions in the first CH3 domain variant polypeptide consist of Y349C, T366V, D399Q, and F405L, and optionally the amino acid substitutions in the second CH3 domain variant polypeptide consist of S354C, Y407V, S400T, and K409L;
(xxv) the amino acid substitutions in the first CH3 domain variant polypeptide consist of Y349C, Y407V, D399Q, and F405L, and optionally the amino acid substitutions in the second CH3 domain variant polypeptide consist of S354C, T366V, S400T, and K409L;
(xxvi) the amino acid substitutions in the first CH3 domain variant polypeptide consist of Y349C, T366V, S400T, and K409L, and optionally the amino acid substitutions in the second CH3 domain variant polypeptide consist of S354C, Y407V, D399Q, and F405L;
(xxvii) the amino acid substitutions in the first CH3 domain variant polypeptide consist of Y349C, T366Q, K409R, D399Q, and F405L, and optionally the amino acid substitutions in the second CH3 domain variant polypeptide consist of S354C, L368F, S400T, and K409L;
(xxviii) the amino acid substitutions in the first CH3 domain variant polypeptide consist of Y349C, L368F, S400T, and K409L, and optionally the amino acid substitutions in the second CH3 domain variant polypeptide consist of S354C, T366Q, K409R, D399Q, and F405L;
(xxix) the amino acid substitutions in the first CH3 domain variant polypeptide consist of Y349C, Y407V, T366Q, and K409R, and optionally the amino acid substitutions in the second CH3 domain variant polypeptide consist of S354C, T366V and L368F;
(xxx) the amino acid substitutions in the first CH3 domain variant polypeptide consist of Y349C, T366V and L368F, and optionally the amino acid substitutions in the second CH3 domain variant polypeptide consist of S354C, Y407V, T366Q, and K409R;
(xxxi) the amino acid substitutions in the first CH3 domain variant polypeptide consist of Y349C, S364L, T366W, and K409G, and optionally the amino acid substitutions in the second CH3 domain variant polypeptide consist of S354C, L368I, and Y407G; or
(xxxii) the amino acid substitutions in the first CH3 domain variant polypeptide consist of Y349C, L368I and Y407G, and optionally the amino acid substitutions in the second CH3 domain variant polypeptide consist of S354C, S364L, T366W, and K409G.

Embodiment 12. The first CH3 domain variant polypeptide of any one of embodiments 1-2 or 4-9, wherein:
(i) the amino acid substitutions in the first CH3 domain variant polypeptide consist of S354C and T366W, and optionally the amino acid substitutions in the second CH3 domain variant polypeptide consist of Y349C, T366S, and Y407G;
(ii) the amino acid substitutions in the first CH3 domain variant polypeptide consist of S354C, T366S, and Y407G, and optionally the amino acid substitutions in the second CH3 domain variant polypeptide consist of Y349C and T366W;
(iii) the amino acid substitutions in the first CH3 domain variant polypeptide consist of S354C, S364L, T366W, and K409G, and optionally the amino acid substitutions in the second CH3 domain variant polypeptide consist of Y349C, T366S, L368I, and Y407G;
(iv) the amino acid substitutions in the first CH3 domain variant polypeptide consist of S354C, T366S, L368I, and Y407G, and optionally the amino acid substitutions in the second CH3 domain variant polypeptide consist of Y349C, S364L, T366W, and K409G;
(v) the amino acid substitutions in the first CH3 domain variant polypeptide consist of S354C and T366V, and optionally the amino acid substitutions in the second CH3 domain variant polypeptide consist of Y349C and Y407V;
(vi) the amino acid substitutions in the first CH3 domain variant polypeptide consist of S354C and L368F, and optionally the amino acid substitutions in the second CH3 domain variant polypeptide consist of Y349C, T366Q, and K409R;
(vii) the amino acid substitutions in the first CH3 domain variant polypeptide consist of S354C and Y407V, and optionally the amino acid substitutions in the second CH3 domain variant polypeptide consist of Y349C and T366V;
(viii) the amino acid substitutions in the first CH3 domain variant polypeptide consist of S354C, T366Q, and K409R, and optionally the amino acid substitutions in the second CH3 domain variant polypeptide consist of Y349C and L368F;
(ix) the amino acid substitutions in the first CH3 domain variant polypeptide consist of S354C, T366R, and K409G, and optionally the amino acid substitutions in the second CH3 domain variant polypeptide consist of Y349C, L368F, and K370G;
(x) the amino acid substitutions in the first CH3 domain variant polypeptide consist of S354C, L368F, and K370G, and optionally the amino acid substitutions in the second CH3 domain variant polypeptide consist of Y349C, T366R, and K409G;
(xi) the amino acid substitutions in the first CH3 domain variant polypeptide consist of S354C, S400T, and K409L, and optionally the amino acid substitutions in the second CH3 domain variant polypeptide consist of Y349C, D399Q, and F405L;
(xii) the amino acid substitutions in the first CH3 domain variant polypeptide consist of S354C, D399Q, and F405L, and optionally the amino acid substitutions in the second CH3 domain variant polypeptide consist of Y349C, S400T, and K409L;
(xiii) the amino acid substitutions in the first CH3 domain variant polypeptide consist of S354C, S364D, Y407V, and K409G, and optionally the amino acid substitutions in the second CH3 domain variant polypeptide consist of Y349C, T366V, L368S, and K370Y;
(xiv) the amino acid substitutions in the first CH3 domain variant polypeptide consist of S354C, T366V, L368S, and K370Y, and optionally the amino acid substitutions in the second CH3 domain variant polypeptide consist of Y349C, S364D, Y407V, and K409G;
(xv) the amino acid substitutions in the first CH3 domain variant polypeptide consist of S354C, T366W, S400T, and K409L, and optionally the amino acid substitutions in the second CH3 domain variant polypeptide consist of Y349C, T366S, L368A, Y407V, D399Q, and F405L;
(xvi) the amino acid substitutions in the first CH3 domain variant polypeptide consist of S354C, T366S, L368A, Y407V, D399Q, and F405L, and optionally the amino acid substitutions in the second CH3 domain variant polypeptide consist of Y349C, T366W, S400T, and K409L;
(xvii) the amino acid substitutions in the first CH3 domain variant polypeptide consist of S354C, T366W, S400T, and K409L, and optionally the amino acid substitutions in the second CH3 domain variant polypeptide consist of Y349C, T366S, Y407G, D399Q, and F405L;
(xviii) the amino acid substitutions in the first CH3 domain variant polypeptide consist of S354C, T366S, Y407G, D399Q, and F405L, and optionally the amino acid substitutions in the second CH3 domain variant polypeptide consist of Y349C, T366W, S400T, and K409L;
(xix) the amino acid substitutions in the first CH3 domain variant polypeptide consist of S354C, T366W, D399Q, and F405L, and optionally the amino acid substitutions in the second CH3 domain variant polypeptide consist of Y349C, T366S, L368A, Y407V, S400T, and K409L;
(xx) the amino acid substitutions in the first CH3 domain variant polypeptide consist of S354C, T366S, L368A, Y407V, S400T, and K409L, and optionally the amino acid substitutions in the second CH3 domain variant polypeptide consist of Y349C, T366W, D399Q, and F405L;
(xxi) the amino acid substitutions in the first CH3 domain variant polypeptide consist of S354C, T366W, D399Q, and F405L, and optionally the amino acid substitutions in the second CH3 domain variant polypeptide consist of Y349C, T366S, Y407G, S400T, and K409L;
(xxii) the amino acid substitutions in the first CH3 domain variant polypeptide consist of S354C, T366S, Y407G, S400T, and K409L, and optionally the amino acid substitutions in the second CH3 domain variant polypeptide consist of Y349C, T366W, D399Q, and F405L;
(xxiii) the amino acid substitutions in the first CH3 domain variant polypeptide consist of S354C, Y407V, S400T, and K409L, and optionally the amino acid substitutions in the second CH3 domain variant polypeptide consist of Y349C, T366V, D399Q, and F405L;
(xxiv) the amino acid substitutions in the first CH3 domain variant polypeptide consist of S354C, T366V, D399Q, and F405L, and optionally the amino acid substitutions in the second CH3 domain variant polypeptide consist of Y349C, Y407V, S400T, and K409L;
(xxv) the amino acid substitutions in the first CH3 domain variant polypeptide consist of S354C, Y407V, D399Q, and F405L, and optionally the amino acid substitutions in the second CH3 domain variant polypeptide consist of Y349C, T366V, S400T, and K409L;
(xxvi) the amino acid substitutions in the first CH3 domain variant polypeptide consist of S354C, T366V, S400T, and K409L, and optionally the amino acid substitutions in the second CH3 domain variant polypeptide consist of Y349C, Y407V, D399Q, and F405L;
(xxvii) the amino acid substitutions in the first CH3 domain variant polypeptide consist of S354C, T366Q, K409R, D399Q, and F405L, and optionally the amino acid substitutions in the second CH3 domain variant polypeptide consist of Y349C, L368F, S400T, and K409L;
(xxviii) the amino acid substitutions in the first CH3 domain variant polypeptide consist of S354C, L368F, S400T, and K409L, and optionally the amino acid substitutions in the second CH3 domain variant polypeptide consist of Y349C, T366Q, K409R, D399Q, and F405L;
(xxix) the amino acid substitutions in the first CH3 domain variant polypeptide consist of S354C, Y407V, T366Q, and K409R, and optionally the amino acid substitutions in the second CH3 domain variant polypeptide consist of Y349C, T366V, and L368F;
(xxx) the amino acid substitutions in the first CH3 domain variant polypeptide consist of S354C, T366V and L368F, and optionally the amino acid substitutions in the second CH3 domain variant polypeptide consist of Y349C, Y407V, T366Q, and K409R;
(xxxi) the amino acid substitutions in the first CH3 domain variant polypeptide consist of S354C, S364L, T366W, and K409G, and optionally the amino acid substitutions in the second CH3 domain variant polypeptide consist of Y349C, L368I, and Y407G; or
(xxxii) the amino acid substitutions in the first CH3 domain variant polypeptide consist of S354C, L368I and Y407G, and optionally the amino acid substitutions in the second CH3 domain variant polypeptide consist of Y349C, S364L, T366W, and K409G.

Embodiment 13. The first CH3 domain variant polypeptide of any one of embodiments 1-2 or 4-9, wherein:
(1) the amino acid substitution in the first CH3 domain variant polypeptide consists of T366W, and optionally the amino acid substitutions in the second CH3 domain variant polypeptide consist of T366S and Y407G;
(2) the amino acid substitutions in the first CH3 domain variant polypeptide consist of T366S and Y407G, and optionally the amino acid substitution in the second CH3 domain variant polypeptide consists of T366W;
(3) the amino acid substitutions in the first CH3 domain variant polypeptide consist of S364L, T366W, and K409G, and optionally the amino acid substitutions in the second CH3 domain variant polypeptide consist of T366S, L368I, and Y407G;
(4) the amino acid substitutions in the first CH3 domain variant polypeptide consist of T366S, L368I, and Y407G, and optionally the amino acid substitutions in the second CH3 domain variant polypeptide consist of S364L, T366W, and K409G.
(5) the amino acid substitutions in the first CH3 domain variant polypeptide consist of S354C and T366W, and optionally the amino acid substitutions in the second CH3 domain variant polypeptide consist of Y349C, T366S, and Y407G;
(6) the amino acid substitutions in the first CH3 domain variant polypeptide consist of S354C, T366S, and Y407G, and optionally the amino acid substitutions in the second CH3 domain variant polypeptide consist of Y349C and T366W;
(7) the amino acid substitutions in the first CH3 domain variant polypeptide consist of S354C, S364L, T366W, and K409G, and optionally the amino acid substitutions in the second CH3 domain variant polypeptide consist of Y349C, T366S, L368I, and Y407G;
(8) the amino acid substitutions in the first CH3 domain variant polypeptide consist of S354C, T366S, L368I, and Y407G, and optionally the amino acid substitutions in the second CH3 domain variant polypeptide consist of Y349C, S364L, T366W, and K409G.
(9) the amino acid substitutions in the first CH3 domain variant polypeptide consist of Y349C and T366W, and optionally the amino acid substitutions in the second CH3 domain variant polypeptide consist of S354C, T366S, and Y407G;
(10) the amino acid substitutions in the first CH3 domain variant polypeptide consist of Y349C, T366S, and Y407G, and optionally the amino acid substitutions in the second CH3 domain variant polypeptide consist of S354C, S354C, and T366W;
(11) the amino acid substitutions in the first CH3 domain variant polypeptide consist of Y349C, S364L, T366W, and K409G, and optionally the amino acid substitutions in the second CH3 domain variant polypeptide consist of S354C, T366S, L368I, and Y407G;
(12) the amino acid substitutions in the first CH3 domain variant polypeptide consist of Y349C, T366S, L368I, and Y407G, and optionally the amino acid substitutions in the second CH3 domain variant polypeptide consist of S354C, S364L, T366W, and K409G.

Embodiment 14. The first CH3 domain variant polypeptide of any one of embodiments 1-2 or 4-9, comprising the amino acid sequence according to:
(I) SEQ ID NO: 11, 21, 31, 41, 51, 61, 71, 81, 91, 101, 111, 121, 131, 141, 151, or 161, optionally wherein the second CH3 domain variant polypeptide comprises the amino acid sequence according to SEQ ID NO: 12, 22, 32, 42, 52, 62, 72, 82, 92, 102, 112, 122, 132, 142, 152, or 162, respectively;
(II) SEQ ID NO: 12, 22, 32, 42, 52, 62, 72, 82, 92, 102, 112, 122, 132, 142, 152, or 162, optionally wherein the second CH3 domain variant polypeptide comprises the amino acid sequence according to SEQ ID NO: 11, 21, 31, 41, 51, 61, 71, 81, 91, 101, 111, 121, 131, 141, 151, or 161, respectively;
(III) SEQ ID NO: 13, 23, 33, 43, 53, 63, 73, 83, 93, 103, 113, 123, 133, 143, 153, or 163, optionally wherein the second CH3 domain variant polypeptide comprises the amino acid sequence according to SEQ ID NO: 14, 24, 34, 44, 54, 64, 74, 84, 94, 104, 114, 124, 134, 144, 154, or 164, respectively;
(IV) SEQ ID NO: 14, 24, 34, 44, 54, 64, 74, 84, 94, 104, 114, 124, 134, 144, 154, or 164, optionally wherein the second CH3 domain variant polypeptide comprises the amino acid sequence according to SEQ ID NO: 13, 23, 33, 43, 53, 63, 73, 83, 93, 103, 113, 123, 133, 143, 153, or 163, respectively;
(V) SEQ ID NO: 15, 25, 35, 45, 55, 65, 75, 85, 95, 105, 115, 125, 135, 145, 155, or 165, optionally wherein the second CH3 domain variant polypeptide comprises the amino acid sequence according to SEQ ID NO: 16, 26, 36, 46, 56, 66, 76, 86, 96, 106, 116, 126, 136, 146, 156, or 166, respectively; or
(VI) SEQ ID NO: 16, 26, 36, 46, 56, 66, 76, 86, 96, 106, 116, 126, 136, 146, 156, or 166, optionally wherein the second CH3 domain variant polypeptide comprises the amino acid sequence according to SEQ ID NO: 15, 25, 35, 45, 55, 65, 75, 85, 95, 105, 115, 125, 135, 145, 155, or 165, respectively.

Embodiment 15. The first CH3 domain variant polypeptide of any one of embodiments 1-2 or 4-9, comprising the amino acid sequence according to:
(I) SEQ ID NO: 11 or 71, optionally wherein the second CH3 domain variant polypeptide comprises the amino acid sequence according to SEQ ID NO: 12 or 72, respectively;
(II) SEQ ID NO: 12 or 72, optionally wherein the second CH3 domain variant polypeptide comprises the amino acid sequence according to SEQ ID NO: 11 or 71, respectively;
(III) SEQ ID NO: 13 or 73, optionally wherein the second CH3 domain variant polypeptide comprises the amino acid sequence according to SEQ ID NO: 14 or 74, respectively;
(IV) SEQ ID NO: 14 or 74, optionally wherein the second CH3 domain variant polypeptide comprises the amino acid sequence according to SEQ ID NO: 13 or 73, respectively;
(V) SEQ ID NO: 15 or 75, optionally wherein the second CH3 domain variant polypeptide comprises the amino acid sequence according to SEQ ID NO: 16 or 76, respectively; or
(VI) SEQ ID NO: 16 or 76, optionally wherein the second CH3 domain variant polypeptide comprises the amino acid sequence according to SEQ ID NO: 15 or 75, respectively.

Embodiment 16. An immunoglobulin polypeptide comprising at least one first CH3 domain variant polypeptide of any one of embodiments 1-15.

Embodiment 17. A molecule comprising at least a first polypeptide and a second polypeptide, wherein:
(A) the first polypeptide comprises the first CH3 domain variant polypeptide of any one of embodiments 1-15; and
(B) the second polypeptide comprises the second CH3 domain variant polypeptide of any one of embodiments 1-15,
further wherein the first CH3 domain variant polypeptide and the second CH3 domain variant polypeptide differ by at least one amino acid,
and wherein the first polypeptide and the second polypeptide are bound to or paired with each other, optionally via a disulfide bond(s).

Embodiment 18. A multi-specific antibody or antigen-binding antibody fragment, which comprises:
(A) a first polypeptide comprising a first antigen-binding domain and the first CH3 domain variant polypeptide of any one of embodiments 1-15;
(B) a second polypeptide comprising a second antigen-binding domain and a second CH3 domain variant polypeptide according to the second CH3 domain of any one of embodiments 1-15;
(C) a third polypeptide comprising a third antigen-binding domain;
(D) a fourth polypeptide comprising a fourth antigen-binding domain,
   and wherein:
(a) the first CH3 domain variant polypeptide and the second CH3 domain variant polypeptide differ at least by one amino acid; and
(b) the first polypeptide and the second polypeptide are bound to or paired with each other,
   further wherein:
(I) the first antigen-binding domain and the third antigen-binding domain form a binding site specific for the first epitope; and
(II) the second antigen-binding domain and the fourth antigen-binding domain form a binding site specific for the second epitope which is different from the first epitope.

Embodiment 19. The multi-specific antibody or antigen-binding antibody fragment of embodiment 18,
wherein:
   (I) (I-i) the first antigen-binding domain is an immunoglobulin heavy chain variable region (VH) domain and the third antigen-binding domain is an immunoglobulin light chain variable (VL) domain or (I-ii) the first antigen-binding domain is a VL domain and the third antigen-binding domain is a VH domain; and/or
   (II) (II-i) the second antigen-binding domain is a VH domain and the fourth antigen-binding domain is a VL domain or (II-ii) the second antigen-binding domain is a VL domain and the fourth antigen-binding domain is a VH domain,
   optionally wherein the multi-specific antibody or antigen-binding antibody fragment is bispecific.

Embodiment 20. A polynucleotide or polypeptides encoding:
(i) the first CH3 domain variant polypeptide of any one of embodiments 1-15,
(ii) the polypeptide of embodiment 16;
(iii) the molecule of embodiment 17; and/or
(iv) the multi-specific antibody or antigen-binding antibody fragment of embodiment 18 or 19.

Embodiment 21. A vector comprising the polynucleotide or polynucleotides according to embodiment 20.

Embodiment 22. A cell, which:
(i) comprises the first CH3 domain variant polypeptide of any one of embodiments 1-15;
(ii) comprises the polypeptide of embodiment 16;
(iii) comprises the molecule of embodiment 17;
(iv) comprises the multi-specific antibody or antigen-binding antibody fragment of embodiment 18 or 19,
(v) comprises the polynucleotide or polynucleotides according to embodiment 20; and/or
(vi) comprises the vector according to embodiment 21.

Embodiment 23. A composition, comprising:
(I) (i) the first CH3 domain variant polypeptide of any one of embodiments 1-15,
(ii) the polypeptide of embodiment 16,
(iii) the molecule of embodiment 17,
(iv) the multi-specific antibody or antigen-binding antibody fragment of embodiment 18 or 19,
(v) the polynucleotide or polynucleotides according to embodiment 20,
(vi) the vector according to embodiment 21, and/or
(vii) the cell of embodiment 22; and
(II) a pharmaceutically acceptable carrier.

Embodiment 24. A method of generating a CH3 domain variant library, comprising incorporating a mutation at or randomizing the nucleic acid at one or more pre-determined nucleotide positions, wherein the one or more pre-determined nucleotide positions are within the codon(s) encoding the amino acid at one or more of pre-determined CH3 domain positions selected from positions 364, 366, 368, 370, 399, 400, 405, 407, and/or 409, according to EU numbering,
optionally wherein the one or more mutations are generated via a degenerate codon, optionally a degenerate RMW codon representing six naturally occurring amino acids (D, T, A, E, K, and N) or a degenerate NNK codon representing all 20 naturally occurring amino acid residues,
further optionally wherein the library is for identifying one or more sets of a first CH3 domain variant polypeptide and a second CH3 domain variant polypeptide, wherein the first CH3 domain variant polypeptide preferentially forms a heterodimer with the second CH3 domain variant polypeptide which differs from the first CH3 domain variant polypeptide by at least one amino acid.

Embodiment 25. A method of identifying one or more sets of a first CH3 domain variant polypeptide and a second CH3 domain variant polypeptide, wherein the first CH3 domain variant polypeptide preferentially forms a heterodimer with the second CH3 domain variant polypeptide which differs from the first CH3 domain variant polypeptide by at least one amino acid substitution, the method comprising:
(a) co-expressing or combining (a-1) a first polypeptide or a first set of polypeptides each comprising a CH3 domain variant polypeptide expressed from a first CH3 domain variant library according to the CH3 domain variant library according to embodiment 24 and (a-2) a second polypeptide or a second set of polypeptides each comprising a CH3 domain variant polypeptide expressed from a second CH3 domain variant library according to the CH3 domain variant library of embodiment 24;
(b) quantifying the amount of the CH3 domain variant polypeptide homodimers and heterodimers; and
(c) selecting one or more sets of a first CH3 domain variant polypeptide and a second CH3 domain variant polypeptide which provide a desired percentage of heterodimers, optionally an equivalent or higher heterodimer percentage of heterodimers, optionally relative to a reference CH3 domain variant polypeptide set,
wherein the first CH3 domain variant library and the second CH3 domain library differ by at least one pre-determined CH3 domain position,
   optionally wherein
(I) the CH3 domain variant polypeptide of the first polypeptide or of the first set of polypeptides comprises the substitution S354C and the CH3 domain variant polypeptide of the first polypeptide or of the first set of polypeptides comprises the substitution Y349C; or
(II) the CH3 domain variant polypeptide of the first polypeptide or of the first set of polypeptides comprises the substitution Y349C and the CH3 domain variant polypeptide of the first polypeptide or of the first set of polypeptides comprises the substitution S354C.

Embodiment 26. The method of embodiment 25, wherein:
(i) the one or more predetermined CH3 domain positions of the first CH3 domain library comprise or consist of position 366, and the one or more predetermined CH3 domain positions of the second CH3 domain library comprise or consist of positions 366 and 407;
(ii) the one or more predetermined CH3 domain positions of the first CH3 domain library comprise or consist of positions 366 and 407, and the one or more predetermined CH3 domain positions of the second CH3 domain library comprise or consist of position 366;
(iii) the one or more predetermined CH3 domain positions of the first CH3 domain library comprise or consist of positions 364, 366, and 409, and the one or more predetermined CH3 domain positions of the second CH3 domain library comprise or consist of positions 366, 368, and 407;
(iv) the one or more predetermined CH3 domain positions of the first CH3 domain library comprise or consist of positions 366, 368, and 407, and the one or more predetermined CH3 domain positions of the second CH3 domain library comprise or consist of positions 364, 366, and 409;
(v) the one or more predetermined CH3 domain positions of the first CH3 domain library comprise or consist of position 366, and the one or more predetermined CH3 domain positions of the second CH3 domain library comprise or consist of position 407;
(vi) the one or more predetermined CH3 domain positions of the first CH3 domain library comprise or consist of position 368, and the one or more predetermined CH3 domain positions of the second CH3 domain library comprise or consist of positions 366 and 409;
(vii) the one or more predetermined CH3 domain positions of the first CH3 domain library comprise or consist of position 407, and the one or more predetermined CH3 domain positions of the second CH3 domain library comprise or consist of position 366;
(viii) the one or more predetermined CH3 domain positions of the first CH3 domain library comprise or consist of positions 366 and 409, and the one or more predetermined CH3 domain positions of the second CH3 domain library comprise or consist of position 368;
(ix) the one or more predetermined CH3 domain positions of the first CH3 domain library comprise or consist of positions 366 and 409, and the one or more predetermined CH3 domain positions of the second CH3 domain library comprise or consist of positions 368 and 370;
(x) the one or more predetermined CH3 domain positions of the first CH3 domain library comprise or consist of positions 368 and 370, and the one or more predetermined CH3 domain positions of the second CH3 domain library comprise or consist of positions 366 and 409;
(xi) the one or more predetermined CH3 domain positions of the first CH3 domain library comprise or consist of positions 400 and 409, and the one or more predetermined CH3 domain positions of the second CH3 domain library comprise or consist of positions 399 and F405;
(xii) the one or more predetermined CH3 domain positions of the first CH3 domain library comprise or consist of positions 399 and 405, and the one or more predetermined CH3 domain positions of the second CH3 domain library comprise or consist of positions 400 and 409;
(xiii) the one or more predetermined CH3 domain positions of the first CH3 domain library comprise or consist of positions 364, 407, and 409, and the one or more predetermined CH3 domain positions of the second CH3 domain library comprise or consist of positions 366, 368, and 370;
(xiv) the one or more predetermined CH3 domain positions of the first CH3 domain library comprise or consist of positions 366, 368, and 370, and the one or more predetermined CH3 domain positions of the second CH3 domain library comprise or consist of positions 364, 407, and 409;
(xv) the one or more predetermined CH3 domain positions of the first CH3 domain library comprise or consist of positions 366 and 368, and the one or more predetermined CH3 domain positions of the second CH3 domain library comprise or consist of positions 366, 407, and 409;
(xiv) the one or more predetermined CH3 domain positions of the first CH3 domain library comprise or consist of positions 366, 407, and 409, and the one or more predetermined CH3 domain positions of the second CH3 domain library comprise or consist of positions 366 and 368;
(xv) the one or more predetermined CH3 domain positions of the first CH3 domain library comprise or consist of positions 368 and 407, and the one or more predetermined CH3 domain positions of the second CH3 domain library comprise or consist of positions 364, 366, and 409;
(xvi) the one or more predetermined CH3 domain positions of the first CH3 domain library comprise or consist of positions 364, 366, and 409, and the one or more predetermined CH3 domain positions of the second CH3 domain library comprise or consist of positions 368 and 407;
(xvii) the one or more predetermined CH3 domain positions of the first CH3 domain library comprise or consist of positions 366, 399, and 405, and the one or more predetermined CH3 domain positions of the second CH3 domain library comprise or consist of positions 400, 407, and 409, at positions 366, 400, 407, and 409, or at positions 366, 368, 400, 407, and 409;
(xviii) the one or more predetermined CH3 domain positions of the first CH3 domain library comprise or consist of positions 400, 407, and 409, at positions 366, 400, 407, and 409, or at positions 366, 368, 400, 407, and 409, and the one or more predetermined CH3 domain positions of the second CH3 domain library comprise or consist of positions 366, 399, and 405;
(xix) the one or more predetermined CH3 domain positions of the first CH3 domain library comprise or consist of positions 366, 400, and 409, and the one or more predetermined CH3 domain positions of the second CH3 domain library comprise or consist of positions 399, 405, and 407, at positions 366, 399, 405, and 407, or at positions 366, 368, 399, 405, and 407;
(xx) the one or more predetermined CH3 domain positions of the first CH3 domain library comprise or consist of positions 399, 405, and 407, at positions 366, 399, 405, and 407, or at positions 366, 368, 399, 405, and 407, and the one or more predetermined CH3 domain positions of the second CH3 domain library comprise or consist of positions 366, 400, and 409;
(xxi) the one or more predetermined CH3 domain positions of the first CH3 domain library comprise or consist of positions 368, 400, and 409, and the one or more predetermined CH3 domain positions of the second CH3 domain library comprise or consist of positions 366, 399, 405, and 409; or
(xxii) the one or more predetermined CH3 domain positions of the first CH3 domain library comprise or consist of positions 366, 399, 405, and 409, and the one or more predetermined CH3 domain positions of the second CH3 domain library comprise or consist of positions 368, 400, and 409.

Embodiment 27. The method of embodiment 25 or 26, wherein:
(a-1) the first polypeptide or each polypeptide of the first set of polypeptides comprises or linked to a first label; and
(a-2) the second polypeptide or each of the second set of polypeptides comprises or linked to a second label.

Embodiment 28. The method of embodiment 27, wherein the quantifying step (b) comprises detecting the first label and/or the second label.

Embodiment 29. The method of any one of embodiments 25-28, wherein the quantifying step (b) comprises at least one of liquid chromatography-mass spectrometry (LC-MS), AlphaLISA®, ion exchange chromatography (IEX), and/or flow cytometry.

### APPENDIX

APPENDIX TABLE A
Heavy chain A sequences of antibodies of Table 9

| | | | | Heavy Chain A | |
|---|---|---|---|---|---|
| | | CH3 | Specificity | | SEQ |
| | Antibody | Set | (Chain A/ | | ID |
| Index | Name | Name | Chain B) | Sequence | NO: |
| 1 | ADI-59166 | WT | CD3/HER2 | QVQLVQSGAEVKKPGASVKVSCKASGFNIKDYYMHWVRQAPGQRL | 211 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCLVKGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 2 | ADI-59167 | QR-F | | QVQLVQSGAEVKKPGASVKVSCKASGFNIKDYYMHWVRQAPGQRL | 221 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLQCLVKGFYPSDIA | |
| | | | | VEWESNGQPENNYKTTPPVLDSDGSFFLYSRLTVDKSRWQQGNVFS | |
| | | | | CSVMHEALHNHYTQKSLSLSPG | |
| 3 | ADI-59168 | RG-FG | | QVQLVQSGAEVKKPGASVKVSCKASGFNIKDYYMHWVRQAPGQRL | 231 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLRCLVKGFYPSDIA | |
| | | | | VEWESNGQPENNYKTTPPVLDSDGSFFLYSGLTVDKSRWQQGNVFS | |
| | | | | CSVMHEALHNHYTQKSLSLSPG | |
| 4 | ADI-59169 | TL-QL | | QVQLVQSGAEVKKPGASVKVSCKASGFNIKDYYMHWVRQAPGQRL | 241 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCLVKGFYPSDIA | |
| | | | | VEWESNGQPENNYKTTPPVLDTDGSFFLYSLLTVDKSRWQQGNVFSC | |
| | | | | SVMHEALHNHYTQKSLSLSPG | |
| 5 | ADI-59170 | DVG- | | QVQLVQSGAEVKKPGASVKVSCKASGFNIKDYYMHWVRQAPGQRL | 251 |
| | | VSY | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVDLTCLVKGFYPSDIA | |
| | | | | VEWESNGQPENNYKTTPPVLDSDGSFFLVSGLTVDKSRWQQGNVFS | |
| | | | | CSVMHEALHNHYTQKSLSLSPG | |
| 6 | ADI-59171 | LWG- | | QVQLVQSGAEVKKPGASVKVSCKASGFNIKDYYMHWVRQAPGQRL | 261 |
| | | SIG | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVLLWCLVKGFYPSDI | |
| | | | | AVEWESNGQPENNYKTTPPVLDSDGSFFLYSGLTVDKSRWQQGNVF | |
| | | | | SCSVMHEALHNHYTQKSLSLSPG | |
| 7 | ADI-59172 | QR-F | | QVQLVQSGAEVKKPGASVKVSCKASGFNIKDYYMHWVRQAPGQRL | 271 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCFVKGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 8 | ADI-59173 | RG-FG | | QVQLVQSGAEVKKPGASVKVSCKASGFNIKDYYMHWVRQAPGQRL | 281 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCFVGGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 9 | ADI-59174 | TL-QL | | QVQLVQSGAEVKKPGASVKVSCKASGFNIKDYYMHWVRQAPGQRL | 291 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCLVKGFYPSDIA | |
| | | | | VEWESNGQPENNYKTTPPVLQSDGSFLLYSKLTVDKSRWQQGNVFS | |
| | | | | CSVMHEALHNHYTQKSLSLSPG | |
| 10 | ADI-59175 | DVG- | | QVQLVQSGAEVKKPGASVKVSCKASGFNIKDYYMHWVRQAPGQRL | 301 |
| | | VSY | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLVCSVYGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 11 | ADI-59176 | LWG- | | QVQLVQSGAEVKKPGASVKVSCKASGFNIKDYYMHWVRQAPGQRL | 311 |
| | | SIG | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLSCIVKGFYPSDIA | |
| | | | | VEWESNGQPENNYKTTPPVLDSDGSFFLGSKLTVDKSRWQQGNVFS | |
| | | | | CSVMHEALHNHYTQKSLSLSPG | |
| 12 | ADI-59177 | WT | | QVQLVQSGAEVKKPGASVKVSCKASGFNIKDYYMHWVRQAPGQRL | 321 |
| | | (354/ | | EWMGWIDLEEGNTIYDAKFQGRVTITRDTSASTAYMELSSLRSEDTA | |
| | | 349) | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVSLTCLVKGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 13 | ADI-59178 | QR-F | | QVQLVQSGAEVKKPGASVKVSCKASGFNIKDYYMHWVRQAPGQRL | 331 |
| | | (354/ | | EWMGWIDLEEGNTIYDAKFQGRVTITRDTSASTAYMELSSLRSEDTA | |
| | | 349) | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVSLQCLVKGFYPSDI | |
| | | | | AVEWESNGQPENNYKTTPPVLDSDGSFFLYSRLTVDKSRWQQGNVF | |
| | | | | SCSVMHEALHNHYTQKSLSLSPG | |
| 14 | ADI-59179 | RG-FG | | QVQLVQSGAEVKKPGASVKVSCKASGFNIKDYYMHWVRQAPGQRL | 341 |
| | | (354/ | | EWMGWIDLEEGNTIYDAKFQGRVTITRDTSASTAYMELSSLRSEDTA | |
| | | 349) | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVSLRCLVKGFYPSDIA | |
| | | | | VEWESNGQPENNYKTTPPVLDSDGSFFLYSGLTVDKSRWQQGNVFS | |
| | | | | CSVMHEALHNHYTQKSLSLSPG | |
| 15 | ADI-59180 | TL-QL | | QVQLVQSGAEVKKPGASVKVSCKASGFNIKDYYMHWVRQAPGQRL | 351 |
| | | (354/ | | EWMGWIDLEEGNTIYDAKFQGRVTITRDTSASTAYMELSSLRSEDTA | |
| | | 349) | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVSLTCLVKGFYPSDIA | |
| | | | | VEWESNGQPENNYKTTPPVLDTDGSFFLYSLLTVDKSRWQQGNVFSC | |
| | | | | SVMHEALHNHYTQKSLSLSPG | |
| 16 | ADI-59181 | DVG- | | QVQLVQSGAEVKKPGASVKVSCKASGFNIKDYYMHWVRQAPGQRL | 361 |
| | | VSY | | EWMGWIDLEEGNTIYDAKFQGRVTITRDTSASTAYMELSSLRSEDTA | |
| | | (354/ | | VYYCARDAYGRYFYDVWGQGTLVTVSSASTKGPSVFPLAPSSKSTSGG | |
| | | 349) | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVDLTCLVKGFYPSDI | |
| | | | | AVEWESNGQPENNYKTTPPVLDSDGSFFLVSGLTVDKSRWQQGNVF | |
| | | | | SCSVMHEALHNHYTQKSLSLSPG | |
| 17 | ADI-59182 | LWG- | | QVQLVQSGAEVKKPGASVKVSCKASGFNIKDYYMHWVRQAPGQRL | 371 |
| | | SIG | | EWMGWIDLEEGNTIYDAKFQGRVTITRDTSASTAYMELSSLRSEDTA | |
| | | (354/ | | VYYCARDAYGRYFYDVWGQGTLVTVSSASTKGPSVFPLAPSSKSTSGG | |
| | | 349) | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPCRDELTKNQVLLWCLVKGFYPSDI | |
| | | | | AVEWESNGQPENNYKTTPPVLDSDGSFFLYSGLTVDKSRWQQGNVF | |
| | | | | SCSVMHEALHNHYTQKSLSLSPG | |
| 18 | ADI-59183 | WT | CD20/CD3 | EVQLVESGGGLVQPGRSLRLSCVASGFTFNDYAMHWVRQAPGKGLE | 381 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCLVK | |
| | | | | GFYPSDIAVEWESNGQPENNYKTTPPVLDSDGSFFLYSKLTVDKSRW | |
| | | | | QQGNVFSCSVMHEALHNHYTQKSLSLSPG | |
| 19 | ADI-59184 | QR-F | | EVQLVESGGGLVQPGRSLRLSCVASGFTFNDYAMHWVRQAPGKGLE | 391 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLQCLVK | |
| | | | | GFYPSDIAVEWESNGQPENNYKTTPPVLDSDGSFFLYSRLTVDKSRW | |
| | | | | QQGNVFSCSVMHEALHNHYTQKSLSLSPG | |
| 20 | ADI-59185 | RG-FG | | EVQLVESGGGLVQPGRSLRLSCVASGFTFNDYAMHWVRQAPGKGLE | 401 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLRCLVK | |
| | | | | GFYPSDIAVEWESNGQPENNYKTTPPVLDSDGSFFLYSGLTVDKSRW | |
| | | | | QQGNVFSCSVMHEALHNHYTQKSLSLSPG | |
| 21 | ADI-59186 | TL-QL | | EVQLVESGGGLVQPGRSLRLSCVASGFTFNDYAMHWVRQAPGKGLE | 411 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCLVK | |
| | | | | GFYPSDIAVEWESNGQPENNYKTTPPVLDTDGSFFLYSLLTVDKSRW | |
| | | | | QQGNVFSCSVMHEALHNHYTQKSLSLSPG | |
| 22 | ADI-59187 | DVG- | | EVQLVESGGGLVQPGRSLRLSCVASGFTFNDYAMHWVRQAPGKGLE | 421 |
| | | VSY | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVDLTCLVK | |
| | | | | GFYPSDIAVEWESNGQPENNYKTTPPVLDSDGSFFLVSGLTVDKSRW | |
| | | | | QQGNVFSCSVMHEALHNHYTQKSLSLSPG | |
| 23 | ADI-59188 | LWG- | | EVQLVESGGGLVQPGRSLRLSCVASGFTFNDYAMHWVRQAPGKGLE | 431 |
| | | SIG | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVLLWCLVK | |
| | | | | GFYPSDIAVEWESNGQPENNYKTTPPVLDSDGSFFLYSGLTVDKSRW | |
| | | | | QQGNVFSCSVMHEALHNHYTQKSLSLSPG | |
| 24 | ADI-59189 | QR-F | | EVQLVESGGGLVQPGRSLRLSCVASGFTFNDYAMHWVRQAPGKGLE | 441 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCFVK | |
| | | | | GFYPSDIAVEWESNGQPENNYKTTPPVLDSDGSFFLYSKLTVDKSRW | |
| | | | | QQGNVFSCSVMHEALHNHYTQKSLSLSPG | |
| 25 | ADI-59190 | RG-FG | | EVQLVESGGGLVQPGRSLRLSCVASGFTFNDYAMHWVRQAPGKGLE | 451 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCFVG | |
| | | | | GFYPSDIAVEWESNGQPENNYKTTPPVLDSDGSFFLYSKLTVDKSRW | |
| | | | | QQGNVFSCSVMHEALHNHYTQKSLSLSPG | |
| 26 | ADI-59191 | TL-QL | | EVQLVESGGGLVQPGRSLRLSCVASGFTFNDYAMHWVRQAPGKGLE | 461 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCLVK | |
| | | | | GFYPSDIAVEWESNGQPENNYKTTPPVLQSDGSFLLYSKLTVDKSRW | |
| | | | | QQGNVFSCSVMHEALHNHYTQKSLSLSPG | |
| 27 | ADI-59192 | DVG- | | EVQLVESGGGLVQPGRSLRLSCVASGFTFNDYAMHWVRQAPGKGLE | 471 |
| | | VSY | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLVCSVY | |
| | | | | GFYPSDIAVEWESNGQPENNYKTTPPVLDSDGSFFLYSKLTVDKSRW | |
| | | | | QQGNVFSCSVMHEALHNHYTQKSLSLSPG | |
| 28 | ADI-59193 | LWG- | | EVQLVESGGGLVQPGRSLRLSCVASGFTFNDYAMHWVRQAPGKGLE | 481 |
| | | SIG | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLSCIVKG | |
| | | | | FYPSDIAVEWESNGQPENNYKTTPPVLDSDGSFFLGSKLTVDKSRWQ | |
| | | | | QGNVFSCSVMHEALHNHYTQKSLSLSPG | |
| 29 | ADI-59194 | WT | HEL/BCMA | EVQLVESGGGLVKPGGSLRLSCAASGFTFSSYSMNWVRQAPGKGLE | 491 |
| | | | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVSLTCLVKGFYPSDIAVEWE | |
| | | | | SNGQPENNYKTTPPVLDSDGSFFLYSKLTVDKSRWQQGNVFSCSVM | |
| | | | | HEALHNHYTQKSLSLSPG | |
| 30 | ADI-59195 | QR-F | | EVQLVESGGGLVKPGGSLRLSCAASGFTFSSYSMNWVRQAPGKGLE | 501 |
| | | | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVSLQCLVKGFYPSDIAVEW | |
| | | | | ESNGQPENNYKTTPPVLDSDGSFFLYSRLTVDKSRWQQGNVFSCSV | |
| | | | | MHEALHNHYTQKSLSLSPG | |
| 31 | ADI-59196 | RG-FG | | EVQLVESGGGLVKPGGSLRLSCAASGFTFSSYSMNWVRQAPGKGLE | 511 |
| | | | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVSLRCLVKGFYPSDIAVEWE | |
| | | | | SNGQPENNYKTTPPVLDSDGSFFLYSGLTVDKSRWQQGNVFSCSVM | |
| | | | | HEALHNHYTQKSLSLSPG | |
| 32 | ADI-59197 | TL-QL | | EVQLVESGGGLVKPGGSLRLSCAASGFTFSSYSMNWVRQAPGKGLE | 521 |
| | | | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVSLTCLVKGFYPSDIAVEWE | |
| | | | | SNGQPENNYKTTPPVLDTDGSFFLYSLLTVDKSRWQQGNVFSCSVM | |
| | | | | HEALHNHYTQKSLSLSPG | |
| 33 | ADI-59198 | DVG- | | EVQLVESGGGLVKPGGSLRLSCAASGFTFSSYSMNWVRQAPGKGLE | 531 |
| | | VSY | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVDLTCLVKGFYPSDIAVEWE | |
| | | | | SNGQPENNYKTTPPVLDSDGSFFLVSGLTVDKSRWQQGNVFSCSVM | |
| | | | | HEALHNHYTQKSLSLSPG | |
| 34 | ADI-59199 | LWG- | | EVQLVESGGGLVKPGGSLRLSCAASGFTFSSYSMNWVRQAPGKGLE | 541 |
| | | SIG | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVLLWCLVKGFYPSDIAVEW | |
| | | | | ESNGQPENNYKTTPPVLDSDGSFFLYSGLTVDKSRWQQGNVFSCSV | |
| | | | | MHEALHNHYTQKSLSLSPG | |
| 35 | ADI-59200 | KiH | CD3/HER2 | QVQLVQSGAEVKKPGASVKVSCKASGFNIKDYYMHWVRQAPGQRL | 551 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPELL | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALP | |
| | | | | APIEKTISKAKGQPREPQVYTLPPSRDELTKNQVSLSCAVKGFYPSDIAV | |
| | | | | EWESNGQPENNYKTTPPVLDSDGSFFLVSKLTVDKSRWQQGNVFSC | |
| | | | | SVMHEALHNHYTQKSLSLSPG | |
| 36 | ADI-59201 | WT | CD3/None | QVQLVQSGAEVKKPGASVKVSCKASGFNIKDYYMHWVRQAPGQRL | 561 |
| | | | | EWMGWIDLEEGNTIYDAKFQGRVTITRDTSASTAYMELSSLRSEDTA | |
| | | | | VYYCARDAYGRYFYDVWGQGTLVTVSSASTKGPSVFPLAPSSKSTSGG | |
| | | | | TAALGCLVKDYFPEPVTVSWNSGALTSGVHTFPAVLQSSGLYSLSSVV | |
| | | | | TVPSSSLGTQTYICNVNHKPSNTKVDKKVEPKSCDKTHTCPPCPAPEA | |
| | | | | AGGPSVFLFPPKPKDTLMISRTPEVTCVVVDVSHEDPEVKFNWYVDG | |
| | | | | VEVHNAKTKPREEQYNSTYRVVSVLTVLHQDWLNGKEYKCKVSNKAL | |
| | | | | AAPIEKTISKAKGQPREPQVYTLPPSRDELTKNQVSLTCLVKGFYPSDIA | |
| | | | | VEWESNGQPENNYKTTPPVLDSDGSFFLYSKLTVDKSRWQQGNVFS | |
| | | | | CSVMHEALHNHYTQKSLSLSPG | |
| 37 | ADI-59202 | WT | HER2/None | QVQLVQSGAEVKKPGASVKVSCKASGYAFPNYGISWVRQAPGQGLE | 571 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 38 | ADI-59203 | WT | CD20/None | EVQLVESGGGLVQPGRSLRLSCVASGFTFNDYAMHWVRQAPGKGLE | 581 |
| | | | | WVSVISWNSDSIGYADSVKGRFTISRDNAKNSLYLQMHSLRAEDTALY | |
| | | | | YCAKDNHYGSGSYYYYQYGMDVWGQGTTVTVSSASTKGPSVFPLAP | |
| | | | | SSKSTSGGTAALGCLVKDYFPEPVTVSWNSGALTSGVHTFPAVLQSSG | |
| | | | | LYSLSSVVTVPSSSLGTQTYICNVNHKPSNTKVDKKVEPKSCDKTHTCP | |
| | | | | PCPAPEAAGGPSVFLFPPKPKDTLMISRTPEVTCVVVDVSHEDPEVKF | |
| | | | | NWYVDGVEVHNAKTKPREEQYNSTYRVVSVLTVLHQDWLNGKEYKC | |
| | | | | KVSNKALAAPIEKTISKAKGQPREPQVYTLPPSRDELTKNQVSLTCLVK | |
| | | | | GFYPSDIAVEWESNGQPENNYKTTPPVLDSDGSFFLYSKLTVDKSRW | |
| | | | | QQGNVFSCSVMHEALHNHYTQKSLSLSPG | |
| 39 | ADI-59204 | WT | CD3/None | EVQLVESGGGLVQPGRSLRLSCAASGFTFDDYTMHWVRQAPGKGLE | 591 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 40 | ADI-59205 | WT | HEL/None | EVQLVESGGGLVKPGGSLRLSCAASGFTFSSYSMNWVRQAPGKGLE | 601 |
| | | | | WVSSISSSSSYIYYADSVKGRFTISRDNAKNSLYLQMNSLRAEDTAVYY | |
| | | | | CARSHSSLFLDYWGQGTLVTVSSASTKGPSVFPLAPSSKSTSGGTAAL | |
| | | | | GCLVKDYFPEPVTVSWNSGALTSGVHTFPAVLQSSGLYSLSSVVTVPS | |
| | | | | SSLGTQTYICNVNHKPSNTKVDKKVEPKSCDKTHTCPPCPAPEAAGGP | |
| | | | | SVFLFPPKPKDTLMISRTPEVTCVVVDVSHEDPEVKFNWYVDGVEVH | |
| | | | | NAKTKPREEQYNSTYRVVSVLTVLHQDWLNGKEYKCKVSNKALAAPIE | |
| | | | | KTISKAKGQPREPQVYTLPPSRDELTKNQVSLTCLVKGFYPSDIAVEWE | |
| | | | | SNGQPENNYKTTPPVLDSDGSFFLYSKLTVDKSRWQQGNVFSCSVM | |
| | | | | HEALHNHYTQKSLSLSPG | |
| 41 | ADI-59206 | WT | BCMA/None | QVKLEESGGGLVQAGRSLRLSCAASEHTFSSHVMGWFRQAPGKERE | 611 |
| | | | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLTCLVKGFYPSDIAV | |
| | | | | EWESNGQPENNYKTTPPVLDSDGSFFLYSKLTVDKSRWQQGNVFSCS | |
| | | | | VMHEALHNHYTQKSLSLSPG | |

APPENDIX TABLE B
Heavy chain B sequences of antibodies of Table 9

| | | CH3 | Specificity | | |
|---|---|---|---|---|---|
| | Antibody | Set | (Chain A/ | | SEQ |
| Index | Name | Name | Chain B) | Sequence | ID NO: |
| 1 | ADI-59166 | WT | CD3/HER2 | QVQLVQSGAEVKKPGASVKVSCKASGYAFPNYGISWVRQAPGQGLE | 212 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 2 | ADI-59167 | QR-F | | QVQLVQSGAEVKKPGASVKVSCKASGYAFPNYGISWVRQAPGQGLE | 222 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCFVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 3 | ADI-59168 | RG-FG | | QVQLVQSGAEVKKPGASVKVSCKASGYAFPNYGISWVRQAPGQGLE | 232 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCFVGGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 4 | ADI-59169 | TL-QL | | QVQLVQSGAEVKKPGASVKVSCKASGYAFPNYGISWVRQAPGQGLE | 242 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLQSDGSFLLYSKLTVDKSRWQQG | |
| | | | | NVFSCSVMHEALHNHYTQKSLSLSPG | |
| 5 | ADI-59170 | DVG- | | QVQLVQSGAEVKKPGASVKVSCKASGYAFPNYGISWVRQAPGQGLE | 252 |
| | | VSY | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLVCSVYGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 6 | ADI-59171 | LWG- | | QVQLVQSGAEVKKPGASVKVSCKASGYAFPNYGISWVRQAPGQGLE | 262 |
| | | SIG | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLSCIVKGFYP | |
| | | | | SDIAVEWESNGQPENNYKTTPPVLDSDGSFFLGSKLTVDKSRWQQG | |
| | | | | NVFSCSVMHEALHNHYTQKSLSLSPG | |
| 7 | ADI-59172 | QR-F | | QVQLVQSGAEVKKPGASVKVSCKASGYAFPNYGISWVRQAPGQGLE | 272 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLQCLVKGFY | |
| | | | | PSDIAVEWESNGQPENNYKTTPPVLDSDGSFFLYSRLTVDKSRWQQG | |
| | | | | NVFSCSVMHEALHNHYTQKSLSLSPG | |
| 8 | ADI-59173 | RG-FG | | QVQLVQSGAEVKKPGASVKVSCKASGYAFPNYGISWVRQAPGQGLE | 282 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLRCLVKGFY | |
| | | | | PSDIAVEWESNGQPENNYKTTPPVLDSDGSFFLYSGLTVDKSRWQQG | |
| | | | | NVFSCSVMHEALHNHYTQKSLSLSPG | |
| 9 | ADI-59174 | TL-QL | | QVQLVQSGAEVKKPGASVKVSCKASGYAFPNYGISWVRQAPGQGLE | 292 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDTDGSFFLYSLLTVDKSRWQQG | |
| | | | | NVFSCSVMHEALHNHYTQKSLSLSPG | |
| 10 | ADI-59175 | DVG- | | QVQLVQSGAEVKKPGASVKVSCKASGYAFPNYGISWVRQAPGQGLE | 302 |
| | | VSY | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVDLTCLVKGFY | |
| | | | | PSDIAVEWESNGQPENNYKTTPPVLDSDGSFFLVSGLTVDKSRWQQ | |
| | | | | GNVFSCSVMHEALHNHYTQKSLSLSPG | |
| 11 | ADI-59176 | LWG- | | QVQLVQSGAEVKKPGASVKVSCKASGYAFPNYGISWVRQAPGQGLE | 312 |
| | | SIG | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVLLWCLVKGFY | |
| | | | | PSDIAVEWESNGQPENNYKTTPPVLDSDGSFFLYSGLTVDKSRWQQG | |
| | | | | NVFSCSVMHEALHNHYTQKSLSLSPG | |
| 12 | ADI-59177 | WT | | QVQLVQSGAEVKKPGASVKVSCKASGYAFPNYGISWVRQAPGQGLE | 322 |
| | | (354/ | | WMGWIVAYGGNTVYAQKLQGRVTMTTDTSTSTAYMELRSLRSDDT | |
| | | 349) | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLTCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 13 | ADI-59178 | QR-F | | QVQLVQSGAEVKKPGASVKVSCKASGYAFPNYGISWVRQAPGQGLE | 332 |
| | | (354/ | | WMGWIVAYGGNTVYAQKLQGRVTMTTDTSTSTAYMELRSLRSDDT | |
| | | 349) | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLTCFVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 14 | ADI-59179 | RG-FG | | QVQLVQSGAEVKKPGASVKVSCKASGYAFPNYGISWVRQAPGQGLE | 342 |
| | | (354/ | | WMGWIVAYGGNTVYAQKLQGRVTMTTDTSTSTAYMELRSLRSDDT | |
| | | 349) | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLTCFVGGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 15 | ADI-59180 | TL-QL | | QVQLVQSGAEVKKPGASVKVSCKASGYAFPNYGISWVRQAPGQGLE | 352 |
| | | (354/ | | WMGWIVAYGGNTVYAQKLQGRVTMTTDTSTSTAYMELRSLRSDDT | |
| | | 349) | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLTCLVKGFY | |
| | | | | PSDIAVEWESNGQPENNYKTTPPVLQSDGSFLLYSKLTVDKSRWQQG | |
| | | | | NVFSCSVMHEALHNHYTQKSLSLSPG | |
| 16 | ADI-59181 | DVG- | | QVQLVQSGAEVKKPGASVKVSCKASGYAFPNYGISWVRQAPGQGLE | 362 |
| | | VSY | | WMGWIVAYGGNTVYAQKLQGRVTMTTDTSTSTAYMELRSLRSDDT | |
| | | (354/ | | AVYYCARDGGQPIDYGMDVWGQGTTVTVSSASTKGPSVFPLAPSSK | |
| | | 349) | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLVCSVYGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 17 | ADI-59182 | LWG- | | QVQLVQSGAEVKKPGASVKVSCKASGYAFPNYGISWVRQAPGQGLE | 372 |
| | | SIG | | WMGWIVAYGGNTVYAQKLQGRVTMTTDTSTSTAYMELRSLRSDDT | |
| | | (354/ | | AVYYCARDGGQPIDYGMDVWGQGTTVTVSSASTKGPSVFPLAPSSK | |
| | | 349) | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVCTLPPSRDELTKNQVSLSCIVKGFYP | |
| | | | | SDIAVEWESNGQPENNYKTTPPVLDSDGSFFLGSKLTVDKSRWQQG | |
| | | | | NVFSCSVMHEALHNHYTQKSLSLSPG | |
| 18 | ADI-59183 | WT | CD20/CD3 | EVQLVESGGGLVQPGRSLRLSCAASGFTFDDYTMHWVRQAPGKGLE | 382 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 19 | ADI-59184 | QR-F | | EVQLVESGGGLVQPGRSLRLSCAASGFTFDDYTMHWVRQAPGKGLE | 392 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCFVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 20 | ADI-59185 | RG-FG | | EVQLVESGGGLVQPGRSLRLSCAASGFTFDDYTMHWVRQAPGKGLE | 402 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCFVGGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 21 | ADI-59186 | TL-QL | | EVQLVESGGGLVQPGRSLRLSCAASGFTFDDYTMHWVRQAPGKGLE | 412 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLQSDGSFLLYSKLTVDKSRWQQG | |
| | | | | NVFSCSVMHEALHNHYTQKSLSLSPG | |
| 22 | ADI-59187 | DVG- | | EVQLVESGGGLVQPGRSLRLSCAASGFTFDDYTMHWVRQAPGKGLE | 422 |
| | | VSY | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLVCSVYGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 23 | ADI-59188 | LWG- | | EVQLVESGGGLVQPGRSLRLSCAASGFTFDDYTMHWVRQAPGKGLE | 432 |
| | | SIG | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLSCIVKGFYP | |
| | | | | SDIAVEWESNGQPENNYKTTPPVLDSDGSFFLGSKLTVDKSRWQQG | |
| | | | | NVFSCSVMHEALHNHYTQKSLSLSPG | |
| 24 | ADI-59189 | QR-F | | EVQLVESGGGLVQPGRSLRLSCAASGFTFDDYTMHWVRQAPGKGLE | 442 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLQCLVKGFY | |
| | | | | PSDIAVEWESNGQPENNYKTTPPVLDSDGSFFLYSRLTVDKSRWQQG | |
| | | | | NVFSCSVMHEALHNHYTQKSLSLSPG | |
| 25 | ADI-59190 | RG-FG | | EVQLVESGGGLVQPGRSLRLSCAASGFTFDDYTMHWVRQAPGKGLE | 452 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLRCLVKGFY | |
| | | | | PSDIAVEWESNGQPENNYKTTPPVLDSDGSFFLYSGLTVDKSRWQQG | |
| | | | | NVFSCSVMHEALHNHYTQKSLSLSPG | |
| 26 | ADI-59191 | TL-QL | | EVQLVESGGGLVQPGRSLRLSCAASGFTFDDYTMHWVRQAPGKGLE | 462 |
| | | | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVSLTCLVKGFY | |
| | | | | PSDIAVEWESNGQPENNYKTTPPVLDTDGSFFLYSLLTVDKSRWQQG | |
| | | | | NVFSCSVMHEALHNHYTQKSLSLSPG | |
| 27 | ADI-59192 | DVG- | | EVQLVESGGGLVQPGRSLRLSCAASGFTFDDYTMHWVRQAPGKGLE | 472 |
| | | VSY | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVDLTCLVKGFY | |
| | | | | PSDIAVEWESNGQPENNYKTTPPVLDSDGSFFLVSGLTVDKSRWQQ | |
| | | | | GNVFSCSVMHEALHNHYTQKSLSLSPG | |
| 28 | ADI-59193 | LWG- | | EVQLVESGGGLVQPGRSLRLSCAASGFTFDDYTMHWVRQAPGKGLE | 482 |
| | | SIG | | WVSGISWNSGSIGYADSVKGRFTISRDNAKKSLYLQMNSLRAEDTALY | |
| | | | | YCAKDNSGYGHYYYGMDVWGQGTTVTVASASTKGPSVFPLAPSSKS | |
| | | | | TSGGTAALGCLVKDYFPEPVTVSWNSGALTSGVHTFPAVLQSSGLYSL | |
| | | | | SSVVTVPSSSLGTQTYICNVNHKPSNTKVDKKVEPKSCDKTHTCPPCP | |
| | | | | APEAAGGPSVFLFPPKPKDTLMISRTPEVTCVVVDVSHEDPEVKFNW | |
| | | | | YVDGVEVHNAKTKPREEQYNSTYRVVSVLTVLHQDWLNGKEYKCKVS | |
| | | | | NKALAAPIEKTISKAKGQPREPQVYTLPPSRDELTKNQVLLWCLVKGFY | |
| | | | | PSDIAVEWESNGQPENNYKTTPPVLDSDGSFFLYSGLTVDKSRWQQG | |
| | | | | NVFSCSVMHEALHNHYTQKSLSLSPG | |
| 29 | ADI-59194 | WT | HEL/BCMA | QVKLEESGGGLVQAGRSLRLSCAASEHTFSSHVMGWFRQAPGKERE | 492 |
| | | | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLTCLVKGFYPSDIAV | |
| | | | | EWESNGQPENNYKTTPPVLDSDGSFFLYSKLTVDKSRWQQGNVFSCS | |
| | | | | VMHEALHNHYTQKSLSLSPG | |
| 30 | ADI-59195 | QR-F | | QVKLEESGGGLVQAGRSLRLSCAASEHTFSSHVMGWFRQAPGKERE | 502 |
| | | | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLTCFVKGFYPSDIAV | |
| | | | | EWESNGQPENNYKTTPPVLDSDGSFFLYSKLTVDKSRWQQGNVFSCS | |
| | | | | VMHEALHNHYTQKSLSLSPG | |
| 31 | ADI-59196 | RG-FG | | QVKLEESGGGLVQAGRSLRLSCAASEHTFSSHVMGWFRQAPGKERE | 512 |
| | | | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLTCFVGGFYPSDIAV | |
| | | | | EWESNGQPENNYKTTPPVLDSDGSFFLYSKLTVDKSRWQQGNVFSCS | |
| | | | | VMHEALHNHYTQKSLSLSPG | |
| 32 | ADI-59197 | TL-QL | | QVKLEESGGGLVQAGRSLRLSCAASEHTFSSHVMGWFRQAPGKERE | 522 |
| | | | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLTCLVKGFYPSDIAV | |
| | | | | EWESNGQPENNYKTTPPVLQSDGSFLLYSKLTVDKSRWQQGNVFSCS | |
| | | | | VMHEALHNHYTQKSLSLSPG | |
| 33 | ADI-59198 | DVG- | | QVKLEESGGGLVQAGRSLRLSCAASEHTFSSHVMGWFRQAPGKERE | 532 |
| | | VSY | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLVCSVYGFYPSDIAV | |
| | | | | EWESNGQPENNYKTTPPVLDSDGSFFLYSKLTVDKSRWQQGNVFSCS | |
| | | | | VMHEALHNHYTQKSLSLSPG | |
| 34 | ADI-59199 | LWG- | | QVKLEESGGGLVQAGRSLRLSCAASEHTFSSHVMGWFRQAPGKERE | 542 |
| | | SIG | | SVAVIGWRDISTSYADSVKGRFTISRDNAKKTLYLQMNSLKPEDTAVY | |
| | | | | YCAARRIDAADFDSWGQGTQVTVSSGSEPKSSDKTHTCPPCPAPEAA | |
| | | | | GGPSVFLFPPKPKDTLMISRTPEVTCVVVDVSHEDPEVKFNWYVDGV | |
| | | | | EVHNAKTKPREEQYNSTYRVVSVLTVLHQDWLNGKEYKCKVSNKALA | |
| | | | | APIEKTISKAKGQPREPQVYTLPPSRDELTKNQVSLSCIVKGFYPSDIAV | |
| | | | | EWESNGQPENNYKTTPPVLDSDGSFFLGSKLTVDKSRWQQGNVFSC | |
| | | | | SVMHEALHNHYTQKSLSLSPG | |
| 35 | ADI-59200 | KIH | CD3/HER2 | QVQLVQSGAEVKKPGASVKVSCKASGYAFPNYGISWVRQAPGQGLE | 552 |
| | | | | WMGWIVAYGGNTVYAQKLQGRVTMTTDTSTSTAYMELRSLRSDDT | |
| | | | | AVYYCARDGGQPIDYGMDVWGQGTTVTVSSASTKGPSVFPLAPSSK | |
| | | | | STSGGTAALGCLVKDYFPEPVTVSWNSGALTSGVHTFPAVLQSSGLYS | |
| | | | | LSSVVTVPSSSLGTQTYICNVNHKPSNTKVDKKVEPKSCDKTHTCPPCP | |
| | | | | APELLGGPSVFLFPPKPKDTLMISRTPEVTCVVVDVSHEDPEVKFNWY | |
| | | | | VDGVEVHNAKTKPREEQYNSTYRVVSVLTVLHQDWLNGKEYKCKVS | |
| | | | | NKALPAPIEKTISKAKGQPREPQVYTLPPSRDELTKNQVSLWCLVKGFY | |
| | | | | PSDIAVEWESNGQPENNYKTTPPVLDSDGSFFLYSKLTVDKSRWQQG | |
| | | | | NVFSCSVMHEALHNHYTQKSLSLSPG | |
| 36 | ADI-59201 | WT | CD3/None | | |
| 37 | ADI-59202 | WT | HER2/None | N.A. | |
| 38 | ADI-59203 | WT | CD20/None | N.A. | |
| 39 | ADI-59204 | WT | CD3/None | N.A. | |
| 40 | ADI-59205 | WT | HEL/None | N.A. | |
| 41 | ADI-59206 | WT | BCMA/None | N.A. | |

APPENDIX TABLE C
Light chain A sequences of antibodies of Table 9

| | | CH3 | Specificity | Light Chain A | |
|---|---|---|---|---|---|
| | Antibody | Set | (Chain A / | | SEQ |
| Index | Name | Name | Chain B) | Sequence | ID NO: |
| 1 | ADI-59166 | WT | CD3/HER2 | DIVMTQSPDSLAVSLGERATINCKSSQSLLNARTGKNYLAWYQQK | 213 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 2 | ADI-59167 | QR-F | | DIVMTQSPDSLAVSLGERATINCKSSQSLLNARTGKNYLAWYQQK | 223 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 3 | ADI-59168 | RG-FG | | DIVMTQSPDSLAVSLGERATINCKSSQSLLNARTGKNYLAWYQQK | 233 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 4 | ADI-59169 | TL-QL | | DIVMTQSPDSLAVSLGERATINCKSSQSLLNARTGKNYLAWYQQK | 243 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 5 | ADI-59170 | DVG- | | DIVMTQSPDSLAVSLGERATINCKSSQSLLNARTGKNYLAWYQQK | 253 |
| | | VSY | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 6 | ADI-59171 | LWG- | | DIVMTQSPDSLAVSLGERATINCKSSQSLLNARTGKNYLAWYQQK | 263 |
| | | SIG | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 7 | ADI-59172 | QR-F | | DIVMTQSPDSLAVSLGERATINCKSSQSLLNARTGKNYLAWYQQK | 273 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 8 | ADI-59173 | RG-FG | | DIVMTQSPDSLAVSLGERATINCKSSQSLLNARTGKNYLAWYQQK | 283 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 9 | ADI-59174 | TL-QL | | DIVMTQSPDSLAVSLGERATINCKSSQSLLNARTGKNYLAWYQQK | 293 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 10 | ADI-59175 | DVG- | | DIVMTQSPDSLAVSLGERATINCKSSQSLLNARTGKNYLAWYQQK | 303 |
| | | VSY | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 11 | ADI-59176 | LWG- | | DIVMTQSPDSLAVSLGERATINCKSSQSLLNARTGKNYLAWYQQK | 313 |
| | | SIG | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 12 | ADI-59177 | WT | | DIVMTQSPDSLAVSLGERATINCKSSQSLLNARTGKNYLAWYQQK | 323 |
| | | (354/ | | PGQPPKLLIYWASTRESGVPDRFSGSGSGTDFTLTISSLQAEDVAVY | |
| | | 349) | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 13 | ADI-59178 | QR-F | | DIVMTQSPDSLAVSLGERATINCKSSQSLLNARTGKNYLAWYQQK | 333 |
| | | (354/ | | PGQPPKLLIYWASTRESGVPDRFSGSGSGTDFTLTISSLQAEDVAVY | |
| | | 349) | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 14 | ADI-59179 | RG-FG | | DIVMTQSPDSLAVSLGERATINCKSSQSLLNARTGKNYLAWYQQK | 343 |
| | | (354/ | | PGQPPKLLIYWASTRESGVPDRFSGSGSGTDFTLTISSLQAEDVAVY | |
| | | 349) | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 15 | ADI-59180 | TL-QL | | DIVMTQSPDSLAVSLGERATINCKSSQSLLNARTGKNYLAWYQQK | 353 |
| | | (354/ | | PGQPPKLLIYWASTRESGVPDRFSGSGSGTDFTLTISSLQAEDVAVY | |
| | | 349) | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 16 | ADI-59181 | DVG- | | DIVMTQSPDSLAVSLGERATINCKSSQSLLNARTGKNYLAWYQQK | 363 |
| | | VSY | | PGQPPKLLIYWASTRESGVPDRFSGSGSGTDFTLTISSLQAEDVAVY | |
| | | (354/ | | YCKQSYSLRTFGGGTKVEIKRTVAAPSVFIFPPSDEQLKSGTASVVCL | |
| | | 349) | | LNNFYPREAKVQWKVDNALQSGNSQESVTEQDSKDSTYSLSSTLTL | |
| | | | | SKADYEKHKVYACEVTHQGLSSPVTKSFNRGEC | |
| 17 | ADI-59182 | LWG- | | DIVMTQSPDSLAVSLGERATINCKSSQSLLNARTGKNYLAWYQQK | 373 |
| | | SIG | | PGQPPKLLIYWASTRESGVPDRFSGSGSGTDFTLTISSLQAEDVAVY | |
| | | (354/ | | YCKQSYSLRTFGGGTKVEIKRTVAAPSVFIFPPSDEQLKSGTASVVCL | |
| | | 349) | | LNNFYPREAKVQWKVDNALQSGNSQESVTEQDSKDSTYSLSSTLTL | |
| | | | | SKADYEKHKVYACEVTHQGLSSPVTKSFNRGEC | |
| 18 | ADI-59183 | WT | CD20/CD3 | EIVMTQSPATLSVSPGERATLSCRASQSVSSNLAWYQQKPGQAPR | 383 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 19 | ADI-59184 | QR-F | | EIVMTQSPATLSVSPGERATLSCRASQSVSSNLAWYQQKPGQAPR | 393 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 20 | ADI-59185 | RG-FG | | EIVMTQSPATLSVSPGERATLSCRASQSVSSNLAWYQQKPGQAPR | 403 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 21 | ADI-59186 | TL-QL | | EIVMTQSPATLSVSPGERATLSCRASQSVSSNLAWYQQKPGQAPR | 413 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 22 | ADI-59187 | DVG- | | EIVMTQSPATLSVSPGERATLSCRASQSVSSNLAWYQQKPGQAPR | 423 |
| | | VSY | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 23 | ADI-59188 | LWG- | | EIVMTQSPATLSVSPGERATLSCRASQSVSSNLAWYQQKPGQAPR | 433 |
| | | SIG | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 24 | ADI-59189 | QR-F | | EIVMTQSPATLSVSPGERATLSCRASQSVSSNLAWYQQKPGQAPR | 443 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 25 | ADI-59190 | RG-FG | | EIVMTQSPATLSVSPGERATLSCRASQSVSSNLAWYQQKPGQAPR | 453 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 26 | ADI-59191 | TL-QL | | EIVMTQSPATLSVSPGERATLSCRASQSVSSNLAWYQQKPGQAPR | 463 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 27 | ADI-59192 | DVG- | | EIVMTQSPATLSVSPGERATLSCRASQSVSSNLAWYQQKPGQAPR | 473 |
| | | VSY | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 28 | ADI-59193 | LWG- | | EIVMTQSPATLSVSPGERATLSCRASQSVSSNLAWYQQKPGQAPR | 483 |
| | | SIG | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 29 | ADI-59194 | WT | HEL/BCMA | DIQMTQSPSSLSASVGDRVTITCQASQDISNYLNWYQQKPGKAPK | 493 |
| | | | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 30 | ADI-59195 | QR-F | | DIQMTQSPSSLSASVGDRVTITCQASQDISNYLNWYQQKPGKAPK | 503 |
| | | | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 31 | ADI-59196 | RG-FG | | DIQMTQSPSSLSASVGDRVTITCQASQDISNYLNWYQQKPGKAPK | 513 |
| | | | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 32 | ADI-59197 | TL-QL | | DIQMTQSPSSLSASVGDRVTITCQASQDISNYLNWYQQKPGKAPK | 523 |
| | | | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 33 | ADI-59198 | DVG- | | DIQMTQSPSSLSASVGDRVTITCQASQDISNYLNWYQQKPGKAPK | 533 |
| | | VSY | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 34 | ADI-59199 | LWG- | | DIQMTQSPSSLSASVGDRVTITCQASQDISNYLNWYQQKPGKAPK | 543 |
| | | SIG | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 35 | ADI-59200 | KiH | CD3/HER2 | DIVMTQSPDSLAVSLGERATINCKSSQSLLNARTGKNYLAWYQQK | 553 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 36 | ADI-59201 | WT | CD3/None | DIVMTQSPDSLAVSLGERATINCKSSQSLLNARTGKNYLAWYQQK | 563 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 37 | ADI-59202 | WT | HER2/None | DIVMTQSPDSLAVSLGERATINCKSSQSLLNARTGKNYLAWYQQK | 573 |
| | | | | PGQPPKLLIYWASTRESGVPDRFSGSGSGTDFTLTISSLQAEDVAVY | |
| | | | | YCKQSYSLRTFGGGTKVEIKRTVAAPSVFIFPPSDEQLKSGTASVVCL | |
| | | | | LNNFYPREAKVQWKVDNALQSGNSQESVTEQDSKDSTYSLSSTLTL | |
| | | | | SKADYEKHKVYACEVTHQGLSSPVTKSFNRGEC | |
| 38 | ADI-59203 | WT | CD20/None | EIVMTQSPATLSVSPGERATLSCRASQSVSSNLAWYQQKPGQAPR | 583 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 39 | ADI-59204 | WT | CD3/None | EIVMTQSPATLSVSPGERATLSCRASQSVSSNLAWYQQKPGQAPR | 593 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 40 | ADI-59205 | WT | HEL/None | DIQMTQSPSSLSASVGDRVTITCQASQDISNYLNWYQQKPGKAPK | 594 |
| | | | | LLIYDASNLETGVPSRFSGSGSGTDFTFTISSLQPEDIATYYCQQAFN | |
| | | | | YPLTFGGGTKVEIKRTVAAPSVFIFPPSDEQLKSGTASVVCLLNNFYP | |
| | | | | REAKVQWKVDNALQSGNSQESVTEQDSKDSTYSLSSTLTLSKADYE | |
| | | | | KHKVYACEVTHQGLSSPVTKSFNRGEC | |
| 41 | ADI-59206 | WT | BCMA/None | N.A. | |

APPENDIX TABLE D
Light chain B sequences of antibodies of Table 9

| | | | Specificity | Light Chain B | |
|---|---|---|---|---|---|
| | Antibody | CH3 Set | (Chain A/ | | SEQ |
| Index | Name | Name | Chain B) | Sequence | ID NO: |
| 1 | ADI-59166 | WT | CD3/HER2 | DIVMTQSPDSLAVSLGERATINCKSSQSLLNARTGKNYLAWYQQKP | 214 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 2 | ADI-59167 | QR-F | | DIVMTQSPDSLAVSLGERATINCKSSQSLLNARTGKNYLAWYQQKP | 224 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 3 | ADI-59168 | RG-FG | | DIVMTQSPDSLAVSLGERATINCKSSQSLLNARTGKNYLAWYQQKP | 234 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 4 | ADI-59169 | TL-QL | | DIVMTQSPDSLAVSLGERATINCKSSQSLLNARTGKNYLAWYQQKP | 244 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 5 | ADI-59170 | DVG- | | DIVMTQSPDSLAVSLGERATINCKSSQSLLNARTGKNYLAWYQQKP | 254 |
| | | VSY | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 6 | ADI-59171 | LWG- | | DIVMTQSPDSLAVSLGERATINCKSSQSLLNARTGKNYLAWYQQKP | 264 |
| | | SIG | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 7 | ADI-59172 | QR-F | | DIVMTQSPDSLAVSLGERATINCKSSQSLLNARTGKNYLAWYQQKP | 274 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 8 | ADI-59173 | RG-FG | | DIVMTQSPDSLAVSLGERATINCKSSQSLLNARTGKNYLAWYQQKP | 284 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 9 | ADI-59174 | TL-QL | | DIVMTQSPDSLAVSLGERATINCKSSQSLLNARTGKNYLAWYQQKP | 294 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 10 | ADI-59175 | DVG- | | DIVMTQSPDSLAVSLGERATINCKSSQSLLNARTGKNYLAWYQQKP | 304 |
| | | VSY | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 11 | ADI-59176 | LWG- | | DIVMTQSPDSLAVSLGERATINCKSSQSLLNARTGKNYLAWYQQKP | 314 |
| | | SIG | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 12 | ADI-59177 | WT | | DIVMTQSPDSLAVSLGERATINCKSSQSLLNARTGKNYLAWYQQKP | 324 |
| | | (354/ | | GQPPKLLIYWASTRESGVPDRFSGSGSGTDFTLTISSLQAEDVAVYY | |
| | | 349) | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 13 | ADI-59178 | QR-F | | DIVMTQSPDSLAVSLGERATINCKSSQSLLNARTGKNYLAWYQQKP | 334 |
| | | (354/ | | GQPPKLLIYWASTRESGVPDRFSGSGSGTDFTLTISSLQAEDVAVYY | |
| | | 349) | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 14 | ADI-59179 | RG-FG | | DIVMTQSPDSLAVSLGERATINCKSSQSLLNARTGKNYLAWYQQKP | 344 |
| | | (354/ | | GQPPKLLIYWASTRESGVPDRFSGSGSGTDFTLTISSLQAEDVAVYY | |
| | | 349) | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 15 | ADI-59180 | TL-QL | | DIVMTQSPDSLAVSLGERATINCKSSQSLLNARTGKNYLAWYQQKP | 354 |
| | | (354/ | | GQPPKLLIYWASTRESGVPDRFSGSGSGTDFTLTISSLQAEDVAVYY | |
| | | 349) | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 16 | ADI-59181 | DVG- | | DIVMTQSPDSLAVSLGERATINCKSSQSLLNARTGKNYLAWYQQKP | 364 |
| | | VSY | | GQPPKLLIYWASTRESGVPDRFSGSGSGTDFTLTISSLQAEDVAVYY | |
| | | (354/ | | CKQSYSLRTFGGGTKVEIKRTVAAPSVFIFPPSDEQLKSGTASVVCLL | |
| | | 349) | | NNFYPREAKVQWKVDNALQSGNSQESVTEQDSKDSTYSLSSTLTLS | |
| | | | | KADYEKHKVYACEVTHQGLSSPVTKSFNRGEC | |
| 17 | ADI-59182 | LWG- | | DIVMTQSPDSLAVSLGERATINCKSSQSLLNARTGKNYLAWYQQKP | 374 |
| | | SIG | | GQPPKLLIYWASTRESGVPDRFSGSGSGTDFTLTISSLQAEDVAVYY | |
| | | (354/ | | CKQSYSLRTFGGGTKVEIKRTVAAPSVFIFPPSDEQLKSGTASVVCLL | |
| | | 349) | | NNFYPREAKVQWKVDNALQSGNSQESVTEQDSKDSTYSLSSTLTLS | |
| | | | | KADYEKHKVYACEVTHQGLSSPVTKSFNRGEC | |
| 18 | ADI-59183 | WT | CD20/CD3 | EIVMTQSPATLSVSPGERATLSCRASQSVSSNLAWYQQKPGQAPR | 384 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 19 | ADI-59184 | QR-F | | EIVMTQSPATLSVSPGERATLSCRASQSVSSNLAWYQQKPGQAPR | 394 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 20 | ADI-59185 | RG-FG | | EIVMTQSPATLSVSPGERATLSCRASQSVSSNLAWYQQKPGQAPR | 404 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 21 | ADI-59186 | TL-QL | | EIVMTQSPATLSVSPGERATLSCRASQSVSSNLAWYQQKPGQAPR | 414 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 22 | ADI-59187 | DVG- | | EIVMTQSPATLSVSPGERATLSCRASQSVSSNLAWYQQKPGQAPR | 424 |
| | | VSY | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 23 | ADI-59188 | LWG- | | EIVMTQSPATLSVSPGERATLSCRASQSVSSNLAWYQQKPGQAPR | 434 |
| | | SIG | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 24 | ADI-59189 | QR-F | | EIVMTQSPATLSVSPGERATLSCRASQSVSSNLAWYQQKPGQAPR | 444 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 25 | ADI-59190 | RG-FG | | EIVMTQSPATLSVSPGERATLSCRASQSVSSNLAWYQQKPGQAPR | 454 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 26 | ADI-59191 | TL-QL | | EIVMTQSPATLSVSPGERATLSCRASQSVSSNLAWYQQKPGQAPR | 464 |
| | | | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 27 | ADI-59192 | DVG- | | EIVMTQSPATLSVSPGERATLSCRASQSVSSNLAWYQQKPGQAPR | 474 |
| | | VSY | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 28 | ADI-59193 | LWG- | | EIVMTQSPATLSVSPGERATLSCRASQSVSSNLAWYQQKPGQAPR | 484 |
| | | SIG | | LLIYGASTRATGIPARFSGSGSGTEFTLTISSLQSEDFAVYYCQHYIN | |
| | | | | WPLTFGGGTKVEIKRTVAAPSVFIFPPSDEQLKSGTASVVCLLNNFY | |
| | | | | PREAKVQWKVDNALQSGNSQESVTEQDSKDSTYSLSSTLTLSKADY | |
| | | | | EKHKVYACEVTHQGLSSPVTKSFNRGEC | |
| 29 | ADI-59194 | WT | HEL/BCMA | | |
| 30 | ADI-59195 | QR-F | | N.A. | |
| 31 | ADI-59196 | RG-FG | | N.A. | |
| 32 | ADI-59197 | TL-QL | | N.A. | |
| 33 | ADI-59198 | DVG- | | N.A. | |
| | | VSY | | | |
| 34 | ADI-59199 | LWG- | | N.A. | |
| | | SIG | | | |
| 35 | ADI-59200 | KiH | CD3/HER2 | DIVMTQSPDSLAVSLGERATINCKSSQSLLNARTGKNYLAWYQQKP | 554 |
| | | | | GQPPKLLIYWASTRESGVPDRFSGSGSGTDFTLTISSLQAEDVAVYY | |
| | | | | CKQSYSLRTFGGGTKVEIKRTVAAPSVFIFPPSDEQLKSGTASVVCLL | |
| | | | | NNFYPREAKVQWKVDNALQSGNSQESVTEQDSKDSTYSLSSTLTLS | |
| | | | | KADYEKHKVYACEVTHQGLSSPVTKSFNRGEC | |
| 36 | ADI-59201 | WT | CD3/None | N.A. | |
| 37 | ADI-59202 | WT | HER2/None | N.A. | |
| 38 | ADI-59203 | WT | CD20/None | N.A. | |
| 39 | ADI-59204 | WT | CD3/None | N.A. | |
| 40 | ADI-59205 | WT | HEL/None | N.A. | |
| 41 | ADI-59206 | WT | BCMA/None | N.A. | |

APPENDIX TABLE E
CH3 domain variant sequences (without CH3 disulfide substitution)

| CH3 | CH3 | | | | |
|---|---|---|---|---|---|
| Set | Set | | CH3 | SEQ | |
| Index | Name | Chain | substitutions | ID NO: | Sequence |
| 0 | WT | A | None | 1 | GQPREPQVYTLPPSRDELTKNQVSLTCLVKGFYPSDIAVEW |
| | | | | | ESNGQPENNYKTTPPVLDSDGSFFLYSKLTVDKSRWQQG |
| | | | | | NVFSCSVMHEALHNHYTQKSLSLSPG |
| | | B | None | 1 | GQPREPQVYTLPPSRDELTKNQVSLTCLVKGFYPSDIAVEW |
| | | | | | ESNGQPENNYKTTPPVLDSDGSFFLYSKLTVDKSRWQQG |
| | | | | | NVFSCSVMHEALHNHYTQKSLSLSPG |
| 1 | W-SG | A | T366W | 11 | GQPREPQVYTLPPSRDELTKNQVSLWCLVKGFYPSDIAVE |
| | | | | | WESNGQPENNYKTTPPVLDSDGSFFLYSKLTVDKSRWQQ |
| | | | | | GNVFSCSVMHEALHNHYTQKSLSLSPG |
| | | B | T366S Y407G | 12 | GQPREPQVYTLPPSRDELTKNQVSLSCLVKGFYPSDIAVEW |
| | | | | | ESNGQPENNYKTTPPVLDSDGSFFLGSKLTVDKSRWQQG |
| | | | | | NVFSCSVMHEALHNHYTQKSLSLSPG |
| 2 | V-V | A | T366V | 21 | GQPREPQVYTLPPSRDELTKNQVSLVCLVKGFYPSDIAVE |
| | | | | | WESNGQPENNYKTTPPVLDSDGSFFLYSKLTVDKSRWQQ |
| | | | | | GNVFSCSVMHEALHNHYTQKSLSLSPG |
| | | B | Y407V | 22 | GQPREPQVYTLPPSRDELTKNQVSLVCLVKGFYPSDIAVE |
| | | | | | WESNGQPENNYKTTPPVLDSDGSFFLVSKLTVDKSRWQQ |
| | | | | | GNVFSCSVMHEALHNHYTQKSLSLSPG |
| 3 | QR-F | A | T366Q | 31 | GQPREPQVYTLPPSRDELTKNQVSLQCLVKGFYPSDIAVE |
| | | | K409R | | WESNGQPENNYKTTPPVLDSDGSFFLYSRLTVDKSRWQQ |
| | | | | | GNVFSCSVMHEALHNHYTQKSLSLSPG |
| | | B | L368F | 32 | GQPREPQVYTLPPSRDELTKNQVSLTCFVKGFYPSDIAVEW |
| | | | | | ESNGQPENNYKTTPPVLDSDGSFFLYSKLTVDKSRWQQG |
| | | | | | NVFSCSVMHEALHNHYTQKSLSLSPG |
| 4 | RG-FG | A | T366R | 41 | GQPREPQVYTLPPSRDELTKNQVSLRCLVKGFYPSDIAVEW |
| | | | K409G | | ESNGQPENNYKTTPPVLDSDGSFFLYSGLTVDKSRWQQG |
| | | | | | NVFSCSVMHEALHNHYTQKSLSLSPG |
| | | B | L368F K370G | 42 | GQPREPQVYTLPPSRDELTKNQVSLTCFVGGFYPSDIAVE |
| | | | | | WESNGQPENNYKTTPPVLDSDGSFFLYSKLTVDKSRWQQ |
| | | | | | GNVFSCSVMHEALHNHYTQKSLSLSPG |
| 5 | TL-QL | A | S400T K409L | 51 | GQPREPQVYTLPPSRDELTKNQVSLTCLVKGFYPSDIAVEW |
| | | | | | ESNGQPENNYKTTPPVLDTDGSFFLYSLLTVDKSRWQQGN |
| | | | | | VFSCSVMHEALHNHYTQKSLSLSPG |
| | | B | D399Q | 52 | GQPREPQVYTLPPSRDELTKNQVSLTCLVKGFYPSDIAVEW |
| | | | F405L | | ESNGQPENNYKTTPPVLQSDGSFLLYSKLTVDKSRWQQG |
| | | | | | NVFSCSVMHEALHNHYTQKSLSLSPG |
| 6 | DVG- | A | S364D Y407V | 61 | GQPREPQVYTLPPSRDELTKNQVDLTCLVKGFYPSDIAVE |
| | VSY | | K409G | | WESNGQPENNYKTTPPVLDSDGSFFLVSGLTVDKSRWQQ |
| | | | | | GNVFSCSVMHEALHNHYTQKSLSLSPG |
| | | B | T366V L368S | 62 | GQPREPQVYTLPPSRDELTKNQVSLVCSVYGFYPSDIAVE |
| | | | K370Y | | WESNGQPENNYKTTPPVLDSDGSFFLYSKLTVDKSRWQQ |
| | | | | | GNVFSCSVMHEALHNHYTQKSLSLSPG |
| 7 | LWG- | A | S364L | 71 | GQPREPQVYTLPPSRDELTKNQVLLWCLVKGFYPSDIAVE |
| | SIG | | T366W | | WESNGQPENNYKTTPPVLDSDGSFFLYSGLTVDKSRWQQ |
| | | | K409G | | GNVFSCSVMHEALHNHYTQKSLSLSPG |
| | | B | T366S L3681 | 72 | GQPREPQVYTLPPSRDELTKNQVSLSCIVKGFYPSDIAVEW |
| | | | Y407G | | ESNGQPENNYKTTPPVLDSDGSFFLGSKLTVDKSRWQQG |
| | | | | | NVFSCSVMHEALHNHYTQKSLSLSPG |
| 8 | WTL- | A | T366W | 81 | GQPREPQVYTLPPSRDELTKNQVSLWCLVKGFYPSDIAVE |
| | SAVQL | | S400T K409L | | WESNGQPENNYKTTPPVLDTDGSFFLYSLLTVDKSRWQQ |
| | | | | | GNVFSCSVMHEALHNHYTQKSLSLSPG |
| | | B | T366S L368A | 82 | GQPREPQVYTLPPSRDELTKNQVSLSCAVKGFYPSDIAVE |
| | | | Y407V | | WESNGQPENNYKTTPPVLQSDGSFLLVSKLTVDKSRWQQ |
| | | | D399Q | | GNVFSCSVMHEALHNHYTQKSLSLSPG |
| | | | F405L | | |
| 9 | WTL- | A | T366W | 91 | GQPREPQVYTLPPSRDELTKNQVSLWCLVKGFYPSDIAVE |
| | SGQL | | S400T K409L | | WESNGQPENNYKTTPPVLDTDGSFFLYSLLTVDKSRWQQ |
| | | | | | GNVFSCSVMHEALHNHYTQKSLSLSPG |
| | | B | T366S Y407G | 92 | GQPREPQVYTLPPSRDELTKNQVSLSCLVKGFYPSDIAVEW |
| | | | D399Q | | ESNGQPENNYKTTPPVLQSDGSFLLGSKLTVDKSRWQQG |
| | | | F405L | | NVFSCSVMHEALHNHYTQKSLSLSPG |
| 10 | WQL- | A | T366W | 101 | GQPREPQVYTLPPSRDELTKNQVSLWCLVKGFYPSDIAVE |
| | SAVTL | | D399Q | | WESNGQPENNYKTTPPVLQSDGSFLLYSKLTVDKSRWQQ |
| | | | F405L | | GNVFSCSVMHEALHNHYTQKSLSLSPG |
| | | B | T366S L368A | 102 | GQPREPQVYTLPPSRDELTKNQVSLSCAVKGFYPSDIAVE |
| | | | Y407V S400T | | WESNGQPENNYKTTPPVLDTDGSFFLVSLLTVDKSRWQQ |
| | | | K409L | | GNVFSCSVMHEALHNHYTQKSLSLSPG |
| 11 | WQL- | A | T366W | 111 | GQPREPQVYTLPPSRDELTKNQVSLWCLVKGFYPSDIAVE |
| | SGTL | | D399Q | | WESNGQPENNYKTTPPVLQSDGSFLLYSKLTVDKSRWQQ |
| | | | F405L | | GNVFSCSVMHEALHNHYTQKSLSLSPG |
| | | B | T366S Y407G | 112 | GQPREPQVYTLPPSRDELTKNQVSLSCLVKGFYPSDIAVEW |
| | | | S400T K409L | | ESNGQPENNYKTTPPVLDTDGSFFLGSLLTVDKSRWQQG |
| | | | | | NVFSCSVMHEALHNHYTQKSLSLSPG |
| 12 | VTL- | A | Y407V S400T | 121 | GQPREPQVYTLPPSRDELTKNQVSLTCLVKGFYPSDIAVEW |
| | VQL | | K409L | | ESNGQPENNYKTTPPVLDTDGSFFLVSLLTVDKSRWQQG |
| | | | | | NVFSCSVMHEALHNHYTQKSLSLSPG |
| | | B | T366V | 122 | GQPREPQVYTLPPSRDELTKNQVSLVCLVKGFYPSDIAVE |
| | | | D399Q | | WESNGQPENNYKTTPPVLQSDGSFLLYSKLTVDKSRWQQ |
| | | | F405L | | GNVFSCSVMHEALHNHYTQKSLSLSPG |
| 13 | VQL- | A | Y407V | 131 | GQPREPQVYTLPPSRDELTKNQVSLTCLVKGFYPSDIAVEW |
| | VTL | | D399Q | | ESNGQPENNYKTTPPVLQSDGSFLLVSKLTVDKSRWQQG |
| | | | F405L | | NVFSCSVMHEALHNHYTQKSLSLSPG |
| | | B | T366V S400T | 132 | GQPREPQVYTLPPSRDELTKNQVSLVCLVKGFYPSDIAVE |
| | | | K409L | | WESNGQPENNYKTTPPVLDTDGSFFLYSLLTVDKSRWQQ |
| | | | | | GNVFSCSVMHEALHNHYTQKSLSLSPG |
| 14 | QRQL- | A | T366Q | 141 | GQPREPQVYTLPPSRDELTKNQVSLQCLVKGFYPSDIAVE |
| | FTL | | K409R | | WESNGQPENNYKTTPPVLQSDGSFLLYSRLTVDKSRWQQ |
| | | | D399Q | | GNVFSCSVMHEALHNHYTQKSLSLSPG |
| | | | F405L | | |
| | | B | L368F S400T | 142 | GQPREPQVYTLPPSRDELTKNQVSLTCFVKGFYPSDIAVEW |
| | | | K409L | | ESNGQPENNYKTTPPVLDTDGSFFLYSLLTVDKSRWQQGN |
| | | | | | VFSCSVMHEALHNHYTQKSLSLSPG |
| 15 | VQR- | A | Y407V | 151 | GQPREPQVYTLPPSRDELTKNQVSLQCLVKGFYPSDIAVE |
| | VF | | T366Q | | WESNGQPENNYKTTPPVLDSDGSFFLVSRLTVDKSRWQQ |
| | | | K409R | | GNVFSCSVMHEALHNHYTQKSLSLSPG |
| | | B | T366V L368F | 152 | GQPREPQVYTLPPSRDELTKNQVSLVCFVKGFYPSDIAVE |
| | | | | | WESNGQPENNYKTTPPVLDSDGSFFLYSKLTVDKSRWQQ |
| | | | | | GNVFSCSVMHEALHNHYTQKSLSLSPG |
| 16 | LWG- | A | S364L | | GQPREPQVYTLPPSRDELTKNQVLLWCLVKGFYPSDIAVE |
| | IG | | T366W | 161 | WESNGQPENNYKTTPPVLDSDGSFFLYSGLTVDKSRWQQ |
| | | | K409G | | GNVFSCSVMHEALHNHYTQKSLSLSPG |
| | | B | | | GQPREPQVYTLPPSRDELTKNQVSLTCIVKGFYPSDIAVEW |
| | | | L3681 Y407G | 162 | ESNGQPENNYKTTPPVLDSDGSFFLGSKLTVDKSRWQQG |
| | | | | | NVFSCSVMHEALHNHYTQKSLSLSPG |

Underlined amino acids are substitutions relative to the wild-type CH3 domain sequence of SEQ ID
NO: 1.

APPENDIX TABLE F
CH3 domain variant sequences
(with the 349/354 CH3 disulfide substitutions)

| CH3 Set | CH3 Set | | CH3 | SEQ | |
|---|---|---|---|---|---|
| Index | Name | Chain | substitutions | ID NO: | |
| 1 | W-SG | A | Y349C | 13 | GQPREPQVCTLPPSRDELTKNQVSLWCLVKGFYPS |
| (349/354) | (349/354) | | T366W | | DIAVEWESNGQPENNYKTTPPVLDSDGSFFLYSKLT |
| | | | | | VDKSRWQQGNVFSCSVMHEALHNHYTQKSLSLSPG |
| | | B | S354C T366S | 14 | GQPREPQVYTLPPCRDELTKNQVSLSCLVKGFYPSD |
| | | | Y407G | | IAVEWESNGQPENNYKTTPPVLDSDGSFFLGSKLTV |
| | | | | | DKSRWQQGNVFSCSVMHEALHNHYTQKSLSLSPG |
| 2 | V-V | A | Y349C | 23 | GQPREPQVCTLPPSRDELTKNQVSLVCLVKGFYPSD |
| (349/354) | (349/354 | | T366V | | IAVEWESNGQPENNYKTTPPVLDSDGSFFLYSKLTV |
| | | | | | DKSRWQQGNVFSCSVMHEALHNHYTQKSLSLSPG |
| | | B | S354C | 24 | GQPREPQVYTLPPCRDELTKNQVSLVCLVKGFYPSD |
| | | | Y407V | | IAVEWESNGQPENNYKTTPPVLDSDGSFFLVSKLTV |
| | | | | | DKSRWQQGNVFSCSVMHEALHNHYTQKSLSLSPG |
| 3 | QR-F | A | Y349C | 33 | GQPREPQVCTLPPSRDELTKNQVSLQCLVKGFYPSD |
| (349/354) | (349/354 | | T366Q | | IAVEWESNGQPENNYKTTPPVLDSDGSFFLYSRLTV |
| | | | K409R | | DKSRWQQGNVFSCSVMHEALHNHYTQKSLSLSPG |
| | | B | S354C L368F | 34 | GQPREPQVYTLPPCRDELTKNQVSLTCFVKGFYPSD |
| | | | | | IAVEWESNGQPENNYKTTPPVLDSDGSFFLYSKLTV |
| | | | | | DKSRWQQGNVFSCSVMHEALHNHYTQKSLSLSPG |
| 4 | RG-FG | A | Y349C | 43 | GQPREPQVCTLPPSRDELTKNQVSLRCLVKGFYPSD |
| (349/354) | (349/354 | | T366R | | IAVEWESNGQPENNYKTTPPVLDSDGSFFLYSGLTV |
| | | | K409G | | DKSRWQQGNVFSCSVMHEALHNHYTQKSLSLSPG |
| | | B | S354C L368F | 44 | GQPREPQVYTLPPCRDELTKNQVSLTCFVGGFYPSD |
| | | | K370G | | IAVEWESNGQPENNYKTTPPVLDSDGSFFLYSKLTV |
| | | | | | DKSRWQQGNVFSCSVMHEALHNHYTQKSLSLSPG |
| 5 | TL-QL | A | Y349C S400T | 53 | GQPREPQVCTLPPSRDELTKNQVSLTCLVKGFYPSDI |
| (349/354) | (349/354 | | K409L | | AVEWESNGQPENNYKTTPPVLDTDGSFFLYSLLTVD |
| | | | | | KSRWQQGNVFSCSVMHEALHNHYTQKSLSLSPG |
| | | B | S354C | 54 | GQPREPQVYTLPPCRDELTKNQVSLTCLVKGFYPSD |
| | | | D399Q | | IAVEWESNGQPENNYKTTPPVLQSDGSFLLYSKLTV |
| | | | F405L | | DKSRWQQGNVFSCSVMHEALHNHYTQKSLSLSPG |
| 6 | DVG-VSY | A | Y349C | 63 | GQPREPQVCTLPPSRDELTKNQVDLTCLVKGFYPSD |
| (349/354) | (349/354 | | S364D | | IAVEWESNGQPENNYKTTPPVLDSDGSFFLVSGLTV |
| | | | Y407V | | DKSRWQQGNVFSCSVMHEALHNHYTQKSLSLSPG |
| | | | K409G | | |
| | | B | S354C | 64 | GQPREPQVYTLPPCRDELTKNQVSLVCSVYGFYPSD |
| | | | T366V L368S | | IAVEWESNGQPENNYKTTPPVLDSDGSFFLYSKLTV |
| | | | K370Y | | DKSRWQQGNVFSCSVMHEALHNHYTQKSLSLSPG |
| 7 | LWG-SIG | A | Y349C S364L | 73 | GQPREPQVCTLPPSRDELTKNQVLLWCLVKGFYPS |
| (349/354) | (349/354) | | T366W | | DIAVEWESNGQPENNYKTTPPVLDSDGSFFLYSGLT |
| | | | K409G | | VDKSRWQQGNVFSCSVMHEALHNHYTQKSLSLSPG |
| | | B | S354C T366S | 74 | GQPREPQVYTLPPCRDELTKNQVSLSCIVKGFYPSDI |
| | | | L3681 Y407G | | AVEWESNGQPENNYKTTPPVLDSDGSFFLGSKLTV |
| | | | | | DKSRWQQGNVFSCSVMHEALHNHYTQKSLSLSPG |
| 8 | WTL- | A | Y349C | 83 | GQPREPQVCTLPPSRDELTKNQVSLWCLVKGFYPS |
| (349/354) | SAVQL | | T366W | | DIAVEWESNGQPENNYKTTPPVLDTDGSFFLYSLLT |
| | (349/354) | | S400T K409L | | VDKSRWQQGNVFSCSVMHEALHNHYTQKSLSLSPG |
| | | B | S354C T366S | 84 | GQPREPQVYTLPPCRDELTKNQVSLSCAVKGFYPSD |
| | | | L368A | | IAVEWESNGQPENNYKTTPPVLQSDGSFLLVSKLTV |
| | | | Y407V | | DKSRWQQGNVFSCSVMHEALHNHYTQKSLSLSPG |
| | | | D399Q | | |
| | | | F405L | | |
| 9 | WTL- | A | Y349C | 93 | GQPREPQVCTLPPSRDELTKNQVSLWCLVKGFYPS |
| (349/354) | SGQL | | T366W | | DIAVEWESNGQPENNYKTTPPVLDTDGSFFLYSLLT |
| | (349/354) | | S400T K409L | | VDKSRWQQGNVFSCSVMHEALHNHYTQKSLSLSPG |
| | | B | S354C T366S | 94 | GQPREPQVYTLPPCRDELTKNQVSLSCLVKGFYPSD |
| | | | Y407G | | IAVEWESNGQPENNYKTTPPVLQSDGSFLLGSKLTV |
| | | | D399Q | | DKSRWQQGNVFSCSVMHEALHNHYTQKSLSLSPG |
| | | | F405L | | |
| 10 | WQL- | A | Y349C | 103 | GQPREPQVCTLPPSRDELTKNQVSLWCLVKGFYPS |
| (349/354) | SAVTL | | T366W | | DIAVEWESNGQPENNYKTTPPVLQSDGSFLLYSKLT |
| | (349/354) | | D399Q | | VDKSRWQQGNVFSCSVMHEALHNHYTQKSLSLSP |
| | | | F405L | | G |
| | | B | S354C T366S | 104 | GQPREPQVYTLPPCRDELTKNQVSLSCAVKGFYPSD |
| | | | L368A | | IAVEWESNGQPENNYKTTPPVLDTDGSFFLVSLLTV |
| | | | Y407V | | DKSRWQQGNVFSCSVMHEALHNHYTQKSLSLSPG |
| | | | S400T K409L | | |
| 11 | WQL- | A | Y349C | 113 | GQPREPQVCTLPPSRDELTKNQVSLWCLVKGFYPS |
| (349/354) | SGTL | | T366W | | DIAVEWESNGQPENNYKTTPPVLQSDGSFLLYSKLT |
| | (349/354) | | D399Q | | VDKSRWQQGNVFSCSVMHEALHNHYTQKSLSLSP |
| | | | F405L | | G |
| | | B | S354C T366S | 114 | GQPREPQVYTLPPCRDELTKNQVSLSCLVKGFYPSD |
| | | | Y407G | | IAVEWESNGQPENNYKTTPPVLDTDGSFFLGSLLTV |
| | | | S400T K409L | | DKSRWQQGNVFSCSVMHEALHNHYTQKSLSLSPG |
| 12 | VTL-VQL | A | Y349C | 123 | GQPREPQVCTLPPSRDELTKNQVSLTCLVKGFYPSDI |
| (349/354) | (349/354) | | Y407V | | AVEWESNGQPENNYKTTPPVLDTDGSFFLVSLLTV |
| | | | S400T K409L | | DKSRWQQGNVFSCSVMHEALHNHYTQKSLSLSPG |
| | | B | S354C | 124 | GQPREPQVYTLPPCRDELTKNQVSLVCLVKGFYPSD |
| | | | T366V | | IAVEWESNGQPENNYKTTPPVLQSDGSFLLYSKLTV |
| | | | D399Q | | DKSRWQQGNVFSCSVMHEALHNHYTQKSLSLSPG |
| | | | F405L | | |
| 13 | VQL-VTL | A | Y349C | 133 | GQPREPQVCTLPPSRDELTKNQVSLTCLVKGFYPSDI |
| (349/354) | (349/354) | | Y407V | | AVEWESNGQPENNYKTTPPVLQSDGSFLLVSKLTV |
| | | | D399Q | | DKSRWQQGNVFSCSVMHEALHNHYTQKSLSLSPG |
| | | | F405L | | |
| | | B | S354C | 134 | GQPREPQVYTLPPCRDELTKNQVSLVCLVKGFYPSD |
| | | | T366V | | IAVEWESNGQPENNYKTTPPVLDTDGSFFLYSLLTV |
| | | | S400T K409L | | DKSRWQQGNVFSCSVMHEALHNHYTQKSLSLSPG |
| 14 | QRQL-FTL | A | Y349C | 143 | GQPREPQVCTLPPSRDELTKNQVSLQCLVKGFYPSD |
| (349/354) | (349/354) | | T366Q | | IAVEWESNGQPENNYKTTPPVLQSDGSFLLYSRLTV |
| | | | K409R | | DKSRWQQGNVFSCSVMHEALHNHYTQKSLSLSPG |
| | | | D399Q | | |
| | | | F405L | | |
| | | B | S354C L368F | 144 | GQPREPQVYTLPPCRDELTKNQVSLTCFVKGFYPSD |
| | | | S400T K409L | | IAVEWESNGQPENNYKTTPPVLDTDGSFFLYSLLTV |
| | | | | | DKSRWQQGNVFSCSVMHEALHNHYTQKSLSLSPG |
| 15 | VQR-VF | A | Y349C | 153 | GQPREPQVCTLPPSRDELTKNQVSLQCLVKGFYPSD |
| (349/354) | (349/354) | | Y407V | | IAVEWESNGQPENNYKTTPPVLDSDGSFFLVSRLTV |
| | | | T366Q | | DKSRWQQGNVFSCSVMHEALHNHYTQKSLSLSPG |
| | | | K409R | | |
| | | B | S354C | 154 | GQPREPQVYTLPPCRDELTKNQVSLVCFVKGFYPSD |
| | | | T366V L368F | | IAVEWESNGQPENNYKTTPPVLDSDGSFFLYSKLTV |
| | | | | | DKSRWQQGNVFSCSVMHEALHNHYTQKSLSLSPG |
| 16 | LWG-IG | A | Y349C S364L | 163 | GQPREPQVYCLPPSRDELTKNQVLLWCLVKGFYPS |
| (349/354) | (349/354) | | T366W | | DIAVEWESNGQPENNYKTTPPVLDSDGSFFLYSGLT |
| | | | K409G | | VDKSRWQQGNVFSCSVMHEALHNHYTQKSLSLSPG |
| | | B | S354C L3681 | 164 | GQPREPQVYTLPPCRDELTKNQVSLTCIVKGFYPSDI |
| | | | Y407G | | AVEWESNGQPENNYKTTPPVLDSDGSFFLGSKLTV |
| | | | | | DKSRWQQGNVFSCSVMHEALHNHYTQKSLSLSPG |

Underlined amino acids are substitutions relative to the wild-type CH3 domain sequence of SEQ ID
NO: 1.

APPENDIX TABLE G
CH3 domain variant sequences (with the 354/349 CH3 disulfide substitutions)

| CH3 Set | CH3 Set | | CH3 | SEQ | |
|---|---|---|---|---|---|
| Index | Name | Chain | substitutions | ID NO: | Sequence |
| 1 | W-SG | A | S354C | 15 | GQPREPQVYTLPPCRDELTKNQVSLWCLVKGFYPS |
| (354/349) | (354/349) | | T366W | | DIAVEWESNGQPENNYKTTPPVLDSDGSFFLYSKLT |
| | | | | | VDKSRWQQGNVFSCSVMHEALHNHYTQKSLSLSPG |
| | | B | Y349C T366S | 16 | GQPREPQVCTLPPSRDELTKNQVSLSCLVKGFYPSD |
| | | | Y407G | | IAVEWESNGQPENNYKTTPPVLDSDGSFFLGSKLT |
| | | | | | VDKSRWQQGNVFSCSVMHEALHNHYTQKSLSLSPG |
| 2 | V-V | A | S354C T366V | 25 | GQPREPQVYTLPPCRDELTKNQVSLVCLVKGFYPS |
| (354/349) | (354/349) | | | | DIAVEWESNGQPENNYKTTPPVLDSDGSFFLYSKLT |
| | | | | | VDKSRWQQGNVFSCSVMHEALHNHYTQKSLSLSPG |
| | | B | Y349C Y407V | 26 | GQPREPQVCTLPPSRDELTKNQVSLVCLVKGFYPS |
| | | | | | DIAVEWESNGQPENNYKTTPPVLDSDGSFFLVSKL |
| | | | | | TVDKSRWQQGNVFSCSVMHEALHNHYTQKSLSLS |
| | | | | | PG |
| 3 | QR-F | A | S354C | 35 | GQPREPQVYTLPPCRDELTKNQVSLQCLVKGFYPS |
| (354/349) | (354/349) | | T366Q | | DIAVEWESNGQPENNYKTTPPVLDSDGSFFLYSRL |
| | | | K409R | | TVDKSRWQQGNVFSCSVMHEALHNHYTQKSLSLS |
| | | | | | PG |
| | | B | Y349C L368F | 36 | GQPREPQVCTLPPSRDELTKNQVSLTCFVKGFYPSD |
| | | | | | IAVEWESNGQPENNYKTTPPVLDSDGSFFLYSKLTV |
| | | | | | DKSRWQQGNVFSCSVMHEALHNHYTQKSLSLSPG |
| 4 | RG-FG | A | S354C T366R | 45 | GQPREPQVYTLPPCRDELTKNQVSLRCLVKGFYPS |
| (354/349) | (354/349) | | K409G | | DIAVEWESNGQPENNYKTTPPVLDSDGSFFLYSGL |
| | | | | | TVDKSRWQQGNVFSCSVMHEALHNHYTQKSLSLS |
| | | | | | PG |
| | | B | Y349C L368F | 46 | GQPREPQVCTLPPSRDELTKNQVSLTCFVGGFYPS |
| | | | K370G | | DIAVEWESNGQPENNYKTTPPVLDSDGSFFLYSKLT |
| | | | | | VDKSRWQQGNVFSCSVMHEALHNHYTQKSLSLSPG |
| 5 | TL-QL | A | S354C S400T | 55 | GQPREPQVYTLPPCRDELTKNQVSLTCLVKGFYPSD |
| (354/349) | (354/349) | | K409L | | IAVEWESNGQPENNYKTTPPVLDTDGSFFLYSLLTV |
| | | | | | DKSRWQQGNVFSCSVMHEALHNHYTQKSLSLSPG |
| | | B | Y349C | 56 | GQPREPQVCTLPPSRDELTKNQVSLTCLVKGFYPSD |
| | | | D399Q | | IAVEWESNGQPENNYKTTPPVLQSDGSFLLYSKLTV |
| | | | F405L | | DKSRWQQGNVFSCSVMHEALHNHYTQKSLSLSPG |
| 6 | DVG-VSY | A | S354C S364D | 65 | GQPREPQVYTLPPCRDELTKNQVDLTCLVKGFYPS |
| (354/349) | (354/349) | | Y407V | | DIAVEWESNGQPENNYKTTPPVLDSDGSFFLVSGL |
| | | | K409G | | TVDKSRWQQGNVFSCSVMHEALHNHYTQKSLSLS |
| | | | | | PG |
| | | B | Y349C T366V | 66 | GQPREPQVCTLPPSRDELTKNQVSLVCSVYGFYPS |
| | | | L368S K370Y | | DIAVEWESNGQPENNYKTTPPVLDSDGSFFLYSKLT |
| | | | | | VDKSRWQQGNVFSCSVMHEALHNHYTQKSLSLSPG |
| 7 | LWG-SIG | A | S354C S364L | 75 | GQPREPQVYTLPPCRDELTKNQVLLWCLVKGFYPS |
| (354/349) | (354/349) | | T366W | | DIAVEWESNGQPENNYKTTPPVLDSDGSFFLYSGL |
| | | | K409G | | TVDKSRWQQGNVFSCSVMHEALHNHYTQKSLSLS |
| | | | | | PG |
| | | B | Y349C T366S | 76 | GQPREPQVCTLPPSRDELTKNQVSLSCIVKGFYPSD |
| | | | L3681 Y407G | | IAVEWESNGQPENNYKTTPPVLDSDGSFFLGSKLT |
| | | | | | VDKSRWQQGNVFSCSVMHEALHNHYTQKSLSLSPG |
| 8 | WTL- | A | S354C | 85 | GQPREPQVYTLPPCRDELTKNQVSLWCLVKGFYPS |
| (354/349) | SAVQL | | T366W | | DIAVEWESNGQPENNYKTTPPVLDTDGSFFLYSLLT |
| | (354/349) | | S400T K409L | | VDKSRWQQGNVFSCSVMHEALHNHYTQKSLSLSPG |
| | | B | Y349C T366S | 86 | GQPREPQVCTLPPSRDELTKNQVSLSCAVKGFYPS |
| | | | L368A Y407V | | DIAVEWESNGQPENNYKTTPPVLQSDGSFLLVSKL |
| | | | D399Q | | TVDKSRWQQGNVFSCSVMHEALHNHYTQKSLSLS |
| | | | F405L | | PG |
| 9 | WTL- | A | S354C | 95 | GQPREPQVYTLPPCRDELTKNQVSLWCLVKGFYPS |
| (354/349) | SGQL | | T366W | | DIAVEWESNGQPENNYKTTPPVLDTDGSFFLYSLLT |
| | (354/349) | | S400T K409L | | VDKSRWQQGNVFSCSVMHEALHNHYTQKSLSLSPG |
| | | B | Y349C T366S | 96 | GQPREPQVCTLPPSRDELTKNQVSLSCLVKGFYPSD |
| | | | Y407G | | IAVEWESNGQPENNYKTTPPVLQSDGSFLLGSKLT |
| | | | D399Q | | VDKSRWQQGNVFSCSVMHEALHNHYTQKSLSLSP |
| | | | F405L | | G |
| 10 | WQL- | A | S354C | 105 | GQPREPQVYTLPPCRDELTKNQVSLWCLVKGFYPS |
| (354/349) | SAVTL | | T366W | | DIAVEWESNGQPENNYKTTPPVLQSDGSFLLYSKL |
| | (354/349) | | D399Q | | TVDKSRWQQGNVFSCSVMHEALHNHYTQKSLSLS |
| | | | F405L | | PG |
| | | B | Y349C T366S | 106 | GQPREPQVCTLPPSRDELTKNQVSLSCAVKGFYPS |
| | | | L368A Y407V | | DIAVEWESNGQPENNYKTTPPVLDTDGSFFLVSLL |
| | | | S400T K409L | | TVDKSRWQQGNVFSCSVMHEALHNHYTQKSLSLS |
| | | | | | PG |
| 11 | WQL- | A | S354C | 115 | GQPREPQVYTLPPCRDELTKNQVSLWCLVKGFYPS |
| (354/349) | SGTL | | T366W | | DIAVEWESNGQPENNYKTTPPVLQSDGSFLLYSKL |
| | (354/349) | | D399Q | | TVDKSRWQQGNVFSCSVMHEALHNHYTQKSLSLS |
| | | | F405L | | PG |
| | | B | Y349C T366S | 116 | GQPREPQVCTLPPSRDELTKNQVSLSCLVKGFYPSD |
| | | | Y407G S400T | | IAVEWESNGQPENNYKTTPPVLDTDGSFFLGSLLT |
| | | | K409L | | VDKSRWQQGNVFSCSVMHEALHNHYTQKSLSLSPG |
| 12 | VTL-VQL | A | S354C Y407V | 125 | GQPREPQVYTLPPCRDELTKNQVSLTCLVKGFYPSD |
| (354/349) | (354/349) | | S400T K409L | | IAVEWESNGQPENNYKTTPPVLDTDGSFFLVSLLTV |
| | | | | | DKSRWQQGNVFSCSVMHEALHNHYTQKSLSLSPG |
| | | B | Y349C T366V | 126 | GQPREPQVCTLPPSRDELTKNQVSLVCLVKGFYPS |
| | | | D399Q | | DIAVEWESNGQPENNYKTTPPVLQSDGSFLLYSKL |
| | | | F405L | | TVDKSRWQQGNVFSCSVMHEALHNHYTQKSLSLS |
| | | | | | PG |
| 13 | VQL-VTL | A | S354C Y407V | 135 | GQPREPQVYTLPPCRDELTKNQVSLTCLVKGFYPSD |
| (354/349) | (354/349) | | D399Q | | IAVEWESNGQPENNYKTTPPVLQSDGSFLLVSKLT |
| | | | F405L | | VDKSRWQQGNVFSCSVMHEALHNHYTQKSLSLSPG |
| | | B | Y349C T366V | 136 | GQPREPQVCTLPPSRDELTKNQVSLVCLVKGFYPS |
| | | | S400T K409L | | DIAVEWESNGQPENNYKTTPPVLDTDGSFFLYSLLT |
| | | | | | VDKSRWQQGNVFSCSVMHEALHNHYTQKSLSLSPG |
| 14 | QRQL-FTL | A | S354C | 145 | GQPREPQVYTLPPCRDELTKNQVSLQCLVKGFYPS |
| (354/349) | (354/349) | | T366Q | | DIAVEWESNGQPENNYKTTPPVLQSDGSFLLYSRL |
| | | | K409R | | TVDKSRWQQGNVFSCSVMHEALHNHYTQKSLSLS |
| | | | D399Q | | PG |
| | | | F405L | | |
| | | B | Y349C L368F | 146 | GQPREPQVCTLPPSRDELTKNQVSLTCFVKGFYPSD |
| | | | S400T K409L | | IAVEWESNGQPENNYKTTPPVLDTDGSFFLYSLLTV |
| | | | | | DKSRWQQGNVFSCSVMHEALHNHYTQKSLSLSPG |
| 15 | VQR-VF | A | S354C Y407V | 155 | GQPREPQVYTLPPCRDELTKNQVSLQCLVKGFYPS |
| (354/349) | (354/349) | | T366Q | | DIAVEWESNGQPENNYKTTPPVLDSDGSFFLVSRL |
| | | | K409R | | TVDKSRWQQGNVFSCSVMHEALHNHYTQKSLSLS |
| | | | | | PG |
| | | B | Y349C T366V | 156 | GQPREPQVCTLPPSRDELTKNQVSLVCFVKGFYPS |
| | | | L368F | | DIAVEWESNGQPENNYKTTPPVLDSDGSFFLYSKLT |
| | | | | | VDKSRWQQGNVFSCSVMHEALHNHYTQKSLSLSPG |
| 16 | LWG-IG | A | S354C S364L | 165 | GQPREPQVYTLPPCRDELTKNQVLLWCLVKGFYPS |
| (354/349) | (354/349) | | T366W | | DIAVEWESNGQPENNYKTTPPVLDSDGSFFLYSGL |
| | | | K409G | | TVDKSRWQQGNVFSCSVMHEALHNHYTQKSLSLS |
| | | | | | PG |
| | | B | Y349C L3681 | 166 | GQPREPQVCTLPPSRDELTKNQVSLTCIVKGFYPSD |
| | | | Y407G | | IAVEWESNGQPENNYKTTPPVLDSDGSFFLGSKLT |
| | | | | | VDKSRWQQGNVFSCSVMHEALHNHYTQKSLSLSPG |

Underlined amino acids are substitutions relative to the wild-type CH3 domain sequence of SEQ ID
NO: 1.

What is claimed is:

1. A method of producing a heteromeric molecule, wherein the heteromeric molecule comprises:
(A) a first polypeptide comprising a first variant CH3 domain polypeptide, the first variant CH3 domain polypeptide comprising a T366V substitution, according to EU numbering; and
(B) a second polypeptide comprising a second variant CH3 domain polypeptide, the second variant CH3 domain polypeptide comprising a Y407V substitution according to EU numbering;
wherein the first polypeptide and the second polypeptide are bound to or paired with each other optionally via at least one disulfide bond,
the method comprising:
(i) incubating in a reducing environment (i-1) a first parent molecule comprising at least two of the first polypeptides bound to or paired with each other optionally via at least one disulfide bond and (i-2) a second parent molecule comprising at least two of the second polypeptides bound to or paired with each other optionally via at least one disulfide bond;
(ii) placing the incubation product of step (i) in a less reducing or non-reducing environment, thereby forming the heteromeric molecule;
optionally wherein:
(a) the first variant CH3 domain polypeptide is derived from a CH3 domain of a human IgG and/or the second variant CH3 domain polypeptide is derived from a CH3 domain of a human IgG, optionally wherein the T366V substitution is relative to a CH3 domain of a human IgG and/or the Y407V substitution is relative to a CH3 domain of a human IgG;
(b) the first variant CH3 domain polypeptide is derived from a CH3 domain of a human IgG1 and/or the second variant CH3 domain polypeptide is derived from a CH3 domain of a human IgG1, optionally wherein the T366V substitution is relative to SEQ ID NO: 1, 2, 3, or 4 and/or the Y407V substitution is relative to SEQ ID NO: 1, 2, 3, or 4;
(c) the first variant CH3 domain polypeptide is derived from a CH3 domain of a human IgG2 and/or the second variant CH3 domain polypeptide is derived from a CH3 domain of a human IgG2, optionally wherein the T366V substitution is relative to SEQ ID NO: 722 and/or the Y407V substitution is relative to SEQ ID NO: 722;
(d) the first variant CH3 domain polypeptide is derived from a CH3 domain of a human IgG3 and/or the second variant CH3 domain polypeptide is derived from a CH3 domain of a human IgG3, optionally wherein the T366V substitution is relative to SEQ ID NO: 723 and/or the Y407V substitution is relative to SEQ ID NO: 723; and/or
(e) the first variant CH3 domain polypeptide is derived from a CH3 domain of a human IgG4 and/or the second variant CH3 domain polypeptide is derived from a CH3 domain of a human IgG4, optionally wherein the T366V substitution is relative to SEQ ID NO: 724 and/or the Y407V substitution is relative to SEQ ID NO: 724.
and optionally wherein the heteromeric molecule comprises one or more of the following features:
(A) the first polypeptide further comprises a first antigen-binding domain;
(B) the second polypeptide further comprises a second antigen-binding domain;
(C) the heteromeric molecule further comprises a third polypeptide optionally comprising a third antigen-binding domain, optionally wherein the third polypeptide is bound to or paired with the first polypeptide; and/or
(D) the heteromeric molecule further comprises a fourth polypeptide optionally comprising a fourth antigen-binding domain, optionally wherein the fourth polypeptide is bound to or paired with the second polypeptide,
further optionally wherein the heteromeric molecule is a multi-specific antibody or antigen-binding antibody fragment and optionally comprises a structure depicted in any one of FIGS. 2-8; optionally wherein the heteromeric molecule comprises (a) an IgG or (b) an IgG and one or more scFvs conjugated to the IgG, further optionally comprising IgG1, IgG2, IgG3 or IgG4 constant regions.

2. The method of claim 1, which comprises one or more of the following features:
(I) (I-1-i) the first polypeptide comprises a first antigen-binding domain which forms a first antigen-binding site specific for a first epitope and/or (I-1-ii) the heteromeric molecule comprises a third polypeptide comprising a third antigen-binding domain which forms a third antigen-binding site specific for a third epitope, optionally wherein the first epitope is the same as or different from the third epitope; or
(I-2) the first polypeptide comprises a first antigen-binding domain and the heteromeric molecule comprises a third polypeptide comprising a third antigen-binding domain, wherein the first antigen-binding domain and the third antigen-binding domain form a first antigen-binding site specific for a first epitope; and/or
(II) (II-1-i) the second polypeptide comprises a second antigen-binding domain which forms a second antigen-binding site specific for a second epitope and/or (I-1-ii) the heteromeric molecule comprises a fourth polypeptide comprising a fourth antigen-binding domain which forms a fourth antigen-binding site specific for a fourth epitope, optionally wherein the second epitope is the same as or different from the fourth epitope; or
(II-2) the second polypeptide comprises a second antigen-binding domain and the heteromeric molecule comprises a fourth polypeptide comprising a fourth antigen-binding domain, wherein the second antigen-binding domain and the fourth antigen-binding domain form a second antigen-binding site specific for a second epitope.

3. The method of claim 1, wherein the step (i) comprises one or more of the following features:
(a) the incubating is performed at a temperature between about 15° C. and about 40° C., between about 20° C. and about 40° C., between about 25° C. and about 35° C., between about 28° C. and about 32° C., or between about 29° C. and about 31° C., or at about 30° C.;
(b) the incubating is performed for about 30 minutes to about 20 hours, for about 1 hour to about 15 hours, for about 2 hours to about 10 hours, for about 3 hours to about 7 hours, or for about 4 hours to about 6 hours, or for about 5 hours;
(c) the incubating is performed at about 30° C. for about 5 hours;
(d) the reducing environment comprises at least one reducing agent, optionally at least one mildly reducing agent;
(e) the reducing environment comprises at least one reducing agent selected from 2-mercaptoethylamine (2-MEA), b-mercapto-ethanol (BME), L-cysteine, dithiothreitol (DTT), or dithionite;
(f) the reducing environment comprises at least one reducing agent selected from:
about 25 to about 125 mM, about 50 mM to about 100 mM, about 70 to about 80 mM, or about 75 mM of 2-MEA,
about 20 to about 500 μM, about 40 to about 250 μM, about 80 to about 150 μM, about 90 to about 120 μM, or about 100 μM of BME,
about 20 to about 500 μM, about 40 to about 250 μM, about 80 to about 150 μM,
about 90 to about 120 μM, or about 100 μM of L-cysteine,
about 15 to about 400 μM, about 20 to about 200 μM, about 25 to about 100 μM,
about 30 to about 70 μM, or about 50 μM of DTT, or
about 20 to about 500 μM, about 40 to about 250 μM, about 80 to about 150 μM,
about 90 to about 120 μM, or about 100 μM of dithionite;
(g) the reducing environment comprises at least 2-MEA, optionally at about 75 mM;
(h) the at least two of the first polypeptides are bound to or paired with each other via at least one disulfide bond and/or the at least two of the second polypeptides are bound to or paired with each other via at least one disulfide bond;
(i) the first antibody and/or the second antibody is/are produced in a mammalian cell, a yeast cell, an insect cell, a plant cell, or a bacterial cell; and/or
(j) the first antibody and/or the second antibody is/are produced in a Chinese hamster ovary (CHO) cell or a Human embryonic kidney (HEK) cell.

4. The method of claim 1, wherein the step (ii) comprises one or more of the following features:
(a) the placing is performed by buffer exchange optionally into phosphate buffered saline (PBS);
(b) the placing is performed by buffer exchange via desalting optionally into PBS;
(c) the placing is performed by buffer exchange via diafiltration optionally into PBS; and/or
(d) the placing is performed by addition of an oxidizing agent.

5. The method of claim 1, further comprising:
(iii) incubating the product of step (ii) in the less reducing or non-reducing environment, optionally at a temperature between about 1° C. and about 20° C., between about 2° C. and about 10° C., between about 3° C. and about 5° C., or at about 4° C., optionally for about 12 hour to about 154 hours, for about 24 hours to about 96 hours, for about 36 hours to about 72 hours, or for about 48 hours; and/or
(iv) analyzing the amount of the multi-specific antibody or antigen-binding antibody fragment in the product of step (ii) and/or step (iii) and/or purifying the multi-specific antibody or antigen-binding antibody fragment from the product of step (ii) and/or step (iii), optionally wherein the analyzing and/or purifying is performed via chromatography, optionally LC-MS, IEX, and/or SEC.

6. The method of claim 1, wherein the heteromeric molecule comprises a multi-specific antibody.

7. The method of claim 6, wherein the first polypeptide comprises a first antibody heavy chain and the second polypeptide comprises a second antibody heavy chain, and wherein the first antibody heavy chain is associated with a first antibody light chain and the second antibody heavy chain is associated with a second antibody light chain.

8. The method of claim 7, wherein the multi-specific antibody comprises a third polypeptide comprising a third antigen-binding domain and/or a fourth polypeptide comprising a fourth antigen-binding domain.

9. The method of claim 8, wherein the third antigen-binding domain is associated with the first antibody heavy chain, the second antibody heavy chain, the first antibody light chain, or the second antibody light chain and/or the fourth antigen-binding domain is associated with the first antibody heavy chain, the second antibody heavy chain, the first antibody light chain, or the second antibody light chain.

10. The method of claim 9, where association of the third antigen binding domain and/or the association of the fourth antigen-binding domain comprises a linker, optionally a flexible linker, further optionally wherein the flexible linker comprises or consists of:
(i) the amino acid sequence selected from the group consisting of GGGGS (SEQ ID NO: 715), GGGS (SEQ ID NO: 716), GGGGGS (SEQ ID NO: 717), G, GG, GGG, GS, SG, GGS, GSG, SGG, GSS, SGS, and SSG;
(ii) multiple repeats, optionally two, three, four, or five repeats, of the amino acid sequence selected from the group consisting of SEQ ID NO: 715, SEQ ID NO: 716, SEQ ID NO: 717, G, GG, GGG, GS, SG, GGS, GSG, SGG, GSS, SGS, and SSG;
(iii) a (G5S)n linker, a (G4S)n linker, a (G3S)n linker, a (G2S)n linker, a (GS)n linker, or a (G)n linker, wherein n is a natural number, optionally selected from 1-20, further optionally 2, 3, 4, or 5; and/or
(iv) the amino acid sequence of GGGGSGGGGS (SEQ ID NO: 718) or GGGGSGGGGSGGGGS (SEQ ID NO: 719).

11. The method of claim 8, wherein the third antigen-binding domain and/or the fourth antigen-binding domain comprises a Fab or single chain Fv (scFv).

12. The method of claim 11, wherein the third antigen-binding domain comprises a scFv, wherein the scFv comprises a heavy chain variable domain and a light chain variable domain, wherein the heavy chain variable domain and light chain variable domain are linked by a disulfide bond and/or a linker, optionally a flexible linker, further optionally wherein the linker comprises or consists of:
(i) the amino acid sequence selected from the group consisting of GGGGS (SEQ ID NO: 715), GGGS (SEQ ID NO: 716), GGGGGS (SEQ ID NO: 717), G, GG, GGG, GS, SG, GGS, GSG, SGG, GSS, SGS, and SSG;
(ii) multiple repeats, optionally two, three, four, or five repeats, of the amino acid sequence selected from the group consisting of SEQ ID NO: 715, SEQ ID NO: 716, SEQ ID NO: 717, G, GG, GGG, GS, SG, GGS, GSG, SGG, GSS, SGS, and SSG;
(iii) a (G5S)n linker, a (G4S)n linker, a (G3S)n linker, a (G2S)n linker, a (GS)n linker, or a (G)n linker, wherein n is a natural number, optionally selected from 1-20, further optionally 2, 3, 4, or 5; and/or
(iv) the amino acid sequence of GGGGSGGGGS (SEQ ID NO: 718) or GGGGSGGGGSGGGGS (SEQ ID NO: 719).

13. The method of claim 6, wherein multi-specific antibody comprises a biparatopic antibody.

14. The method of claim 1, wherein the first parent molecule comprises a first IgG and the second parent molecule comprises a second IgG.

15. The method of claim 14, wherein:
each of the at least two of the first polypeptides of the first IgG comprise a first antibody heavy chain comprising a first antigen-binding domain which forms a first antigen-binding site for a first epitope; and
each of the at least two of the second polypeptides of the second IgG comprise a second antibody heavy chain comprising a second antigen binding domain which forms a second antigen-binding site for a second epitope.

16. The method of claim 15, wherein the first epitope and the second epitope are part of different antigens.

17. The method of claim 15, wherein the first epitope and the second epitope are part of the same antigen.

18. The method of claim 15, wherein the heteromeric molecule comprises an IgG comprising the first antibody heavy chain and the second antibody heavy chain.

19. The method of claim 1, wherein:
(A) (I) (i) said T366V substitution is the only substitution in the first variant CH3 domain polypeptide, optionally relative to a CH3 domain of a human IgG, further
optionally relative to:
(i-1) a CH3 domain of a human IgG1, optionally the amino acid sequence of SEQ ID NO: 1, 2, 3, or 4;
(i-2) a CH3 domain of a human IgG2, optionally the amino acid sequence of SEQ ID NO: 722;
(i-3) a CH3 domain of a human IgG3, optionally the amino acid sequence of SEQ ID NO: 723; or
(i-4) a CH3 domain of a human IgG4, optionally the amino acid sequence of SEQ ID NO: 724; and/or
(ii) said Y407V substitution is the only substitution in the second variant CH3 domain polypeptide, optionally relative to a CH3 domain of a human IgG, further optionally relative to:
(i-1) a CH3 domain of a human IgG1, optionally the amino acid sequence of SEQ ID NO: 1, 2, 3, or 4;
(i-2) a CH3 domain of a human IgG2, optionally the amino acid sequence of SEQ ID NO: 722;
(i-3) a CH3 domain of a human IgG3, optionally the amino acid sequence of SEQ ID NO: 723; or
(i-4) a CH3 domain of a human IgG4, optionally the amino acid sequence of SEQ ID NO: 724; or
(II) the first and second variant CH3 domain polypeptides are further modified to comprise one or more variant CH3 domain sets, optionally any of the variant CH3 domain sets described in the specification, further optionally any of the variant CH3 domain sets described in any of the Tables; and/or
(B) the heteromeric molecule comprises one or more CH2 domains, optionally wherein one or more of said CH2 domains comprise one or more amino acid modifications, optionally wherein the one or more amino acid modifications comprise or consist or:
(a) one or more Fc-silencing modifications;
(b) one or more FcRn affinity-enhancing and/or half-life-extending modifications; and/or
(c) any of the following modifications, according to EU numbering: L234A, L235A, and P329A substitutions; L234A, L235A, and P329G substitutions; L234A and L235A substitutions; D265A and P329A substitutions; N297A substitution; M252Y, S254T, and T256E substitutions; and/or M428L and N434S substitutions.

20. A heteromeric molecule produced by the method of claim 1, optionally wherein the heteromeric molecule is a multi-specific antibody or antigen-binding antibody fragment, further optionally wherein the multi-specific antibody or antigen-binding antibody fragment comprises a structure depicted in any one of FIGS. 2-8 and/or an IgG, further optionally an IgG1, IgG2, IgG3 or IgG4.


SEQUENCE LISTING (724 sequences)

SEQ ID NO: 1 - protein, 106 aa, synthetic construct [sequence omitted]

SEQ ID NO: 2 - protein, 106 aa, Homo sapiens
  source 1..106: note = Alternative CH3 domain reference sequence, 356E and 358M
GQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 3 - protein, 106 aa, Homo sapiens
  source 1..106: note = Alternative CH3 domain reference sequence, 422I
GQPREPQVYTLPPSRDELTKNQVSLTCLVKGFYPSDIAVEWESNGQPENNYKTTPPVLDSDGSFFLYSKLTVDKSRWQQGNIFSCSVMHEALHNHYTQKSLSLSPG

SEQ ID NO: 4 - protein, 106 aa, Homo sapiens
  source 1..106: note = Alternative CH3 domain reference sequence, 431G
GQPREPQVYTLPPSRDELTKNQVSLTCLVKGFYPSDIAVEWESNGQPENNYKTTPPVLDSDGSFFLYSKLTVDKSRWQQGNVFSCSVMHEGLHNHYTQKSLSLSPG

SEQ ID NO: 5 - protein, 103 aa, Homo sapiens
  source 1..103: note = Alternative CH1 domain reference sequence, 214R
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSC

SEQ ID NO: 6 - protein, 103 aa, Homo sapiens
  source 1..103: note = CH1 domain reference sequence
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 7 - protein, 110 aa, Homo sapiens
  source 1..110: note = CH2 domain reference sequence
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 8 - protein, 107 aa, Homo sapiens [sequence omitted]

SEQ ID NO: 9 - protein, 106 aa, Homo sapiens
  source 1..106: note = CL lambda domain reference sequence
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 10 - skipped

SEQ ID NO: 11 - protein, 106 aa, synthetic construct
  REGION 1..106: note = CH3 domain variant sequence
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 12 - protein, 106 aa, synthetic construct
  REGION 1..106: note = CH3 domain variant sequence
GQPREPQVYTLPPSRDELTKNQVSLSCLVKGFYPSDIAVEWESNGQPENNYKTTPPVLDSDGSFFLGSKLTVDKSRWQQGNVFSCSVMHEALHNHYTQKSLSLSPG

SEQ ID NO: 13 - protein, 106 aa, synthetic construct
  REGION 1..106: note = CH3 domain variant sequence
GQPREPQVCTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 106 aa, synthetic construct
  REGION 1..106: note = CH3 domain variant sequence
GQPREPQVYTLPPCRDELTKNQVSLSCLVKGFYPSDIAVEWESNGQPENNYKTTPPVLDSDGSFFLGSKLTVDKSRWQQGNVFSCSVMHEALHNHYTQKSLSLSPG

SEQ ID NO: 15 - protein, 106 aa, synthetic construct
  REGION 1..106: note = CH3 domain variant sequence
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 106 aa, synthetic construct
  REGION 1..106: note = CH3 domain variant sequence
GQPREPQVCTLPPSRDELTKNQVSLSCLVKGFYPSDIAVEWESNGQPENNYKTTPPVLDSDGSFFLGSKLTVDKSRWQQGNVFSCSVMHEALHNHYTQKSLSLSPG

SEQ ID NO: 17-20 - skipped (4 entries)

SEQ ID NO: 21 - protein, 106 aa, synthetic construct
  REGION 1..106: note = CH3 domain variant sequence
GQPREPQVYTLPPSRDELTKNQVSLVCLVKGFYPSDIAVEWESNGQPENNYKTTPPVLDSDGSFFLYSKLTVDKSRWQQGNVFSCSVMHEALHNHYTQKSLSLSPG

SEQ ID NO: 22 - protein, 106 aa, synthetic construct
  REGION 1..106: note = CH3 domain variant sequence
GQPREPQVYTLPPSRDELTKNQVSLVCLVKGFYPSDIAVEWESNGQPENNYKTTPPVLDSDGSFFLVSKLTVDKSRWQQGNVFSCSVMHEALHNHYTQKSLSLSPG

SEQ ID NO: 23 - protein, 106 aa, synthetic construct
  REGION 1..106: note = CH3 domain variant sequence
GQPREPQVCTLPPSRDELTKNQVSLVCLVKGFYPSDIAVEWESNGQPENNYKTTPPVLDSDGSFFLYSKLTVDKSRWQQGNVFSCSVMHEALHNHYTQKSLSLSPG

SEQ ID NO: 24 - protein, 106 aa, synthetic construct
  REGION 1..106: note = CH3 domain variant sequence
GQPREPQVYTLPPCRDELTKNQVSLVCLVKGFYPSDIAVEWESNGQPENNYKTTPPVLDSDGSFFLVSKLTVDKSRWQQGNVFSCSVMHEALHNHYTQKSLSLSPG

SEQ ID NO: 25 - protein, 106 aa, synthetic construct
  REGION 1..106: note = CH3 domain variant sequence
GQPREPQVYTLPPCRDELTKNQVSLVCLVKGFYPSDIAVEWESNGQPENNYKTTPPVLDSDGSFFLYSKLTVDKSRWQQGNVFSCSVMHEALHNHYTQKSLSLSPG

SEQ ID NO: 26 - protein, 106 aa, synthetic construct
  REGION 1..106: note = CH3 domain variant sequence
GQPREPQVCTLPPSRDELTKNQVSLVCLVKGFYPSDIAVEWESNGQPENNYKTTPPVLDSDGSFFLVSKLTVDKSRWQQGNVFSCSVMHEALHNHYTQKSLSLSPG

SEQ ID NO: 27-30 - skipped (4 entries)

SEQ ID NO: 31 - protein, 106 aa, synthetic construct
  REGION 1..106: note = CH3 domain variant sequence
GQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 32 - protein, 106 aa, synthetic construct
  REGION 1..106: note = CH3 domain variant sequence
GQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 33 - protein, 106 aa, synthetic construct
  REGION 1..106: note = CH3 domain variant sequence
GQPREPQVCTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 34 - protein, 106 aa, synthetic construct
  REGION 1..106: note = CH3 domain variant sequence
GQPREPQVYTLPPCRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 35 - protein, 106 aa, synthetic construct
  REGION 1..106: note = CH3 domain variant sequence
GQPREPQVYTLPPCRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 36 - protein, 106 aa, synthetic construct
  REGION 1..106: note = CH3 domain variant sequence
GQPREPQVCTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 37-40 - skipped (4 entries)

SEQ ID NO: 41 - protein, 106 aa, synthetic construct
  REGION 1..106: note = CH3 domain variant sequence
GQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 42 - protein, 106 aa, synthetic construct
  REGION 1..106: note = CH3 domain variant sequence
GQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 43 - protein, 106 aa, synthetic construct
  REGION 1..106: note = CH3 domain variant sequence
GQPREPQVCTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 44 - protein, 106 aa, synthetic construct
  REGION 1..106: note = CH3 domain variant sequence
GQPREPQVYTLPPCRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 106 aa, synthetic construct
  REGION 1..106: note = CH3 domain variant sequence
GQPREPQVYTLPPCRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 46 - protein, 106 aa, synthetic construct
  REGION 1..106: note = CH3 domain variant sequence
GQPREPQVCTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 47-50 - skipped (4 entries)

SEQ ID NO: 51 - protein, 106 aa, synthetic construct
  REGION 1..106: note = CH3 domain variant sequence
GQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 52 - protein, 106 aa, synthetic construct
  REGION 1..106: note = CH3 domain variant sequence
GQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 53 - protein, 106 aa, synthetic construct
  REGION 1..106: note = CH3 domain variant sequence
GQPREPQVCTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 54 - protein, 106 aa, synthetic construct
  REGION 1..106: note = CH3 domain variant sequence
GQPREPQVYTLPPCRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 55 - protein, 106 aa, synthetic construct
  REGION 1..106: note = CH3 domain variant sequence
GQPREPQVYTLPPCRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 56 - protein, 106 aa, synthetic construct
  REGION 1..106: note = CH3 domain variant sequence
GQPREPQVCTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 57-60 - skipped (4 entries)

SEQ ID NO: 61 - protein, 106 aa, synthetic construct
  REGION 1..106: note = CH3 domain variant sequence
GQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 62 - protein, 106 aa, synthetic construct
  REGION 1..106: note = CH3 domain variant sequence
GQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 63 - protein, 106 aa, synthetic construct
  REGION 1..106: note = CH3 domain variant sequence
GQPREPQVCTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 64 - protein, 106 aa, synthetic construct
  REGION 1..106: note = CH3 domain variant sequence
GQPREPQVYTLPPCRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 65 - protein, 106 aa, synthetic construct
  REGION 1..106: note = CH3 domain variant sequence
GQPREPQVYTLPPCRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 66 - protein, 106 aa, synthetic construct
  REGION 1..106: note = CH3 domain variant sequence
GQPREPQVCTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 67-70 - skipped (4 entries)

SEQ ID NO: 71 - protein, 106 aa, synthetic construct
  REGION 1..106: note = CH3 domain variant sequence
GQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 72 - protein, 106 aa, synthetic construct
  REGION 1..106: note = CH3 domain variant sequence
GQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 73 - protein, 106 aa, synthetic construct
  REGION 1..106: note = CH3 domain variant sequence
GQPREPQVCTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 74 - protein, 106 aa, synthetic construct
  REGION 1..106: note = CH3 domain variant sequence
GQPREPQVYTLPPCRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 75 - protein, 106 aa, synthetic construct
  REGION 1..106: note = CH3 domain variant sequence
GQPREPQVYTLPPCRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 76 - protein, 106 aa, synthetic construct
  REGION 1..106: note = CH3 domain variant sequence
GQPREPQVCTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 77-80 - skipped (4 entries)

SEQ ID NO: 81 - protein, 106 aa, synthetic construct
  REGION 1..106: note = CH3 domain variant sequence
GQPREPQVYTLPPSRDELTKNQVSLWCLVKGFYPSDIAVEWESNGQPENNYKTTPPVLDTDGSFFLYSLLTVDKSRWQQGNVFSCSVMHEALHNHYTQKSLSLSPG

SEQ ID NO: 82 - protein, 106 aa, synthetic construct
  REGION 1..106: note = CH3 domain variant sequence
GQPREPQVYTLPPSRDELTKNQVSLSCAVKGFYPSDIAVEWESNGQPENNYKTTPPVLQSDGSFLLVSKLTVDKSRWQQGNVFSCSVMHEALHNHYTQKSLSLSPG

SEQ ID NO: 83 - protein, 106 aa, synthetic construct
  REGION 1..106: note = CH3 domain variant sequence
GQPREPQVCTLPPSRDELTKNQVSLWCLVKGFYPSDIAVEWESNGQPENNYKTTPPVLDTDGSFFLYSLLTVDKSRWQQGNVFSCSVMHEALHNHYTQKSLSLSPG

SEQ ID NO: 84 - protein, 106 aa, synthetic construct
  REGION 1..106: note = CH3 domain variant sequence
GQPREPQVYTLPPCRDELTKNQVSLSCAVKGFYPSDIAVEWESNGQPENNYKTTPPVLQSDGSFLLVSKLTVDKSRWQQGNVFSCSVMHEALHNHYTQKSLSLSPG

SEQ ID NO: 85 - protein, 106 aa, synthetic construct
  REGION 1..106: note = CH3 domain variant sequence
GQPREPQVYTLPPCRDELTKNQVSLWCLVKGFYPSDIAVEWESNGQPENNYKTTPPVLDTDGSFFLYSLLTVDKSRWQQGNVFSCSVMHEALHNHYTQKSLSLSPG

SEQ ID NO: 86 - protein, 106 aa, synthetic construct
  REGION 1..106: note = CH3 domain variant sequence
GQPREPQVCTLPPSRDELTKNQVSLSCAVKGFYPSDIAVEWESNGQPENNYKTTPPVLQSDGSFLLVSKLTVDKSRWQQGNVFSCSVMHEALHNHYTQKSLSLSPG

SEQ ID NO: 87-90 - skipped (4 entries)

SEQ ID NO: 91 - protein, 106 aa, synthetic construct; identical to SEQ ID NO: 81
  REGION 1..106: note = CH3 domain variant sequence

SEQ ID NO: 92 - protein, 106 aa, synthetic construct
  REGION 1..106: note = CH3 domain variant sequence
GQPREPQVYTLPPSRDELTKNQVSLSCLVKGFYPSDIAVEWESNGQPENNYKTTPPVLQSDGSFLLGSKLTVDKSRWQQGNVFSCSVMHEALHNHYTQKSLSLSPG

SEQ ID NO: 93 - protein, 106 aa, synthetic construct; identical to SEQ ID NO: 83
  REGION 1..106: note = CH3 domain variant sequence

SEQ ID NO: 94 - protein, 106 aa, synthetic construct
  REGION 1..106: note = CH3 domain variant sequence
GQPREPQVYTLPPCRDELTKNQVSLSCLVKGFYPSDIAVEWESNGQPENNYKTTPPVLQSDGSFLLGSKLTVDKSRWQQGNVFSCSVMHEALHNHYTQKSLSLSPG

SEQ ID NO: 95 - protein, 106 aa, synthetic construct; identical to SEQ ID NO: 85
  REGION 1..106: note = CH3 domain variant sequence

SEQ ID NO: 96 - protein, 106 aa, synthetic construct
  REGION 1..106: note = CH3 domain variant sequence
GQPREPQVCTLPPSRDELTKNQVSLSCLVKGFYPSDIAVEWESNGQPENNYKTTPPVLQSDGSFLLGSKLTVDKSRWQQGNVFSCSVMHEALHNHYTQKSLSLSPG

SEQ ID NO: 97-100 - skipped (4 entries)

SEQ ID NO: 101 - protein, 106 aa, synthetic construct
  REGION 1..106: note = CH3 domain variant sequence
GQPREPQVYTLPPSRDELTKNQVSLWCLVKGFYPSDIAVEWESNGQPENNYKTTPPVLQSDGSFLLYSKLTVDKSRWQQGNVFSCSVMHEALHNHYTQKSLSLSPG

SEQ ID NO: 102 - protein, 106 aa, synthetic construct
  REGION 1..106: note = CH3 domain variant sequence
GQPREPQVYTLPPSRDELTKNQVSLSCAVKGFYPSDIAVEWESNGQPENNYKTTPPVLDTDGSFFLVSLLTVDKSRWQQGNVFSCSVMHEALHNHYTQKSLSLSPG

SEQ ID NO: 103 - protein, 106 aa, synthetic construct
  REGION 1..106: note = CH3 domain variant sequence
GQPREPQVCTLPPSRDELTKNQVSLWCLVKGFYPSDIAVEWESNGQPENNYKTTPPVLQSDGSFLLYSKLTVDKSRWQQGNVFSCSVMHEALHNHYTQKSLSLSPG

SEQ ID NO: 104 - protein, 106 aa, synthetic construct
  REGION 1..106: note = CH3 domain variant sequence
GQPREPQVYTLPPCRDELTKNQVSLSCAVKGFYPSDIAVEWESNGQPENNYKTTPPVLDTDGSFFLVSLLTVDKSRWQQGNVFSCSVMHEALHNHYTQKSLSLSPG

SEQ ID NO: 105 - protein, 106 aa, synthetic construct
  REGION 1..106: note = CH3 domain variant sequence
GQPREPQVYTLPPCRDELTKNQVSLWCLVKGFYPSDIAVEWESNGQPENNYKTTPPVLQSDGSFLLYSKLTVDKSRWQQGNVFSCSVMHEALHNHYTQKSLSLSPG

SEQ ID NO: 106 - protein, 106 aa, synthetic construct
  REGION 1..106: note = CH3 domain variant sequence
GQPREPQVCTLPPSRDELTKNQVSLSCAVKGFYPSDIAVEWESNGQPENNYKTTPPVLDTDGSFFLVSLLTVDKSRWQQGNVFSCSVMHEALHNHYTQKSLSLSPG

SEQ ID NO: 107-110 - skipped (4 entries)

SEQ ID NO: 111 - protein, 106 aa, synthetic construct; identical to SEQ ID NO: 101
  REGION 1..106: note = CH3 domain variant sequence

SEQ ID NO: 112 - protein, 106 aa, synthetic construct
  REGION 1..106: note = CH3 domain variant sequence
GQPREPQVYTLPPSRDELTKNQVSLSCLVKGFYPSDIAVEWESNGQPENNYKTTPPVLDTDGSFFLGSLLTVDKSRWQQGNVFSCSVMHEALHNHYTQKSLSLSPG

SEQ ID NO: 113 - protein, 106 aa, synthetic construct; identical to SEQ ID NO: 103
  REGION 1..106: note = CH3 domain variant sequence

SEQ ID NO: 114 - protein, 106 aa, synthetic construct
  REGION 1..106: note = CH3 domain variant sequence
GQPREPQVYTLPPCRDELTKNQVSLSCLVKGFYPSDIAVEWESNGQPENNYKTTPPVLDTDGSFFLGSLLTVDKSRWQQGNVFSCSVMHEALHNHYTQKSLSLSPG

SEQ ID NO: 115 - protein, 106 aa, synthetic construct; identical to SEQ ID NO: 105
  REGION 1..106: note = CH3 domain variant sequence

SEQ ID NO: 116 - protein, 106 aa, synthetic construct
  REGION 1..106: note = CH3 domain variant sequence
GQPREPQVCTLPPSRDELTKNQVSLSCLVKGFYPSDIAVEWESNGQPENNYKTTPPVLDTDGSFFLGSLLTVDKSRWQQGNVFSCSVMHEALHNHYTQKSLSLSPG

SEQ ID NO: 117-120 - skipped (4 entries)

SEQ ID NO: 121 - protein, 106 aa, synthetic construct
  REGION 1..106: note = CH3 domain variant sequence
GQPREPQVYTLPPSRDELTKNQVSLTCLVKGFYPSDIAVEWESNGQPENNYKTTPPVLDTDGSFFLVSLLTVDKSRWQQGNVFSCSVMHEALHNHYTQKSLSLSPG

SEQ ID NO: 122 - protein, 106 aa, synthetic construct
  REGION 1..106: note = CH3 domain variant sequence
GQPREPQVYTLPPSRDELTKNQVSLVCLVKGFYPSDIAVEWESNGQPENNYKTTPPVLQSDGSFLLYSKLTVDKSRWQQGNVFSCSVMHEALHNHYTQKSLSLSPG

SEQ ID NO: 123 - protein, 106 aa, synthetic construct
  REGION 1..106: note = CH3 domain variant sequence
GQPREPQVCTLPPSRDELTKNQVSLTCLVKGFYPSDIAVEWESNGQPENNYKTTPPVLDTDGSFFLVSLLTVDKSRWQQGNVFSCSVMHEALHNHYTQKSLSLSPG

SEQ ID NO: 124 - protein, 106 aa, synthetic construct
  REGION 1..106: note = CH3 domain variant sequence
GQPREPQVYTLPPCRDELTKNQVSLVCLVKGFYPSDIAVEWESNGQPENNYKTTPPVLQSDGSFLLYSKLTVDKSRWQQGNVFSCSVMHEALHNHYTQKSLSLSPG

SEQ ID NO: 125 - protein, 106 aa, synthetic construct
  REGION 1..106: note = CH3 domain variant sequence
GQPREPQVYTLPPCRDELTKNQVSLTCLVKGFYPSDIAVEWESNGQPENNYKTTPPVLDTDGSFFLVSLLTVDKSRWQQGNVFSCSVMHEALHNHYTQKSLSLSPG

SEQ ID NO: 126 - protein, 106 aa, synthetic construct
  REGION 1..106: note = CH3 domain variant sequence
GQPREPQVCTLPPSRDELTKNQVSLVCLVKGFYPSDIAVEWESNGQPENNYKTTPPVLQSDGSFLLYSKLTVDKSRWQQGNVFSCSVMHEALHNHYTQKSLSLSPG

SEQ ID NO: 127-130 - skipped (4 entries)

SEQ ID NO: 131 - protein, 106 aa, synthetic construct
  REGION 1..106: note = CH3 domain variant sequence
GQPREPQVYTLPPSRDELTKNQVSLTCLVKGFYPSDIAVEWESNGQPENNYKTTPPVLQSDGSFLLVSKLTVDKSRWQQGNVFSCSVMHEALHNHYTQKSLSLSPG

SEQ ID NO: 132 - protein, 106 aa, synthetic construct
  REGION 1..106: note = CH3 domain variant sequence
GQPREPQVYTLPPSRDELTKNQVSLVCLVKGFYPSDIAVEWESNGQPENNYKTTPPVLDTDGSFFLYSLLTVDKSRWQQGNVFSCSVMHEALHNHYTQKSLSLSPG

SEQ ID NO: 133 - protein, 106 aa, synthetic construct
  REGION 1..106: note = CH3 domain variant sequence
GQPREPQVCTLPPSRDELTKNQVSLTCLVKGFYPSDIAVEWESNGQPENNYKTTPPVLQSDGSFLLVSKLTVDKSRWQQGNVFSCSVMHEALHNHYTQKSLSLSPG

SEQ ID NO: 134 - protein, 106 aa, synthetic construct
  REGION 1..106: note = CH3 domain variant sequence
GQPREPQVYTLPPCRDELTKNQVSLVCLVKGFYPSDIAVEWESNGQPENNYKTTPPVLDTDGSFFLYSLLTVDKSRWQQGNVFSCSVMHEALHNHYTQKSLSLSPG

SEQ ID NO: 135 - protein, 106 aa, synthetic construct
  REGION 1..106: note = CH3 domain variant sequence
GQPREPQVYTLPPCRDELTKNQVSLTCLVKGFYPSDIAVEWESNGQPENNYKTTPPVLQSDGSFLLVSKLTVDKSRWQQGNVFSCSVMHEALHNHYTQKSLSLSPG

SEQ ID NO: 136 - protein, 106 aa, synthetic construct
  REGION 1..106: note = CH3 domain variant sequence
GQPREPQVCTLPPSRDELTKNQVSLVCLVKGFYPSDIAVEWESNGQPENNYKTTPPVLDTDGSFFLYSLLTVDKSRWQQGNVFSCSVMHEALHNHYTQKSLSLSPG

SEQ ID NO: 137-140 - skipped (4 entries)

SEQ ID NO: 141 - protein, 106 aa, synthetic construct
  REGION 1..106: note = CH3 domain variant sequence
GQPREPQVYTLPPSRDELTKNQVSLQCLVKGFYPSDIAVEWESNGQPENNYKTTPPVLQSDGSFLLYSRLTVDKSRWQQGNVFSCSVMHEALHNHYTQKSLSLSPG

SEQ ID NO: 142 - protein, 106 aa, synthetic construct
  REGION 1..106: note = CH3 domain variant sequence
GQPREPQVYTLPPSRDELTKNQVSLTCFVKGFYPSDIAVEWESNGQPENNYKTTPPVLDTDGSFFLYSLLTVDKSRWQQGNVFSCSVMHEALHNHYTQKSLSLSPG

SEQ ID NO: 143 - protein, 106 aa, synthetic construct
  REGION 1..106: note = CH3 domain variant sequence
GQPREPQVCTLPPSRDELTKNQVSLQCLVKGFYPSDIAVEWESNGQPENNYKTTPPVLQSDGSFLLYSRLTVDKSRWQQGNVFSCSVMHEALHNHYTQKSLSLSPG

SEQ ID NO: 144 - protein, 106 aa, synthetic construct
  REGION 1..106: note = CH3 domain variant sequence
GQPREPQVYTLPPCRDELTKNQVSLTCFVKGFYPSDIAVEWESNGQPENNYKTTPPVLDTDGSFFLYSLLTVDKSRWQQGNVFSCSVMHEALHNHYTQKSLSLSPG

SEQ ID NO: 145 - protein, 106 aa, synthetic construct
  REGION 1..106: note = CH3 domain variant sequence
GQPREPQVYTLPPCRDELTKNQVSLQCLVKGFYPSDIAVEWESNGQPENNYKTTPPVLQSDGSFLLYSRLTVDKSRWQQGNVFSCSVMHEALHNHYTQKSLSLSPG

SEQ ID NO: 146 - protein, 106 aa, synthetic construct
  REGION 1..106: note = CH3 domain variant sequence
GQPREPQVCTLPPSRDELTKNQVSLTCFVKGFYPSDIAVEWESNGQPENNYKTTPPVLDTDGSFFLYSLLTVDKSRWQQGNVFSCSVMHEALHNHYTQKSLSLSPG

SEQ ID NO: 147-150 - skipped (4 entries)

SEQ ID NO: 151 - protein, 106 aa, synthetic construct
  REGION 1..106: note = CH3 domain variant sequence
GQPREPQVYTLPPSRDELTKNQVSLQCLVKGFYPSDIAVEWESNGQPENNYKTTPPVLDSDGSFFLVSRLTVDKSRWQQGNVFSCSVMHEALHNHYTQKSLSLSPG

SEQ ID NO: 152 - protein, 106 aa, synthetic construct
  REGION 1..106: note = CH3 domain variant sequence
GQPREPQVYTLPPSRDELTKNQVSLVCFVKGFYPSDIAVEWESNGQPENNYKTTPPVLDSDGSFFLYSKLTVDKSRWQQGNVFSCSVMHEALHNHYTQKSLSLSPG

SEQ ID NO: 153 - protein, 106 aa, synthetic construct
  REGION 1..106: note = CH3 domain variant sequence
GQPREPQVCTLPPSRDELTKNQVSLQCLVKGFYPSDIAVEWESNGQPENNYKTTPPVLDSDGSFFLVSRLTVDKSRWQQGNVFSCSVMHEALHNHYTQKSLSLSPG

SEQ ID NO: 154 - protein, 106 aa, synthetic construct
  REGION 1..106: note = CH3 domain variant sequence
GQPREPQVYTLPPCRDELTKNQVSLVCFVKGFYPSDIAVEWESNGQPENNYKTTPPVLDSDGSFFLYSKLTVDKSRWQQGNVFSCSVMHEALHNHYTQKSLSLSPG

SEQ ID NO: 155 - protein, 106 aa, synthetic construct
  REGION 1..106: note = CH3 domain variant sequence
GQPREPQVYTLPPCRDELTKNQVSLQCLVKGFYPSDIAVEWESNGQPENNYKTTPPVLDSDGSFFLVSRLTVDKSRWQQGNVFSCSVMHEALHNHYTQKSLSLSPG

SEQ ID NO: 156 - protein, 106 aa, synthetic construct
  REGION 1..106: note = CH3 domain variant sequence
GQPREPQVCTLPPSRDELTKNQVSLVCFVKGFYPSDIAVEWESNGQPENNYKTTPPVLDSDGSFFLYSKLTVDKSRWQQGNVFSCSVMHEALHNHYTQKSLSLSPG

SEQ ID NO: 157-160 - skipped (4 entries)

SEQ ID NO: 161 - protein, 106 aa, synthetic construct; identical to SEQ ID NO: 71
  REGION 1..106: note = CH3 domain variant sequence

SEQ ID NO: 162 - protein, 106 aa, synthetic construct
  REGION 1..106: note = CH3 domain variant sequence
GQPREPQVYTLPPSRDELTKNQVSLTCIVKGFYPSDIAVEWESNGQPENNYKTTPPVLDSDGSFFLGSKLTVDKSRWQQGNVFSCSVMHEALHNHYTQKSLSLSPG

SEQ ID NO: 163 - protein, 106 aa, synthetic construct
  REGION 1..106: note = CH3 domain variant sequence
GQPREPQVYCLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 164 - protein, 106 aa, synthetic construct
  REGION 1..106: note = CH3 domain variant sequence
GQPREPQVYTLPPCRDELTKNQVSLTCIVKGFYPSDIAVEWESNGQPENNYKTTPPVLDSDGSFFLGSKLTVDKSRWQQGNVFSCSVMHEALHNHYTQKSLSLSPG

SEQ ID NO: 165 - protein, 106 aa, synthetic construct; identical to SEQ ID NO: 75
  REGION 1..106: note = CH3 domain variant sequence

SEQ ID NO: 166 - protein, 106 aa, synthetic construct
  REGION 1..106: note = CH3 domain variant sequence
GQPREPQVCTLPPSRDELTKNQVSLTCIVKGFYPSDIAVEWESNGQPENNYKTTPPVLDSDGSFFLGSKLTVDKSRWQQGNVFSCSVMHEALHNHYTQKSLSLSPG

SEQ ID NO: 167-210 - skipped (44 entries)

SEQ ID NO: 211 - protein, 448 aa, synthetic construct
  REGION 1..448: note = Heavy chain - ADI-59166
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 212 - protein, 450 aa, synthetic construct
  REGION 1..450: note = Heavy chain ADI-59166
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 213 - protein, 219 aa, synthetic construct
  REGION 1..219: note = Light chain ADI-59166
DIVMTQSPDSLAVSLGERATINCKSSQSLLNARTGKNYLAWYQQKPGQPPKLLIYWASTRESGVPDRFSGSGSGTDFTLTISSLQAEDVAVYYCKQSYSLRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 214 - protein, 219 aa, synthetic construct; identical to SEQ ID NO: 213
  REGION 1..219: note = Light chain ADI-59166

SEQ ID NO: 215-220 - skipped (6 entries)

SEQ ID NO: 221 - protein, 448 aa, synthetic construct
  REGION 1..448: note = Heavy chain ADI-59167
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 222 - protein, 450 aa, synthetic construct
  REGION 1..450: note = Heavy chain ADI-59167
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 223 - protein, 219 aa, synthetic construct; identical to SEQ ID NO: 213
  REGION 1..219: note = Light chain ADI-59167

SEQ ID NO: 224 - protein, 219 aa, synthetic construct; identical to SEQ ID NO: 213
  REGION 1..219: note = Light chain ADI-59167

SEQ ID NO: 225-230 - skipped (6 entries)

SEQ ID NO: 231 - protein, 448 aa, synthetic construct
  REGION 1..448: note = Heavy chain ADI-59168
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 232 - protein, 450 aa, synthetic construct
  REGION 1..450: note = Heavy chain ADI-59168
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 233 - protein, 219 aa, synthetic construct; identical to SEQ ID NO: 213
  REGION 1..219: note = Light chain ADI-59168

SEQ ID NO: 234 - protein, 219 aa, synthetic construct; identical to SEQ ID NO: 213
  REGION 1..219: note = Light chain ADI-59168

SEQ ID NO: 235-240 - skipped (6 entries)

SEQ ID NO: 241 - protein, 448 aa, synthetic construct
  REGION 1..448: note = Heavy chain ADI-59169
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 242 - protein, 450 aa, synthetic construct
  REGION 1..450: note = Heavy chain ADI-59169
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 243 - protein, 219 aa, synthetic construct; identical to SEQ ID NO: 213
  REGION 1..219: note = Light chain ADI-59169

SEQ ID NO: 244 - protein, 219 aa, synthetic construct; identical to SEQ ID NO: 213
  REGION 1..219: note = Light chain ADI-59169

SEQ ID NO: 245-250 - skipped (6 entries)

SEQ ID NO: 251 - protein, 448 aa, synthetic construct
  REGION 1..448: note = Heavy chain ADI-59170
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 252 - protein, 450 aa, synthetic construct
  REGION 1..450: note = Heavy chain ADI-59170
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 253 - protein, 219 aa, synthetic construct; identical to SEQ ID NO: 213
  REGION 1..219: note = Light chain ADI-59170

SEQ ID NO: 254 - protein, 219 aa, synthetic construct; identical to SEQ ID NO: 213
  REGION 1..219: note = Light chain ADI-59170

SEQ ID NO: 255-260 - skipped (6 entries)

SEQ ID NO: 261 - protein, 448 aa, synthetic construct
  REGION 1..448: note = Heavy chain ADI-59171
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 262 - protein, 450 aa, synthetic construct
  REGION 1..450: note = Heavy chain ADI-59171
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 263 - protein, 219 aa, synthetic construct; identical to SEQ ID NO: 213
  REGION 1..219: note = Light chain ADI-59171

SEQ ID NO: 264 - protein, 219 aa, synthetic construct; identical to SEQ ID NO: 213
  REGION 1..219: note = Light chain ADI-59171

SEQ ID NO: 265-270 - skipped (6 entries)

SEQ ID NO: 271 - protein, 448 aa, synthetic construct
  REGION 1..448: note = Heavy chain ADI-59172
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 272 - protein, 450 aa, synthetic construct
  REGION 1..450: note = Heavy chain ADI-59172
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 273 - protein, 219 aa, synthetic construct; identical to SEQ ID NO: 213
  REGION 1..219: note = Light chain ADI-59172

SEQ ID NO: 274 - protein, 219 aa, synthetic construct; identical to SEQ ID NO: 213
  REGION 1..219: note = Light chain ADI-59172

SEQ ID NO: 275-280 - skipped (6 entries)

SEQ ID NO: 281 - protein, 448 aa, synthetic construct
  REGION 1..448: note = Heavy chain ADI-59173
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 282 - protein, 450 aa, synthetic construct
  REGION 1..450: note = Heavy chain ADI-59173
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 283 - protein, 219 aa, synthetic construct; identical to SEQ ID NO: 213
  REGION 1..219: note = Light chain ADI-59173

SEQ ID NO: 284 - protein, 219 aa, synthetic construct; identical to SEQ ID NO: 213
  REGION 1..219: note = Light chain ADI-59173

SEQ ID NO: 285-290 - skipped (6 entries)

SEQ ID NO: 291 - protein, 448 aa, synthetic construct
  REGION 1..448: note = Heavy Chain ADI-59174
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 292 - protein, 450 aa, synthetic construct
  REGION 1..450: note = Heavy chain ADI-59174
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 293 - protein, 219 aa, synthetic construct; identical to SEQ ID NO: 213
  REGION 1..219: note = Light chain ADI-59174

SEQ ID NO: 294 - protein, 219 aa, synthetic construct; identical to SEQ ID NO: 213
  REGION 1..219: note = Light chain ADI-59174

SEQ ID NO: 295-300 - skipped (6 entries)

SEQ ID NO: 301 - protein, 448 aa, synthetic construct
  REGION 1..448: note = Heavy Chain ADI-59175
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 302 - protein, 450 aa, synthetic construct
  REGION 1..450: note = Heavy chain ADI-59175
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 303 - protein, 219 aa, synthetic construct; identical to SEQ ID NO: 213
  REGION 1..219: note = Light chain ADI-59175

SEQ ID NO: 304 - protein, 219 aa, synthetic construct; identical to SEQ ID NO: 213
  REGION 1..219: note = Light chain ADI-59175

SEQ ID NO: 305-310 - skipped (6 entries)

SEQ ID NO: 311 - protein, 448 aa, synthetic construct
  REGION 1..448: note = Heavy Chain ADI-59176
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 312 - protein, 450 aa, synthetic construct
  REGION 1..450: note = Heavy chain ADI-59176
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 313 - protein, 219 aa, synthetic construct; identical to SEQ ID NO: 213
  REGION 1..219: note = Light chain ADI-59176

SEQ ID NO: 314 - protein, 219 aa, synthetic construct; identical to SEQ ID NO: 213
  REGION 1..219: note = Light chain ADI-59176

SEQ ID NO: 315-320 - skipped (6 entries)

SEQ ID NO: 321 - protein, 448 aa, synthetic construct
  REGION 1..448: note = Heavy chain ADI-59177
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 322 - protein, 450 aa, synthetic construct
  REGION 1..450: note = Heavy chain ADI-59177
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 323 - protein, 219 aa, synthetic construct; identical to SEQ ID NO: 213
  REGION 1..219: note = Light chain ADI-59177

SEQ ID NO: 324 - protein, 219 aa, synthetic construct; identical to SEQ ID NO: 213
  REGION 1..219: note = Light chain ADI-59177

SEQ ID NO: 325-330 - skipped (6 entries)

SEQ ID NO: 331 - protein, 448 aa, synthetic construct
  REGION 1..448: note = Heavy chain ADI-59178
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 332 - protein, 450 aa, synthetic construct
  REGION 1..450: note = Heavy chain ADI-59178
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 333 - protein, 219 aa, synthetic construct; identical to SEQ ID NO: 213
  REGION 1..219: note = Light chain ADI-59178

SEQ ID NO: 334 - protein, 219 aa, synthetic construct; identical to SEQ ID NO: 213
  REGION 1..219: note = Light chain ADI-59178

SEQ ID NO: 335-340 - skipped (6 entries)

SEQ ID NO: 341 - protein, 448 aa, synthetic construct
  REGION 1..448: note = Heavy chain ADI-59179
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 342 - protein, 450 aa, synthetic construct
  REGION 1..450: note = Heavy chain ADI-59179
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 343 - protein, 219 aa, synthetic construct; identical to SEQ ID NO: 213
  REGION 1..219: note = Light chain ADI-59179

SEQ ID NO: 344 - protein, 219 aa, synthetic construct; identical to SEQ ID NO: 213
  REGION 1..219: note = Light chain ADI-59179

SEQ ID NO: 345-350 - skipped (6 entries)

SEQ ID NO: 351 - protein, 448 aa, synthetic construct
  REGION 1..448: note = Heavy chain ADI-59180
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 352 - protein, 450 aa, synthetic construct
  REGION 1..450: note = Heavy chain ADI-59180
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 353 - protein, 219 aa, synthetic construct; identical to SEQ ID NO: 213
  REGION 1..219: note = Light chain ADI-59180

SEQ ID NO: 354 - protein, 219 aa, synthetic construct; identical to SEQ ID NO: 213
  REGION 1..219: note = Light chain ADI-59180

SEQ ID NO: 355-360 - skipped (6 entries)

SEQ ID NO: 361 - protein, 448 aa, synthetic construct
  REGION 1..448: note = Heavy chain ADI-59181
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 362 - protein, 450 aa, synthetic construct
  REGION 1..450: note = Heavy chain ADI-59181
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 363 - protein, 219 aa, synthetic construct; identical to SEQ ID NO: 213
  REGION 1..219: note = Light chain ADI-59181

SEQ ID NO: 364 - protein, 219 aa, synthetic construct; identical to SEQ ID NO: 213
  REGION 1..219: note = Light chain ADI-59181

SEQ ID NO: 365-370 - skipped (6 entries)

SEQ ID NO: 371 - protein, 448 aa, synthetic construct
  REGION 1..448: note = Heavy chain ADI-59182
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPCRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 372 - protein, 450 aa, synthetic construct
  REGION 1..450: note = Heavy chain ADI-59182
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVCTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 373 - protein, 219 aa, synthetic construct; identical to SEQ ID NO: 213
  REGION 1..219: note = Light chain ADI-59182

SEQ ID NO: 374 - protein, 219 aa, synthetic construct; identical to SEQ ID NO: 213
  REGION 1..219: note = Light chain ADI-59182

SEQ ID NO: 375-380 - skipped (6 entries)

SEQ ID NO: 381 - protein, 456 aa, synthetic construct
  REGION 1..456: note = Heavy chain ADI-59183
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 382 - protein, 452 aa, synthetic construct
  REGION 1..452: note = Heavy chain ADI-59183
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 383 - protein, 214 aa, synthetic construct
  REGION 1..214: note = Light chain ADI-59183
EIVMTQSPATLSVSPGERATLSCRASQSVSSNLAWYQQKPGQAPRLLIYGASTRATGIPARFSGSGSGTEFTLTISSLQSEDFAVYYCQHYINWPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 384 - protein, 214 aa, synthetic construct; identical to SEQ ID NO: 383
  REGION 1..214: note = Light chain ADI-59183

SEQ ID NO: 385-390 - skipped (6 entries)

SEQ ID NO: 391 - protein, 456 aa, synthetic construct
  REGION 1..456: note = Heavy chain ADI-59184
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 392 - protein, 452 aa, synthetic construct
  REGION 1..452: note = Heavy chain ADI-59184
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 393 - protein, 214 aa, synthetic construct; identical to SEQ ID NO: 383
  REGION 1..214: note = Light chain ADI-59184

SEQ ID NO: 394 - protein, 214 aa, synthetic construct; identical to SEQ ID NO: 383
  REGION 1..214: note = Light chain ADI-59184

SEQ ID NO: 395-400 - skipped (6 entries)

SEQ ID NO: 401 - protein, 456 aa, synthetic construct
  REGION 1..456: note = Heavy chain ADI-59185
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 402 - protein, 452 aa, synthetic construct
  REGION 1..452: note = Heavy chain ADI-59185
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 403 - protein, 214 aa, synthetic construct; identical to SEQ ID NO: 383
  REGION 1..214: note = Light chain ADI-59185

SEQ ID NO: 404 - protein, 214 aa, synthetic construct; identical to SEQ ID NO: 383
  REGION 1..214: note = Light chain ADI-59185

SEQ ID NO: 405-410 - skipped (6 entries)

SEQ ID NO: 411 - protein, 456 aa, synthetic construct
  REGION 1..456: note = Heavy chain ADI-59186
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 412 - protein, 452 aa, synthetic construct
  REGION 1..452: note = Heavy chain ADI-59186
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 413 - protein, 214 aa, synthetic construct; identical to SEQ ID NO: 383
  REGION 1..214: note = Light chain ADI-59186

SEQ ID NO: 414 - protein, 214 aa, synthetic construct; identical to SEQ ID NO: 383
  REGION 1..214: note = Light chain ADI-59186

SEQ ID NO: 415-420 - skipped (6 entries)

SEQ ID NO: 421 - protein, 456 aa, synthetic construct
  REGION 1..456: note = Heavy chain ADI-59187
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 422 - protein, 452 aa, synthetic construct
  REGION 1..452: note = Heavy chain ADI-59187
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 423 - protein, 214 aa, synthetic construct; identical to SEQ ID NO: 383
  REGION 1..214: note = Light chain ADI-59187

SEQ ID NO: 424 - protein, 214 aa, synthetic construct; identical to SEQ ID NO: 383
  REGION 1..214: note = Light chain ADI-59187

SEQ ID NO: 425-430 - skipped (6 entries)

SEQ ID NO: 431 - protein, 456 aa, synthetic construct
  REGION 1..456: note = Heavy chain ADI-59188
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 432 - protein, 452 aa, synthetic construct
  REGION 1..452: note = Heavy chain ADI-59188
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 433 - protein, 214 aa, synthetic construct; identical to SEQ ID NO: 383
  REGION 1..214: note = Light chain ADI-59188

SEQ ID NO: 434 - protein, 214 aa, synthetic construct; identical to SEQ ID NO: 383
  REGION 1..214: note = Light chain ADI-59188

SEQ ID NO: 435-440 - skipped (6 entries)

SEQ ID NO: 441 - protein, 456 aa, synthetic construct
  REGION 1..456: note = Heavy chain ADI-59189
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 442 - protein, 452 aa, synthetic construct
  REGION 1..452: note = Heavy chain ADI-59189
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 443 - protein, 214 aa, synthetic construct; identical to SEQ ID NO: 383
  REGION 1..214: note = Light chain ADI-59189

SEQ ID NO: 444 - protein, 214 aa, synthetic construct; identical to SEQ ID NO: 383
  REGION 1..214: note = Light chain ADI-59189

SEQ ID NO: 445-450 - skipped (6 entries)

SEQ ID NO: 451 - protein, 456 aa, synthetic construct
  REGION 1..456: note = Heavy chain ADI-59190
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 452 - protein, 452 aa, synthetic construct
  REGION 1..452: note = Heavy chain ADI-59190
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 453 - protein, 214 aa, synthetic construct; identical to SEQ ID NO: 383
  REGION 1..214: note = Light chain ADI-59190

SEQ ID NO: 454 - protein, 214 aa, synthetic construct; identical to SEQ ID NO: 383
  REGION 1..214: note = Light chain ADI-59190

SEQ ID NO: 455-460 - skipped (6 entries)

SEQ ID NO: 461 - protein, 456 aa, synthetic construct
  REGION 1..456: note = Heavy chain ADI-59191
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 462 - protein, 452 aa, synthetic construct
  REGION 1..452: note = Heavy chain ADI-59191
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 463 - protein, 214 aa, synthetic construct; identical to SEQ ID NO: 383
  REGION 1..214: note = Light chain ADI-59191

SEQ ID NO: 464 - protein, 214 aa, synthetic construct; identical to SEQ ID NO: 383
  REGION 1..214: note = Light chain ADI-59191

SEQ ID NO: 465-470 - skipped (6 entries)

SEQ ID NO: 471 - protein, 456 aa, synthetic construct
  REGION 1..456: note = Heavy chain ADI-59192
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 472 - protein, 452 aa, synthetic construct
  REGION 1..452: note = Heavy chain ADI-59192
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 473 - protein, 214 aa, synthetic construct; identical to SEQ ID NO: 383
  REGION 1..214: note = Light chain ADI-59192

SEQ ID NO: 474 - protein, 214 aa, synthetic construct; identical to SEQ ID NO: 383
  REGION 1..214: note = Light chain ADI-59192

SEQ ID NO: 475-480 - skipped (6 entries)

SEQ ID NO: 481 - protein, 456 aa, synthetic construct
  REGION 1..456: note = Heavy chain ADI-59193
EVQLVESGGGLVQPGRSLRLSCVASGFTFNDYAMHWVRQAPGKGLEWVSVISWNSDSIGYADSVKGRFTISRDNAKNSLYLQMHSLRAEDTALYYCAKDNHYGSGSYYYYQYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 482 - protein, 452 aa, synthetic construct
  REGION 1..452: note = Heavy chain ADI-59193
EVQLVESGGGLVQPGRSLRLSCAASGFTFDDYTMHWVRQAPGKGLEWVSGISWNSGSIGYADSVKGRFTISRDNAKKSLYLQMNSLRAEDTALYYCAKDNSGYGHYYYGMDVWGQGTTVTVASASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 483 - protein, 214 aa, synthetic construct; identical to SEQ ID NO: 383
  REGION 1..214: note = Light chain ADI-59193

SEQ ID NO: 484 - protein, 214 aa, synthetic construct; identical to SEQ ID NO: 383
  REGION 1..214: note = Light chain ADI-59193

SEQ ID NO: 485-490 - skipped (6 entries)

SEQ ID NO: 491 - protein, 447 aa, synthetic construct
  REGION 1..447: note = Heavy chain ADI-59194
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 492 - protein, 352 aa, synthetic construct
  REGION 1..352: note = Heavy chain ADI-59194
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 493 - protein, 214 aa, synthetic construct
  REGION 1..214: note = Light chain ADI-59194
DIQMTQSPSSLSASVGDRVTITCQASQDISNYLNWYQQKPGKAPKLLIYDASNLETGVPSRFSGSGSGTDFTFTISSLQPEDIATYYCQQAFNYPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 494-500 - skipped (7 entries)

SEQ ID NO: 501 - protein, 447 aa, synthetic construct
  REGION 1..447: note = Heavy chain ADI-59195
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLQCLVKGFYPSDIAVEWESNGQPENNYKTTPPVLDSDGSFFLYSRLTVDKSRWQQGNVFSCSVMHEALHNHYTQKSLSLSPG

SEQ ID NO: 502 - protein, 352 aa, synthetic construct
  REGION 1..352: note = Heavy chain ADI-59195
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVKGFYPSDIAVEWESNGQPENNYKTTPPVLDSDGSFFLYSKLTVDKSRWQQGNVFSCSVMHEALHNHYTQKSLSLSPG

SEQ ID NO: 503 - protein, 214 aa, synthetic construct; identical to SEQ ID NO: 493
  REGION 1..214: note = Light chain ADI-59195

SEQ ID NO: 504-510 - skipped (7 entries)

SEQ ID NO: 511 - protein, 447 aa, synthetic construct
  REGION 1..447: note = Heavy chain ADI-59196
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLRCLVKGFYPSDIAVEWESNGQPENNYKTTPPVLDSDGSFFLYSGLTVDKSRWQQGNVFSCSVMHEALHNHYTQKSLSLSPG

SEQ ID NO: 512 - protein, 352 aa, synthetic construct
  REGION 1..352: note = Heavy chain ADI-59196
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCFVGGFYPSDIAVEWESNGQPENNYKTTPPVLDSDGSFFLYSKLTVDKSRWQQGNVFSCSVMHEALHNHYTQKSLSLSPG

SEQ ID NO: 513 - protein, 214 aa, synthetic construct; identical to SEQ ID NO: 493
  REGION 1..214: note = Light chain ADI-59196

SEQ ID NO: 514-520 - skipped (7 entries)

SEQ ID NO: 521 - protein, 447 aa, synthetic construct
  REGION 1..447: note = Heavy chain ADI-59197
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLDTDGSFFLYSLLTVDKSRWQQGNVFSCSVMHEALHNHYTQKSLSLSPG

SEQ ID NO: 522 - protein, 352 aa, synthetic construct
  REGION 1..352: note = Heavy chain ADI-59197
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLTCLVKGFYPSDIAVEWESNGQPENNYKTTPPVLQSDGSFLLYSKLTVDKSRWQQGNVFSCSVMHEALHNHYTQKSLSLSPG

SEQ ID NO: 523 - protein, 214 aa, synthetic construct; identical to SEQ ID NO: 493
  REGION 1..214: note = Light chain - ADI-59197

SEQ ID NO: 524-530 - skipped (7 entries)

SEQ ID NO: 531 - protein, 447 aa, synthetic construct
  REGION 1..447: note = Heavy chain ADI-59198
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVDLTCLVKGFYPSDIAVEWESNGQPENNYKTTPPVLDSDGSFFLVSGLTVDKSRWQQGNVFSCSVMHEALHNHYTQKSLSLSPG

SEQ ID NO: 532 - protein, 352 aa, synthetic construct
  REGION 1..352: note = Heavy chain ADI-59198
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLVCSVYGFYPSDIAVEWESNGQPENNYKTTPPVLDSDGSFFLYSKLTVDKSRWQQGNVFSCSVMHEALHNHYTQKSLSLSPG

SEQ ID NO: 533 - protein, 214 aa, synthetic construct; identical to SEQ ID NO: 493
  REGION 1..214: note = Light chain ADI-59198

SEQ ID NO: 534-540 - skipped (7 entries)

SEQ ID NO: 541 - protein, 447 aa, synthetic construct
  REGION 1..447: note = Heavy chain ADI-59199
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARSHSSLFLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVLLWCLVKGFYPSDIAVEWESNGQPENNYKTTPPVLDSDGSFFLYSGLTVDKSRWQQGNVFSCSVMHEALHNHYTQKSLSLSPG

SEQ ID NO: 542 - protein, 352 aa, synthetic construct
  REGION 1..352: note = Heavy chain ADI-59199
QVKLEESGGGLVQAGRSLRLSCAASEHTFSSHVMGWFRQAPGKERESVAVIGWRDISTSYADSVKGRFTISRDNAKKTLYLQMNSLKPEDTAVYYCAARRIDAADFDSWGQGTQVTVSSGSEPKSSDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALAAPIEKTISKAKGQPREPQVYTLPPSRDELTKNQVSLSCIVKGFYPSDIAVEWESNGQPENNYKTTPPVLDSDGSFFLGSKLTVDKSRWQQGNVFSCSVMHEALHNHYTQKSLSLSPG

SEQ ID NO: 543 - protein, 214 aa, synthetic construct; identical to SEQ ID NO: 493
  REGION 1..214: note = Light chain ADI-59199

SEQ ID NO: 544-550 - skipped (7 entries)

SEQ ID NO: 551 - protein, 448 aa, synthetic construct
  REGION 1..448: note = Heavy chain ADI-59200
QVQLVQSGAEVKKPGASVKVSCKASGFNIKDYYMHWVRQAPGQRLEWMGWIDLEEGNTIYDAKFQGRVTITRDTSASTAYMELSSLRSEDTAVYYCARDAYGRYFYDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPG

SEQ ID NO: 552 - protein, 450 aa, synthetic construct
  REGION 1..450: note = Heavy chain ADI-59200
QVQLVQSGAEVKKPGASVKVSCKASGYAFPNYGISWVRQAPGQGLEWMGWIVAYGGNTVYAQKLQGRVTMTTDTSTSTAYMELRSLRSDDTAVYYCARDGGQPIDYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPG

SEQ ID NO: 553 - protein, 219 aa, synthetic construct; identical to SEQ ID NO: 213
  REGION 1..219: note = Light chain ADI-59200

SEQ ID NO: 554 - protein, 219 aa, synthetic construct; identical to SEQ ID NO: 213
  REGION 1..219: note = Light chain ADI-59200

SEQ ID NO: 555-560 - skipped (6 entries)

SEQ ID NO: 561 - protein, 448 aa, synthetic construct; identical to SEQ ID NO: 211
  REGION 1..448: note = Heavy chain ADI-59201

SEQ ID NO: 562 - skipped

SEQ ID NO: 563 - protein, 219 aa, synthetic construct; identical to SEQ ID NO: 213
  REGION 1..219: note = Light chain ADI-59201

SEQ ID NO: 564-570 - skipped (7 entries)

SEQ ID NO: 571 - protein, 450 aa, synthetic construct; identical to SEQ ID NO: 212
  REGION 1..450: note = Heavy chain ADI-59202

SEQ ID NO: 572 - skipped

SEQ ID NO: 573 - protein, 219 aa, synthetic construct; identical to SEQ ID NO: 213
  REGION 1..219: note = Light chain ADI-59202

SEQ ID NO: 574-580 - skipped (7 entries)

SEQ ID NO: 581 - protein, 456 aa, synthetic construct; identical to SEQ ID NO: 381
  REGION 1..456: note = Heavy chain ADI-59203

SEQ ID NO: 582 - skipped

SEQ ID NO: 583 - protein, 214 aa, synthetic construct; identical to SEQ ID NO: 383
  REGION 1..214: note = Light chain ADI-59203

SEQ ID NO: 584-590 - skipped (7 entries)

SEQ ID NO: 591 - protein, 452 aa, synthetic construct; identical to SEQ ID NO: 382
  REGION 1..452: note = Heavy chain ADI-59204

SEQ ID NO: 592 - skipped

SEQ ID NO: 593 - protein, 214 aa, synthetic construct; identical to SEQ ID NO: 383
  REGION 1..214: note = Light chain ADI-59204

SEQ ID NO: 594 - protein, 214 aa, synthetic construct; identical to SEQ ID NO: 493
  REGION 1..214: note = Light chain ADI-59205

SEQ ID NO: 595-600 - skipped (6 entries)

SEQ ID NO: 601 - protein, 447 aa, synthetic construct; identical to SEQ ID NO: 491
  REGION 1..447: note = Heavy chain ADI-59205

SEQ ID NO: 602-610 - skipped (9 entries)

SEQ ID NO: 611 - protein, 352 aa, synthetic construct; identical to SEQ ID NO: 492
  REGION 1..352: note = Heavy chain ADI-59206

SEQ ID NO: 612-700 - skipped (89 entries)

SEQ ID NO: 701 - protein, 107 aa, synthetic construct
  REGION 1..107: note = Heavy chain KiH amino acid
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 702 - protein, 107 aa, synthetic construct
  REGION 1..107: note = Heavy chain KiH amino acid
GQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 703 - protein, 107 aa, synthetic construct
  REGION 1..107: note = Heavy chain KiH amino acid
  SITE 26: note = misc_feature - Xaa represents any one of the 20 L-amino acids
GQPREPQVYTLPPSRDELTKNQVSLXCLVKGFYPSDIAVEWESNGQPENNYKTTPPVLDSDGSFFLYSKLTVDKSRWQQGNVFSCSVMHEALHNHYTQKSLSLSPGK

SEQ ID NO: 704 - protein, 107 aa, synthetic construct
  REGION 1..107: note = Heavy chain KiH amino acid
GQPREPQVYTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 705 - protein, 107 aa, synthetic construct
  REGION 1..107: note = Heavy chain KiH amino acid
  VAR_SEQ 26: note = misc_feature - Xaa represents any one of the 20 L-amino acids
  VAR_SEQ 28: note = misc_feature - Xaa represents any one of the 20 L-amino acids
  VAR_SEQ 67: note = misc_feature - Xaa represents any one of the 20 L-amino acids
GQPREPQVYTLPPSRDELTKNQVSLXCXVKGFYPSDIAVEWESNGQPENNYKTTPPVLDSDGSFFLXSKLTVDKSRWQQGNVFSCSVMHEALHNHYTQKSLSLSPGK

SEQ ID NO: 706 - protein, 107 aa, synthetic construct; identical to SEQ ID NO: 701
  REGION 1..107: note = Heavy chain KiH amino acid

SEQ ID NO: 707 - protein, 107 aa, synthetic construct; identical to SEQ ID NO: 703
  REGION 1..107: note = Heavy chain KiH amino acid
  VAR_SEQ 26: note = misc_feature - Xaa represents any one of the 20 L-amino acids

SEQ ID NO: 708 - protein, 107 aa, synthetic construct; identical to SEQ ID NO: 705
  REGION 1..107: note = Heavy chain KiH amino acid
  VAR_SEQ 26: note = misc_feature - Xaa represents any one of the 20 L-amino acids
  VAR_SEQ 28: note = misc_feature - Xaa represents any one of the 20 L-amino acids
  VAR_SEQ 67: note = misc_feature - Xaa represents any one of the 20 L-amino acids

SEQ ID NO: 709 - protein, 107 aa, synthetic construct; identical to SEQ ID NO: 701
  REGION 1..107: note = Heavy chain KiH amino acid

SEQ ID NO: 710 - protein, 107 aa, synthetic construct; identical to SEQ ID NO: 703
  REGION 1..107: note = Heavy chain KiH amino acid
  VAR_SEQ 26: note = misc_feature - Xaa represents any one of the 20 L-amino acids

SEQ ID NO: 711 - protein, 107 aa, synthetic construct; identical to SEQ ID NO: 705
  REGION 1..107: note = Heavy chain KiH amino acid
  VAR_SEQ 26: note = misc_feature - Xaa represents any one of the 20 L-amino acids
  VAR_SEQ 28: note = misc_feature - Xaa represents any one of the 20 L-amino acids
  VAR_SEQ 67: note = misc_feature - Xaa represents any one of the 20 L-amino acids

SEQ ID NO: 712 - protein, 107 aa, synthetic construct; identical to SEQ ID NO: 701
  REGION 1..107: note = Heavy chain KiH amino acid

SEQ ID NO: 713 - protein, 107 aa, synthetic construct; identical to SEQ ID NO: 702
  REGION 1..107: note = Heavy chain KiH amino acid

SEQ ID NO: 714 - protein, 107 aa, synthetic construct; identical to SEQ ID NO: 705
  REGION 1..107: note = Heavy chain KiH amino acid
  VAR_SEQ 26: note = misc_feature - Xaa represents any one of the 20 L-amino acids
  VAR_SEQ 28: note = misc_feature - Xaa represents any one of the 20 L-amino acids
  VAR_SEQ 67: note = misc_feature - Xaa represents any one of the 20 L-amino acids

SEQ ID NO: 715 - protein, 5 aa, synthetic construct
  source 1..5: note = G4S linker unit
GGGGS

SEQ ID NO: 716 - protein, 4 aa, synthetic construct
  source 1..4: note = G3S linker unit
GGGS

SEQ ID NO: 717 - protein, 6 aa, synthetic construct
  source 1..6: note = G5S linker unit
GGGGGS

SEQ ID NO: 718 - protein, 10 aa, synthetic construct
  source 1..10: note = (G4S) subscript 2 linker
GGGGSGGGGS

SEQ ID NO: 719 - protein, 15 aa, synthetic construct
  source 1..15: note = (G4S) subscript 3 linker
GGGGSGGGGSGGGGS

SEQ ID NO: 720-721 - skipped (2 entries)

SEQ ID NO: 722 - protein, 106 aa, Homo sapiens
  source 1..106: note = Human IgG2 CH3 domain reference sequence (UniProt P01859)
GQPREPQVYTLPPSREEMTKNQVSLTCLVKGFYPSDISVEWESNGQPENNYKTTPPMLDSDGSFFLYSKLTVDKSRWQQGNVFSCSVMHEALHNHYTQKSLSLSPG

SEQ ID NO: 723 - protein, 106 aa, Homo sapiens
  source 1..106: note = Human IgG3 CH3 domain reference sequence (UniProt P01860)
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPG

SEQ ID NO: 724 - protein, 106 aa, Homo sapiens
  source 1..106: note = Human IgG4 CH3 domain reference sequence (UniProt P01861)
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG